(19) United States
(12) Patent Application Publication
(10) Pub. No.: US 2023/0234942 A1
(43) Pub. Date: Jul. 27, 2023

(54) COMPOUNDS FOR TREATING HUNTINGTON'S DISEASE

(71) Applicant: PTC Therapeutics, Inc., South Plainfield, NJ (US)

(72) Inventors: Nadiya Sydorenko, Princeton, NJ (US); Rauful Alam, Middlesex, NJ (US); Suresh Babu, Pennington, NJ (US); Anuradha Bhattacharyya, Edison, NJ (US); Guangming Chen, Bridgewater, NJ (US); Matteo Chierchia, Jersey City, NJ (US); Gary Mitchell Karp, Princeton Junction, NJ (US); Tom Tuan Luong, San Carlos, CA (US); Anthony R. Mazzotti, New Providence, NJ (US); Young-Choon Moon, Warren, NJ (US); Nicholas Walter Mszar, New Brunswick, NJ (US); Jana Narasimhan, Scotch Plains, NJ (US); Jigar S. Patel, Westfield, NJ (US); Hongyu Ren, Florence, NJ (US); Gang Wang, Edison, NJ (US); Matthew G. Woll, Dunellen, NJ (US); Nanjing Zhang, Princeton, NJ (US); Xiaoyan Zhang, Belle Mead, NJ (US)

(21) Appl. No.: 17/917,450

(22) PCT Filed: Apr. 8, 2021
(86) PCT No.: PCT/US2021/026316

§ 371 Date: Oct. 6, 2022

Related U.S. Application Data
(60) Provisional application No. 63/007,650, filed on Apr. 9, 2020.
(60) Provisional application No. 63/007,660, filed on Apr. 9, 2020.

(51) Int. Cl. C07D 403/14; C07D 487/08; C07D 487/04; C07D 498/04; C07D 401/14; C07D 471/04; C07D 473/00; C07D 491/048; C07D 513/04; C07D 417/14; C07D 405/14; C07D 413/14

(52) U.S. Cl. CPC C07D 403/14; C07D 487/08; C07D 487/04; C07D 498/04; C07D 401/14; C07D 471/04; C07D 473/00; C07D 491/048; C07D 513/04; C07D 417/14; C07D 405/14; C07D 413/14; C07B 2200/05

(57) ABSTRACT

The present description relates to compounds of Formula (I), forms, and pharmaceutical compositions thereof and methods of using such compounds, forms, or compositions thereof for treating or ameliorating Huntington's disease.

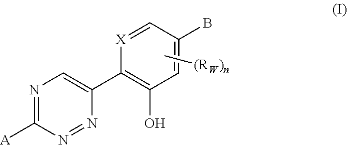

### FIELD OF THE DISCLOSURE

An aspect of the present description relates to compounds, forms, and pharmaceutical compositions thereof and methods of using such compounds, forms, or compositions thereof useful for treating or ameliorating Huntington's disease. In particular, another aspect of the present description relates to substituted monocyclic and bicyclic heteroaryl compounds, forms and pharmaceutical compositions thereof and methods of using such compounds, forms, or compositions thereof for treating or ameliorating Huntington's disease.

### BACKGROUND

Huntington's disease (HD) is a progressive, autosomal dominant neurodegenerative disorder of the brain, having symptoms characterized by involuntary movements, cognitive impairment, and mental deterioration. Death, typically caused by pneumonia or coronary artery disease, usually occurs 13 to 15 years after the onset of symptoms. The prevalence of HD is between three and seven individuals per 100,000 in populations of western European descent. In North America, an estimated 30,000 people have HD, while an additional 200,000 people are at risk of inheriting the disease from an affected parent. The disease is caused by an expansion of uninterrupted trinucleotide CAG repeats in the “mutant” huntingtin (Htt) gene, leading to production of HTT (Htt protein) with an expanded poly-glutamine (polyQ) stretch, also known as a “CAG repeat” sequence. There are no current small molecule therapies targeting the underlying cause of the disease, leaving a high unmet need for medications that can be used for treating or ameliorating HD. Consequently, there remains a need to identify and provide small molecule compounds for treating or ameliorating HD.

International Application Publication No. WO/2019/191229 A1 describes triazine compounds which may be suitable for treating or ameliorating Huntington's disease.

However, there remains a need to develop new compounds which are more potent, as measured by IC₅₀, and have improved pharmacokinetic properties (PK), as measured by such parameters as efflux transport, tissue exposure, absorption, distribution, metabolism, excretion, time to maximum plasma concentration (Tₘₐₓ), maximum concentration (Cₘₐₓ), concentration at 24 hours (C₂₄), area under the concentration-time curve (AUC), and terminal elimination half-life (t_{1/2}).

All other documents referred to herein are incorporated by reference into the present application as though fully set forth herein.

### SUMMARY

An aspect of the present description includes compounds of Formula (I):

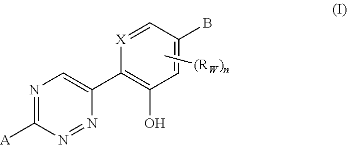

or a form thereof, wherein A, B, X, R_{W}, and n are as defined herein. B may be a monocyclic ring structure, and/or a bicyclic ring structure.

An aspect of the present description includes a method for treating or ameliorating HD in a subject in need thereof comprising, administering to the subject an effective amount of a compound of Formula (I) or a form thereof.

An aspect of the present description includes a method for use of a compound of Formula (I) or a form or composition thereof for treating or ameliorating HD in a subject in need thereof comprising, administering to the subject an effective amount of the compound of Formula (I) or a form or composition thereof.

An aspect of the present description includes a use for a compound of Formula (I) or a form thereof for treating or ameliorating HD in a subject in need thereof comprising, administering to the subject an effective amount of the compound of Formula (I) or a form thereof.

An aspect of the present description includes a use for a compound of Formula (I) or a form thereof in the manufacture of a medicament for treating or ameliorating HD in a subject in need thereof comprising, administering to the subject an effective amount of the medicament.

An aspect of the present description includes a use for a compound of Formula (I) or a form thereof in a combination product with one or more therapeutic agents for treating or ameliorating HD in a subject in need thereof comprising, administering to the subject an effective amount of the compound of Formula (I) or a form thereof in combination with an effective amount of the one or more agents.

### DETAILED DESCRIPTION

A compound of Formula (I) is disclosed:

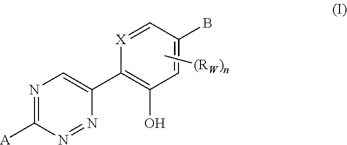

or a salt, solvate, hydrate, ester, prodrug, enantiomer, stereoisomer, rotamer, tautomer, positional isomer, or racemate thereof, wherein:

A is selected from the group consisting of:

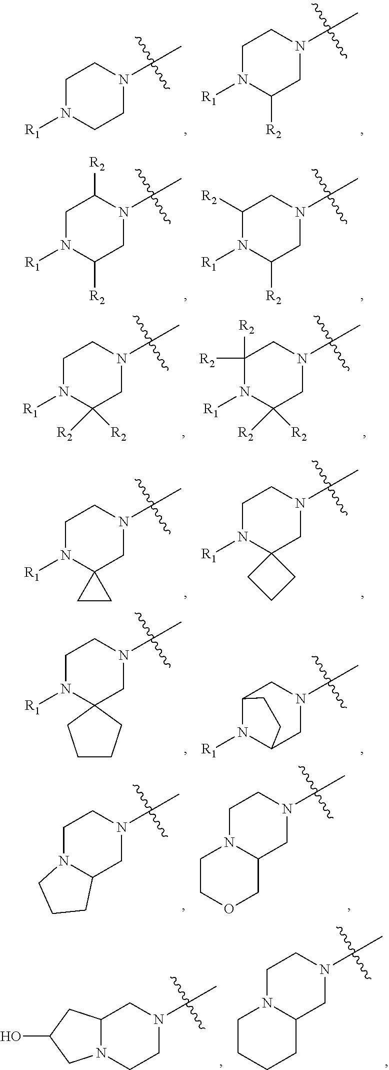

and any stereoisomer thereof;

R₁ is selected from the group consisting of hydrogen, C₁₋₄alkyl, and C₃₋₆cycloalkyl;

R₂ is independently selected from the group consisting of halogen, C₁₋₄alkyl, deutero-C₁₋₄alkyl, halo-C₁₋₄alkyl, hydroxyl-C₁₋₄alkyl, C₁₋₄alkoxy-C₁₋₄alkyl, C₂₋₄alkenyl, C₃₋₆cycloalkyl, phenyl, pyridinyl, and heterocyclyl, wherein heterocyclyl is a 3- to 6-membered carbon atom ring structure radical containing 1 or 2 heteroatom ring members selected from N, O, and S, and wherein each instance of C₁₋₄alkyl, C₃₋₆cycloalkyl, phenyl, pyridinyl, and heterocyclyl is optionally substituted with one or two R₃ substituents;

R₃ is independently selected from the group consisting of halogen, hydroxyl, C₁₋₄alkyl, C₁₋₄alkoxy, and C₃₋₆cycloalkyl;

B is selected from the group consisting of:

phenyl optionally substituted with one or two independently selected R₄ substituents;

heteroaryl, wherein heteroaryl is a 5- or 6-membered monocyclic aromatic carbon atom ring structure radical containing 1, 2, or 3 heteroatoms selected from N, O, and S, optionally substituted with one R₄ substituent, or wherein heteroaryl is a 9- or 10-membered bicyclic aromatic carbon atom ring structure radical having 1, 2, 3, or 4 heteroatom ring members independently selected from N, O, or S, optionally substituted with one or two independently selected R₄ substituents; and

heterocyclyl, wherein heterocyclyl is a 8- to 10-membered bicyclic aromatic carbon atom ring structure radical containing 1, 2, 3, or 4 heteroatom ring members independently selected from N, O, or S, optionally substituted with one or two independently selected R₄ substituents;

R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, amino, C₁₋₄alkyl-amino, (C₁₋₄alkyl)₂-amino, C₃₋₆cycloalkyl, and heterocyclyl, wherein heterocyclyl is a 3- to 6-membered monocyclic carbon atom ring structure radical containing 1 or 2 heteroatom ring members independently selected from N, O, or S;

X is selected from the group consisting of CH, CF, and N;

R_{w} is selected from the group consisting of halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, and halo-C₁₋₄alkoxy; and

n is selected from the group consisting of 0 or 1.

One aspect includes a compound of Formula (I), wherein A is selected from the group consisting of:

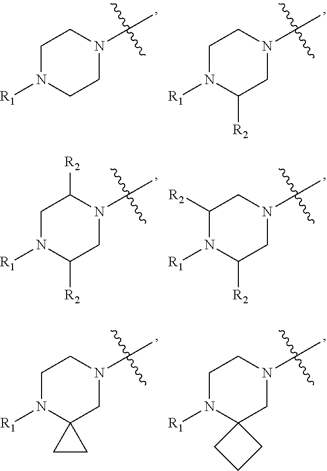

and any stereoisomer thereof.

A may be selected from the group consisting of:

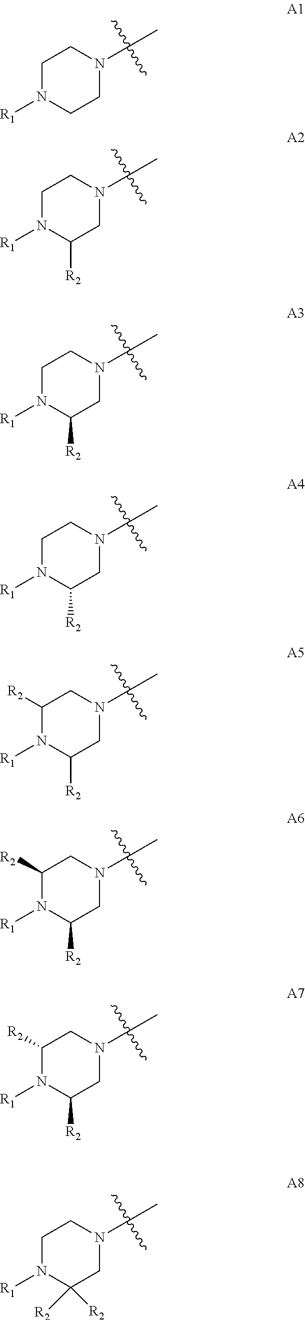

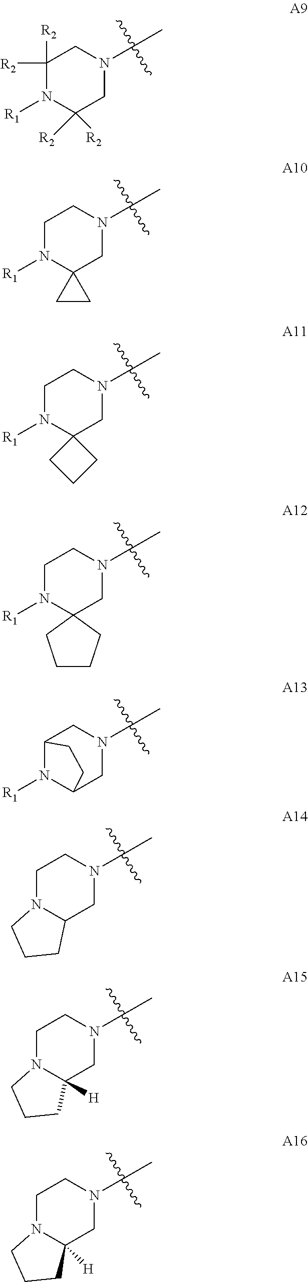

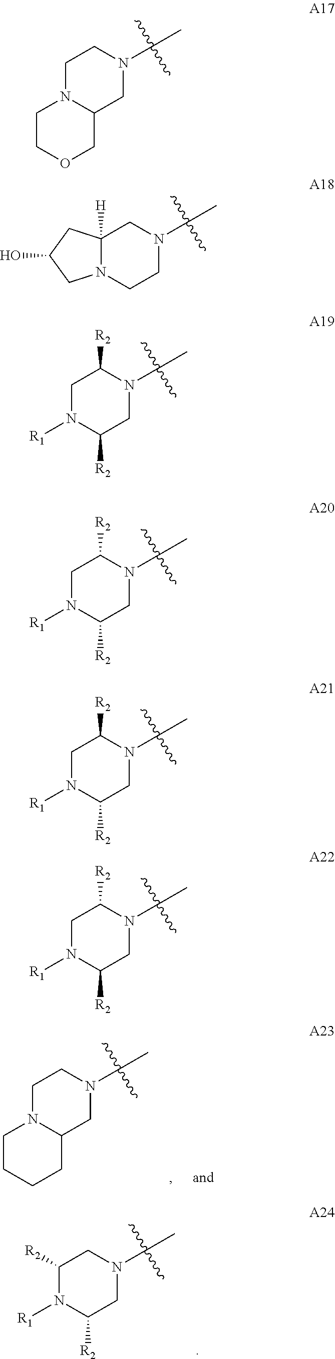

A may be selected from the group consisting of:

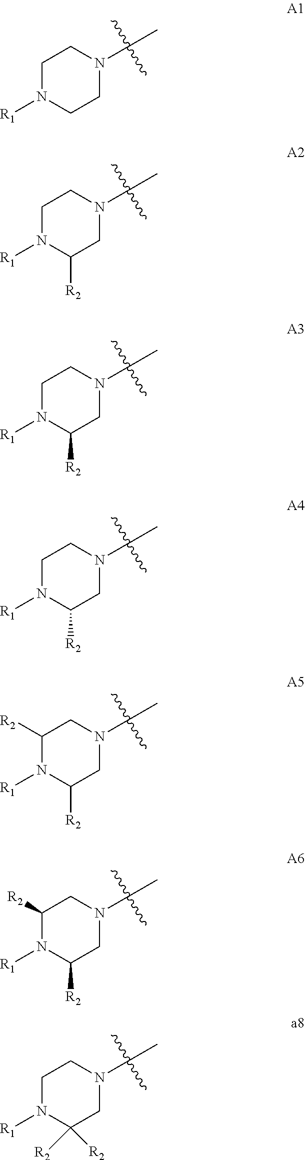

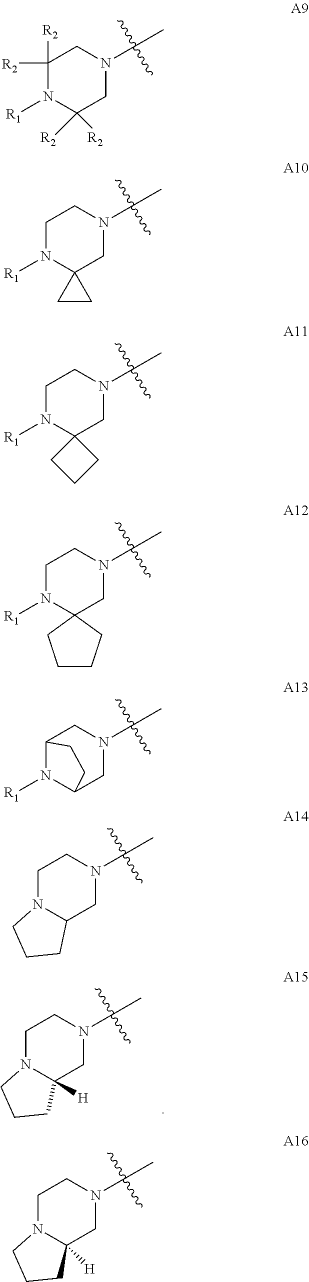

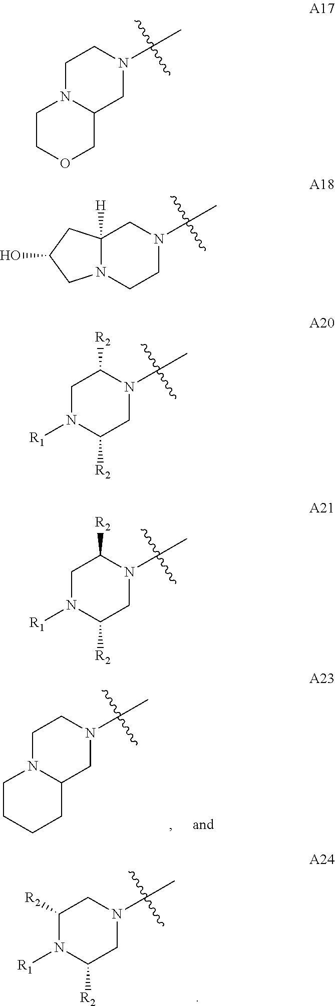

A may be selected from the group consisting of:

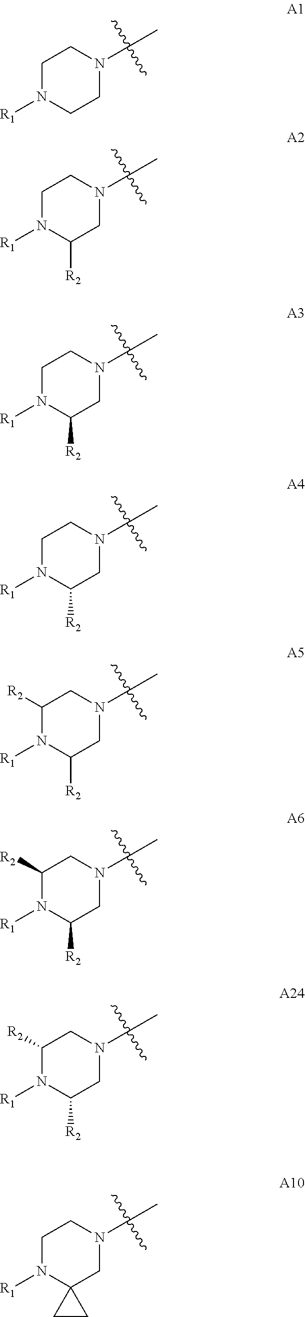

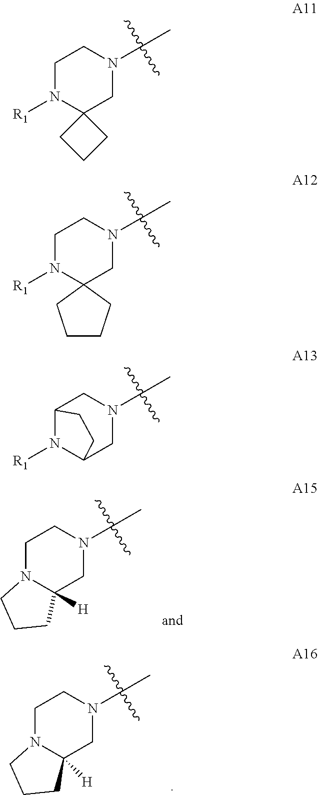

A may be

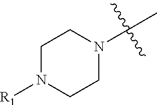

A1, or any stereoisomer thereof.

A may be

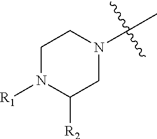

A2, or any stereoisomer thereof.

A may be

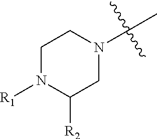

selected from the group consisting of:

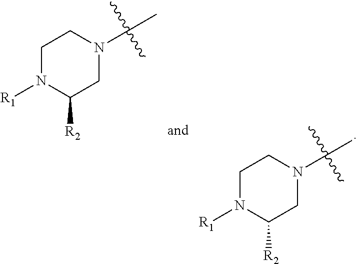

A may be

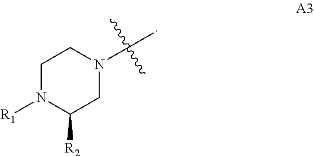

A may be

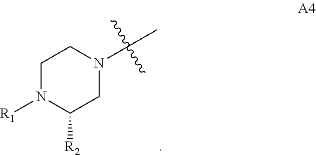

A may be

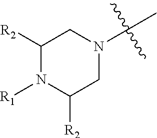

A5, or any stereoisomer thereof.

A may be

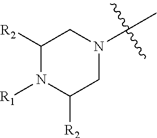

selected from the group consisting of:

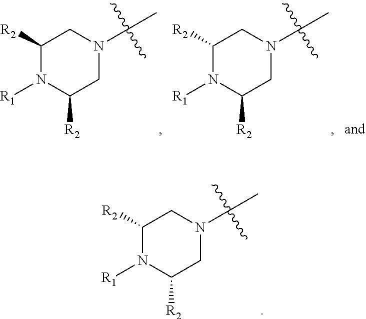

A may be

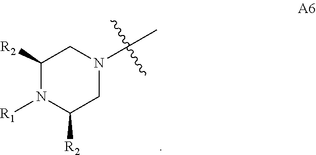

A may be

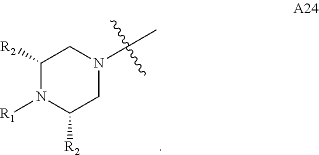

A may be

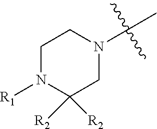

A8, or any stereoisomer thereof.

A may be

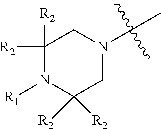

A9, or any stereoisomer thereof.

A may be

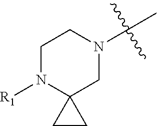

A10, or any stereoisomer thereof.

A may be

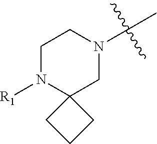

A11, or any stereoisomer thereof.

A may be

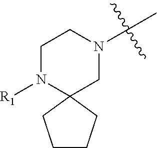

A12, or any stereoisomer thereof.

A may be

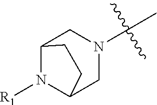

A13, or any stereoisomer thereof.

A may be

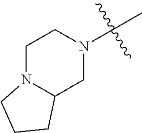 [SMILES: *N1CCN2CCCC2C1]

A14, or any stereoisomer thereof.

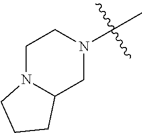 [SMILES: *N1CCN2CCCC2C1]

A may be selected from the group consisting of:

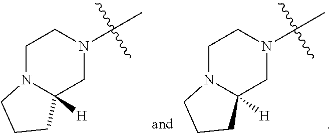

A may be

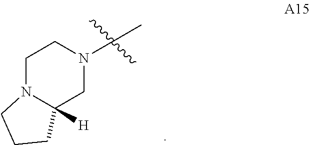

A may be

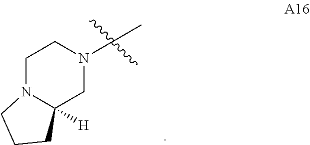 [SMILES: *N1CCN2CCC[C@@H]2C1]

A may be

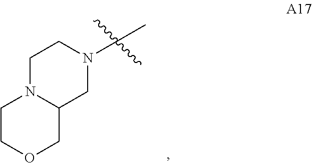 [SMILES: *N1CCN2CCOCC2C1]

or any stereoisomer thereof.

A may be

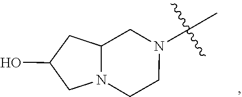 [SMILES: *N1CCN2CC(O)CC2C1]

or a stereoisomer thereof, such as, but not limited to,

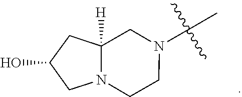 [SMILES: *N1CCN2C[C@H](O)C[C@H]2C1]

A may be

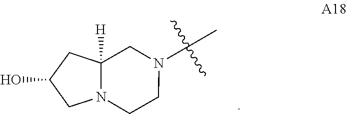 [SMILES: *N1CCN2C[C@H](O)C[C@H]2C1]

A may be

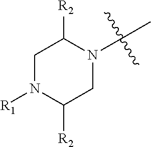

or any stereoisomer thereof.

A may be

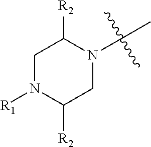

selected from the group consisting of:

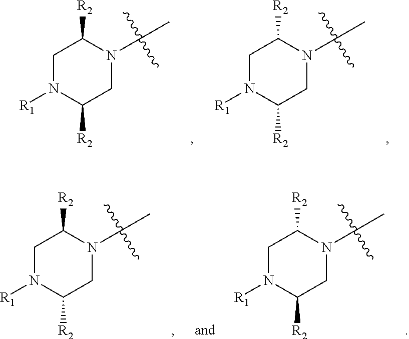

A may be

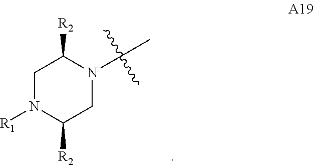

A may be

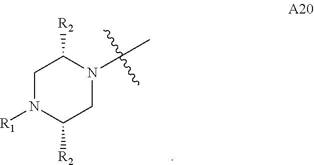

A may be

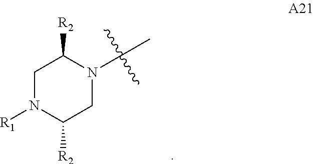

A may be

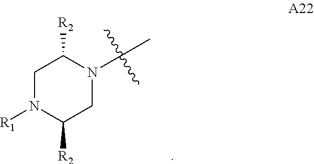

A may be

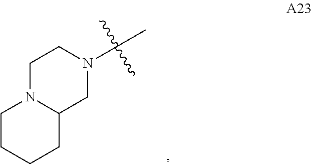 [SMILES: *N1CCN2CCCCC2C1]

or any stereoisomer thereof.

One aspect includes a compound of Formula (I), wherein R₁ is hydrogen or C₁₋₄alkyl. R₁ may be hydrogen. R₁ may be C₁₋₄alkyl selected from methyl, ethyl, propyl, isopropyl, butyl, and tert-butyl. R₁ may be methyl or ethyl. R₁ may be methyl. R₁ may be ethyl. One aspect includes a compound of Formula (I), wherein R₁ is C₃₋₆cycloalkyl selected from cyclopropyl, cyclobutyl, cyclopenyl, and cyclohexyl. R₁ may be cyclopropyl.

One aspect includes a compound of Formula (I), wherein R₂ is independently selected from the group consisting of C₁₋₄alkyl, halo-C₁₋₄alkyl, hydroxyl-C₁₋₄alkyl, C₁₋₄alkoxy-C₁₋₄alkyl, C₂₋₄alkenyl, C₃₋₆cycloalkyl, phenyl, pyridinyl, and heterocyclyl, wherein heterocyclyl is a 3- to 6-membered carbon atom ring structure radical containing 1 or 2 heteroatom ring members selected from N, O, and S, and wherein each instance of C₁₋₄alkyl, C₃₋₆cycloalkyl, phenyl, pyridinyl, and heterocyclyl is optionally substituted with one or two R₃ substituents.

Another aspect includes a compound of Formula (I), wherein R₂ is independently selected from the group consisting of C₁₋₄alkyl, hydroxyl-C₁₋₄alkyl, C₃₋₆cycloalkyl, phenyl, pyridinyl, and heterocyclyl, wherein heterocyclyl is a 3- to 4-membered carbon atom ring structure radical containing 1 heteroatom ring members selected from N, and O, wherein each R₂ is optionally substituted with one R₃ substituent.

Another aspect includes a compound of Formula (I), wherein R₂ is independently selected from the group consisting of C₁₋₄alkyl, hydroxyl-C₁₋₄alkyl, and C₃₋₆cycloalkyl, wherein each R₂ is optionally substituted with one R₃ substituent.

R₂ may be C₁₋₄alkyl selected from the group consisting of methyl, ethyl, propyl, isopropyl, butyl and tert-butyl. R₂ may be methyl. R₂ may be ethyl. R₂ may be propyl. R₂ may be isopropyl. R₂ may be butyl. R₂ may be tert-butyl.

R₂ may be hydroxyl-C₁₋₄alkyl, wherein C₁₋₄alkyl is selected from the group consisting of methyl, ethyl, propyl, isopropyl, and butyl, partially or completely substituted with one or more hydroxyl groups where allowed by available valences. R₂ may be hydroxyl-C₁₋₄alkyl, wherein C₁₋₄alkyl is selected from methyl and isopropyl substituted with one hydroxyl group. R₂ may be hydroxymethyl. R₂ may be isopropyl substituted with one hydroxyl group. R₂ may be 2-hydroxypropan-2-yl.

R₂ may be halo-C₁₋₄alkyl wherein C₁₋₄alkyl is selected from the group consisting of methyl, ethyl, propyl, isopropyl, butyl, and tert-butyl, partially or completely substituted with one or more halogen atoms where allowed by available valences. R₂ may be difluoroethyl.

R₂ may be C₁₋₄alkoxy-C₁₋₄alkyl, wherein C₁₋₄alkyl is selected from the group consisting of methyl, ethyl, propyl, and butyl, partially or completely substituted with one or more C₁₋₄alkoxy groups selected from methoxy, ethoxy, propoxy, and butoxy where allowed by available valences. R₂ may be methoxymethyl.

R₂ may be C₂₋₄alkenyl selected from ethenyl, propenyl, and butenyl. R₂ may be ethenyl.

R₂ may be C₃₋₆cycloalkyl selected from cyclopropyl, cyclobutyl, cyclopenyl, and cyclohexyl optionally substituted with one or two R₃ substituents. R₂ may be C₃₋₆cycloalkyl selected from cyclopropyl and cyclobutyl optionally substituted with one or two R₃ substituents.

R₂ may be cyclopropyl, substituted with zero to two R₃ substituents. R₂ may be unsubstituted cyclopropyl. R₂ may be cyclopropyl unsubstituted or substituted with one R₃ substituent, wherein R₃ is halogen, hydroxyl, C₁₋₄alkyl, or C₁₋₄alkoxy. R₂ may be cyclopropyl unsubstituted or substituted with one R₃ substituent, wherein R₃ is halogen, hydroxyl, methyl, ethyl, methoxy, or ethoxy.

R₂ may be cyclobutyl optionally substituted with one or two R₃ substituents. R₂ may be unsubstituted cyclobutyl.

R₂ may be phenyl optionally substituted with one or two R₃ substituents. R₂ may be unsubstituted phenyl.

R₂ may be pyridinyl optionally substituted with one or two R₃ substituents. R₂ may be unsubstituted pyridinyl. R₂ may be unsubstituted pyridine-4-yl.

R₂ may be C₁₋₄alkyl, halo-C₁₋₄alkyl, hydroxyl-C₁₋₄alkyl, cyclopropyl, cyclobutyl, phenyl, or oxetanyl, optionally substituted with one or two R₃ substituents.

R₂ may be heterocyclyl selected from the group consisting of aziridinyl, oziranyl, thiiranyl, azetidinyl, oxetanyl, thietanyl, pyrrolidinyl, pyrazolinyl, tetrahyrofuranyl, thilolanyl, piperidinyl, piperazinyl, tetrahydro-2H-pyranyl, 1,4-dioxanyl, morpholinyl, and thianyl, wherein each instance of heterocyclyl is optionally substituted with one or two R₃ substituents.

R₂ may be oxetanyl optionally substituted with one or two R₃ substituents.

R₂ may be C₁₋₄alkyl, halo-C₁₋₄alkyl, hydroxyl-C₁₋₄alkyl, cyclopropyl, cyclobutyl, phenyl, or oxetanyl, optionally substituted with one or two R₃ substituents. R₂ may be unsubstituted C₁₋₄alkyl, unsubstituted halo-C₁₋₄alkyl, unsubstituted hydroxyl-C₁₋₄alkyl, unsubstituted cyclopropyl, unsubstituted cyclobutyl, unsubstituted phenyl, or unsubstituted oxetanyl.

One aspect includes a compound of Formula (I), wherein R₃ is independently selected from the group consisting of halogen, hydroxyl, C₁₋₄alkyl, C₁₋₄alkoxy, and C₃₋₆cycloalkyl. R₃ may be independently selected from the group consisting of halogen, hydroxyl, C₁₋₄alkyl, and C₁₋₄alkoxy.

R₃ may be halogen selected from the group consisting of bromo, chloro, fluoro, and iodo. R₃ may be fluoro.

R₃ may be hydroxyl.

R₃ may be C₁₋₄alkyl selected from the group consisting of methyl, ethyl, propyl, isopropyl, butyl, and tert-butyl. R₃ may be methyl or ethyl. R₃ may be methyl.

R₃ may be C₁₋₄alkoxy selected from the group consisting of methoxy, ethoxy, propoxy, isopropoxy, butoxy and tert-butoxy. R₃ may be methoxy. R₃ may be halogen, hydroxyl, C₁₋₄alkyl, or methoxy.

R₃ may be C₃₋₆cycloalkyl selected from the group consisting of cyclopropyl, cyclobutyl, cyclopenyl, and cyclohexyl. R₃ may be cyclopropyl.

One aspect includes a compound of Formula (I), wherein B is selected from the group consisting of:

phenyl optionally substituted with one or two independently selected R₄ substituents;

heteroaryl, wherein heteroaryl is a 5- or 6-membered monocyclic aromatic carbon atom ring structure radical containing 1, 2, or 3 heteroatoms selected from N, O, and S, optionally substituted with one R₄ substituent, or wherein heteroaryl is a 9- or 10-membered aromatic carbon atom ring structure radical having 1, 2, 3, or 4 heteroatom ring members independently selected from N, O, or S, optionally substituted with one or two independently selected R₄ substituents; and

heterocyclyl, wherein heterocyclyl is a 8- to 10-membered bicyclic carbon atom ring structure radical containing 1, 2, 3, or 4 heteroatom ring members independently selected from N, O, or S, optionally substituted with one or two independently selected R₄ substituents.

B may be phenyl optionally substituted with one or two independently selected R₄ substituents. B may be unsubstituted phenyl or phenyl substituted with one R₄ substituent.

One aspect includes a compound of Formula (I), wherein B is heteroaryl, wherein heteroaryl is a 5- or 6-membered monocyclic aromatic carbon atom ring structure radical containing 1, 2, or 3 heteroatoms selected from N, O, and S, optionally substituted with one R₄ substituent. B may be heteroaryl, wherein heteroaryl is a 5- or 6-membered monocyclic aromatic carbon atom ring structure radical containing 1, 2, or 3 N atoms, and optionally a second heteroatom selected from O, and S, optionally substituted with one R₄ substituent. B may be heteroaryl, wherein heteroaryl is a 5- or 6-membered monocyclic aromatic carbon atom ring structure radical containing 2 or 3 heteroatoms selected from N, O, and S, optionally substituted with one R₄ substituent. B may be heteroaryl, wherein heteroaryl is a 5- or 6-membered monocyclic aromatic carbon atom ring structure radical containing 2 or 3 N atoms, and optionally a second heteroatom selected from O, and S, optionally substituted with one R₄ substituent.

B may be heteroaryl selected from the group consisting of furanyl, thiophenyl, 1H-pyrazolyl, 1H-imidazolyl, isoxazolyl, 1,3-thiazolyl, 1,3-oxazolyl, tetrazolyl, 1H-1,2,3-triazolyl, 2H-1,2,3-triazolyl, 1,2,4-oxadiazolyl, 1,3,4-oxadiazolyl, 1,2,3-thiadiazolyl, pyridinyl, pyrimidinyl, pyrazinyl, pyridazinyl, 1H-1,2,4-triazolyl, 1,2,4-thiadiazolyl, 1,3,4-oxadiazolyl, isothiazolyl, 1,2,5-thiadiazolyl, and 1,3,4-thiadiazolyl, optionally substituted with one R₄ substituent.

B may be heteroaryl selected from the group consisting of 1H-pyrazolyl, 1H-imidazolyl, 1,3-thiazolyl, 1,3-oxazolyl, 1H-1,2,3-triazolyl, 2H-1,2,3-triazolyl, pyridinyl, pyrimidinyl, pyrazinyl, 1H-1,2,4-triazolyl, 1,2,4-thiadiazolyl, 1,3,4-oxadiazolyl, isothiazolyl, 1,2,5-thiadiazolyl, and 1,3,4-thiadiazolyl, optionally substituted with one R₄ substituent.

B may be heteroaryl selected from the group consisting of 1H-pyrazolyl, 1H-imidazolyl, 1,3-thiazolyl, 1,3-oxazolyl, 1H-1,2,3-triazolyl, 2H-1,2,3-triazolyl, pyridinyl, pyrimidinyl, pyrazinyl, 1H-1,2,4-triazolyl, 1,2,4-thiadiazolyl, 1,3,4-oxadiazolyl, isothiazolyl, 1,2,5-thiadiazolyl, and 1,3,4-thiadiazolyl, substituted with one R₄ substituent.

B may be heteroaryl selected from the group consisting of furan-2-yl, furan-3-yl, thiophen-2-yl, thiophen-3-yl, 1H-pyrazol-1-yl, 1H-pyrazol-3-yl, 1H-pyrazol-4-yl, 1H-pyrazol-5-yl, 1H-imidazol-1-yl, 1H-imidazol-4-yl, isoxazol-3-yl, isoxazol-4-yl, isoxazol-5-yl, 1,3-thiazol-2-yl, 1,3-thiazol-4-yl, 1,3-thiazol-5-yl, 1,3-oxazol-2-yl, 1,3-oxazol-4-yl, 1,3-oxazol-5-yl, tetrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 2H-1,2,3-triazol-5-yl, 1,2,4-oxadiazol-3-yl, 1,3,4-oxadiazol-2-yl, 1,2,3-thiadiazol-4-yl, 1,2,3-thiadiazol-5-yl, pyridin-2-yl, pyridin-3-yl, pyridin-4-yl, pyrimidin-4-yl, pyrimidin-2-yl, pyrazin-2-yl, pyridazin-3-yl, pyridazin-4-yl, 1H-1,2,4-triazol-3-yl, 1H-1,2,4-triazol-5-yl, 1,2,4-thiadiazol-3-yl, 1,2,4-thiadiazol-5-yl, 1,3,4-oxadiazol-2-yl, isothiazol-3-yl, isothiazol-4-yl, isothiazol-5-yl, 1,2,5-thiadiazol-3-yl, 1,3,4-thiadiazol-2-yl, optionally substituted with one R₄ substituent.

B may be heteroaryl selected from the group consisting of 1H-pyrazol-4-yl, 1H-imidazol-1-yl, 1,3-thiazol-2-yl, 1,3-thiazol-4-yl, 1,3-oxazol-2-yl, 1H-1,2,3-triazol-4-yl, 1H-1,2,3-triazol-5-yl, 2H-1,2,3-triazol-2-yl, 2H-1,2,3-triazol-4-yl, pyridin-3-yl, pyridin-4-yl, pyrimidin-4-yl, pyrimidin-2-yl, pyrazin-2-yl, 1H-1,2,4-triazol-3-yl, 1,2,4-thiadiazol-3-yl, 1,2,4-thiadiazol-5-yl, 1,3,4-oxadiazol-2-yl, isothiazol-3-yl, isothiazol-4-yl, isothiazol-5-yl, 1,2,5-thiadiazol-3-yl, 1,3,4-thiadiazol-2-yl, optionally substituted with one R₄ substituent.

B may be heteroaryl selected from the group consisting of 1H-pyrazol-4-yl, 1H-imidazol-1-yl, 1,3-thiazol-2-yl, 1,3-thiazol-4-yl, 1,3-oxazol-2-yl, 1H-1,2,3-triazol-4-yl, 1H-1,2,3-triazol-5-yl, 2H-1,2,3-triazol-2-yl, 2H-1,2,3-triazol-4-yl, pyridin-3-yl, pyridin-4-yl, pyrimidin-4-yl, pyrimidin-2-yl, pyrazin-2-yl, 1H-1,2,4-triazol-3-yl, 1,2,4-thiadiazol-3-yl, 1,2,4-thiadiazol-5-yl, 1,3,4-oxadiazol-2-yl, isothiazol-3-yl, isothiazol-4-yl, isothiazol-5-yl, 1,2,5-thiadiazol-3-yl, 1,3,4-thiadiazol-2-yl, substituted with one R₄ substituent.

One aspect includes a compound of Formula (I), wherein B is heteroaryl, wherein heteroaryl is a 9- or 10-membered aromatic carbon atom ring structure radical having 1, 2, 3, or 4 heteroatom ring members independently selected from N, O, or S, optionally substituted with one or two independently selected R₄ substituents. B may be heteroaryl, wherein heteroaryl is a 9- or 10-membered bicyclic carbon atom ring structure radical containing at least one N atom ring member, optionally containing a second heteroatom ring member selected from O or S, and optionally substituted with one or two independently selected R₄ substituents. B may be heteroaryl, wherein heteroaryl is a 9- or 10-membered bicyclic carbon atom ring structure radical containing 2, 3 or 4 N atoms, optionally containing a second heteroatom ring member selected from O or S, and optionally substituted with one or two independently selected R₄ substituents. B may be heteroaryl, wherein heteroaryl is a 9-membered bicyclic carbon atom ring structure radical containing 2, 3 or 4 N, optionally substituted with one or two independently selected R₄ substituents. B may be heteroaryl, wherein heteroaryl is a 9-membered bicyclic carbon atom ring structure radical containing 2, 3 or 4 N, optionally containing a second heteroatom ring member selected from O or S, and optionally substituted with one or two independently selected R₄ substituents. B may be heteroaryl, wherein heteroaryl is a unsubstituted 9-membered bicyclic carbon atom ring structure radical containing 2, 3 or 4 N, optionally containing a second heteroatom ring member selected from O or S. B may be heteroaryl, wherein heteroaryl is a 9-membered bicyclic carbon atom ring structure radical containing 2, 3 or 4 N, optionally containing a second heteroatom ring member selected from O or S, and is unsubstituted or substituted with one R₄ substituent.

B may be heteroaryl selected from the group consisting of 1H-indazolyl, 2H-indazolyl, indolizinyl, benzofuranyl, benzothiophenyl, 1H-benzimidazolyl, 1,3-benzoxazolyl, 1,3-benzothiazolyl, 1,3-benzodioxolyl, 1,2,3-benzotriazolyl, 7H-purinyl, furo[3,2-b]pyridinyl, furo[3,2-c]pyridinyl, 1,3-oxazolo[5,4-b]pyridinyl, thieno[3,2-c]pyridinyl, thieno[2,3-d]pyrimidinyl, pyrrolo[1,2-a]pyrimidinyl, pyrrolo[1,2-a]pyrazinyl, pyrrolo[1,2-b]pyridazinyl, pyrazolo[1,5-a]pyridinyl, 1H-pyrrolo[2,3-b]pyridinyl, 5H-pyrrolo[2,3-b]pyrazinyl, 1H-pyrrolo[2,3-c]pyridinyl, 1H-pyrazolo[3,4-b]pyridinyl, 2H-pyrazolo[3,4-b]pyridinyl, 1H-pyrazolo[3,4-b]pyrazinyl, 1H-pyrazolo[3,4-c]pyridinyl, 1H-pyrazolo[4,3-b]pyridinyl, 2H-pyrazolo[4,3-b]pyridinyl, 2H-pyrazolo[4,3-c]pyridinyl, 1H-pyrazolo[4,3-d]pyrimidinyl, pyrazolo[1,5-a]pyrazinyl, imidazo[1,2-a]pyridinyl, imidazo[1,2-a]pyrimidinyl, imidazo[1,2-a]pyrazinyl, imidazo[1,2-b]pyridazinyl, imidazo[1,2-c]pyrimidin-2-yl, 1H-imidazo[4,5-b]pyridinyl, 3H-imidazo[4,5-b]pyridinyl, imidazo[2,1-b][1,3]thiazolyl, imidazo[2,1-b][1,3,4]thiadiazolyl, [1,3]oxazolo[4,5-b]pyridinyl, [1,2,3]triazolo[1,5-a]pyridinyl, 1H-[1,2,3]triazolo[4,5-b]pyridinyl, 3H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, tetrazolo[1,5-a]pyridinyl, tetrazolo[1,5-b]pyridazinyl, thiazolo[4,5-b]pyrazinyl, thiazolo[5,4-c]pyridinyl, quinolinyl, isoquinolinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, optionally substituted with one or two independently selected R₄ substituents.

B may be heteroaryl selected from the group consisting of 2H-indazolyl, 7H-purinyl, furo[3,2-b]pyridinyl, pyrazolo[1,5-a]pyridinyl, 2H-pyrazolo[3,4-b]pyridinyl, 1H-pyrazolo[4,3-b]pyridinyl, imidazo[1,2-a]pyridinyl, imidazo[1,2-a]pyrazinyl, imidazo[1,2-b]pyridazinyl, [1,2,3]triazolo[1,5-a]pyridinyl, 1H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, thiazolo[4,5-b]pyrazinyl, thiazolo[5,4-c]pyridinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, optionally substituted with one or two independently selected R₄ substituents.

B may be heteroaryl selected from the group consisting of 2H-indazolyl, 7H-purinyl, furo[3,2-b]pyridinyl, pyrazolo[1,5-a]pyridinyl, 2H-pyrazolo[3,4-b]pyridinyl, 1H-pyrazolo[4,3-b]pyridinyl, imidazo[1,2-a]pyridinyl, imidazo[1,2-a]pyrazinyl, imidazo[1,2-b]pyridazinyl, [1,2,3]triazolo[1,5-a]pyridinyl, 1H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, thiazolo[4,5-b]pyrazinyl, thiazolo[5,4-c]pyridinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, substituted with one R₄ substituent.

B may be heteroaryl selected from the group consisting of 2H-indazolyl, 7H-purinyl, furo[3,2-b]pyridinyl, pyrazolo[1,5-a]pyridinyl, 2H-pyrazolo[3,4-b]pyridinyl, 1H-pyrazolo[4,3-b]pyridinyl, imidazo[1,2-a]pyridinyl, imidazo[1,2-a]pyrazinyl, imidazo[1,2-b]pyridazinyl, [1,2,3]triazolo[1,5-a]pyridinyl, 1H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, thiazolo[4,5-b]pyrazinyl, thiazolo[5,4-c]pyridinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, substituted with two independently selected R₄ substituents.

B may be heteroaryl selected from the group consisting of 1H-indazol-5-yl, 2H-indazol-5-yl, indolizin-2-yl, benzofuran-2-yl, benzofuran-5-yl, benzothiophen-2-yl, benzothiophen-3-yl, 1H-benzimidazol-2-yl, 1H-benzimidazol-5-yl, 1H-benzimidazol-6-yl, 1,3-benzoxazol-2-yl, 1,3-benzoxazol-5-yl, 1,3-benzoxazol-6-yl, 1,3-benzothiazol-2-yl, 1,3-benzothiazol-5-yl, 1,3-benzothiazol-6-yl, 1,3-benzodioxol-5-yl, 1,2,3-benzotriazol-5-yl, 7H-purin-2-yl, furo[3,2-b]pyridin-2-yl, furo[3,2-c]pyridin-2-yl, furo[2,3-c]pyridin-2-yl, 1,3-oxazolo[5,4-b]pyridine-5-yl, thieno[3,2-c]pyridin-2-yl, thieno[2,3-d]pyrimidin-6-yl, pyrrolo[1,2-a]pyrimidin-7-yl, pyrrolo[1,2-a]pyrazin-7-yl, pyrrolo[1,2-b]pyridazin-2-yl, pyrazolo[1,5-a]pyridin-2-yl, pyrazolo[1,5-a]pyridin-3-yl, pyrazolo[1,5-a]pyridin-5-yl, 1H-pyrrolo[2,3-b]pyridin-5-yl, 5H-pyrrolo[2,3-b]pyrazin-2-yl, 1H-pyrrolo[2,3-c]pyridin-4-yl, 1H-pyrazolo[3,4-b]pyridin-5-yl, 1H-pyrazolo[3,4-b]pyridin-6-yl, 2H-pyrazolo[3,4-b]pyridin-5-yl, 1H-pyrazolo[3,4-b]pyrazin-5-yl, 1H-pyrazolo[3,4-c]pyridin-1-yl, 1H-pyrazolo[3,4-c]pyridin-5-yl, 1H-pyrazolo[4,3-b]pyridin-1-yl, 1H-pyrazolo[4,3-b]pyridin-5-yl, 1H-pyrazolo[4,3-b]pyridin-6-yl, 2H-pyrazolo[4,3-b]pyridin-5-yl, 2H-pyrazolo[4,3-c]pyridin-5-yl, 1H-pyrazolo[4,3-d]pyrimidin-5-yl, pyrazolo[1,5-a]pyrazin-2-yl, imidazo[1,2-a]pyridin-2-yl, imidazo[1,2-a]pyridin-6-yl, imidazo[1,2-a]pyrimidin-2-yl, imidazo[1,2-a]pyrimidin-6-yl, imidazo[1,2-a]pyrazin-2-yl, imidazo[1,2-a]pyrazin-6-yl, imidazo[1,2-b]pyridazin-2-yl, imidazo[1,2-b]pyridazin-6-yl, imidazo[1,2-c]pyrimidin-2-yl, 1H-imidazo[4,5-b]pyridin-5-yl, 3H-imidazo[4,5-b]pyridin-5-yl, imidazo[2,1-b][1,3]thiazol-6-yl, imidazo[2,1-b][1,3,4]thiadiazol-6-yl, [1,3]oxazolo[4,5-b]pyridin-2-yl, [1,2,3]triazolo[1,5-a]pyridin-5-yl, [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, 3H-[1,2,3]triazolo[4,5-b]pyridin-5-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, tetrazolo[1,5-a]pyridin-7-yl, tetrazolo[1,5-b]pyridazin-7-yl, thiazolo[4,5-b]pyrazin-2-yl, thiazolo[5,4-c]pyridin-2-yl, tetrazolo[1,5-a]pyridin-7-yl, tetrazolo[1,5-b]pyridazin-7-yl, quinolin-6-yl, isoquinolin-6-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, optionally substituted with one or two independently selected R₄ substituents.

B may be heteroaryl selected from the group consisting of 1H-indazol-5-yl, 7H-purin-2-yl, furo[3,2-b]pyridin-2-yl, pyrazolo[1,5-a]pyridin-3-yl, 2H-pyrazolo[3,4-b]pyridin-5-yl, 1H-pyrazolo[4,3-b]pyridin-1-yl, imidazo[1,2-a]pyridin-6-yl, imidazo[1,2-a]pyrazin-6-yl, imidazo[1,2-b]pyridazin-6-yl [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, thiazolo[4,5-b]pyrazin-2-yl, thiazolo[5,4-c]pyridin-2-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, optionally substituted with one or two independently selected R₄ substituents.

B may be heteroaryl selected from the group consisting of 1H-indazol-5-yl, 7H-purin-2-yl, furo[3,2-b]pyridin-2-yl, pyrazolo[1,5-a]pyridin-3-yl, 2H-pyrazolo[3,4-b]pyridin-5-yl, 1H-pyrazolo[4,3-b]pyridin-1-yl, imidazo[1,2-a]pyridin-6-yl, imidazo[1,2-a]pyrazin-6-yl, imidazo[1,2-b]pyridazin-6-yl [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, thiazolo[4,5-b]pyrazin-2-yl, thiazolo[5,4-c]pyridin-2-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, substituted with one R₄ substituent.

B may be heteroaryl selected from the group consisting of 1H-indazol-5-yl, 7H-purin-2-yl, furo[3,2-b]pyridin-2-yl, pyrazolo[1,5-a]pyridin-3-yl, 2H-pyrazolo[3,4-b]pyridin-5-yl, 1H-pyrazolo[4,3-b]pyridin-1-yl, imidazo[1,2-a]pyridin-6-yl, imidazo[1,2-a]pyrazin-6-yl, imidazo[1,2-b]pyridazin-6-yl [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, thiazolo[4,5-b]pyrazin-2-yl, thiazolo[5,4-c]pyridin-2-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, substituted with two independently selected R₄ substituents.

One aspect includes a compound of Formula (I), wherein B is heterocyclyl, wherein heterocyclyl is a 8- to 10-membered bicyclic carbon atom ring structure radical containing 1, 2, 3, or 4 heteroatom ring members independently selected from N, O, or S, optionally substituted with one or two independently selected R₄ substituents. B may be heterocyclyl, wherein heterocyclyl is a 8- or 9-membered bicyclic carbon atom ring structure radical containing at least one N, optionally substituted with one or two independently selected R₄ substituents. B may be heterocyclyl, wherein heterocyclyl is a 8- or 9-membered bicyclic carbon atom ring structure radical containing 2, 3 or 4 N, optionally substituted with one or two independently selected R₄ substituents. B may be heterocyclyl, wherein heterocyclyl is a 9-membered bicyclic carbon atom ring structure radical containing 2, 3 or 4 N, optionally substituted with one or two independently selected R₄ substituents, or heterocyclyl is a 8 membered bicyclic carbon atom ring structure radical containing 2, or 3 N, optionally substituted with one or two independently selected R₄ substituents. B may be heterocyclyl, wherein heterocyclyl is a 8- or 9-membered bicyclic carbon atom ring structure radical containing 2, 3 or 4 N, and optionally containing a second heteroatom ring member selected from O or S, optionally substituted with one or two independently selected R₄ substituents. B may be heterocyclyl, wherein heterocyclyl is a 9-membered bicyclic carbon atom ring structure radical containing 2, 3 or 4 N, and optionally containing a second heteroatom ring member selected from O or S, optionally substituted with one or two independently selected R₄ substituents. B may be heterocyclyl, wherein heterocyclyl is a unsubstituted 8- or 9-membered bicyclic carbon atom ring structure radical containing 2, 3 or 4 N, optionally containing a second heteroatom ring member selected from O or S. B may be heterocyclyl, wherein heterocyclyl is a 8- or 9-membered bicyclic carbon atom ring structure radical containing 2, 3 or 4 N, optionally containing a second heteroatom ring member selected from O or S, and is unsubstituted or substituted with one R₄ substituent.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazolyl, 5,6-dihydro-4H-pyrrolo[1,2-b]pyrazolyl, 4,5,6,7-tetrahydropyrazolo[1,5-a]pyridinyl, 5,6-dihydro-4H-pyrrolo[1,2-b]pyrazolyl, 6,7-dihydro-5H-pyrrolo[1,2-a]imidazolyl, 5,6-dihydro-4H-pyrrolo[1,2-b]pyrazolyl, and 5,6-dihydro-4H-pyrrolo[1,2-b]pyrazolyl, optionally substituted with one or two independently selected R₄ substituents.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazolyl, 5,6-dihydro-4H-pyrrolo[1,2-b]pyrazolyl, 4,5,6,7-tetrahydropyrazolo[1,5-a]pyridinyl, 5,6-dihydro-4H-pyrrolo[1,2-b]pyrazolyl, 6,7-dihydro-5H-pyrrolo[1,2-a]imidazolyl, 5,6-dihydro-4H-pyrrolo[1,2-b]pyrazolyl, and 5,6-dihydro-4H-pyrrolo[1,2-b]pyrazolyl, substituted with two independently selected R₄ substituents.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazolyl, 5,6-dihydro-4H-pyrrolo[1,2-b]pyrazolyl, 4,5,6,7-tetrahydropyrazolo[1,5-a]pyridinyl, 5,6-dihydro-4H-pyrrolo[1,2-b]pyrazolyl, 6,7-dihydro-5H-pyrrolo[1,2-a]imidazolyl, 5,6-dihydro-4H-pyrrolo[1,2-b]pyrazolyl, and 5,6-dihydro-4H-pyrrolo[1,2-b]pyrazolyl, substituted with one R₄ substituent.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazol-7-yl, 5,6-dihydro-4H-pyrrolo[1,2-b]pyrazol-3-yl, 4,5,6,7-tetrahydropyrazolo[1,5-a]pyridin-3-yl, 5,6-dihydro-4H-pyrrolo[1,2-b]pyrazol-3-yl, 6,7-dihydro-5H-pyrrolo[1,2-a]imidazol-3-yl, 5,6-dihydro-4H-pyrrolo[1,2-b]pyrazol-3-yl, and 5,6-dihydro-4H-pyrrolo[1,2-b]pyrazol-3-yl, optionally substituted with one or two independently selected R₄ substituents.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazol-7-yl, 5,6-dihydro-4H-pyrrolo[1,2-b]pyrazol-3-yl, 4,5,6,7-tetrahydropyrazolo[1,5-a]pyridin-3-yl, 5,6-dihydro-4H-pyrrolo[1,2-b]pyrazol-3-yl, 6,7-dihydro-5H-pyrrolo[1,2-a]imidazol-3-yl, 5,6-dihydro-4H-pyrrolo[1,2-b]pyrazol-3-yl, and 5,6-dihydro-4H-pyrrolo[1,2-b]pyrazol-3-yl, substituted with one R₄ substituent.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazol-7-yl, 5,6-dihydro-4H-pyrrolo[1,2-b]pyrazol-3-yl, 4,5,6,7-tetrahydropyrazolo[1,5-a]pyridin-3-yl, 5,6-dihydro-4H-pyrrolo[1,2-b]pyrazol-3-yl, 6,7-dihydro-5H-pyrrolo[1,2-a]imidazol-3-yl, 5,6-dihydro-4H-pyrrolo[1,2-b]pyrazol-3-yl, and 5,6-dihydro-4H-pyrrolo[1,2-b]pyrazol-3-yl, substituted with two independently selected R₄ substituents.

B may be selected from the group consisting of:

phenyl optionally substituted with one or two independently selected R₄ substituents;

heteroaryl,

wherein heteroaryl is a 5- or 6-membered monocyclic aromatic carbon atom ring structure radical containing at least one N atom (or at least 2 N atoms), and optionally a second heteroatom selected from O, and S, optionally substituted with one R₄ substituent, or wherein heteroaryl is a 9-membered bicyclic carbon atom ring structure radical containing at least 2 N atoms, optionally containing a second heteroatom ring member selected from O or S, and optionally substituted with one or two independently selected R₄ substituents; and

heterocyclyl,

wherein heterocyclyl is a 8- or 9-membered bicyclic carbon atom ring structure radical containing at least one N atom (or at least 2 N atoms), optionally containing a second heteroatom ring member selected from O or S, and optionally substituted with one or two independently selected R₄ substituents.

B may be selected from the group consisting of:

phenyl unsubstituted or substituted with one R₄ substituent;

heteroaryl,

wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or

wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents; and

heterocyclyl,

wherein heterocyclyl is a 8- or 9-membered bicyclic carbon atom ring structure radical containing 2, 3 or 4 N, and optionally, when the ring structure contains 2 or 3 N, containing a second heteroatom ring member selected from O or S, optionally substituted with one or two independently selected R₄ substituents. B may be heteroaryl or heterocycl,

wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or

wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents; and

wherein heterocyclyl is a 8- or 9-membered bicyclic carbon atom ring structure radical containing 2, 3 or 4 N, and optionally, when the ring structure contains 2 or 3 N, containing a second heteroatom ring member selected from O or S, optionally substituted with one or two independently selected R₄ substituents.

B may be heteroaryl,

wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or

wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents.

One aspect includes a compound of Formula (I), wherein R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, amino, C₁₋₄alkyl-amino, (C₁₋₄alkyl)₂-amino, C₃₋₆cycloalkyl, and heterocyclyl, wherein heterocyclyl is a 3- to 6-membered monocyclic carbon atom ring structure radical containing 1 or 2 heteroatom ring members independently selected from N, O, or S.

R₄ may be selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, C₁₋₄alkyl-amino, C₃₋₆cycloalkyl, and heterocyclyl.

R₄ may be halogen selected from the group consisting of bromo, chloro, fluoro, and iodo. R₄ may be halogen selected from the group consisting of chloro and fluoro. R₄ may be chloro. R₄ may be fluoro.

R₄ may be cyano.

R₄ may be C₁₋₄alkyl selected from the group consisting of methyl, ethyl, propyl, isopropyl, butyl, and tert-butyl. R₄ may be methyl or ethyl. R₄ may be methyl. R₄ may be ethyl.

R₄ may be deutero-C₁₋₄alkyl wherein C₁₋₄alkyl is selected from the group consisting of methyl, ethyl, propyl, isopropyl, butyl, and tert-butyl partially or completely substituted with one or more deuterium atoms where allowed by available valences. R₄ may be (²H₃)methyl.

R₄ may be halo-C₁₋₄alkyl wherein C₁₋₄alkyl is selected from the group consisting of methyl, ethyl, propyl, isopropyl, butyl, and tert-butyl, partially or completely substituted with one or more halogen atoms where allowed by available valences. R₄ may be halo-C₁₋₄alkyl selected from the group consisting of difluoromethyl and trifluoromethyl. R₄ may be difluoromethyl. R₄ may be trifluoromethyl.

R₄ may be C₁₋₄alkoxy selected from the group consisting of methoxy, ethoxy, propoxy, isopropoxy, butoxy and tert-butoxy. R₄ may be methoxy.

R₄ may be deutero-C₁₋₄alkoxy wherein C₁₋₄alkoxy is selected from the group consisting of methoxy, ethoxy, propoxy, isopropoxy, butoxy and tert-butoxy partially or completely substituted with one or more deuterium atoms where allowed by available valences. R₄ may be (²H₃)methoxy.

R₄ may be C₃₋₆cycloalkyl selected from the group consisting of cyclopropyl, cyclobutyl, cyclopenyl, and cyclohexyl. R₄ may be cyclopropyl.

R₄ may be C₁₋₄alkyl-amino, wherein C₁₋₄alkyl is selected from the group consisting of methyl, ethyl, propyl, isopropyl, butyl, isobutyl, sec-butyl, and tert-butyl. R₄ may be methylamino.

R₄ may be heterocyclyl, wherein heterocyclyl is a 3- to 6-membered monocyclic carbon atom ring structure radical containing 1 or 2 heteroatom ring members independently selected from N, O, or S. R₄ may be heterocyclyl selected from the group consisting of aziridinyl, oziranyl, thiiranyl, azetidinyl, oxetanyl, thietanyl, pyrrolidinyl, pyrazolinyl, tetrahyrofuranyl, thilolanyl, piperidinyl, piperazinyl, tetrahydro-2H-pyranyl, 1,4-dioxanyl, morpholinyl, and thianyl. R₄ is azetidinyl.

R₄ may be selected from the group consisting of halogen, cyano, methyl, ethyl, (²H₃)methyl, (²H₃)ethyl, difluoromethyl, trifluoromethyl, methoxy, ethoxy, (²H₃)methoxy, methylamino, ethylamino, cyclopropyl, and azetidinyl.

Certain aspects include a compound of Formula (I), wherein X is CH. In other aspects, X is CF. In other aspects, X is N.

One aspect includes a compound of Formula (I), wherein R_{w} is selected from the group consisting of halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, and halo-C₁₋₄alkoxy. R_{w} may be selected from the group consisting of halogen and C₁₋₄alkyl. R_{w} may be halogen selected from the group consisting of bromo, chloro, fluoro, and iodo. R_{w} may be fluoro. R_{w} may be C₁₋₄alkyl selected from methyl, ethyl, propyl, isopropyl, and tert-butyl. R_{w} may be methyl. R_{w} may be fluoro, chloro, bromo, methyl or ethyl.

Certain aspects include a compound of Formula (I), wherein n is 0. In other aspects, n is 1.

One aspect includes a compound of Formula (I), wherein n is 0, and R₁ is hydrogen or C₁₋₄alkyl. In one aspect, n is 0, and X is C.

One aspect includes a compound of Formula (I), wherein n is 0, R₂ is C₁₋₄alkyl, halo-C₁₋₄alkyl, hydroxyl-C₁₋₄alkyl, cyclopropyl, cyclobutyl, phenyl, or oxetanyl, optionally substituted with one or two R₃ substituents, and R₁ is hydrogen or C₁₋₄alkyl.

One aspect includes a compound of Formula (I), wherein A is A1, A2, A3, A4, A5 or A6, n is 0, and R₁ is hydrogen or C₁₋₄alkyl. In another aspect, A is A1, A2, A3, A4, A5 or A6,

n is 0, X is C, and R₁ is hydrogen or C₁₋₄alkyl.

One aspect includes a compound of Formula (I), wherein:

n is 0;

X is C;

R₂ is C₁₋₄alkyl, halo-C₁₋₄alkyl, hydroxyl-C₁₋₄alkyl, cyclopropyl, cyclobutyl, phenyl, or oxetanyl, optionally substituted with one or two R₃ substituents;

R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, C₁₋₄alkyl-amino, C₃₋₆cycloalkyl, and heterocyclyl;

R₁ is hydrogen or C₁₋₄alkyl; and

B is selected from the group consisting of:

phenyl unsubstituted or substituted with one R₄ substituent;

heteroaryl,

wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or

wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents; and

heterocyclyl,

wherein heterocyclyl is a 8- or 9-membered bicyclic carbon atom ring structure radical containing 2, 3 or 4 N, and optionally, when the ring structure contains 2 or 3 N, containing a second heteroatom ring member selected from O or S, optionally substituted with one or two independently selected R₄ substituents.

One aspect includes a compound of Formula (I), wherein:

n is 0;

X is C;

R₂ is C₁₋₄alkyl, halo-C₁₋₄alkyl, hydroxyl-C₁₋₄alkyl, cyclopropyl, cyclobutyl, phenyl, or oxetanyl, optionally substituted with one or two R₃ substituents;

R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, C₁₋₄alkyl-amino, C₃₋₆cycloalkyl, and heterocyclyl;

R₁ is hydrogen or C₁₋₄alkyl;

A is A1-A24; and

B is selected from the group consisting of:

phenyl unsubstituted or substituted with one R₄ substituent;

heteroaryl,

wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or

wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents; and

heterocyclyl,

wherein heterocyclyl is a 8- or 9-membered bicyclic carbon atom ring structure radical containing 2, 3 or 4 N, and optionally, when the ring structure contains 2 or 3 N, containing a second heteroatom ring member selected from O or S, optionally substituted with one or two independently selected R₄ substituents.

One aspect includes a compound of Formula (I), wherein:

n is 0;

X is C;

R₂ is C₁₋₄alkyl, halo-C₁₋₄alkyl, hydroxyl-C₁₋₄alkyl, cyclopropyl, cyclobutyl, phenyl, or oxetanyl, optionally substituted with one or two R₃ substituents;

R₄ is selected from the group consisting of halogen, cyano, methyl, ethyl, (²H₃)methyl, (²H₃)ethyl, difluoromethyl, trifluoromethyl, methoxy, ethoxy, (²H₃)methoxy, methylamino, ethylamino, cyclopropyl, and azetidinyl;

R₁ is hydrogen or C₁₋₄alkyl;

A is A1-A6; and

B is selected from the group consisting of heteroaryl and heterocycyl, wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or

wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents; and

wherein heterocyclyl is a 8- or 9-membered bicyclic carbon atom ring structure radical containing 2, 3 or 4 N, and optionally, when the ring structure contains 2 or 3 N, containing a second heteroatom ring member selected from O or S, optionally substituted with one or two independently selected R₄ substituents.

In an embodiment thereof, B is unsubstituted. In another embodiment thereof, B is heteroaryl, unsubstituted or substituted with one R₄ substituent. In another embodiment thereof, B is the 9-membered bicyclic carbon atom ring structure radical. In another embodiment thereof, B is the 5- or 6-membered monocyclic aromatic carbon atom ring structure radical. In an embodiment thereof, B is heterocycl, unsubstituted or substituted with one R₄ substituent.

Another aspect includes a compound of Formula (I), or a form thereof:

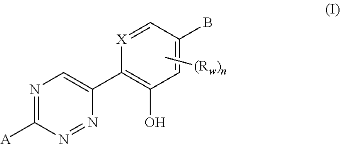

wherein:

A is selected from the group consisting of:

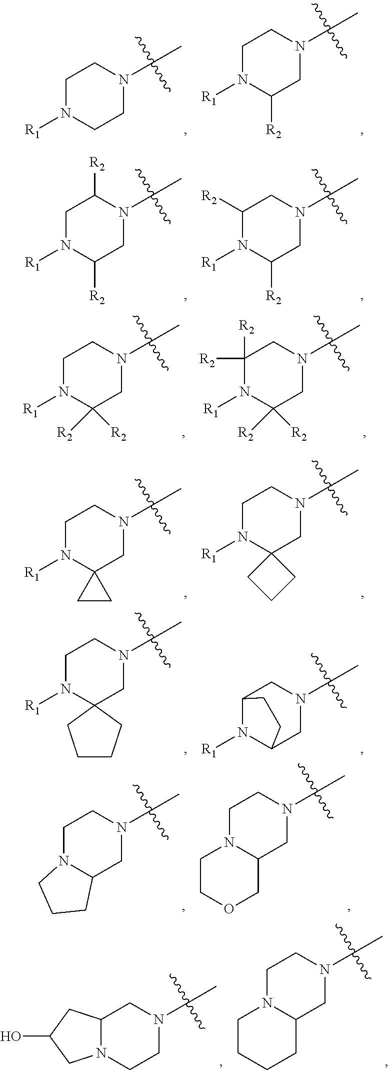

and any stereoisomer thereof;
R₁ is selected from the group consisting of hydrogen, C₁₋₄alkyl, and C₃₋₆cycloalkyl;
R₂ is independently selected from the group consisting of halogen, C₁₋₄alkyl, deutero-C₁₋₄alkyl, halo-C₁₋₄alkyl, hydroxyl-C₁₋₄alkyl, C₁₋₄alkoxy-C₁₋₄alkyl, C₂₋₄alkenyl, C₃₋₆cycloalkyl, phenyl, pyridinyl, and heterocyclyl, wherein heterocyclyl is a 3- to 6-membered carbon atom ring structure radical containing 1 or 2 heteroatom ring members selected from N, O, and S, and
wherein each instance of C₁₋₄alkyl, C₃₋₆cycloalkyl, phenyl, pyridinyl, and heterocyclyl is optionally substituted with one or two R₃ substituents;
R₃ is independently selected from the group consisting of halogen, hydroxyl, C₁₋₄alkyl, C₁₋₄alkoxy, and C₃₋₆cycloalkyl;
B is selected from the group consisting of:
phenyl optionally substituted with one or two independently selected R₄ substituents; and
heteroaryl, wherein heteroaryl is a 5- or 6-membered monocyclic aromatic carbon atom ring structure radical containing 1, 2, or 3 heteroatoms selected from N, O, and S, optionally substituted with one R₄ substituent;
R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, amino, C₁₋₄alkyl-amino, (C₁₋₄alkyl)₂-amino, C₃₋₆cycloalkyl, and heterocyclyl, wherein heterocyclyl is a 3- to 6-membered monocyclic carbon atom ring structure radical containing 1 or 2 heteroatom ring members independently selected from N, O, or S;
X is selected from the group consisting of CH, CF, and N;
R_{w} is selected from the group consisting of halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, and halo-C₁₋₄alkoxy; and
n is selected from the group consisting of 0 or 1;
wherein a form of the compound is selected from the group consisting of a salt, hydrate, solvate, and tautomer form thereof.

Except where provided, each of the terms and definitions for this aspect are the same as defined above.

In an aspect thereof, B may be a 5- or 6-membered monocyclic aromatic carbon atom ring structure radical containing 1, 2, or 3 heteroatoms selected from N, O, and S. B may be selected from the group consisting of: phenyl optionally substituted with one or two independently selected R₄ substituents; and heteroaryl, wherein heteroaryl is a 5- or 6-membered monocyclic aromatic carbon atom ring structure radical containing at least one N atom. B may be selected from the group consisting of: phenyl optionally substituted with one R₄ substituent; and heteroaryl, wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent. B may be heteroaryl, wherein heteroaryl is a 5- or 6-membered monocyclic aromatic carbon atom ring structure radical containing 1, 2, or 3 N atoms, optionally substituted with one R₄ substituent.

One aspect includes a compound of Formula (I), wherein B is selected from the group consisting of:

phenyl optionally substituted with one or two independently selected R₄ substituents;

heteroaryl, wherein heteroaryl is a 5- or 6-membered monocyclic aromatic carbon atom ring structure radical containing 1, 2, or 3 heteroatoms selected from N, O, and S, optionally substituted with one R₄ substituent.

B may be phenyl optionally substituted with one or two independently selected R₄ substituents. B may be unsubstituted phenyl or phenyl substituted with one R₄ substituent.

One aspect includes a compound of Formula (I), wherein B is heteroaryl, wherein heteroaryl is a 5- or 6-membered monocyclic aromatic carbon atom ring structure radical containing 1, 2, or 3 heteroatoms selected from N, O, and S, optionally substituted with one R₄ substituent. B may be heteroaryl, wherein heteroaryl is a 5- or 6-membered monocyclic aromatic carbon atom ring structure radical containing 1, 2, or 3 N atoms, and optionally a second heteroatom selected from O, and S, optionally substituted with one R₄ substituent. B may be heteroaryl, wherein heteroaryl is a 5- or 6-membered monocyclic aromatic carbon atom ring structure radical containing 2 or 3 heteroatoms selected from N, O, and S, optionally substituted with one R₄ substituent. B may be heteroaryl, wherein heteroaryl is a 5- or 6-membered monocyclic aromatic carbon atom ring structure radical containing 2 or 3 N atoms, and optionally a second heteroatom selected from O, and S, optionally substituted with one R₄ substituent.

B may be heteroaryl selected from the group consisting of furanyl, thiophenyl, 1H-pyrazolyl, 1H-imidazolyl, isoxazolyl, 1,3-thiazolyl, 1,3-oxazolyl, tetrazolyl, 1H-1,2,3-triazolyl, 2H-1,2,3-triazolyl, 1,2,4-oxadiazolyl, 1,3,4-oxadiazolyl, 1,2,3-thiadiazolyl, pyridinyl, pyrimidinyl, pyrazinyl, pyridazinyl, 1H-1,2,4-triazolyl, 1,2,4-thiadiazolyl, 1,3,4-oxadiazolyl, isothiazolyl, 1,2,5-thiadiazolyl, and 1,3,4-thiadiazolyl, optionally substituted with one R₄ substituent.

B may be heteroaryl selected from the group consisting of 1H-pyrazolyl, 1H-imidazolyl, 1,3-thiazolyl, 1,3-oxazolyl, 1H-1,2,3-triazolyl, 2H-1,2,3-triazolyl, pyridinyl, pyrimidinyl, pyrazinyl, 1H-1,2,4-triazolyl, 1,2,4-thiadiazolyl, 1,3,4-oxadiazolyl, isothiazolyl, 1,2,5-thiadiazolyl, and 1,3,4-thiadiazolyl, optionally substituted with one R₄ substituent.

B may be heteroaryl selected from the group consisting of 1H-pyrazolyl, 1H-imidazolyl, 1,3-thiazolyl, 1,3-oxazolyl, 1H-1,2,3-triazolyl, 2H-1,2,3-triazolyl, pyridinyl, pyrimidinyl, pyrazinyl, 1H-1,2,4-triazolyl, 1,2,4-thiadiazolyl, 1,3,4-oxadiazolyl, isothiazolyl, 1,2,5-thiadiazolyl, and 1,3,4-thiadiazolyl, substituted with one R₄ substituent.

B may be heteroaryl selected from the group consisting of furan-2-yl, furan-3-yl, thiophen-2-yl, thiophen-3-yl, 1H-pyrazol-1-yl, 1H-pyrazol-3-yl, 1H-pyrazol-4-yl, 1H-pyrazol-5-yl, 1H-imidazol-1-yl, 1H-imidazol-4-yl, isoxazol-3-yl, isoxazol-4-yl, isoxazol-5-yl, 1,3-thiazol-2-yl, 1,3-thiazol-4-yl, 1,3-thiazol-5-yl, 1,3-oxazol-2-yl, 1,3-oxazol-4-yl, 1,3-oxazol-5-yl, tetrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 2H-1,2,3-triazol-5-yl, 1,2,4-oxadiazol-3-yl, 1,3,4-oxadiazol-2-yl, 1,2,3-thiadiazol-4-yl, 1,2,3-thiadiazol-5-yl, pyridin-2-yl, pyridin-3-yl, pyridin-4-yl, pyrimidin-4-yl, pyrimidin-2-yl, pyrazin-2-yl, pyridazin-3-yl, pyridazin-4-yl, 1H-1,2,4-triazol-3-yl, 1H-1,2,4-triazol-5-yl, 1,2,4-thiadiazol-3-yl, 1,2,4-thiadiazol-5-yl, 1,3,4-oxadiazol-2-yl, isothiazol-3-yl, isothiazol-4-yl, isothiazol-5-yl, 1,2,5-thiadiazol-3-yl, 1,3,4-thiadiazol-2-yl, optionally substituted with one R₄ substituent.

B may be heteroaryl selected from the group consisting of 1H-pyrazol-4-yl, 1H-imidazol-1-yl, 1,3-thiazol-2-yl, 1,3-thiazol-4-yl, 1,3-oxazol-2-yl, 1H-1,2,3-triazol-4-yl, 1H-1,2,3-triazol-5-yl, 2H-1,2,3-triazol-2-yl, 2H-1,2,3-triazol-4-yl, pyridin-3-yl, pyridin-4-yl, pyrimidin-4-yl, pyrimidin-2-yl, pyrazin-2-yl, 1H-1,2,4-triazol-3-yl, 1,2,4-thiadiazol-3-yl, 1,2,4-thiadiazol-5-yl, 1,3,4-oxadiazol-2-yl, isothiazol-3-yl, isothiazol-4-yl, isothiazol-5-yl, 1,2,5-thiadiazol-3-yl, 1,3,4-thiadiazol-2-yl, optionally substituted with one R₄ substituent.

B may be heteroaryl selected from the group consisting of 1H-pyrazol-4-yl, 1H-imidazol-1-yl, 1,3-thiazol-2-yl, 1,3-thiazol-4-yl, 1,3-oxazol-2-yl, 1H-1,2,3-triazol-4-yl, 1H-1,2,3-triazol-5-yl, 2H-1,2,3-triazol-2-yl, 2H-1,2,3-triazol-4-yl, pyridin-3-yl, pyridin-4-yl, pyrimidin-4-yl, pyrimidin-2-yl, pyrazin-2-yl, 1H-1,2,4-triazol-3-yl, 1,2,4-thiadiazol-3-yl, 1,2,4-thiadiazol-5-yl, 1,3,4-oxadiazol-2-yl, isothiazol-3-yl, isothiazol-4-yl, isothiazol-5-yl, 1,2,5-thiadiazol-3-yl, 1,3,4-thiadiazol-2-yl, substituted with one R₄ substituent.

An aspect of the compound of Formula (I), or a form thereof, is:

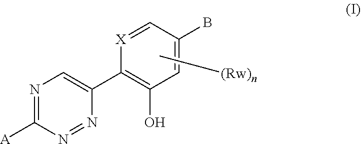

wherein:

A is selected from the group consisting of:

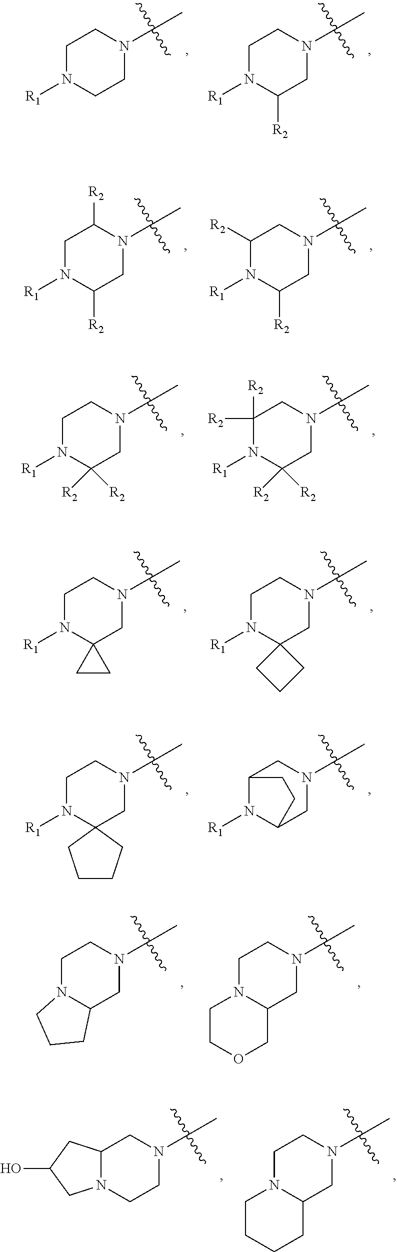

and any stereoisomer thereof;
R₁ is selected from the group consisting of hydrogen, C₁₋₄alkyl, and C₃₋₆cycloalkyl;
R₂ is independently selected from the group consisting of halogen, C₁₋₄alkyl, deutero-C₁₋₄alkyl, halo-C₁₋₄alkyl, hydroxyl-C₁₋₄alkyl, C₁₋₄alkoxy-C₁₋₄alkyl, C₂₋₄alkenyl, C₃₋₆cycloalkyl, phenyl, pyridinyl, and heterocyclyl, wherein heterocyclyl is a 3- to 6-membered carbon atom ring structure radical containing 1 or 2 heteroatom ring members selected from N, O, and S, and
wherein each instance of C₁₋₄alkyl, C₃₋₆cycloalkyl, phenyl, pyridinyl, and heterocyclyl is optionally substituted with one or two R₃ substituents;
R₃ is independently selected from the group consisting of halogen, hydroxyl, C₁₋₄alkyl, C₁₋₄alkoxy, and C₃₋₆cycloalkyl;
B is selected from the group consisting of:
heteroaryl, wherein heteroaryl is a 9- or 10-membered bicyclic aromatic carbon atom ring structure radical having 1, 2, 3, or 4 heteroatom ring members independently selected from N, O, or S, optionally substituted with one or two independently selected R₄ substituents; and
heterocyclyl, wherein heterocyclyl is a 8- to 10-membered bicyclic aromatic carbon atom ring structure radical containing 1, 2, 3, or 4 heteroatom ring members independently selected from N, O, or S, optionally substituted with one or two independently selected R₄ substituents;
R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, amino, C₁₋₄alkyl-amino, (C₁₋₄alkyl)₂-amino, C₃₋₆cycloalkyl, and heterocyclyl, wherein heterocyclyl is a 3- to 6-membered monocyclic carbon atom ring structure radical containing 1 or 2 heteroatom ring members independently selected from N, O, or S;
X is selected from the group consisting of CH, CF, and N;
R_{w} is selected from the group consisting of halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, and halo-C₁₋₄alkoxy; and
n is selected from the group consisting of 0 or 1;
wherein a form of the compound is selected from the group consisting of a salt, hydrate, solvate, and tautomer form thereof.

Except where provided, each of the terms and definitions for this aspect are the same as defined above.

B may be heteroaryl, wherein heteroaryl is a 9- or 10-membered bicyclic aromatic ring system having 1, 2, 3, or 4 heteroatom ring members independently selected from N, O, or S. B may be heteroaryl, wherein heteroaryl is a 9-membered bicyclic carbon atom ring structure radical containing 2, 3 or 4 N atoms, and optionally substituted with one or two independently selected R₄ substituents.

B may be heterocyclyl, wherein heterocyclyl is a 8- to 10-membered bicyclic aromatic carbon atom ring structure radical containing 1, 2, 3, or 4 heteroatom ring members independently selected from N, O, or S.

B may be selected from the group consisting of:

heteroaryl, wherein heteroaryl is a 9-membered bicyclic carbon atom ring structure radical containing at least 2 N atoms; and

heterocyclyl, wherein heterocyclyl is a 8- or 9-membered bicyclic carbon atom ring structure radical containing at least one N atom.

B may be selected from the group consisting of:

heteroaryl, wherein heteroaryl is a 9-membered bicyclic carbon atom ring structure radical containing 2, 3 or 4 N atoms, optionally substituted with one or two independently selected R₄ substituents; and

heterocyclyl, wherein heterocyclyl is a 8- or 9-membered bicyclic carbon atom ring structure radical containing 2, 3 or 4 N, optionally substituted with one or two independently selected R₄ substituents.

One aspect includes a compound of Formula (I), wherein B is heteroaryl, wherein heteroaryl is a 9- or 10-membered aromatic carbon atom ring structure radical having 1, 2, 3, or 4 heteroatom ring members independently selected from N, O, or S, optionally substituted with one or two independently selected R₄ substituents. B may be heteroaryl, wherein heteroaryl is a 9- or 10-membered bicyclic carbon atom ring structure radical containing at least one N atom ring member, optionally containing a second heteroatom ring member selected from O or S, and optionally substituted with one or two independently selected R₄ substituents. B may be heteroaryl, wherein heteroaryl is a 9- or 10-membered bicyclic carbon atom ring structure radical containing 2, 3 or 4 N atoms, optionally containing a second heteroatom ring member selected from O or S, and optionally substituted with one or two independently selected R₄ substituents. B may be heteroaryl, wherein heteroaryl is a 9-membered bicyclic carbon atom ring structure radical containing 2, 3 or 4 N, optionally substituted with one or two independently selected R₄ substituents. B may be heteroaryl, wherein heteroaryl is a 9-membered bicyclic carbon atom ring structure radical containing 2, 3 or 4 N, optionally containing a second heteroatom ring member selected from O or S, and optionally substituted with one or two independently selected R₄ substituents. B may be heteroaryl, wherein heteroaryl is a unsubstituted 9-membered bicyclic carbon atom ring structure radical containing 2, 3 or 4 N, optionally containing a second heteroatom ring member selected from O or S. B may be heteroaryl, wherein heteroaryl is a 9-membered bicyclic carbon atom ring structure radical containing 2, 3 or 4 N, optionally containing a second heteroatom ring member selected from O or S, and is unsubstituted or substituted with one R₄ substituent.

B may be heteroaryl selected from the group consisting of 1H-indazolyl, 2H-indazolyl, indolizinyl, benzofuranyl, benzothiophenyl, 1H-benzimidazolyl, 1,3-benzoxazolyl, 1,3-benzothiazolyl, 1,3-benzodioxolyl, 1,2,3-benzotriazolyl, 7H-purinyl, furo[3,2-b]pyridinyl, furo[3,2-c]pyridinyl, 1,3-oxazolo[5,4-b]pyridinyl, thieno[3,2-c]pyridinyl, thieno[2,3-d]pyrimidinyl, pyrrolo[1,2-a]pyrimidinyl, pyrrolo[1,2-a]pyrazinyl, pyrrolo[1,2-b]pyridazinyl, pyrazolo[1,5-a]pyridinyl, 1H-pyrrolo[2,3-b]pyridinyl, 5H-pyrrolo[2,3-b]pyrazinyl, 1H-pyrrolo[2,3-c]pyridinyl, 1H-pyrazolo[3,4-b]pyridinyl, 2H-pyrazolo[3,4-b]pyridinyl, 1H-pyrazolo[3,4-b]pyrazinyl, 1H-pyrazolo[3,4-c]pyridinyl, 1H-pyrazolo[4,3-b]pyridinyl, 2H-pyrazolo[4,3-b]pyridinyl, 2H-pyrazolo[4,3-c]pyridinyl, 1H-pyrazolo[4,3-d]pyrimidinyl, pyrazolo[1,5-a]pyrazinyl, imidazo[1,2-a]pyridinyl, imidazo[1,2-a]pyrimidinyl, imidazo[1,2-a]pyrazinyl, imidazo[1,2-b]pyridazinyl, imidazo[1,2-c]pyrimidin-2-yl, 1H-imidazo[4,5-b]pyridinyl, 3H-imidazo[4,5-b]pyridinyl, imidazo[2,1-b][1,3]thiazolyl, imidazo[2,1-b][1,3,4]thiadiazolyl, [1,3]oxazolo[4,5-b]pyridinyl, [1,2,3]triazolo[1,5-a]pyridinyl, 1H-[1,2,3]triazolo[4,5-b]pyridinyl, 3H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, tetrazolo[1,5-a]pyridinyl, tetrazolo[1,5-b]pyridazinyl, thiazolo[4,5-b]pyrazinyl, thiazolo[5,4-c]pyridinyl, quinolinyl, isoquinolinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, optionally substituted with one or two independently selected R₄ substituents.

B may be heteroaryl selected from the group consisting of 2H-indazolyl, 7H-purinyl, furo[3,2-b]pyridinyl, pyrazolo[1,5-a]pyridinyl, 2H-pyrazolo[3,4-b]pyridinyl, 1H-pyrazolo[4,3-b]pyridinyl, imidazo[1,2-a]pyridinyl, imidazo[1,2-a]pyrazinyl, imidazo[1,2-b]pyridazinyl, [1,2,3]triazolo[1,5-a]pyridinyl, 1H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, thiazolo[4,5-b]pyrazinyl, thiazolo[5,4-c]pyridinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, optionally substituted with one or two independently selected R₄ substituents.

B may be heteroaryl selected from the group consisting of 2H-indazolyl, 7H-purinyl, furo[3,2-b]pyridinyl, pyrazolo[1,5-a]pyridinyl, 2H-pyrazolo[3,4-b]pyridinyl, 1H-pyrazolo[4,3-b]pyridinyl, imidazo[1,2-a]pyridinyl, imidazo[1,2-a]pyrazinyl, imidazo[1,2-b]pyridazinyl, [1,2,3]triazolo[1,5-a]pyridinyl, 1H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, thiazolo[4,5-b]pyrazinyl, thiazolo[5,4-c]pyridinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, substituted with one R₄ substituent.

B may be heteroaryl selected from the group consisting of 2H-indazolyl, 7H-purinyl, furo[3,2-b]pyridinyl, pyrazolo[1,5-a]pyridinyl, 2H-pyrazolo[3,4-b]pyridinyl, 1H-pyrazolo[4,3-b]pyridinyl, imidazo[1,2-a]pyridinyl, imidazo[1,2-a]pyrazinyl, imidazo[1,2-b]pyridazinyl, [1,2,3]triazolo[1,5-a]pyridinyl, 1H-[1,2,3]triazolo[4,5-b]pyridinyl, [1,2,4]triazolo[1,5-a]pyridinyl, [1,2,4]triazolo[1,5-a]pyrimidinyl, [1,2,4]triazolo[1,5-a]pyrazinyl, [1,2,4]triazolo[1,5-b]pyridazinyl, [1,2,4]triazolo[4,3-a]pyridinyl, thiazolo[4,5-b]pyrazinyl, thiazolo[5,4-c]pyridinyl, [1,2,5]thiadiazolo[3,4-b]pyridinyl, 1H-pyrazolo[3,4-d]pyrimidinyl, 1H-pyrrolo[3,2-b]pyridinyl, 2H-[1,2,3]triazolo[4,5-b]pyridinyl, 2H-[1,2,3]triazolo[4,5-c]pyridinyl, 3H-[1,2,3]triazolo[4,5-b]pyridinyl, 3H-[1,2,3]triazolo[4,5-c]pyridinyl, benzo[c][1,2,5]thiadiazolyl, imidazo[1,5-a]pyridinyl, pyrazolo[1,5-a]pyrimidinyl, and thiazolo[5,4-b]pyridinyl, substituted with two independently selected R₄ substituents.

B may be heteroaryl selected from the group consisting of 1H-indazol-5-yl, 2H-indazol-5-yl, indolizin-2-yl, benzofuran-2-yl, benzofuran-5-yl, benzothiophen-2-yl, benzothiophen-3-yl, 1H-benzimidazol-2-yl, 1H-benzimidazol-5-yl, 1H-benzimidazol-6-yl, 1,3-benzoxazol-2-yl, 1,3-benzoxazol-5-yl, 1,3-benzoxazol-6-yl, 1,3-benzothiazol-2-yl, 1,3-benzothiazol-5-yl, 1,3-benzothiazol-6-yl, 1,3-benzodioxol-5-yl, 1,2,3-benzotriazol-5-yl, 7H-purin-2-yl, furo[3,2-b]pyridin-2-yl, furo[3,2-c]pyridin-2-yl, furo[2,3-c]pyridin-2-yl, 1,3-oxazolo[5,4-b]pyridine-5-yl, thieno[3,2-c]pyridin-2-yl, thieno[2,3-d]pyrimidin-6-yl, pyrrolo[1,2-a]pyrimidin-7-yl, pyrrolo[1,2-a]pyrazin-7-yl, pyrrolo[1,2-b]pyridazin-2-yl, pyrazolo[1,5-a]pyridin-2-yl, pyrazolo[1,5-a]pyridin-3-yl, pyrazolo[1,5-a]pyridin-5-yl, 1H-pyrrolo[2,3-b]pyridin-5-yl, 5H-pyrrolo[2,3-b]pyrazin-2-yl, 1H-pyrrolo[2,3-c]pyridin-4-yl, 1H-pyrazolo[3,4-b]pyridin-5-yl, 1H-pyrazolo[3,4-b]pyridin-6-yl, 2H-pyrazolo[3,4-b]pyridin-5-yl, 1H-pyrazolo[3,4-b]pyrazin-5-yl, 1H-pyrazolo[3,4-c]pyridin-1-yl, 1H-pyrazolo[3,4-c]pyridin-5-yl, 1H-pyrazolo[4,3-b]pyridin-1-yl, 1H-pyrazolo[4,3-b]pyridin-5-yl, 1H-pyrazolo[4,3-b]pyridin-6-yl, 2H-pyrazolo[4,3-b]pyridin-5-yl, 2H-pyrazolo[4,3-c]pyridin-5-yl, 1H-pyrazolo[4,3-d]pyrimidin-5-yl, pyrazolo[1,5-a]pyrazin-2-yl, imidazo[1,2-a]pyridin-2-yl, imidazo[1,2-a]pyridin-6-yl, imidazo[1,2-a]pyrimidin-2-yl, imidazo[1,2-a]pyrimidin-6-yl, imidazo[1,2-a]pyrazin-2-yl, imidazo[1,2-a]pyrazin-6-yl, imidazo[1,2-b]pyridazin-2-yl, imidazo[1,2-b]pyridazin-6-yl, imidazo[1,2-c]pyrimidin-2-yl, 1H-imidazo[4,5-b]pyridin-5-yl, 3H-imidazo[4,5-b]pyridin-5-yl, imidazo[2,1-b][1,3]thiazol-6-yl, imidazo[2,1-b][1,3,4]thiadiazol-6-yl, [1,3]oxazolo[4,5-b]pyridin-2-yl, [1,2,3]triazolo[1,5-a]pyridin-5-yl, [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, 3H-[1,2,3]triazolo[4,5-b]pyridin-5-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, tetrazolo[1,5-a]pyridin-7-yl, tetrazolo[1,5-b]pyridazin-7-yl, thiazolo[4,5-b]pyrazin-2-yl, thiazolo[5,4-c]pyridin-2-yl, tetrazolo[1,5-a]pyridin-7-yl, tetrazolo[1,5-b]pyridazin-7-yl, quinolin-6-yl, isoquinolin-6-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, optionally substituted with one or two independently selected R₄ substituents.

B may be heteroaryl selected from the group consisting of 1H-indazol-5-yl, 7H-purin-2-yl, furo[3,2-b]pyridin-2-yl, pyrazolo[1,5-a]pyridin-3-yl, 2H-pyrazolo[3,4-b]pyridin-5-yl, 1H-pyrazolo[4,3-b]pyridin-1-yl, imidazo[1,2-a]pyridin-6-yl, imidazo[1,2-a]pyrazin-6-yl, imidazo[1,2-b]pyridazin-6-yl [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, thiazolo[4,5-b]pyrazin-2-yl, thiazolo[5,4-c]pyridin-2-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, optionally substituted with one or two independently selected R₄ substituents.

B may be heteroaryl selected from the group consisting of 1H-indazol-5-yl, 7H-purin-2-yl, furo[3,2-b]pyridin-2-yl, pyrazolo[1,5-a]pyridin-3-yl, 2H-pyrazolo[3,4-b]pyridin-5-yl, 1H-pyrazolo[4,3-b]pyridin-1-yl, imidazo[1,2-a]pyridin-6-yl, imidazo[1,2-a]pyrazin-6-yl, imidazo[1,2-b]pyridazin-6-yl [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, thiazolo[4,5-b]pyrazin-2-yl, thiazolo[5,4-c]pyridin-2-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, substituted with one R₄ substituent.

B may be heteroaryl selected from the group consisting of 1H-indazol-5-yl, 7H-purin-2-yl, furo[3,2-b]pyridin-2-yl, pyrazolo[1,5-a]pyridin-3-yl, 2H-pyrazolo[3,4-b]pyridin-5-yl, 1H-pyrazolo[4,3-b]pyridin-1-yl, imidazo[1,2-a]pyridin-6-yl, imidazo[1,2-a]pyrazin-6-yl, imidazo[1,2-b]pyridazin-6-yl [1,2,3]triazolo[1,5-a]pyridin-6-yl, 1H-[1,2,3]triazolo[4,5-b]pyridin-5-yl, 1H-[1,2,3]triazolo[4,5-b]pyridin-6-yl, [1,2,4]triazolo[1,5-a]pyridin-6-yl, [1,2,4]triazolo[1,5-a]pyrimidin-2-yl, [1,2,4]triazolo[1,5-a]pyrimidin-6-yl, [1,2,4]triazolo[1,5-a]pyrazin-6-yl, [1,2,4]triazolo[1,5-b]pyridazin-6-yl, [1,2,4]triazolo[4,3-a]pyridin-6-yl, thiazolo[4,5-b]pyrazin-2-yl, thiazolo[5,4-c]pyridin-2-yl, [1,2,5]thiadiazolo[3,4-b]pyridin-6-yl, 1H-pyrazolo[3,4-d]pyrimidin-1-yl, 1H-pyrrolo[3,2-b]pyridin-1-yl, 2H-[1,2,3]triazolo[4,5-b]pyridin-5-yl, 2H-[1,2,3]triazolo[4,5-b]pyridin-6-yl, 2H-[1,2,3]triazolo[4,5-c]pyridin-6-yl, 2H-[1,2,3]triazolo[4,5-b]pyridin-6-yl, 3H-[1,2,3]triazolo[4,5-c]pyridin-6-yl, benzo[c][1,2,5]thiadiazol-5-yl, imidazo[1,5-a]pyridin-6-yl, imidazo[1,5-a]pyridin-7-yl, pyrazolo[1,5-a]pyrimidin-3-yl, thiazolo[5,4-b]pyridin-2-yl, substituted with two independently selected R₄ substituents.

One aspect includes a compound of Formula (I), wherein B is heterocyclyl, wherein heterocyclyl is a 8- to 10-membered bicyclic carbon atom ring structure radical containing 1, 2, 3, or 4 heteroatom ring members independently selected from N, O, or S, optionally substituted with one or two independently selected R₄ substituents. B may be heterocyclyl, wherein heterocyclyl is a 8- or 9-membered bicyclic carbon atom ring structure radical containing at least one N, optionally substituted with one or two independently selected R₄ substituents. B may be heterocyclyl, wherein heterocyclyl is a 8- or 9-membered bicyclic carbon atom ring structure radical containing 2, 3 or 4 N, optionally substituted with one or two independently selected R₄ substituents. B may be heterocyclyl, wherein heterocyclyl is a 9-membered bicyclic carbon atom ring structure radical containing 2, 3 or 4 N, optionally substituted with one or two independently selected R₄ substituents, or heterocyclyl is a 8 membered bicyclic carbon atom ring structure radical containing 2, or 3 N, optionally substituted with one or two independently selected R₄ substituents. B may be heterocyclyl, wherein heterocyclyl is a 8- or 9-membered bicyclic carbon atom ring structure radical containing 2, 3 or 4 N, and optionally containing a second heteroatom ring member selected from O or S, optionally substituted with one or two independently selected R₄ substituents. B may be heterocyclyl, wherein heterocyclyl is a 9-membered bicyclic carbon atom ring structure radical containing 2, 3 or 4 N, and optionally containing a second heteroatom ring member selected from O or S, optionally substituted with one or two independently selected R₄ substituents. B may be heterocyclyl, wherein heterocyclyl is a unsubstituted 8- or 9-membered bicyclic carbon atom ring structure radical containing 2, 3 or 4 N, optionally containing a second heteroatom ring member selected from O or S. B may be heterocyclyl, wherein heterocyclyl is a 8- or 9-membered bicyclic carbon atom ring structure radical containing 2, 3 or 4 N, optionally containing a second heteroatom ring member selected from O or S, and is unsubstituted or substituted with one R₄ substituent.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazolyl, 5,6-dihydro-4H-pyrrolo[1,2-b]pyrazolyl, 4,5,6,7-tetrahydropyrazolo[1,5-a]pyridinyl, 5,6-dihydro-4H-pyrrolo[1,2-b]pyrazolyl, 6,7-dihydro-5H-pyrrolo[1,2-a]imidazolyl, 5,6-dihydro-4H-pyrrolo[1,2-b]pyrazolyl, and 5,6-dihydro-4H-pyrrolo[1,2-b]pyrazolyl, optionally substituted with one or two independently selected R₄ substituents.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazolyl, 5,6-dihydro-4H-pyrrolo[1,2-b]pyrazolyl, 4,5,6,7-tetrahydropyrazolo[1,5-a]pyridinyl, 5,6-dihydro-4H-pyrrolo[1,2-b]pyrazolyl, 6,7-dihydro-5H-pyrrolo[1,2-a]imidazolyl, 5,6-dihydro-4H-pyrrolo[1,2-b]pyrazolyl, and 5,6-dihydro-4H-pyrrolo[1,2-b]pyrazolyl, substituted with two independently selected R₄ substituents.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazolyl, 5,6-dihydro-4H-pyrrolo[1,2-b]pyrazolyl, 4,5,6,7-tetrahydropyrazolo[1,5-a]pyridinyl, 5,6-dihydro-4H-pyrrolo[1,2-b]pyrazolyl, 6,7-dihydro-5H-pyrrolo[1,2-a]imidazolyl, 5,6-dihydro-4H-pyrrolo[1,2-b]pyrazolyl, and 5,6-dihydro-4H-pyrrolo[1,2-b]pyrazolyl, substituted with one R₄ substituent.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazol-7-yl, 5,6-dihydro-4H-pyrrolo[1,2-b]pyrazol-3-yl, 4,5,6,7-tetrahydropyrazolo[1,5-a]pyridin-3-yl, 5,6-dihydro-4H-pyrrolo[1,2-b]pyrazol-3-yl, 6,7-dihydro-5H-pyrrolo[1,2-a]imidazol-3-yl, 5,6-dihydro-4H-pyrrolo[1,2-b]pyrazol-3-yl, and 5,6-dihydro-4H-pyrrolo[1,2-b]pyrazol-3-yl, optionally substituted with one or two independently selected R₄ substituents.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazol-7-yl, 5,6-dihydro-4H-pyrrolo[1,2-b]pyrazol-3-yl, 4,5,6,7-tetrahydropyrazolo[1,5-a]pyridin-3-yl, 5,6-dihydro-4H-pyrrolo[1,2-b]pyrazol-3-yl, 6,7-dihydro-5H-pyrrolo[1,2-a]imidazol-3-yl, 5,6-dihydro-4H-pyrrolo[1,2-b]pyrazol-3-yl, and 5,6-dihydro-4H-pyrrolo[1,2-b]pyrazol-3-yl, substituted with one R₄ substituent.

B may be heterocyclyl selected from the group consisting of 5,6-dihydro-[1,2,4]triazolo[1,5-a]pyrazin-7(8H)-yl, 2,3-dihydro-1H-imidazo[1,2-b]pyrazol-7-yl, 5,6-dihydro-4H-pyrrolo[1,2-b]pyrazol-3-yl, 4,5,6,7-tetrahydropyrazolo[1,5-a]pyridin-3-yl, 5,6-dihydro-4H-pyrrolo[1,2-b]pyrazol-3-yl, 6,7-dihydro-5H-pyrrolo[1,2-a]imidazol-3-yl, 5,6-dihydro-4H-pyrrolo[1,2-b]pyrazol-3-yl, and 5,6-dihydro-4H-pyrrolo[1,2-b]pyrazol-3-yl, substituted with two independently selected R₄ substituents.

An aspect of the compound of Formula (I) or a form thereof includes a compound selected from the group consisting of, wherein “#” indicates that the compound is a racemic mixture of enantiomers, and wherein indicates that the compound may exist as the opposite enantiomer:

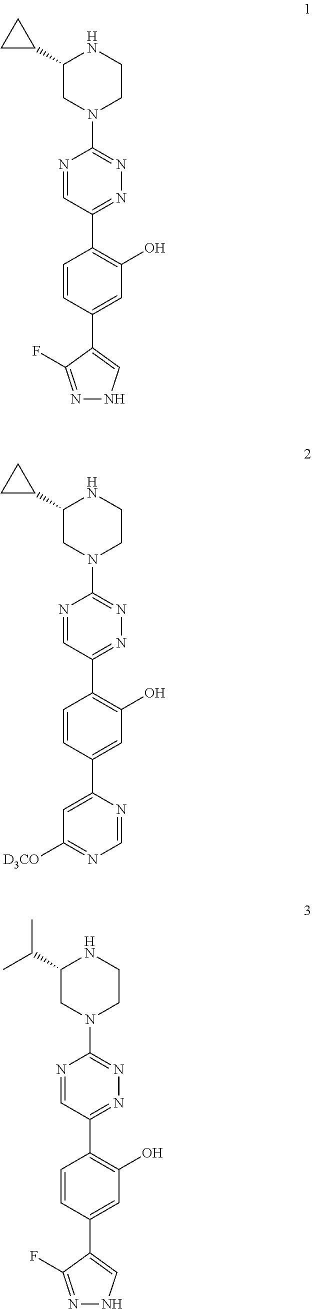

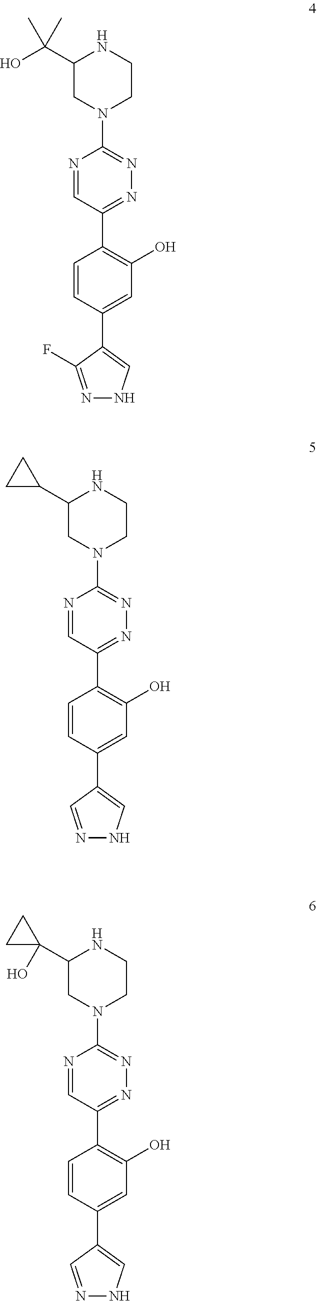 [SMILES: CC(C)(O)C1CN(c2ncc(-c3ccc(-c4c[nH]nc4F)cc3O)nn2)CCN1 ; Oc1cc(-c2cn[nH]c2)ccc1-c1cnc(N2CCNC(C3CC3)C2)nn1 ; Oc1cc(-c2cn[nH]c2)ccc1-c1cnc(N2CCNC(C3(O)CC3)C2)nn1]

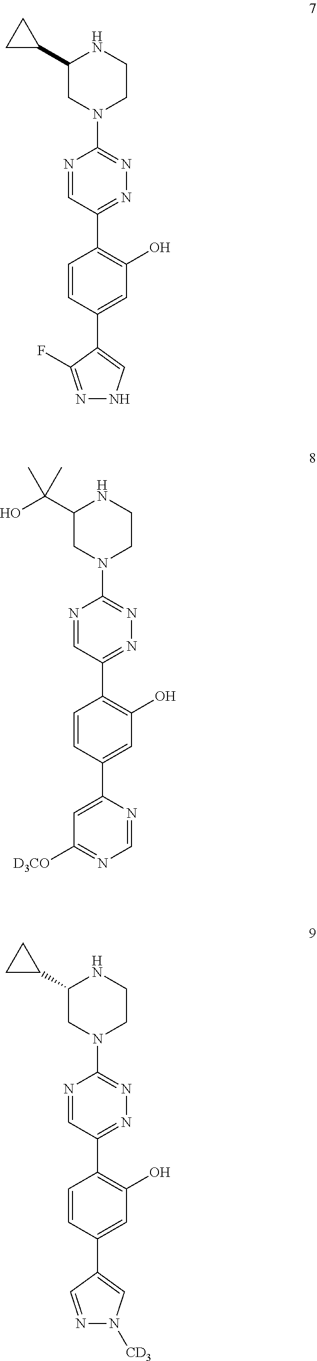

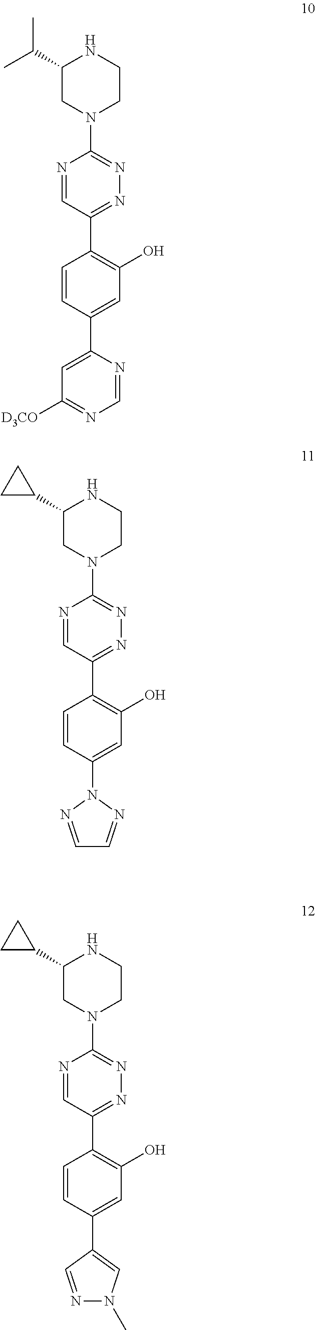

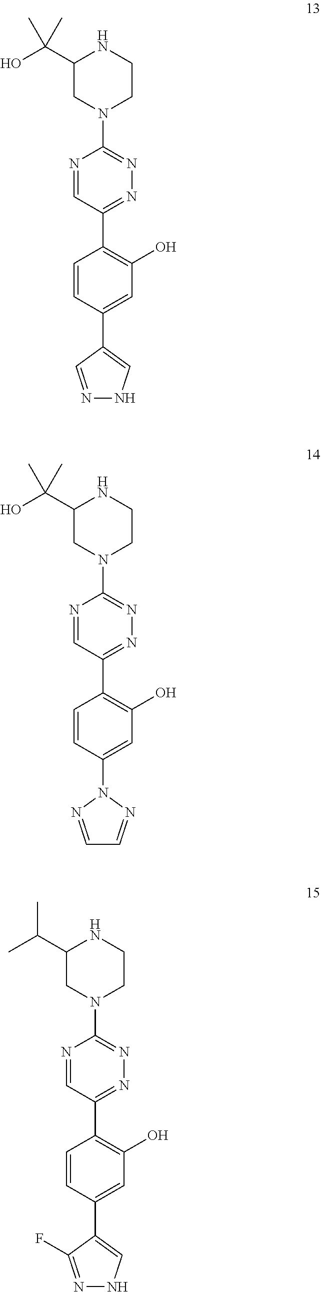 [SMILES: CC(C)(O)C1CN(c2ncc(-c3ccc(-c4cn[nH]c4)cc3O)nn2)CCN1 ; CC(C)(O)C1CN(c2ncc(-c3ccc(-n4nccn4)cc3O)nn2)CCN1 ; CC(C)C1CN(c2ncc(-c3ccc(-c4c[nH]nc4F)cc3O)nn2)CCN1]

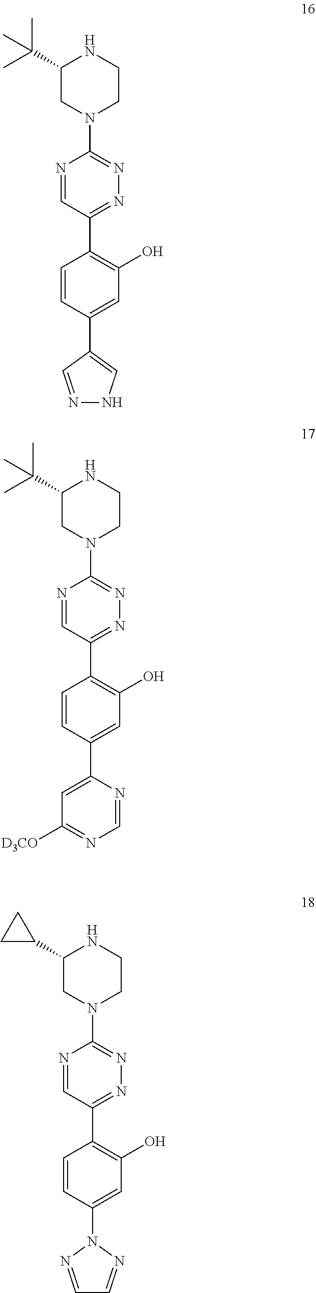

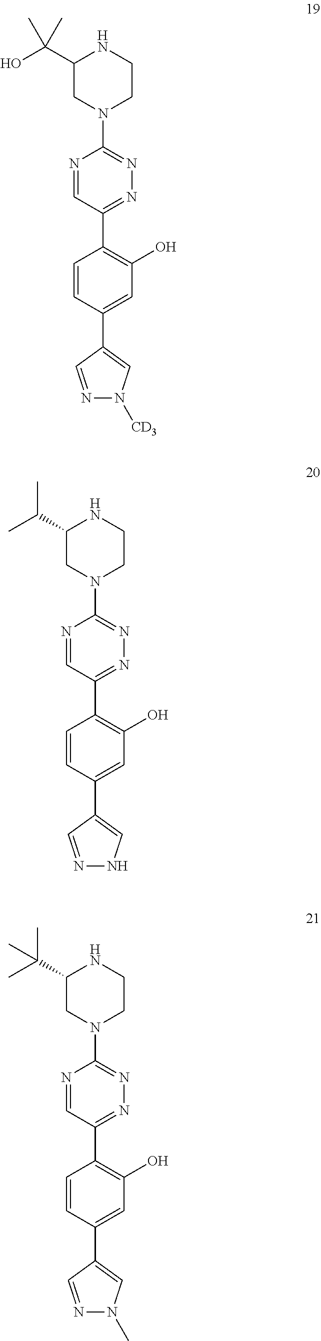 [SMILES: [2H]C([2H])([2H])n1cc(-c2ccc(-c3cnc(N4CCNC(C(C)(C)O)C4)nn3)c(O)c2)cn1 ; CC(C)[C@H]1CN(c2ncc(-c3ccc(-c4cn[nH]c4)cc3O)nn2)CCN1 ; Cn1cc(-c2ccc(-c3cnc(N4CCN[C@@H](C(C)(C)C)C4)nn3)c(O)c2)cn1]

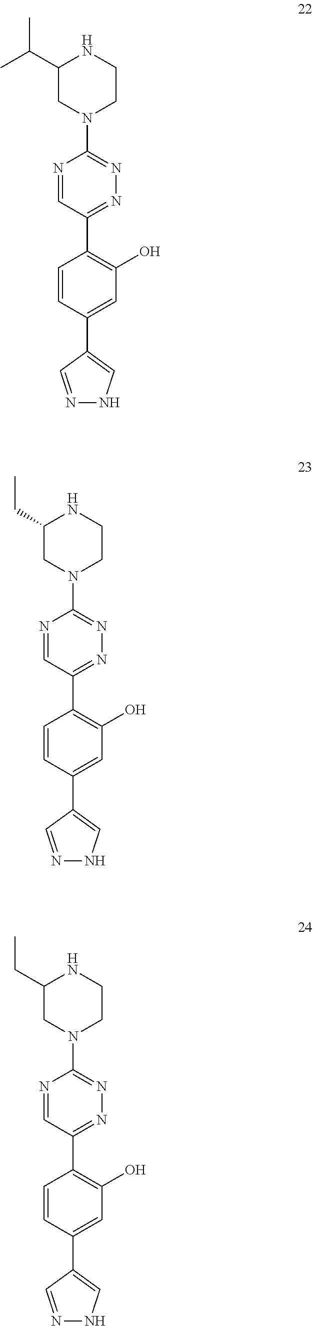 [SMILES: CC(C)C1CN(c2ncc(-c3ccc(-c4cn[nH]c4)cc3O)nn2)CCN1 ; CC[C@H]1CN(c2ncc(-c3ccc(-c4cn[nH]c4)cc3O)nn2)CCN1 ; CCC1CN(c2ncc(-c3ccc(-c4cn[nH]c4)cc3O)nn2)CCN1]

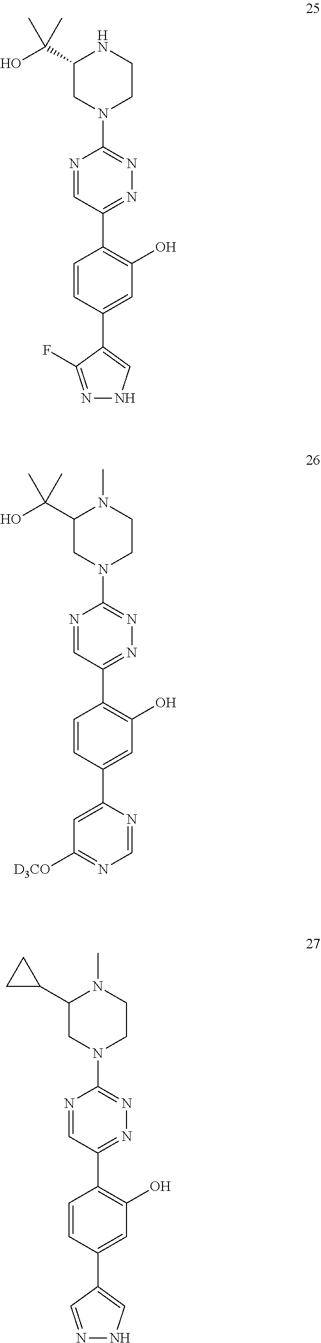

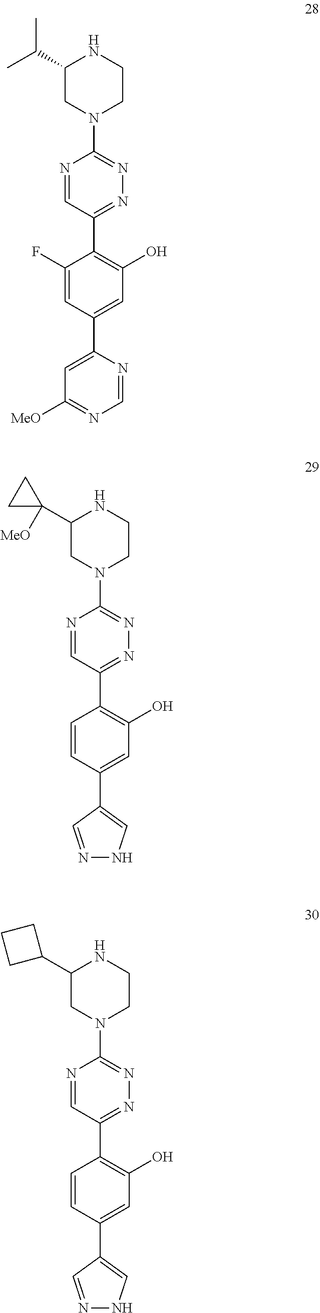 [SMILES: COc1cc(-c2cc(O)c(-c3cnc(N4CCN[C@@H](C(C)C)C4)nn3)c(F)c2)ncn1 ; COC1(C2CN(c3ncc(-c4ccc(-c5cn[nH]c5)cc4O)nn3)CCN2)CC1 ; Oc1cc(-c2cn[nH]c2)ccc1-c1cnc(N2CCNC(C3CCC3)C2)nn1]

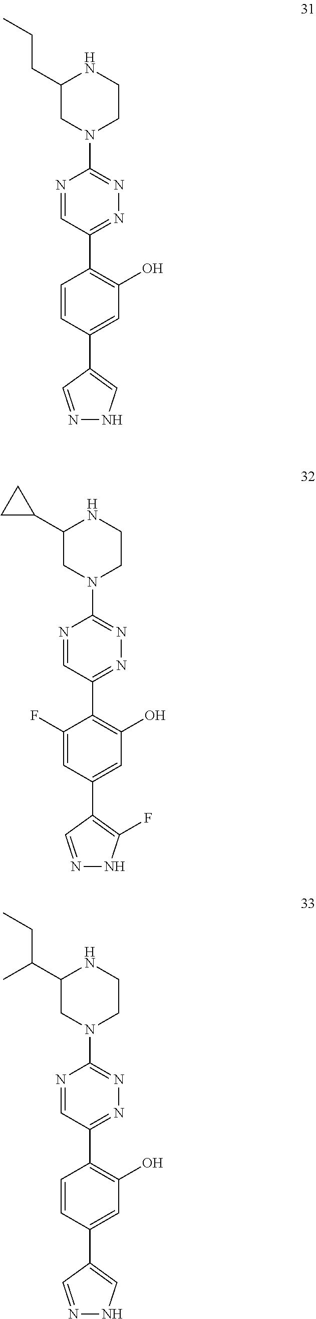 [SMILES: CCCC1CN(c2ncc(-c3ccc(-c4cn[nH]c4)cc3O)nn2)CCN1 ; Oc1cc(-c2cn[nH]c2F)cc(F)c1-c1cnc(N2CCNC(C3CC3)C2)nn1 ; CCC(C)C1CN(c2ncc(-c3ccc(-c4cn[nH]c4)cc3O)nn2)CCN1]

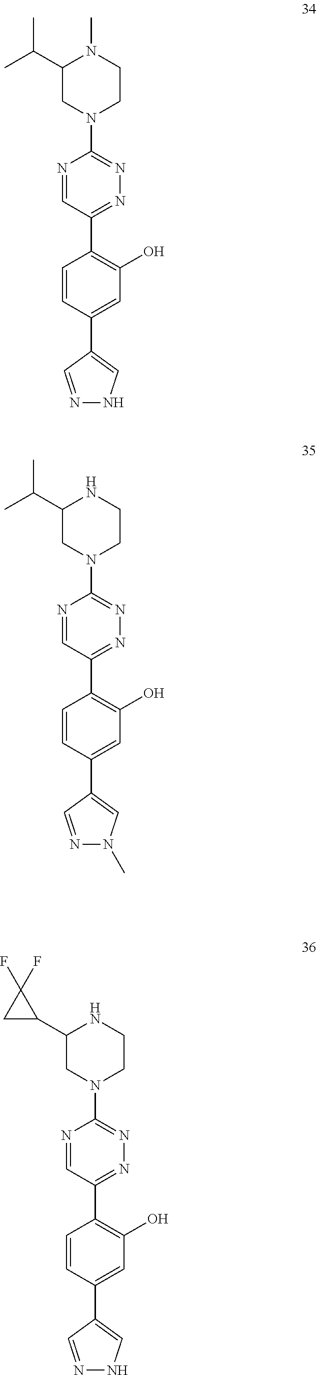 [SMILES: CC(C)C1CN(c2ncc(-c3ccc(-c4cn[nH]c4)cc3O)nn2)CCN1C ; CC(C)C1CN(c2ncc(-c3ccc(-c4cnn(C)c4)cc3O)nn2)CCN1 ; Oc1cc(-c2cn[nH]c2)ccc1-c1cnc(N2CCNC(C3CC3(F)F)C2)nn1]

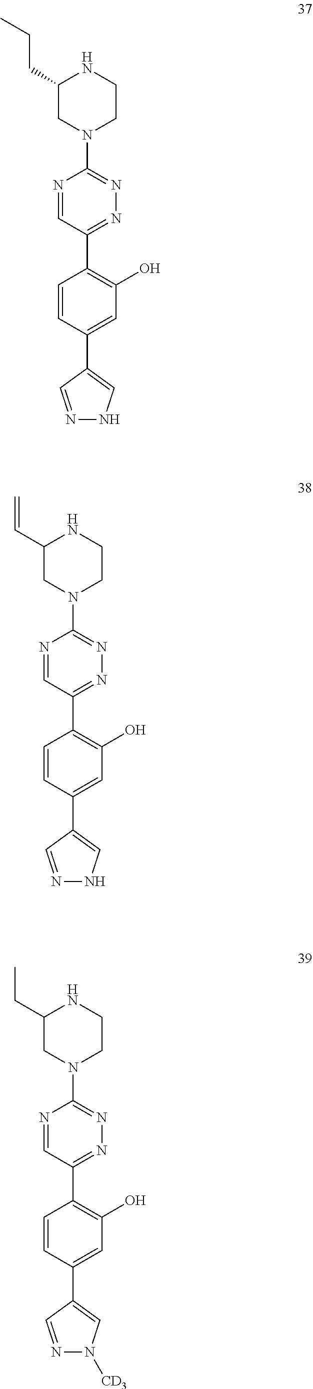 [SMILES: CCC[C@H]1CN(c2ncc(-c3ccc(-c4cn[nH]c4)cc3O)nn2)CCN1 ; C=CC1CN(c2ncc(-c3ccc(-c4cn[nH]c4)cc3O)nn2)CCN1 ; [2H]C([2H])([2H])n1cc(-c2ccc(-c3cnc(N4CCNC(CC)C4)nn3)c(O)c2)cn1]

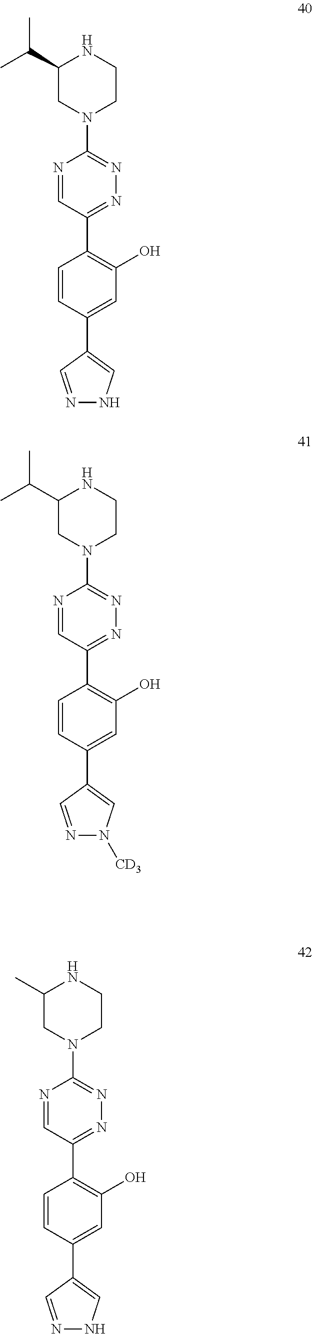 [SMILES: CC(C)[C@@H]1CN(c2ncc(-c3ccc(-c4cn[nH]c4)cc3O)nn2)CCN1 ; [2H]C([2H])([2H])n1cc(-c2ccc(-c3cnc(N4CCNC(C(C)C)C4)nn3)c(O)c2)cn1 ; CC1CN(c2ncc(-c3ccc(-c4cn[nH]c4)cc3O)nn2)CCN1]

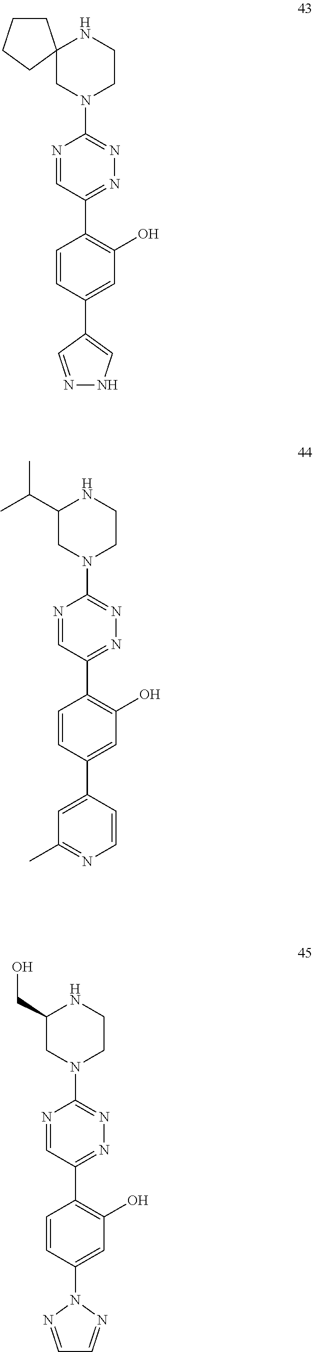 [SMILES: Oc1cc(-c2cn[nH]c2)ccc1-c1cnc(N2CCNC3(CCCC3)C2)nn1 ; Cc1cc(-c2ccc(-c3cnc(N4CCNC(C(C)C)C4)nn3)c(O)c2)ccn1 ; OC[C@@H]1CN(c2ncc(-c3ccc(-n4nccn4)cc3O)nn2)CCN1]

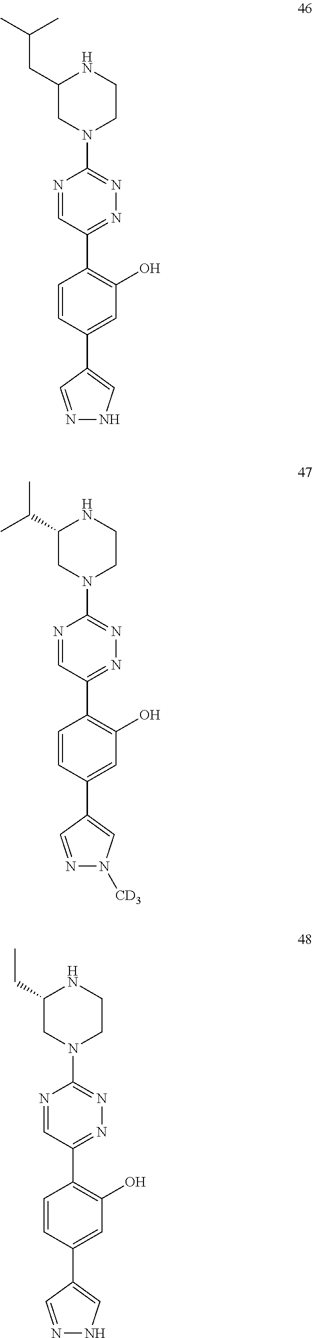 [SMILES: CC(C)CC1CN(c2ncc(-c3ccc(-c4cn[nH]c4)cc3O)nn2)CCN1 ; [2H]C([2H])([2H])n1cc(-c2ccc(-c3cnc(N4CCN[C@@H](C(C)C)C4)nn3)c(O)c2)cn1 ; CC[C@H]1CN(c2ncc(-c3ccc(-c4cn[nH]c4)cc3O)nn2)CCN1]

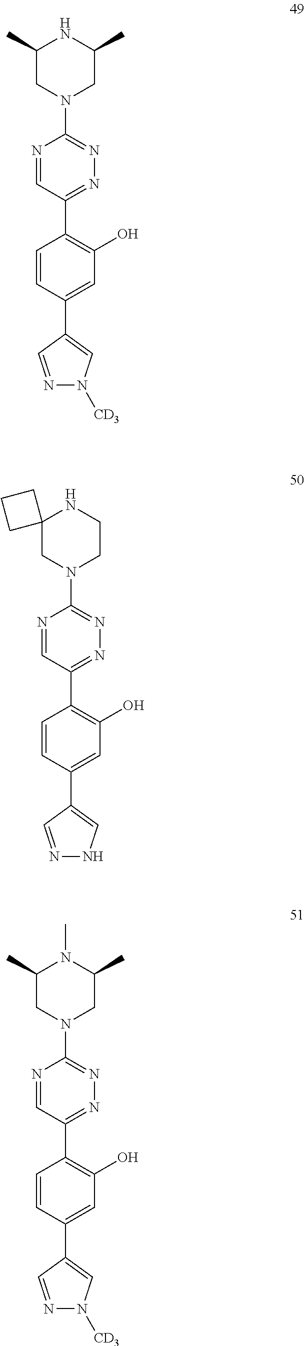

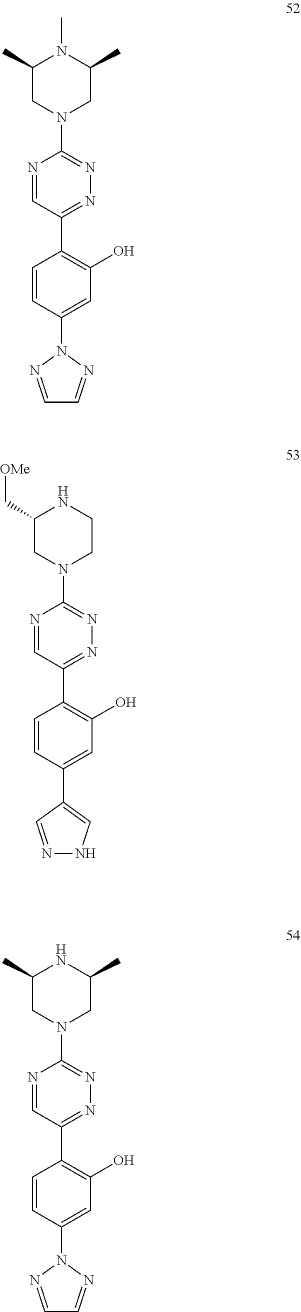

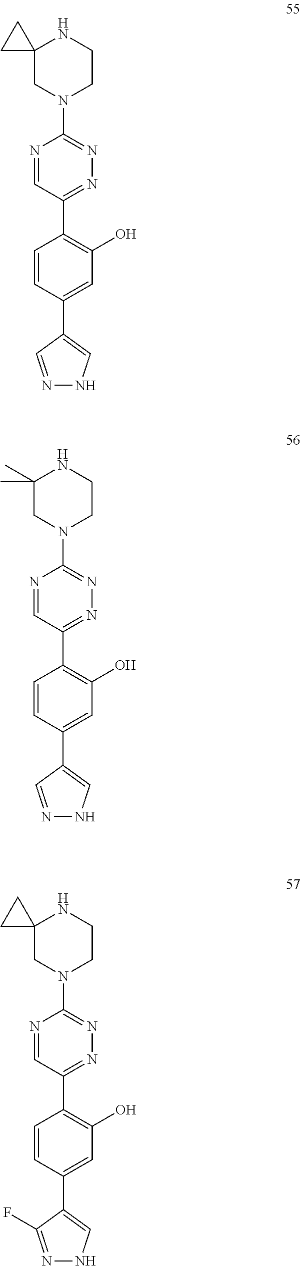 [SMILES: Oc1cc(-c2cn[nH]c2)ccc1-c1cnc(N2CCNC3(CC3)C2)nn1 ; CC1(C)CN(c2ncc(-c3ccc(-c4cn[nH]c4)cc3O)nn2)CCN1 ; Oc1cc(-c2c[nH]nc2F)ccc1-c1cnc(N2CCNC3(CC3)C2)nn1]

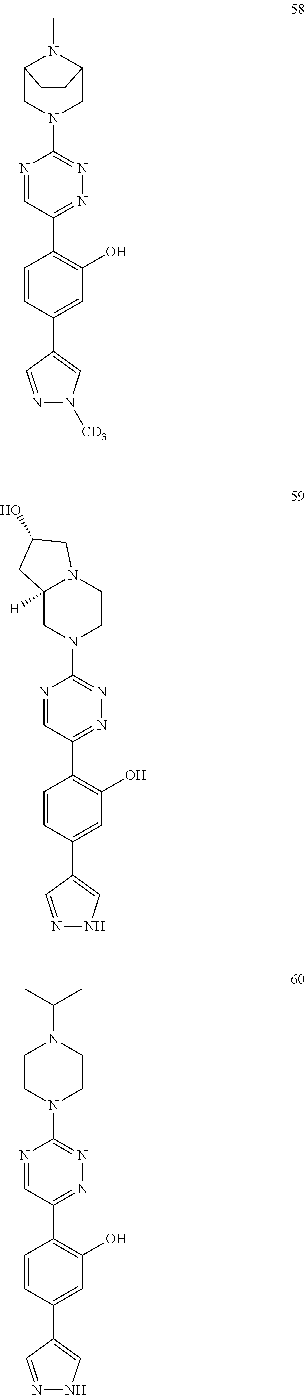 [SMILES: [2H]C([2H])([2H])n1cc(-c2ccc(-c3cnc(N4CC5CCC(C4)N5C)nn3)c(O)c2)cn1 ; Oc1cc(-c2cn[nH]c2)ccc1-c1cnc(N2CCN3C[C@@H](O)C[C@@H]3C2)nn1 ; CC(C)N1CCN(c2ncc(-c3ccc(-c4cn[nH]c4)cc3O)nn2)CC1]

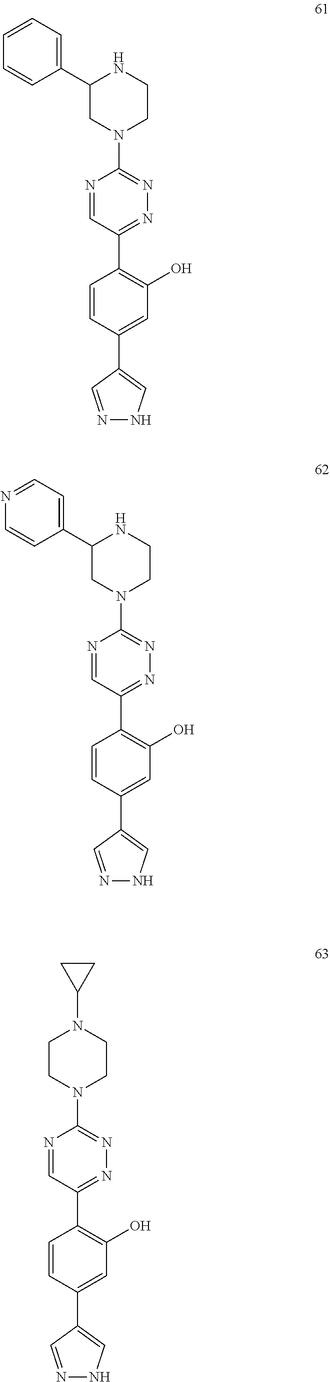 [SMILES: Oc1cc(-c2cn[nH]c2)ccc1-c1cnc(N2CCNC(c3ccccc3)C2)nn1 ; Oc1cc(-c2cn[nH]c2)ccc1-c1cnc(N2CCNC(c3ccncc3)C2)nn1 ; Oc1cc(-c2cn[nH]c2)ccc1-c1cnc(N2CCN(C3CC3)CC2)nn1]

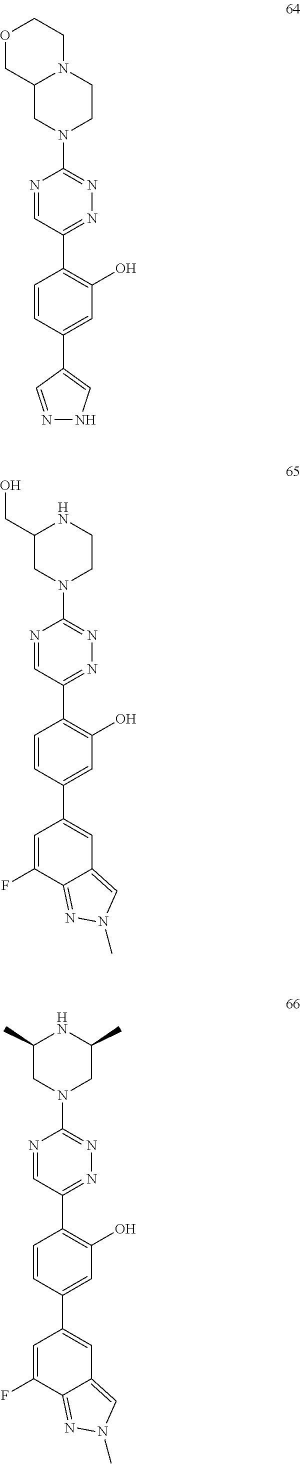

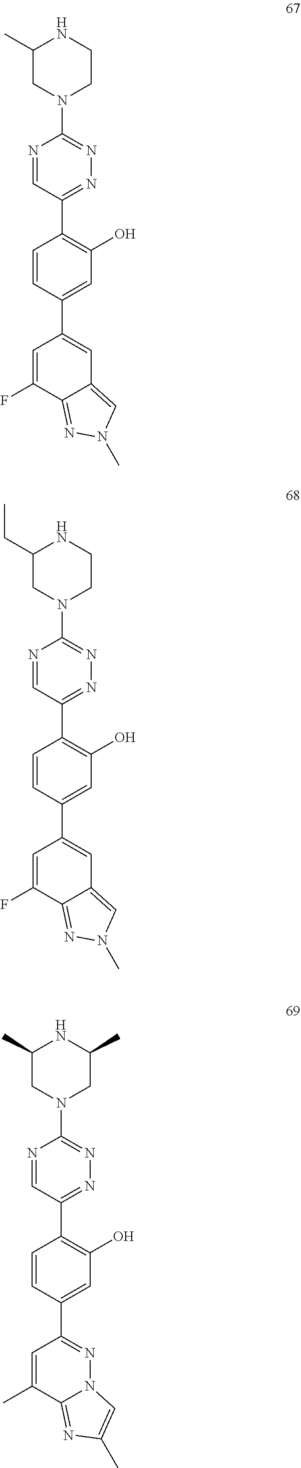

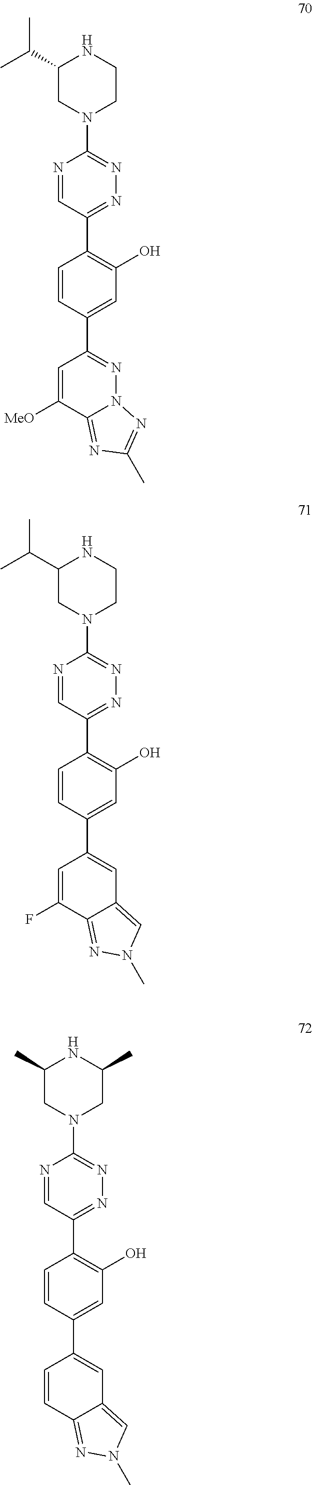

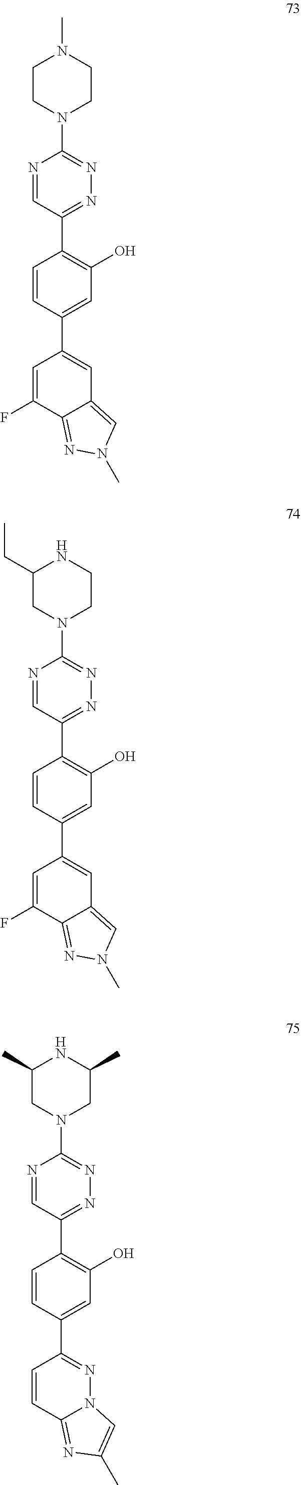

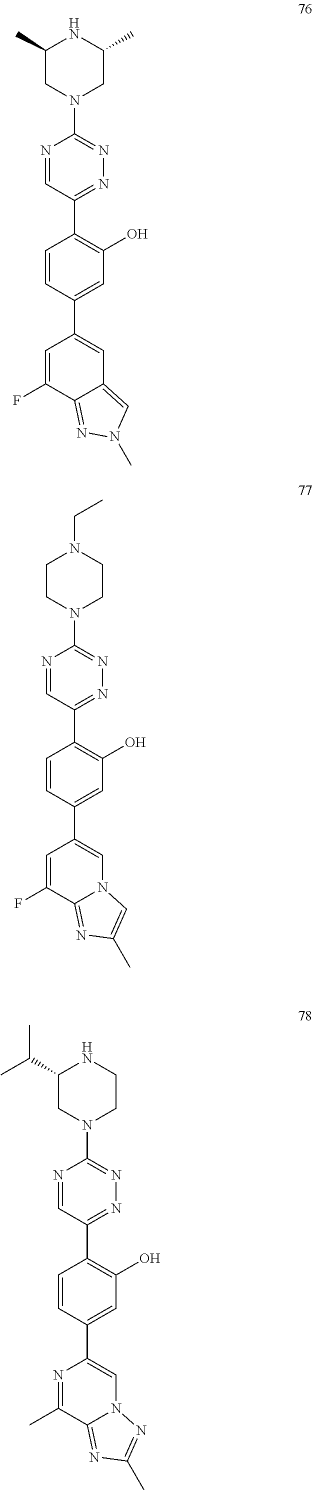

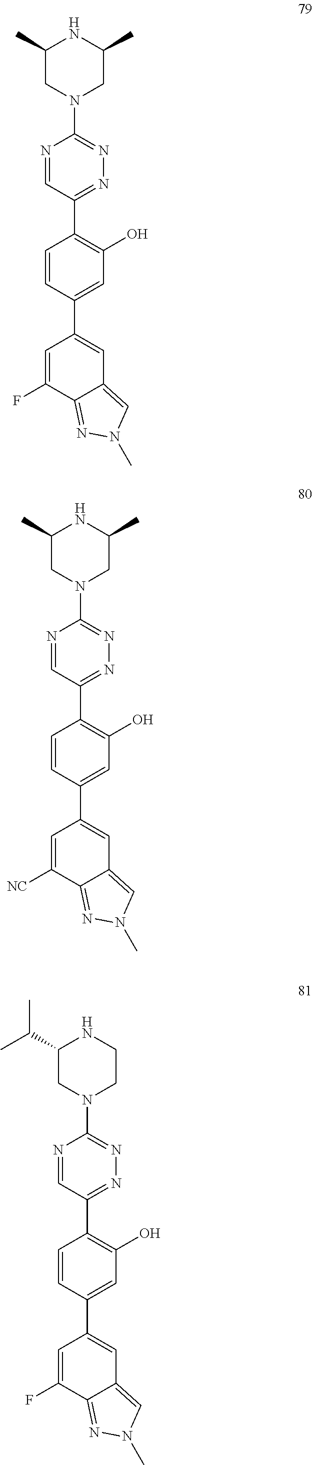

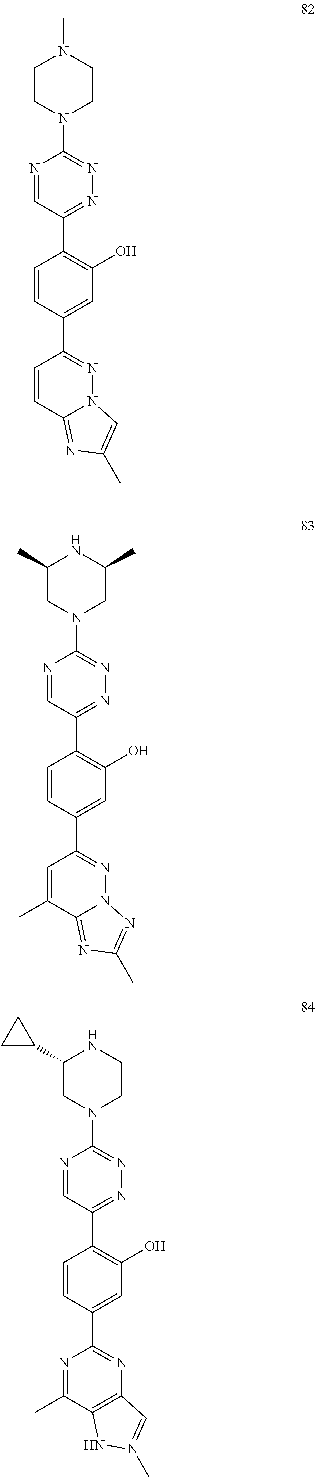

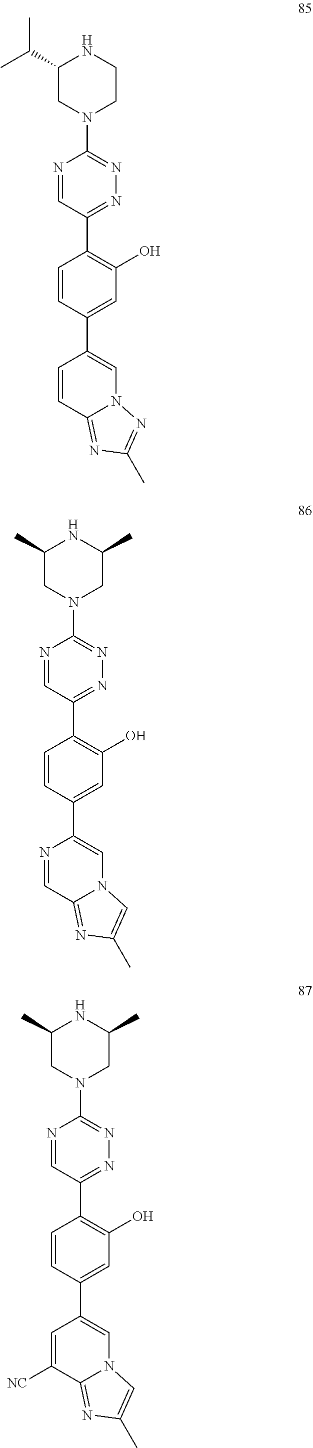

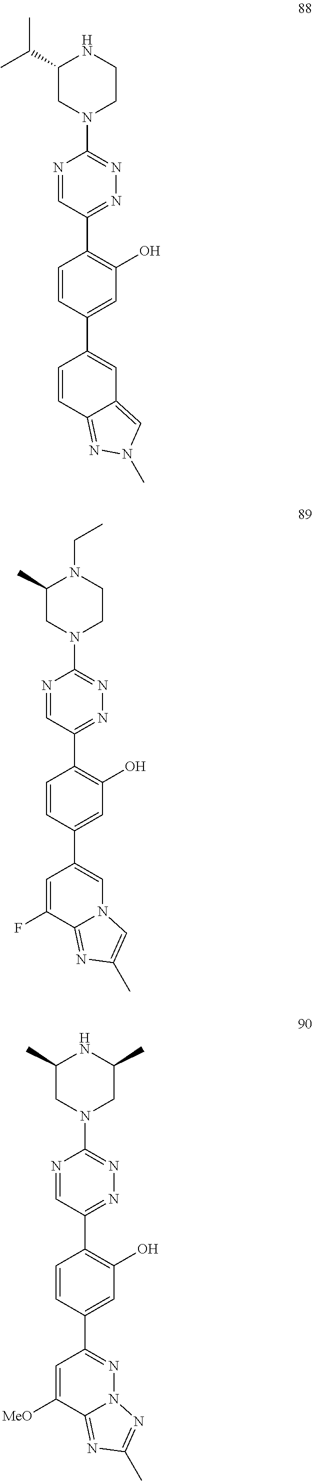

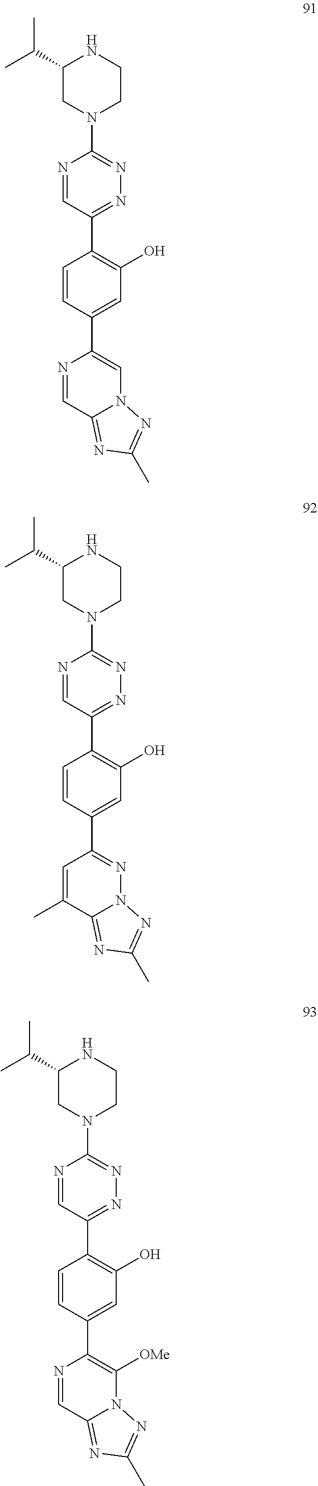 [SMILES: Cc1nc2cnc(-c3ccc(-c4cnc(N5CCN[C@@H](C(C)C)C5)nn4)c(O)c3)cn2n1 ; Cc1nc2c(C)cc(-c3ccc(-c4cnc(N5CCN[C@@H](C(C)C)C5)nn4)c(O)c3)nn2n1 ; COc1c(-c2ccc(-c3cnc(N4CCN[C@@H](C(C)C)C4)nn3)c(O)c2)ncc2nc(C)nn12]

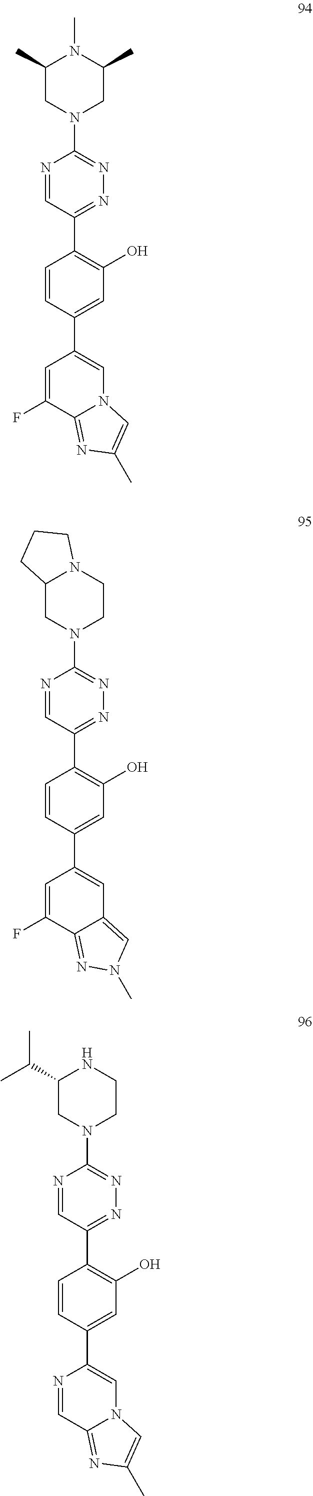

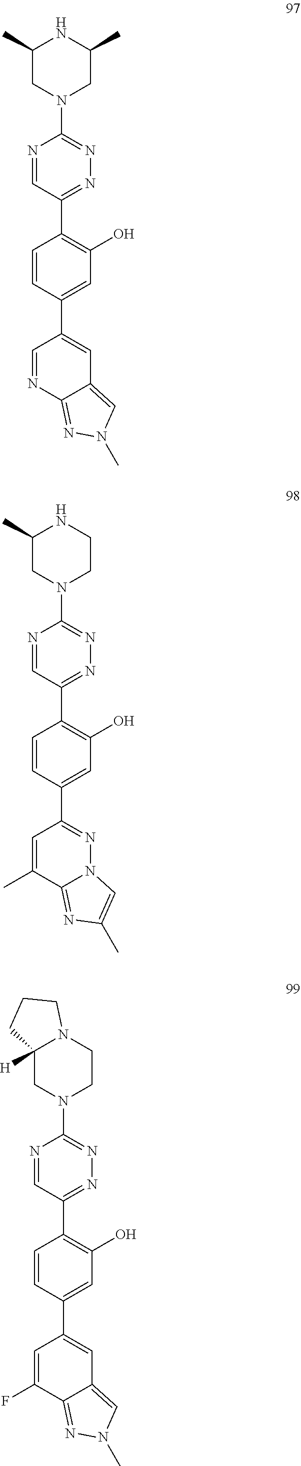

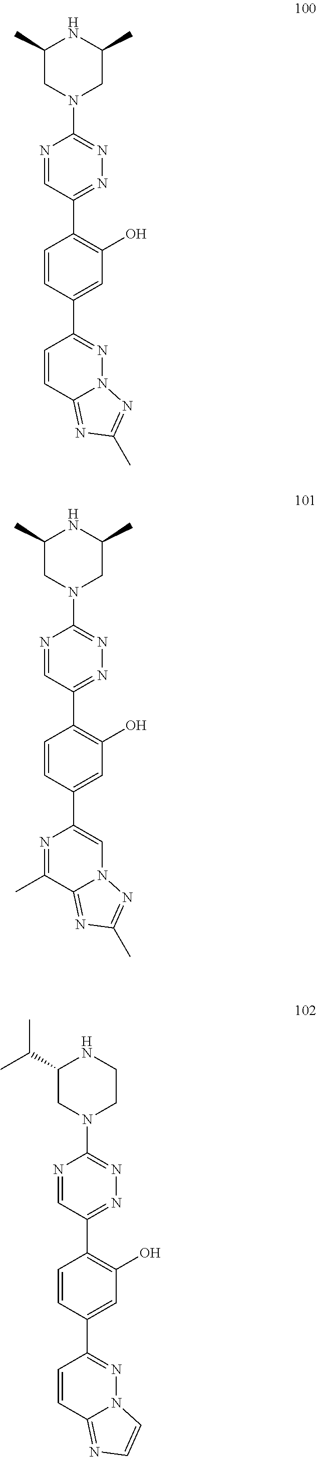

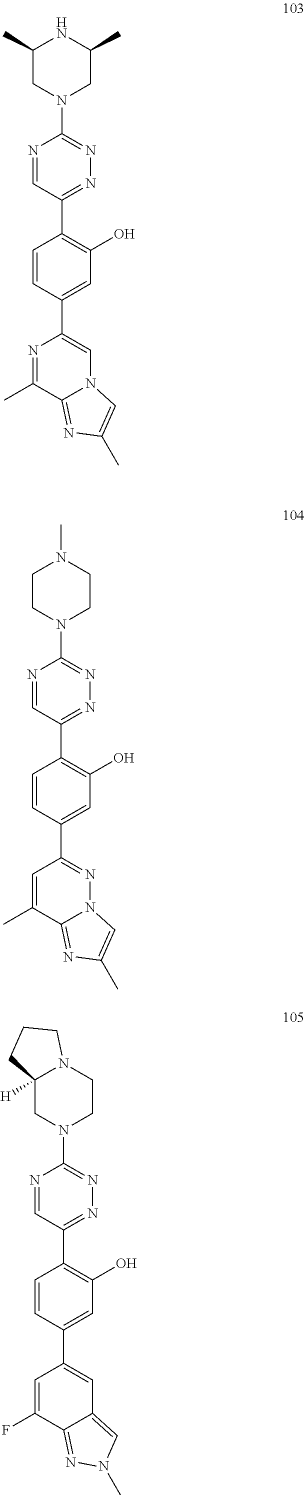

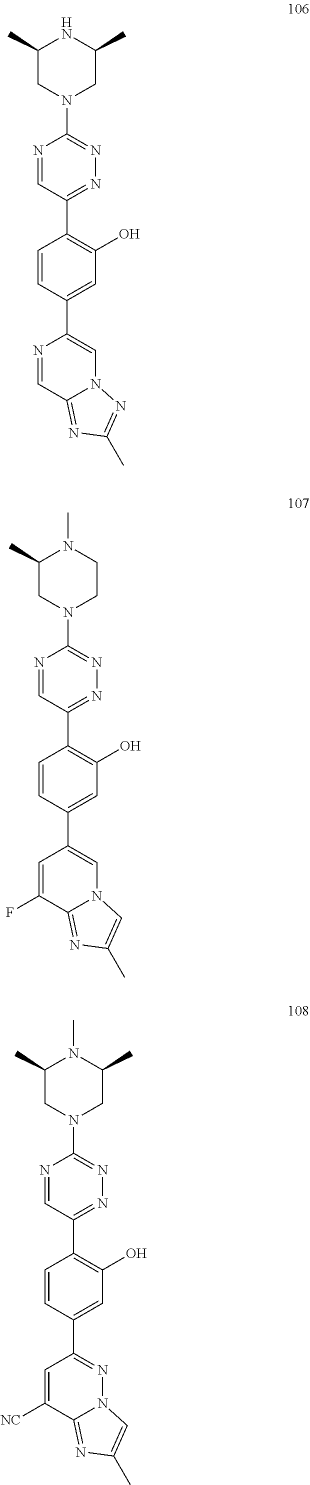

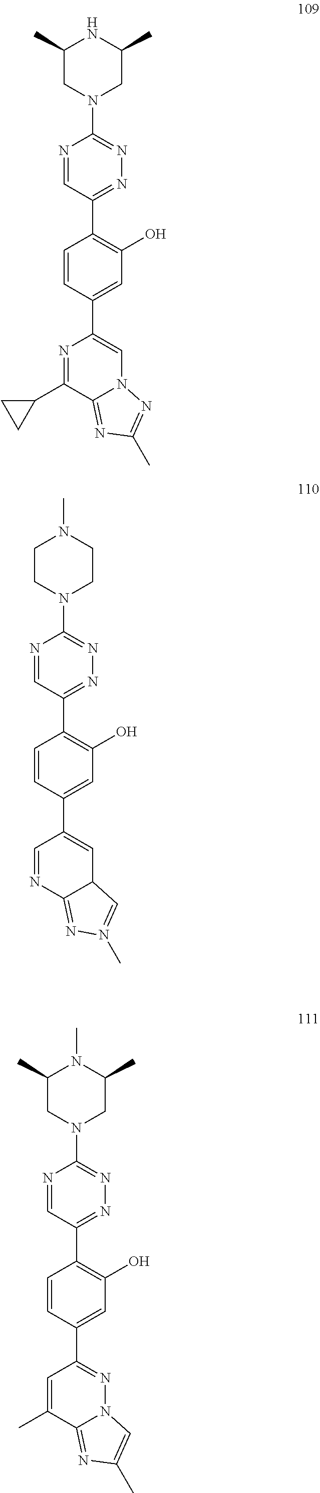

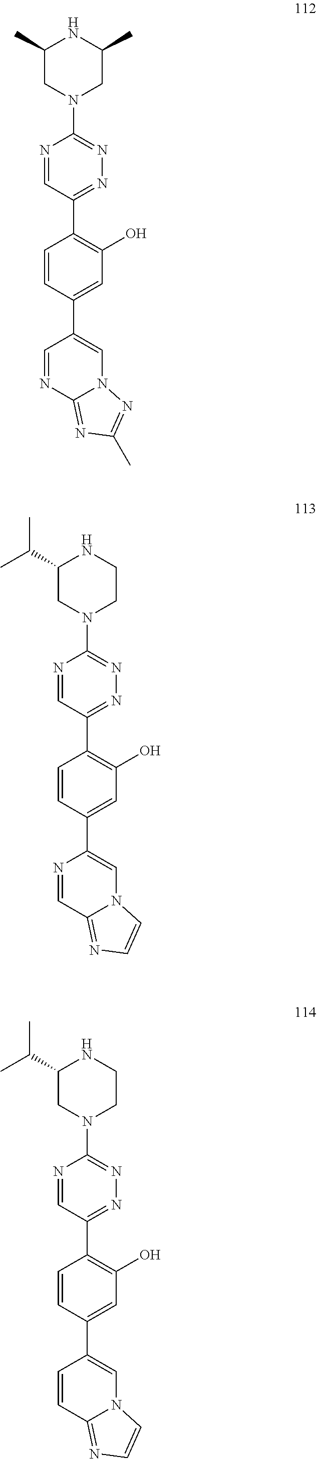 [SMILES: Cc1nc2ncc(-c3ccc(-c4cnc(N5C[C@@H](C)N[C@@H](C)C5)nn4)c(O)c3)cn2n1 ; CC(C)[C@H]1CN(c2ncc(-c3ccc(-c4cn5ccnc5cn4)cc3O)nn2)CCN1 ; CC(C)[C@H]1CN(c2ncc(-c3ccc(-c4ccc5nccn5c4)cc3O)nn2)CCN1]

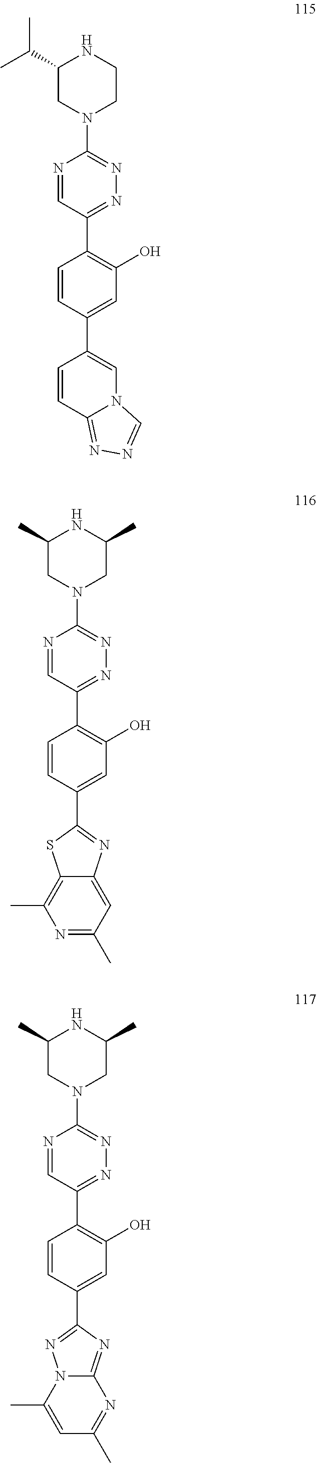

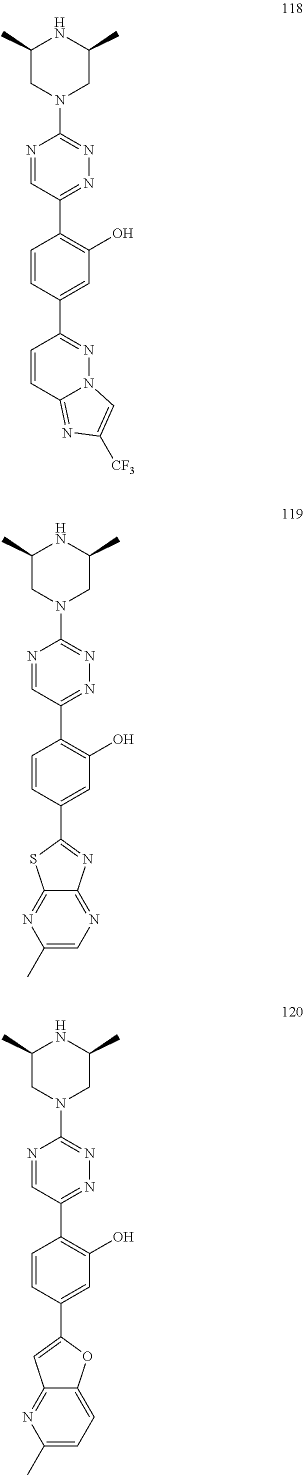

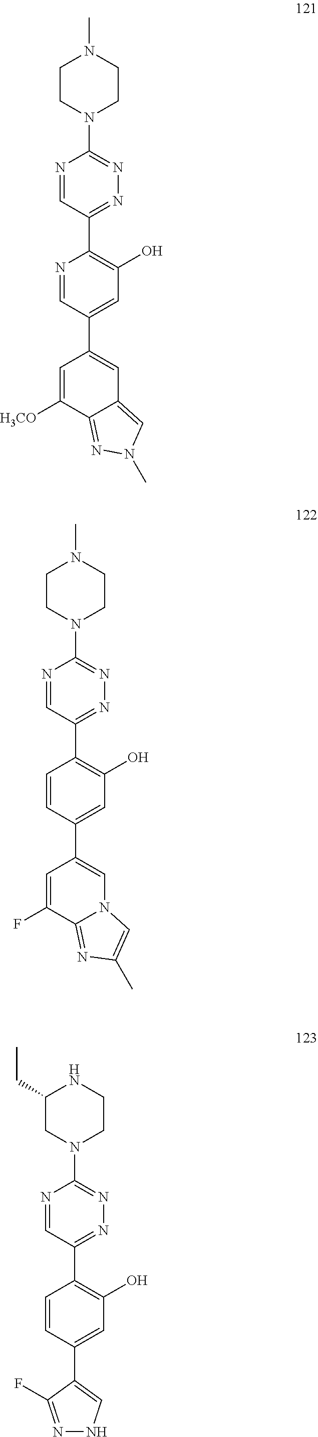 [SMILES: COc1cc(-c2cnc(-c3cnc(N4CCN(C)CC4)nn3)c(O)c2)cc2cn(C)nc12 ; Cc1cn2cc(-c3ccc(-c4cnc(N5CCN(C)CC5)nn4)c(O)c3)cc(F)c2n1 ; CC[C@H]1CN(c2ncc(-c3ccc(-c4c[nH]nc4F)cc3O)nn2)CCN1]

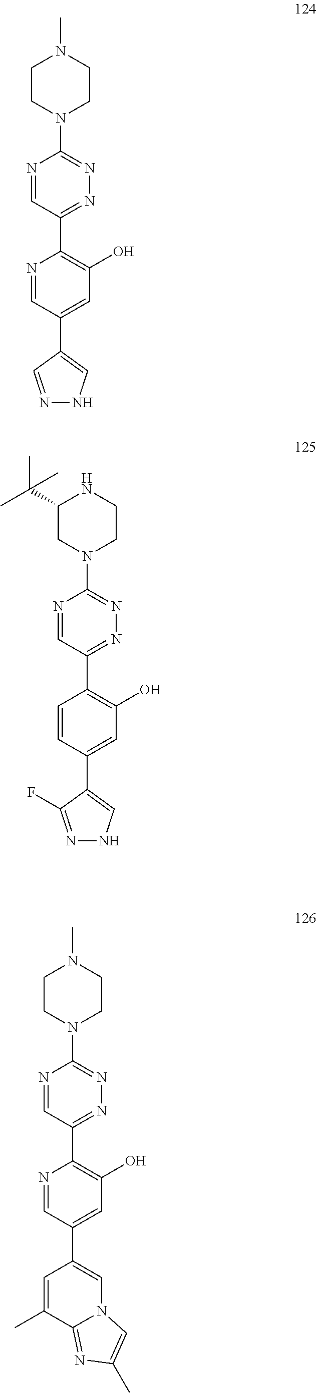 [SMILES: CN1CCN(c2ncc(-c3ncc(-c4cn[nH]c4)cc3O)nn2)CC1 ; CC(C)(C)[C@H]1CN(c2ncc(-c3ccc(-c4c[nH]nc4F)cc3O)nn2)CCN1 ; Cc1cn2cc(-c3cnc(-c4cnc(N5CCN(C)CC5)nn4)c(O)c3)cc(C)c2n1]

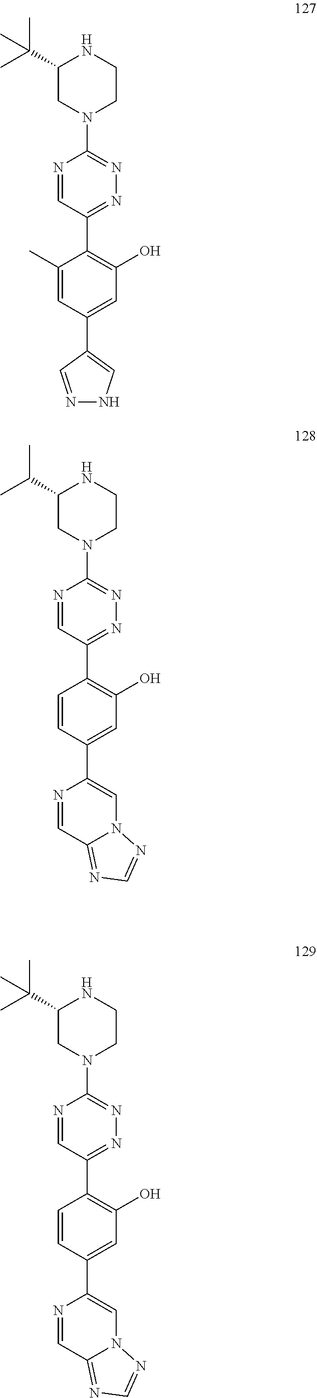 [SMILES: Cc1cc(-c2cn[nH]c2)cc(O)c1-c1cnc(N2CCN[C@@H](C(C)(C)C)C2)nn1 ; CC(C)[C@H]1CN(c2ncc(-c3ccc(-c4cn5ncnc5cn4)cc3O)nn2)CCN1 ; CC(C)(C)[C@H]1CN(c2ncc(-c3ccc(-c4cn5ncnc5cn4)cc3O)nn2)CCN1]

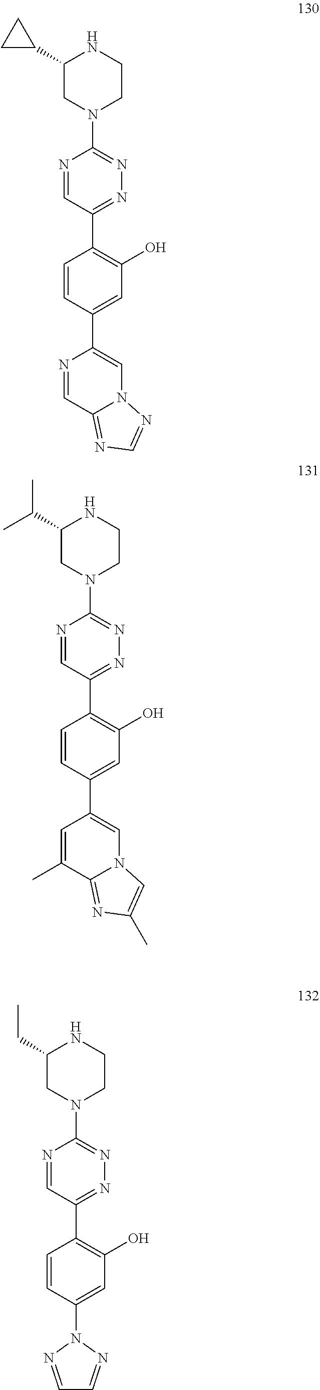 [SMILES: Oc1cc(-c2cn3ncnc3cn2)ccc1-c1cnc(N2CCN[C@@H](C3CC3)C2)nn1 ; Cc1cn2cc(-c3ccc(-c4cnc(N5CCN[C@@H](C(C)C)C5)nn4)c(O)c3)cc(C)c2n1 ; CC[C@H]1CN(c2ncc(-c3ccc(-n4nccn4)cc3O)nn2)CCN1]

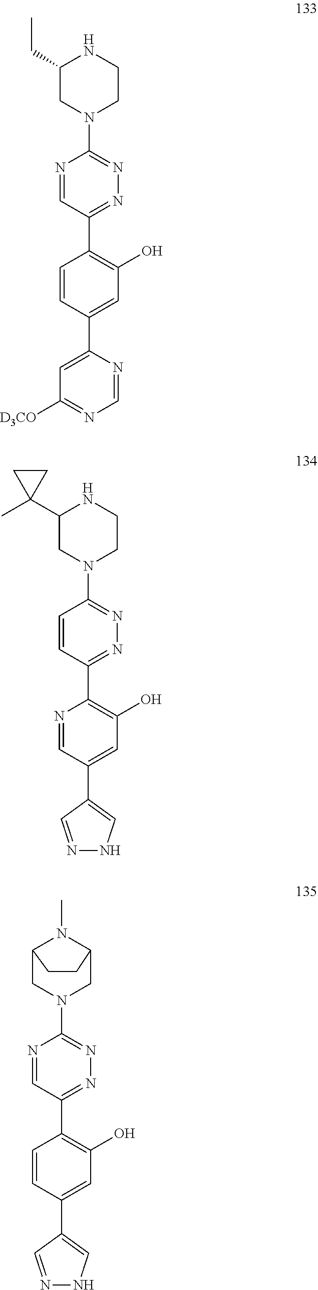

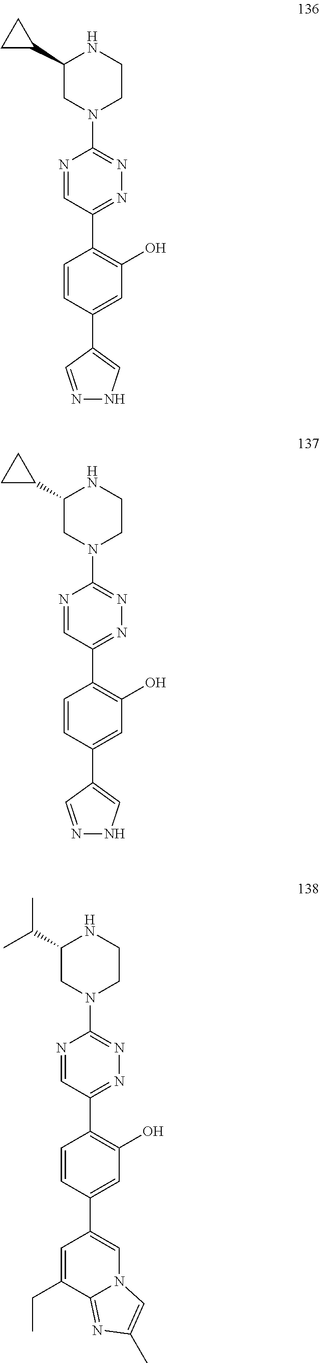 [SMILES: Oc1cc(-c2cn[nH]c2)ccc1-c1cnc(N2CCN[C@H](C3CC3)C2)nn1 ; Oc1cc(-c2cn[nH]c2)ccc1-c1cnc(N2CCN[C@@H](C3CC3)C2)nn1 ; CCc1cc(-c2ccc(-c3cnc(N4CCN[C@@H](C(C)C)C4)nn3)c(O)c2)cn2cc(C)nc12]

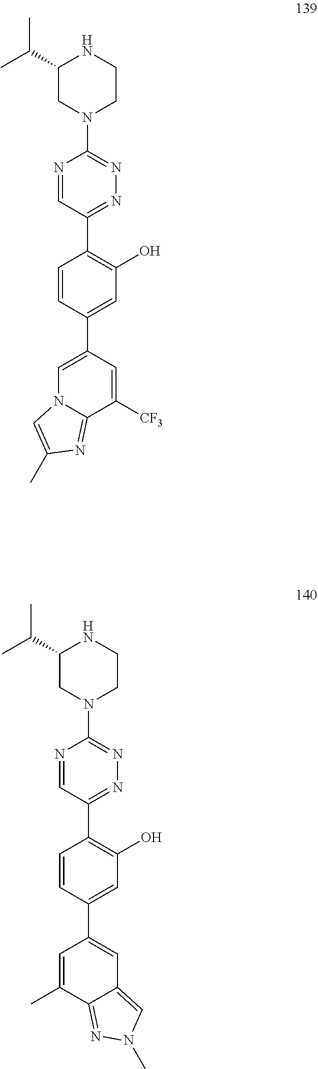 [SMILES: Cc1cn2cc(-c3ccc(-c4cnc(N5CCN[C@@H](C(C)C)C5)nn4)c(O)c3)cc(C(F)(F)F)c2n1 ; Cc1cc(-c2ccc(-c3cnc(N4CCN[C@@H](C(C)C)C4)nn3)c(O)c2)cc2cn(C)nc12]

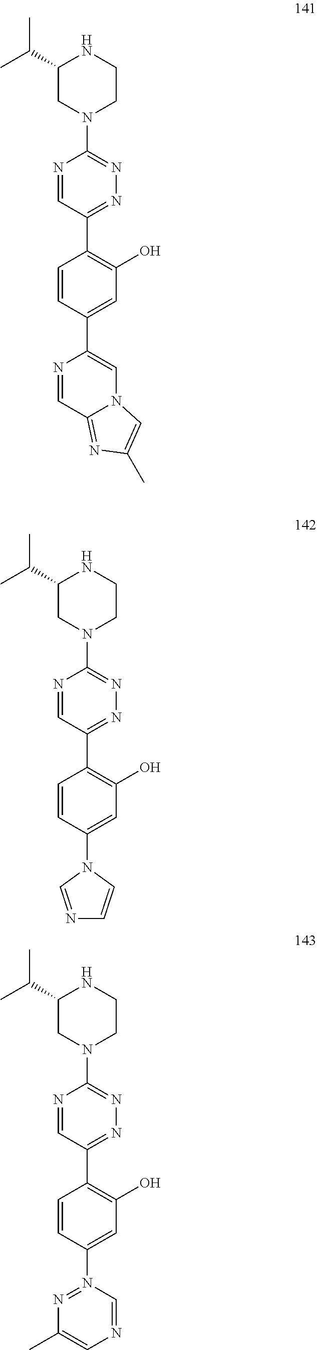

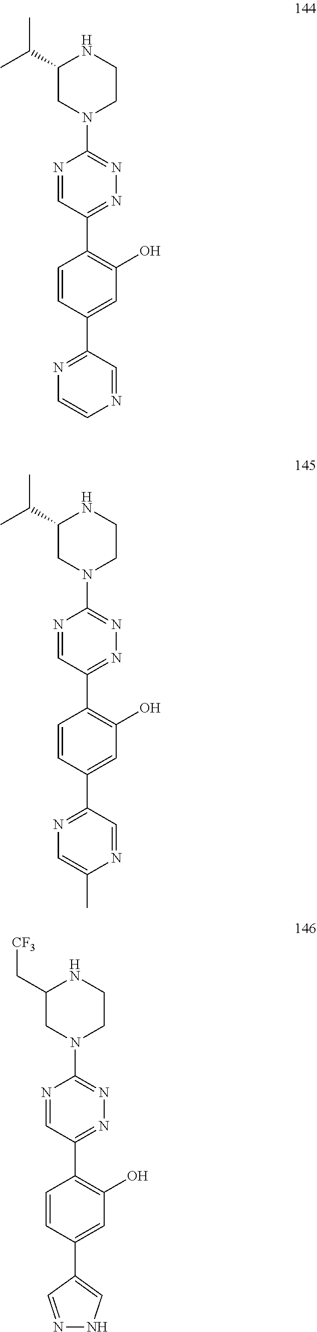 [SMILES: CC(C)[C@H]1CN(c2ncc(-c3ccc(-c4cnccn4)cc3O)nn2)CCN1 ; Cc1cnc(-c2ccc(-c3cnc(N4CCN[C@@H](C(C)C)C4)nn3)c(O)c2)cn1 ; Oc1cc(-c2cn[nH]c2)ccc1-c1cnc(N2CCNC(CC(F)(F)F)C2)nn1]

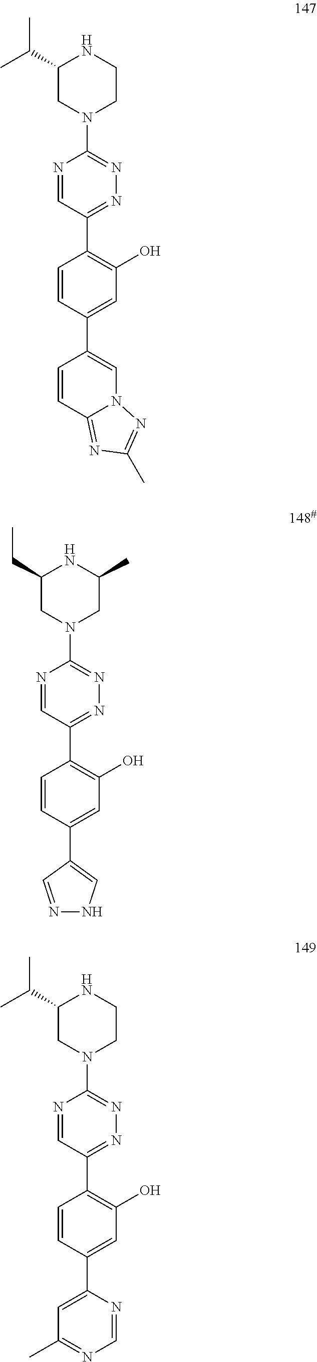

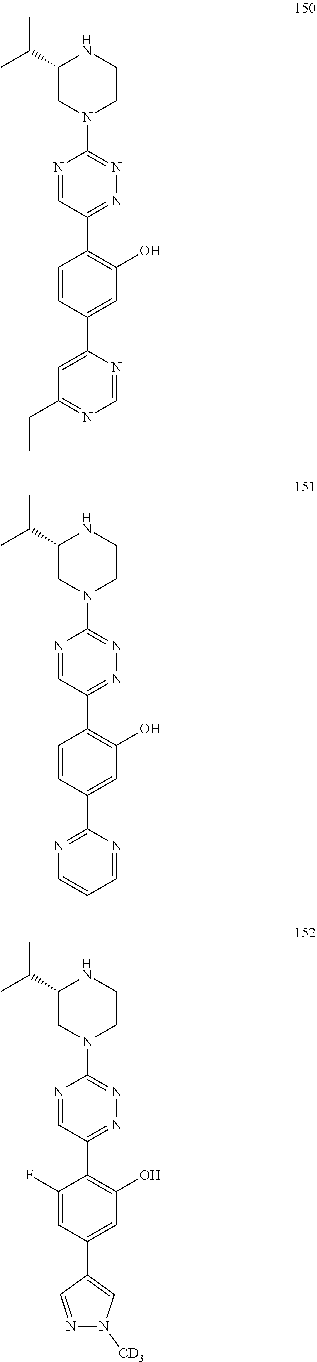

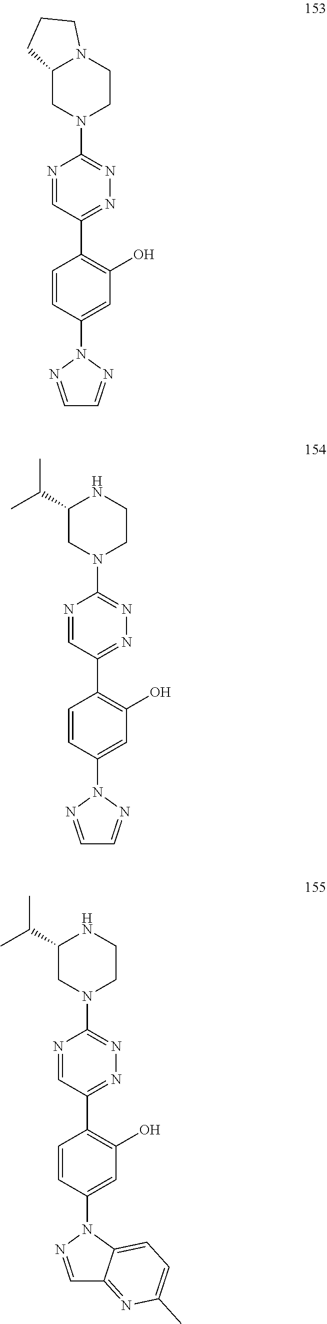 [SMILES: Oc1cc(-n2nccn2)ccc1-c1cnc(N2CCN3CCC[C@H]3C2)nn1 ; CC(C)[C@H]1CN(c2ncc(-c3ccc(-n4nccn4)cc3O)nn2)CCN1 ; Cc1ccc2c(cnn2-c2ccc(-c3cnc(N4CCN[C@@H](C(C)C)C4)nn3)c(O)c2)n1]

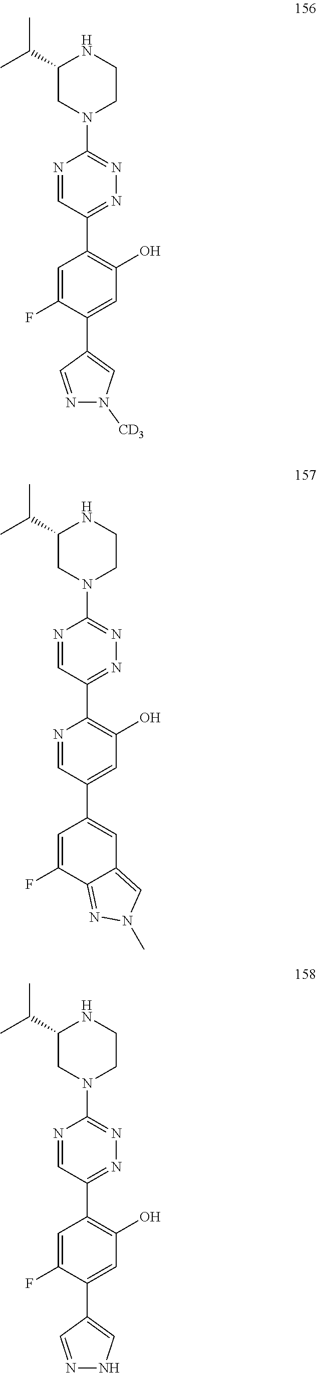 [SMILES: [2H]C([2H])([2H])n1cc(-c2cc(O)c(-c3cnc(N4CCN[C@@H](C(C)C)C4)nn3)cc2F)cn1 ; CC(C)[C@H]1CN(c2ncc(-c3ncc(-c4cc(F)c5nn(C)cc5c4)cc3O)nn2)CCN1 ; CC(C)[C@H]1CN(c2ncc(-c3cc(F)c(-c4cn[nH]c4)cc3O)nn2)CCN1]

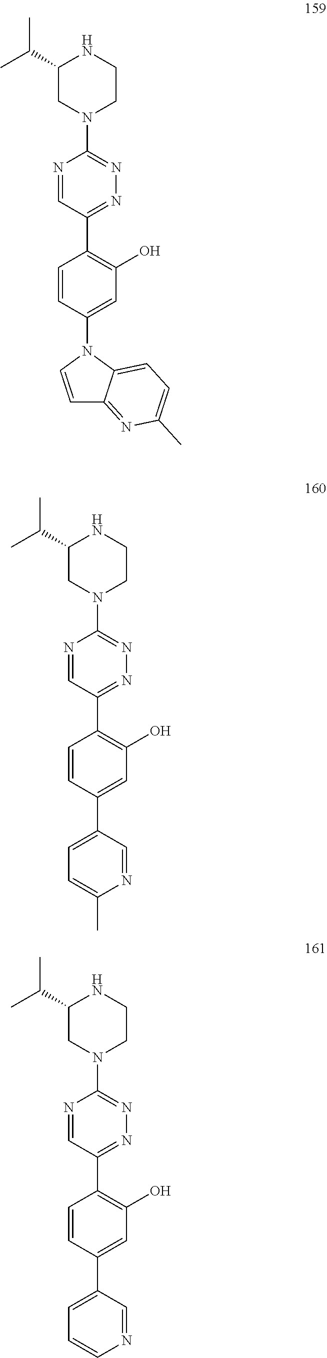

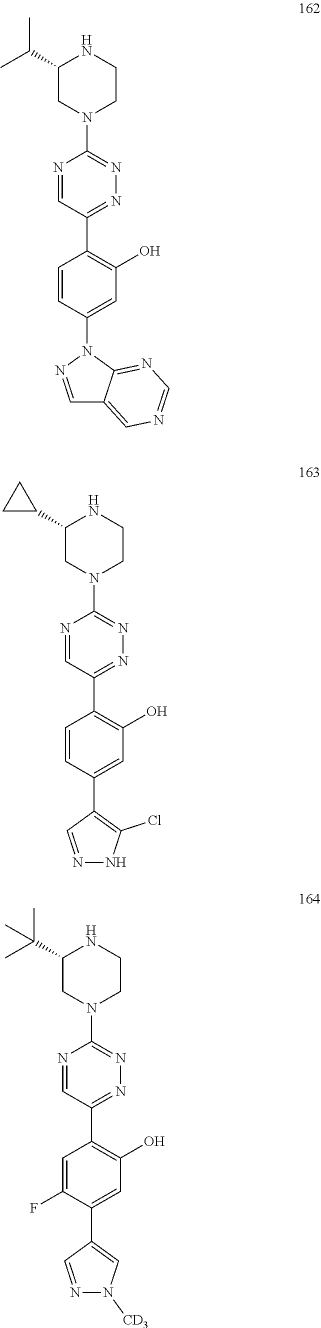 [SMILES: CC(C)[C@H]1CN(c2ncc(-c3ccc(-n4ncc5cncnc54)cc3O)nn2)CCN1 ; Oc1cc(-c2cn[nH]c2Cl)ccc1-c1cnc(N2CCN[C@@H](C3CC3)C2)nn1 ; [2H]C([2H])([2H])n1cc(-c2cc(O)c(-c3cnc(N4CCN[C@@H](C(C)(C)C)C4)nn3)cc2F)cn1]

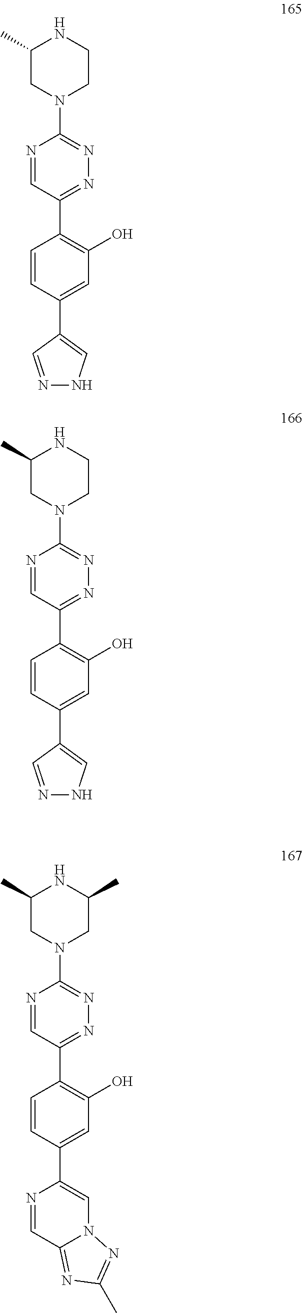

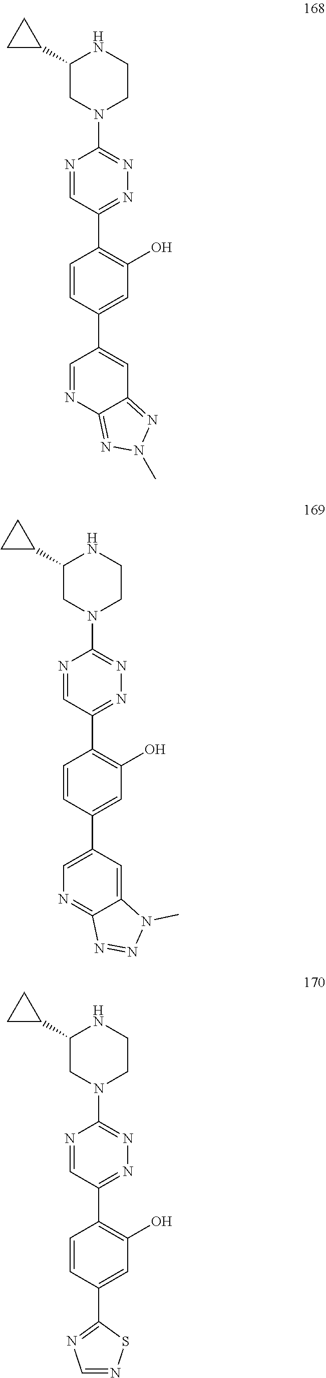 [SMILES: Cn1nc2cc(-c3ccc(-c4cnc(N5CCN[C@@H](C6CC6)C5)nn4)c(O)c3)cnc2n1 ; Cn1nnc2ncc(-c3ccc(-c4cnc(N5CCN[C@@H](C6CC6)C5)nn4)c(O)c3)cc21 ; Oc1cc(-c2ncns2)ccc1-c1cnc(N2CCN[C@@H](C3CC3)C2)nn1]

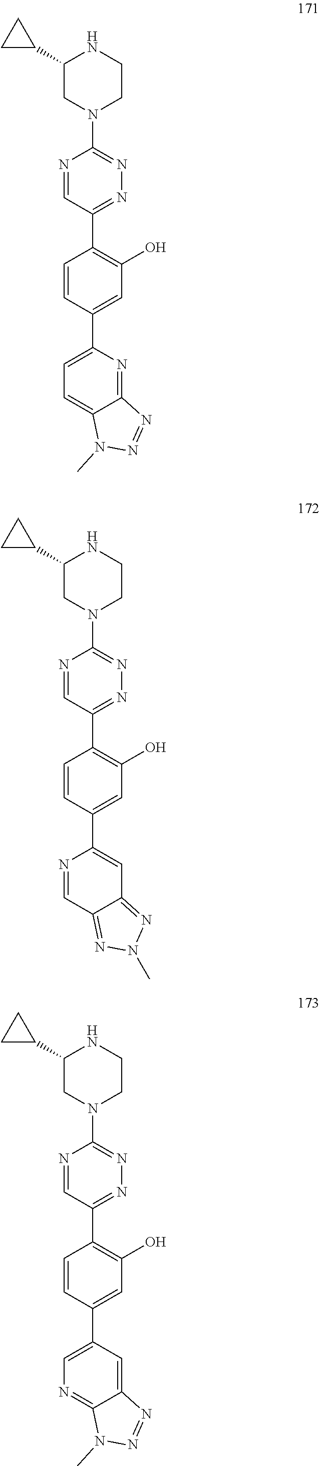 [SMILES: Cn1nnc2nc(-c3ccc(-c4cnc(N5CCN[C@@H](C6CC6)C5)nn4)c(O)c3)ccc21 ; Cn1nc2cnc(-c3ccc(-c4cnc(N5CCN[C@@H](C6CC6)C5)nn4)c(O)c3)cc2n1 ; Cn1nnc2cc(-c3ccc(-c4cnc(N5CCN[C@@H](C6CC6)C5)nn4)c(O)c3)cnc21]

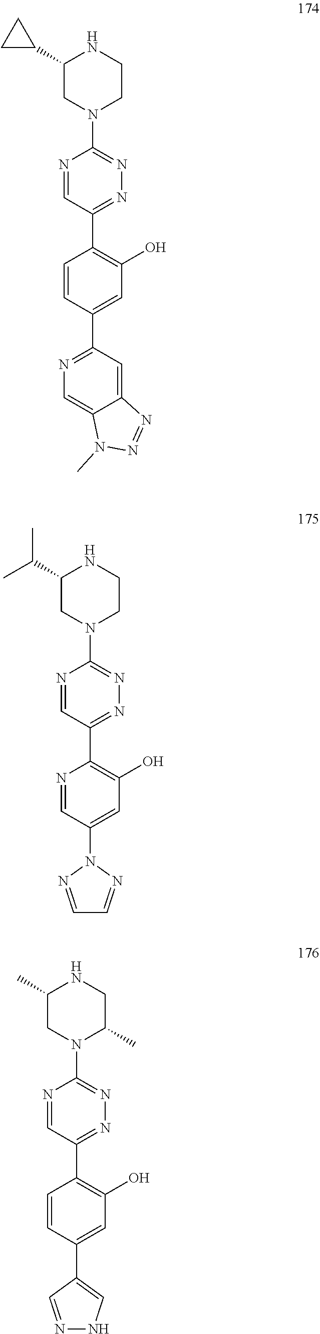

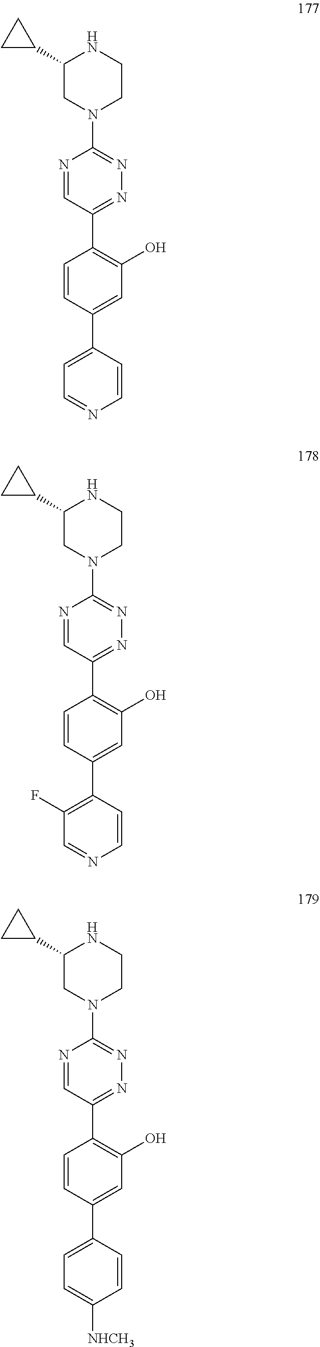 [SMILES: Oc1cc(-c2ccncc2)ccc1-c1cnc(N2CCN[C@@H](C3CC3)C2)nn1 ; Oc1cc(-c2ccncc2F)ccc1-c1cnc(N2CCN[C@@H](C3CC3)C2)nn1 ; CNc1ccc(-c2ccc(-c3cnc(N4CCN[C@@H](C5CC5)C4)nn3)c(O)c2)cc1]

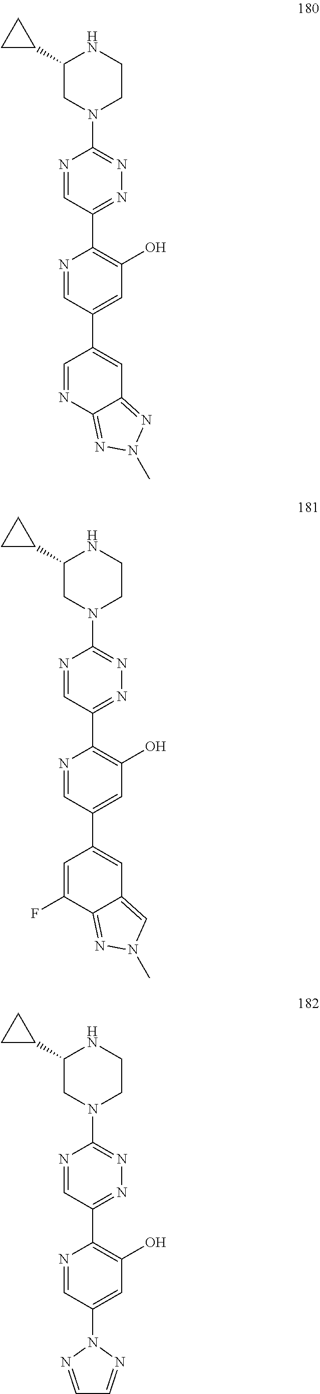 [SMILES: Cn1nc2cc(-c3cnc(-c4cnc(N5CCN[C@@H](C6CC6)C5)nn4)c(O)c3)cnc2n1 ; Cn1cc2cc(-c3cnc(-c4cnc(N5CCN[C@@H](C6CC6)C5)nn4)c(O)c3)cc(F)c2n1 ; Oc1cc(-n2nccn2)cnc1-c1cnc(N2CCN[C@@H](C3CC3)C2)nn1]

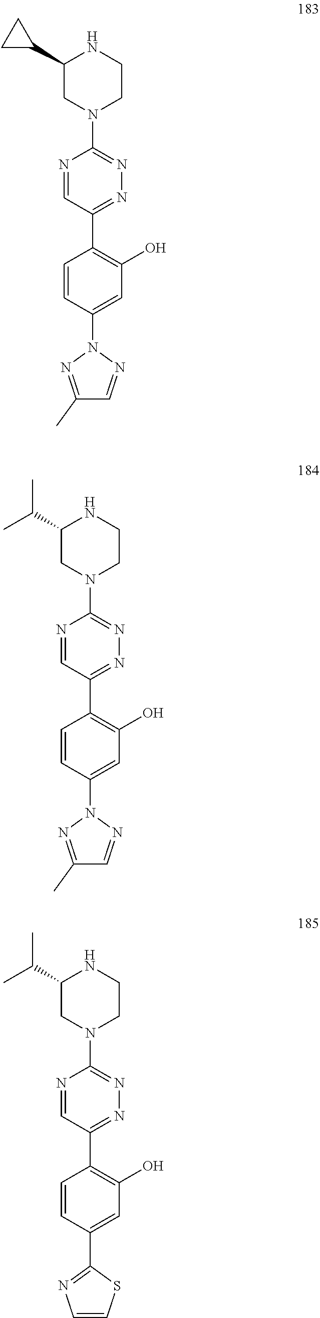 [SMILES: Cc1cnn(-c2ccc(-c3cnc(N4CCN[C@H](C5CC5)C4)nn3)c(O)c2)n1 ; Cc1cnn(-c2ccc(-c3cnc(N4CCN[C@@H](C(C)C)C4)nn3)c(O)c2)n1 ; CC(C)[C@H]1CN(c2ncc(-c3ccc(-c4nccs4)cc3O)nn2)CCN1]

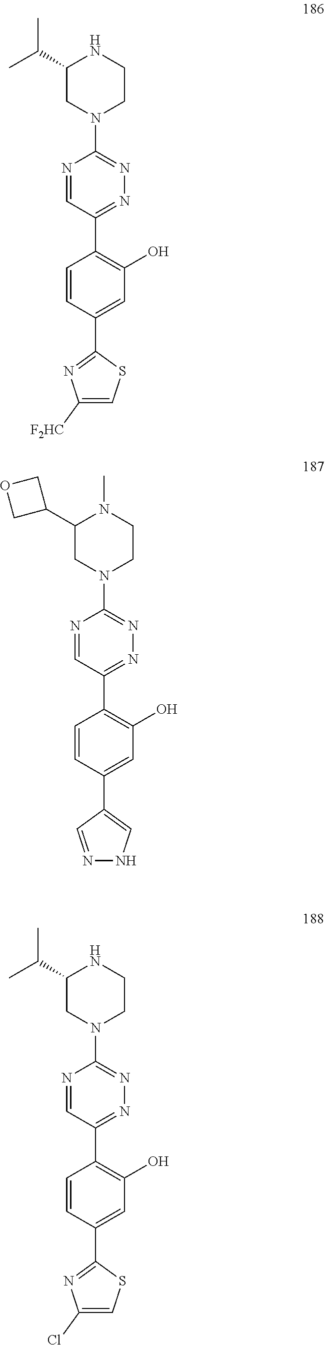 [SMILES: CC(C)[C@H]1CN(c2ncc(-c3ccc(-c4nc(C(F)F)cs4)cc3O)nn2)CCN1 ; CN1CCN(c2ncc(-c3ccc(-c4cn[nH]c4)cc3O)nn2)CC1C1COC1 ; CC(C)[C@H]1CN(c2ncc(-c3ccc(-c4nc(Cl)cs4)cc3O)nn2)CCN1]

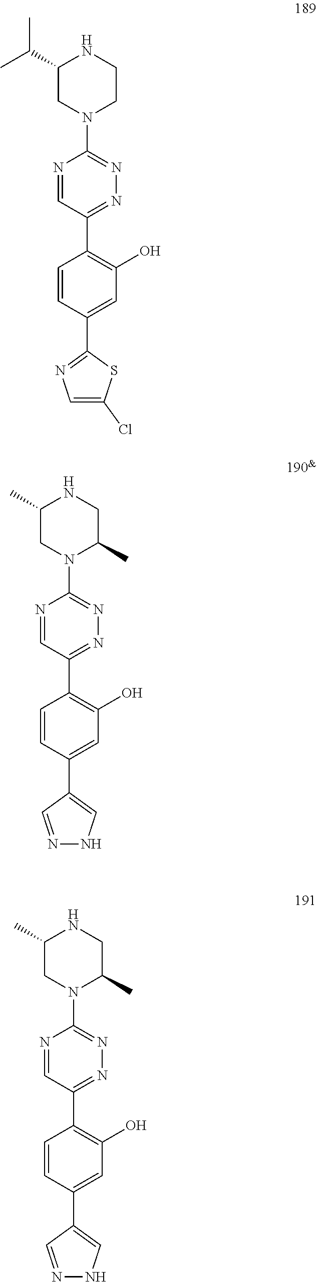

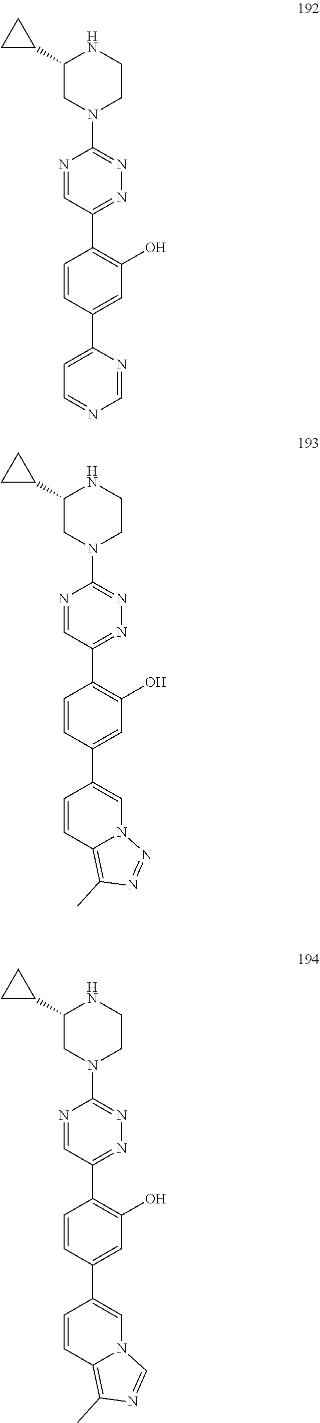 [SMILES: Oc1cc(-c2ccncn2)ccc1-c1cnc(N2CCN[C@@H](C3CC3)C2)nn1 ; Cc1nnn2cc(-c3ccc(-c4cnc(N5CCN[C@@H](C6CC6)C5)nn4)c(O)c3)ccc12 ; Cc1ncn2cc(-c3ccc(-c4cnc(N5CCN[C@@H](C6CC6)C5)nn4)c(O)c3)ccc12]

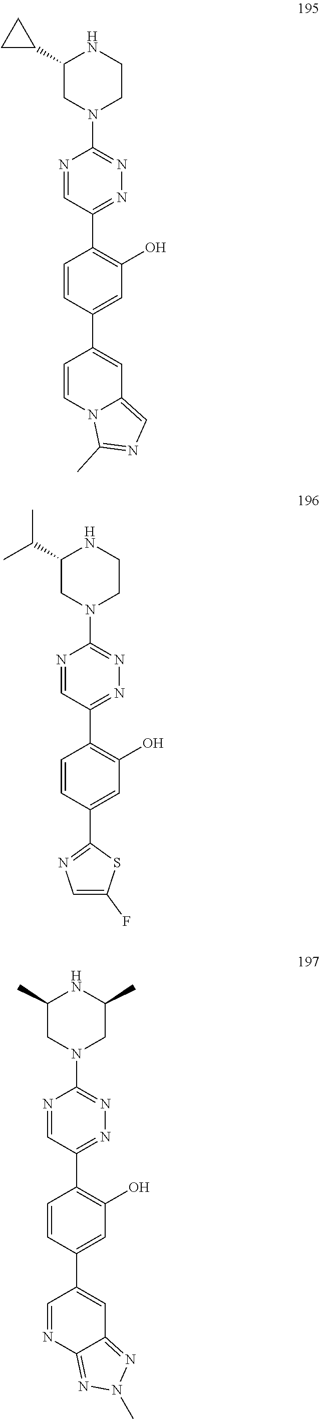

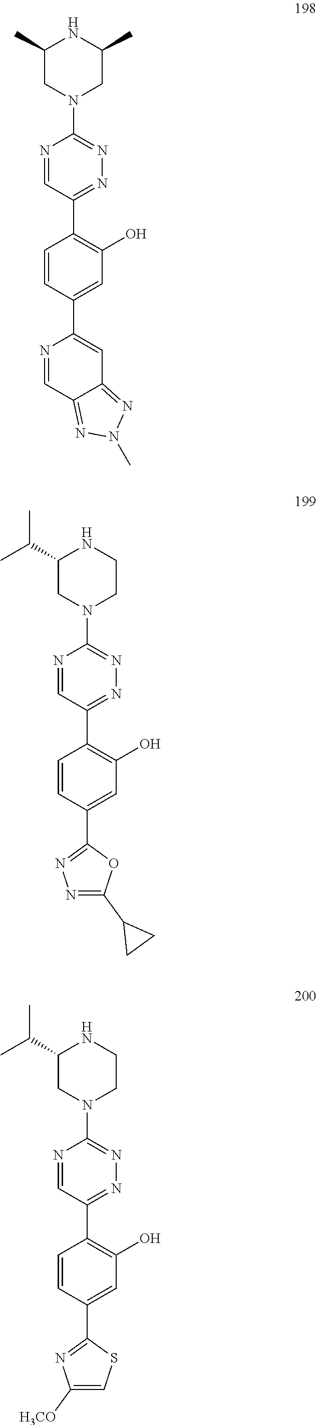

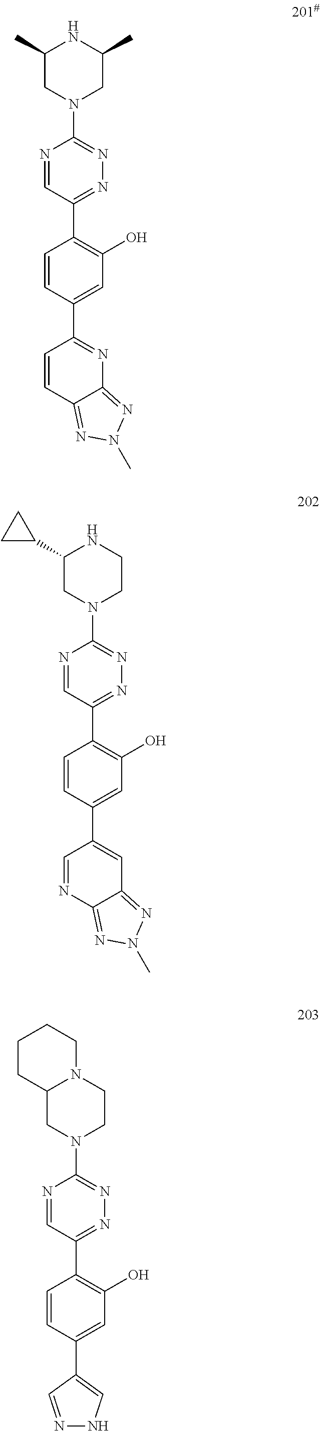

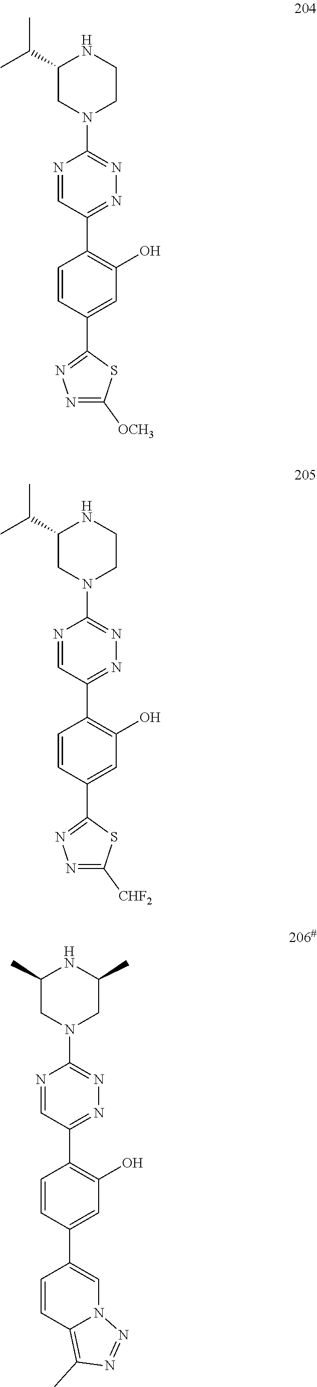

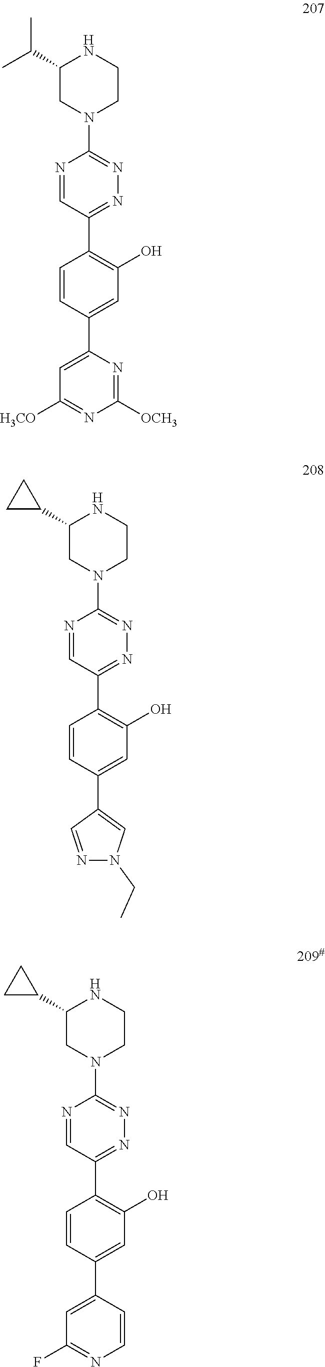 [SMILES: COc1cc(-c2ccc(-c3cnc(N4CCN[C@@H](C(C)C)C4)nn3)c(O)c2)nc(OC)n1 ; CCn1cc(-c2ccc(-c3cnc(N4CCN[C@@H](C5CC5)C4)nn3)c(O)c2)cn1 ; Oc1cc(-c2ccnc(F)c2)ccc1-c1cnc(N2CCN[C@@H](C3CC3)C2)nn1]

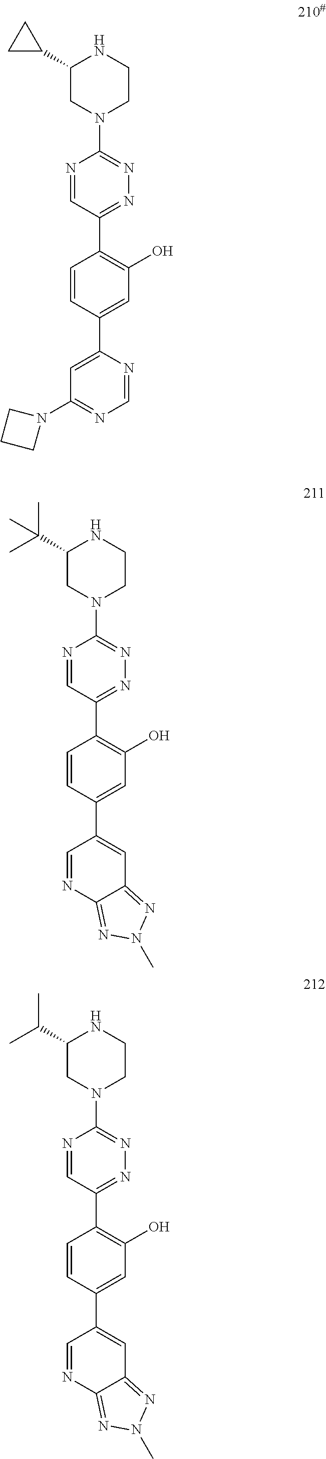 [SMILES: Oc1cc(-c2cc(N3CCC3)ncn2)ccc1-c1cnc(N2CCN[C@@H](C3CC3)C2)nn1 ; Cn1nc2cc(-c3ccc(-c4cnc(N5CCN[C@@H](C(C)(C)C)C5)nn4)c(O)c3)cnc2n1 ; CC(C)[C@H]1CN(c2ncc(-c3ccc(-c4cnc5nn(C)nc5c4)cc3O)nn2)CCN1]

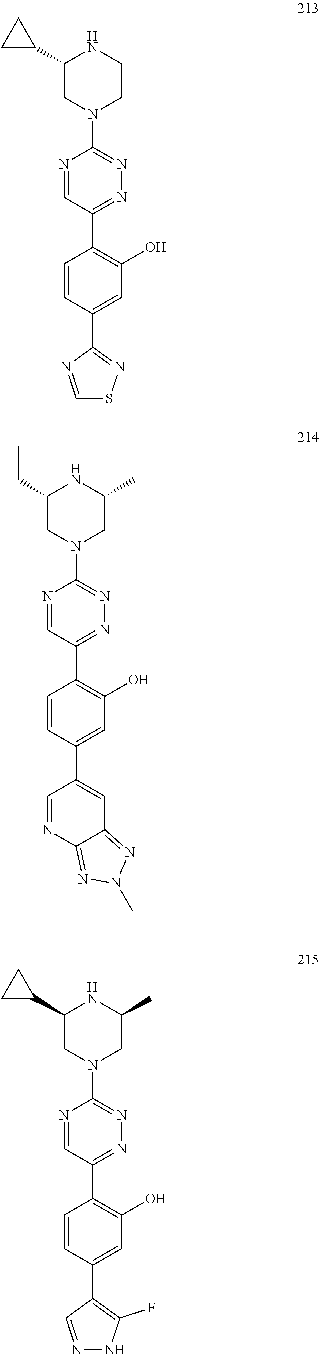 [SMILES: Oc1cc(-c2ncsn2)ccc1-c1cnc(N2CCN[C@@H](C3CC3)C2)nn1 ; CC[C@H]1CN(c2ncc(-c3ccc(-c4cnc5nn(C)nc5c4)cc3O)nn2)C[C@@H](C)N1 ; C[C@H]1CN(c2ncc(-c3ccc(-c4cn[nH]c4F)cc3O)nn2)C[C@@H](C2CC2)N1]

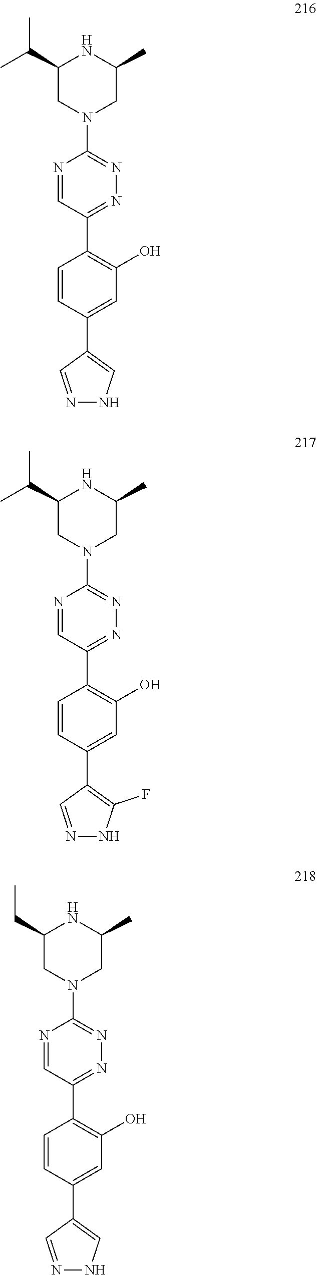

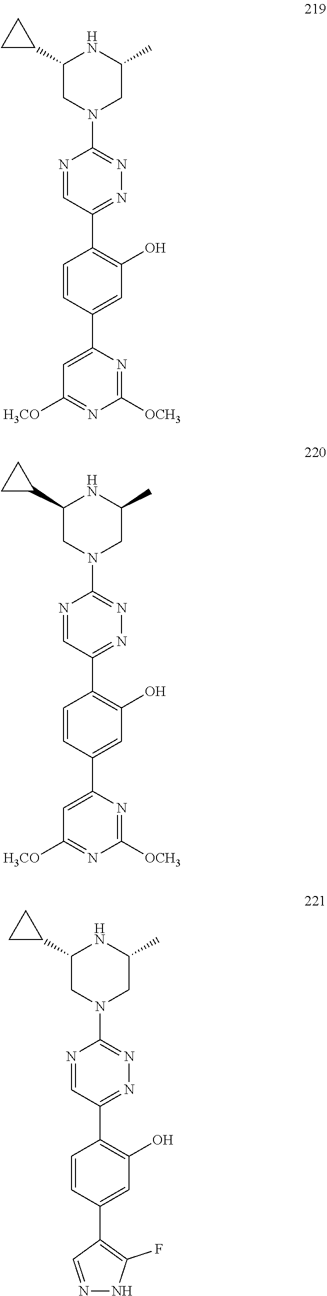 [SMILES: COc1cc(-c2ccc(-c3cnc(N4C[C@@H](C)N[C@@H](C5CC5)C4)nn3)c(O)c2)nc(OC)n1 ; COc1cc(-c2ccc(-c3cnc(N4C[C@@H](C5CC5)N[C@@H](C)C4)nn3)c(O)c2)nc(OC)n1 ; C[C@@H]1CN(c2ncc(-c3ccc(-c4cn[nH]c4F)cc3O)nn2)C[C@H](C2CC2)N1]

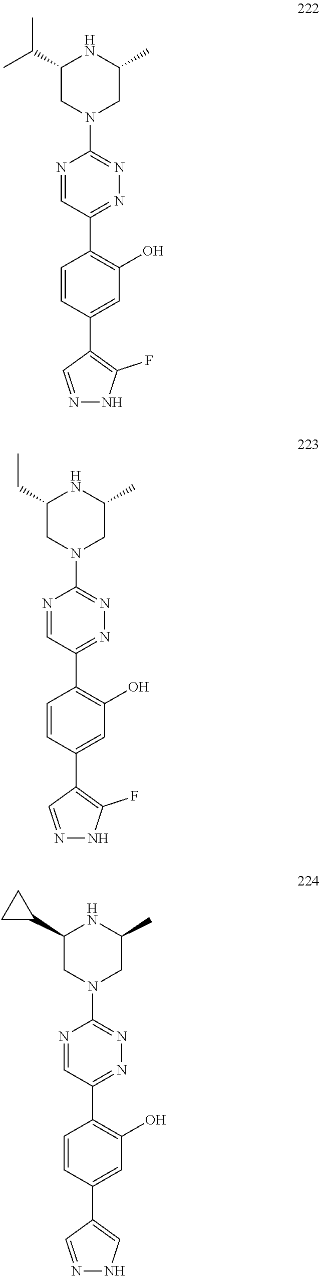 [SMILES: CC(C)[C@H]1CN(c2ncc(-c3ccc(-c4cn[nH]c4F)cc3O)nn2)C[C@@H](C)N1 ; CC[C@H]1CN(c2ncc(-c3ccc(-c4cn[nH]c4F)cc3O)nn2)C[C@@H](C)N1 ; C[C@H]1CN(c2ncc(-c3ccc(-c4cn[nH]c4)cc3O)nn2)C[C@@H](C2CC2)N1]

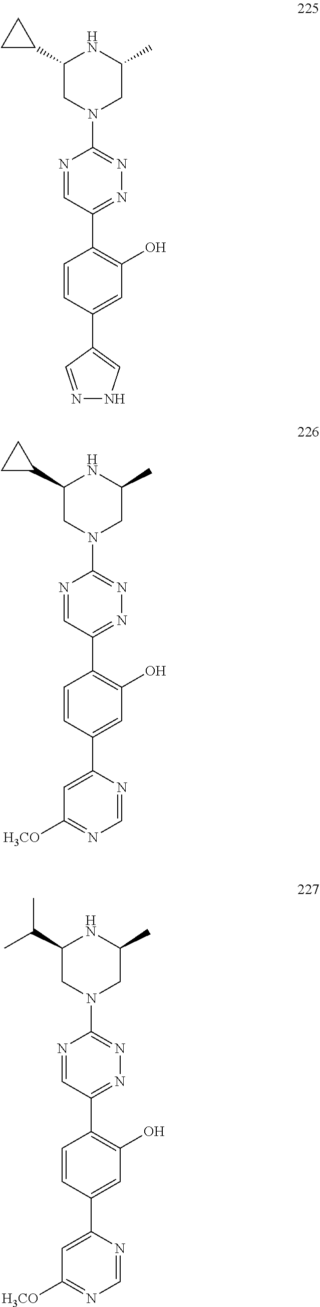

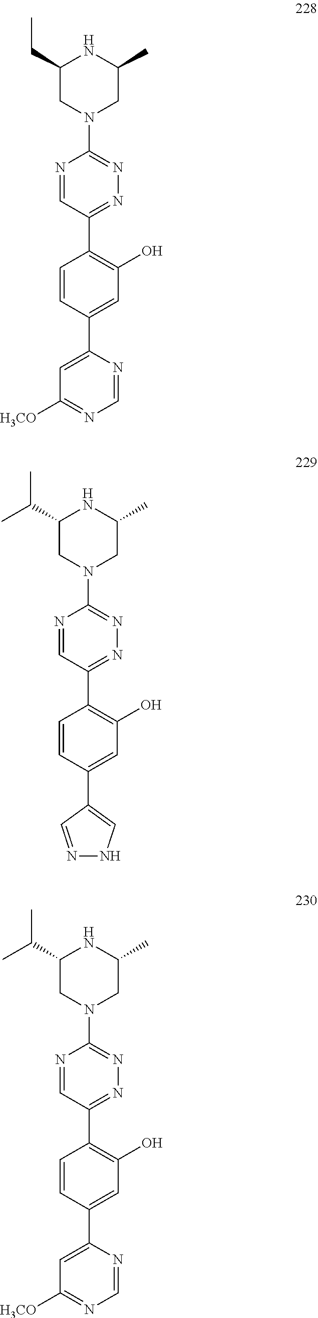

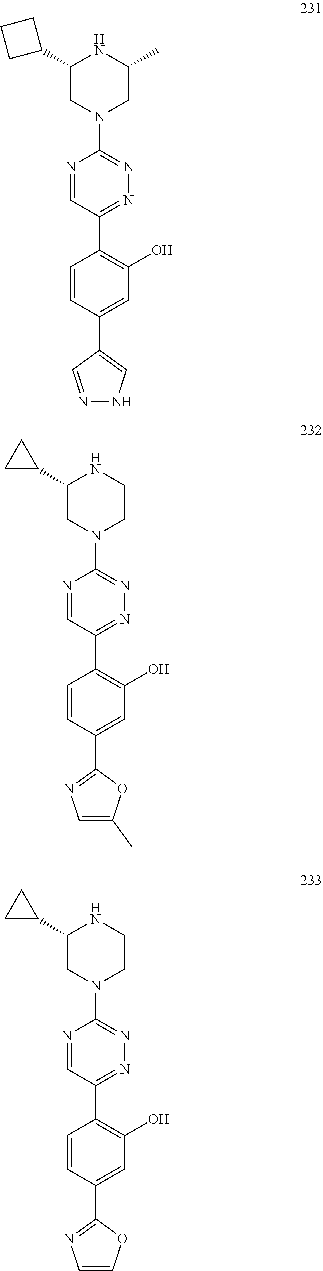 [SMILES: C[C@@H]1CN(c2ncc(-c3ccc(-c4cn[nH]c4)cc3O)nn2)C[C@H](C2CCC2)N1 ; Cc1cnc(-c2ccc(-c3cnc(N4CCN[C@@H](C5CC5)C4)nn3)c(O)c2)o1 ; Oc1cc(-c2ncco2)ccc1-c1cnc(N2CCN[C@@H](C3CC3)C2)nn1]

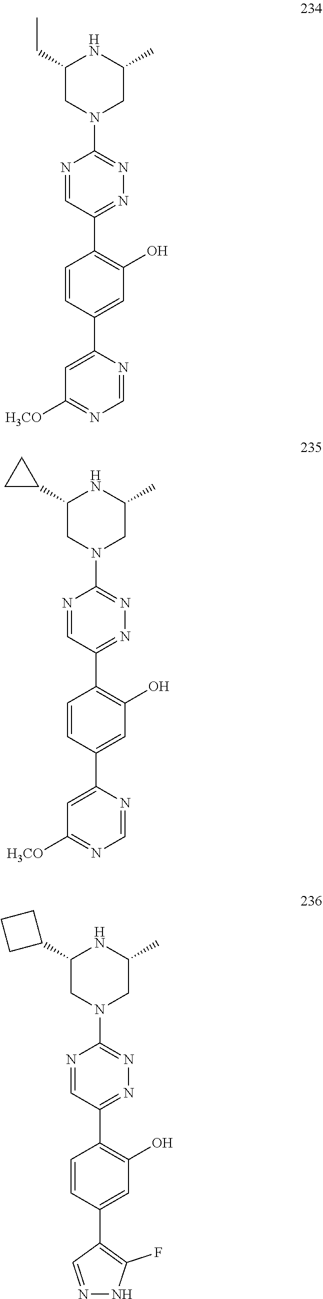

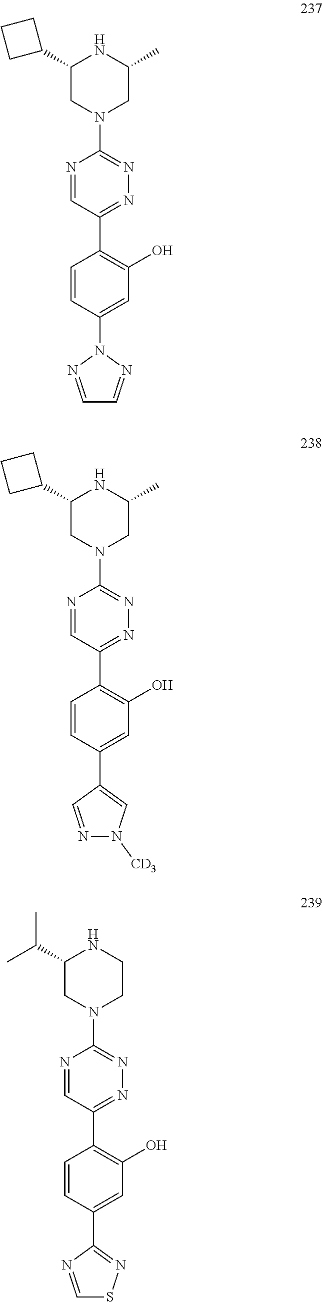

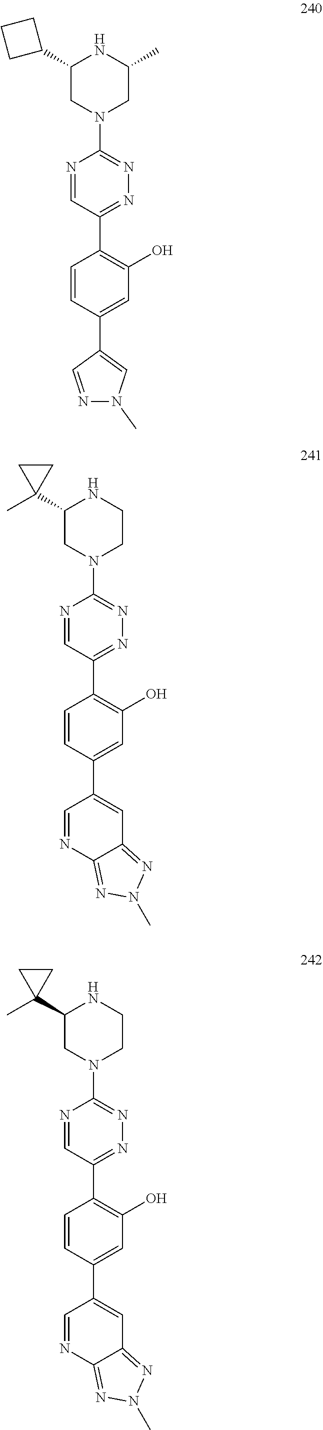

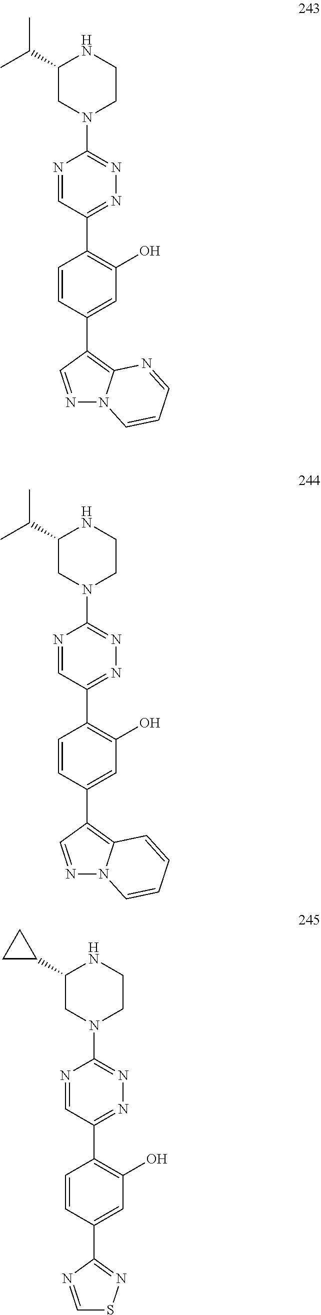 [SMILES: CC(C)[C@H]1CN(c2ncc(-c3ccc(-c4cnn5cccnc45)cc3O)nn2)CCN1 ; CC(C)[C@H]1CN(c2ncc(-c3ccc(-c4cnn5ccccc45)cc3O)nn2)CCN1 ; Oc1cc(-c2ncsn2)ccc1-c1cnc(N2CCN[C@@H](C3CC3)C2)nn1]

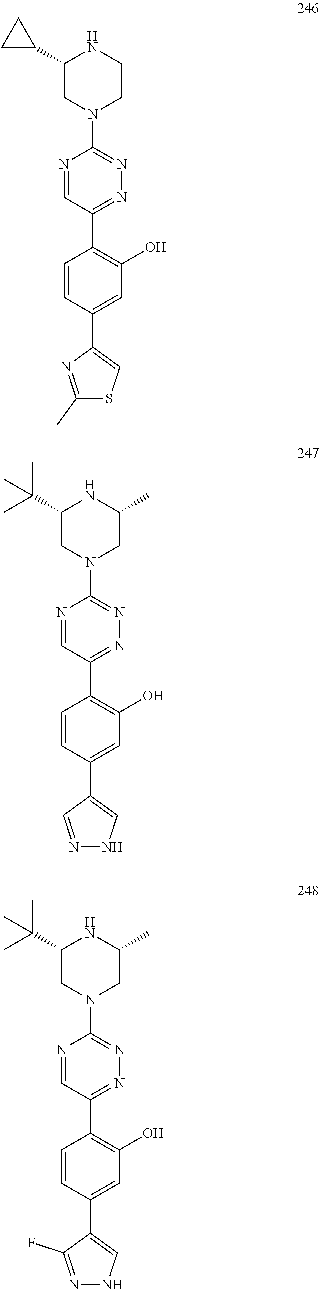

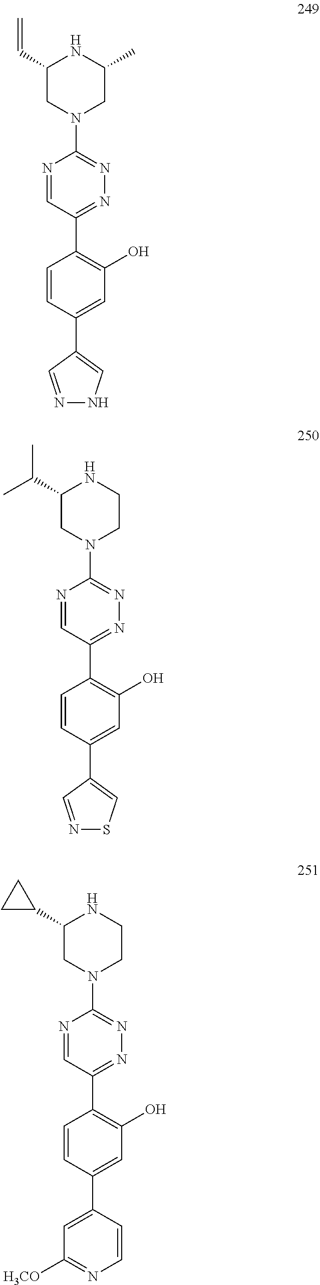

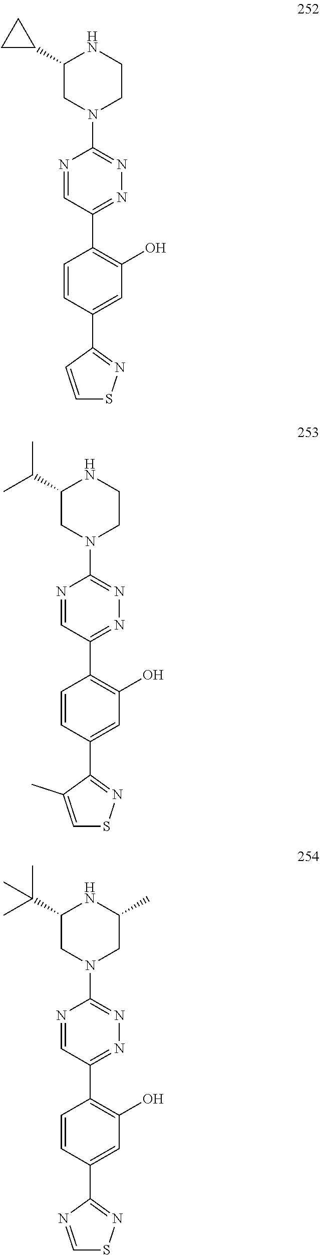 [SMILES: Oc1cc(-c2ccsn2)ccc1-c1cnc(N2CCN[C@@H](C3CC3)C2)nn1 ; Cc1csnc1-c1ccc(-c2cnc(N3CCN[C@@H](C(C)C)C3)nn2)c(O)c1 ; C[C@@H]1CN(c2ncc(-c3ccc(-c4ncsn4)cc3O)nn2)C[C@H](C(C)(C)C)N1]

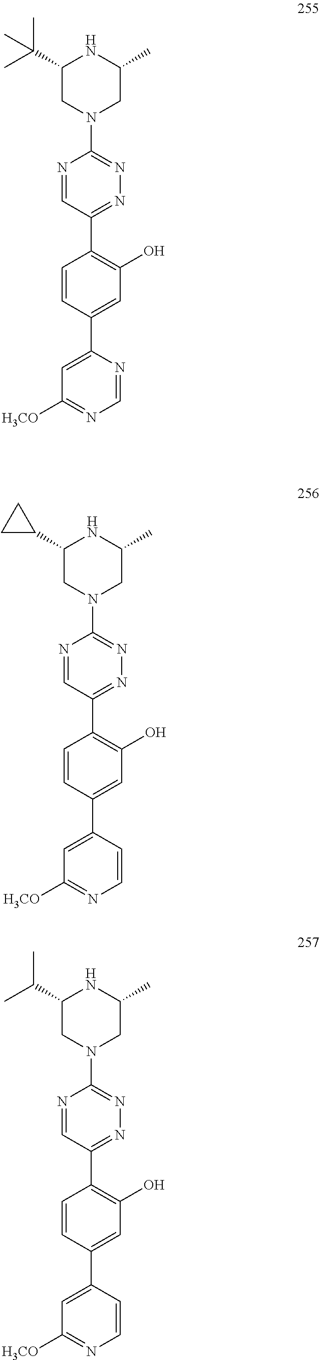

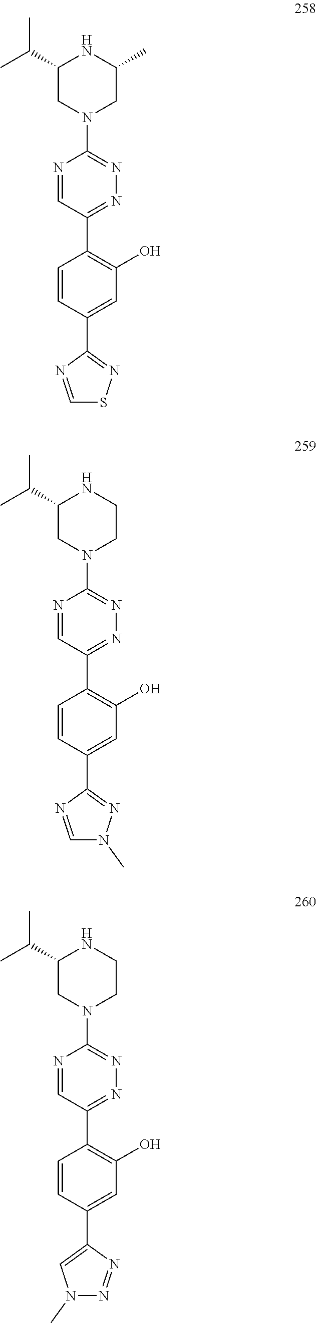

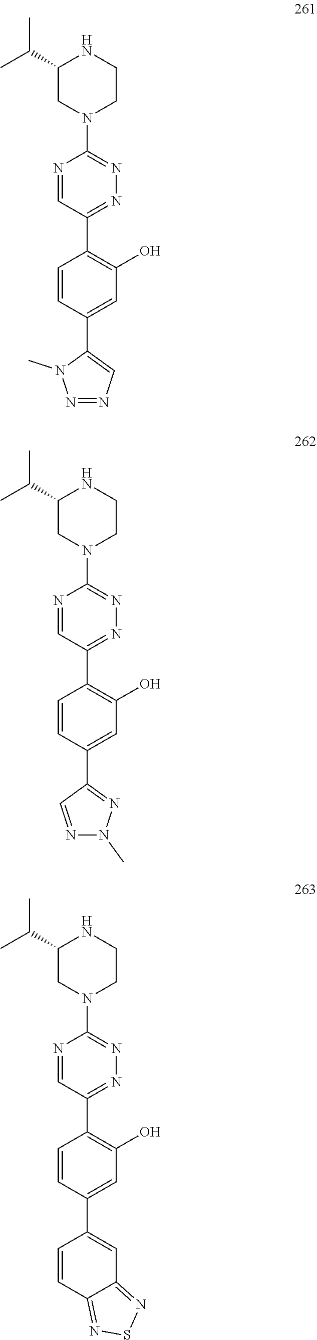 [SMILES: CC(C)[C@H]1CN(c2ncc(-c3ccc(-c4cnnn4C)cc3O)nn2)CCN1 ; CC(C)[C@H]1CN(c2ncc(-c3ccc(-c4cnn(C)n4)cc3O)nn2)CCN1 ; CC(C)[C@H]1CN(c2ncc(-c3ccc(-c4ccc5nsnc5c4)cc3O)nn2)CCN1]

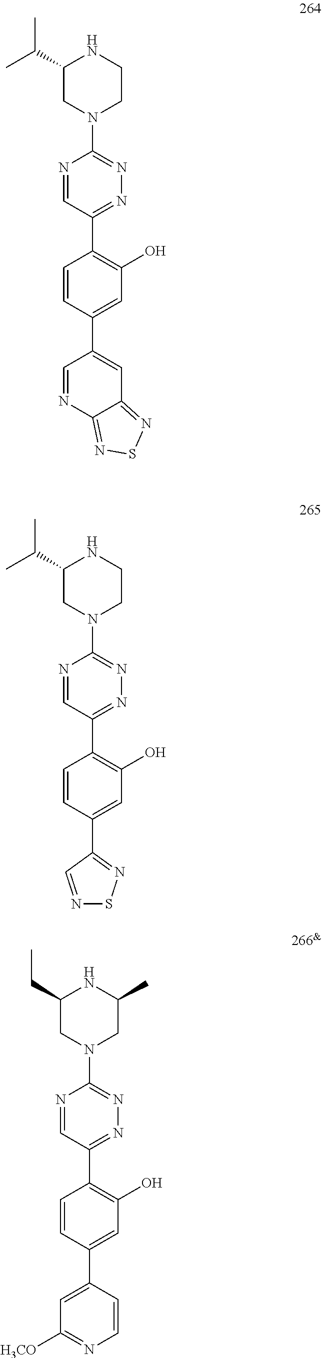 [SMILES: CC(C)[C@H]1CN(c2ncc(-c3ccc(-c4cnc5nsnc5c4)cc3O)nn2)CCN1 ; CC(C)[C@H]1CN(c2ncc(-c3ccc(-c4cnsn4)cc3O)nn2)CCN1 ; CC[C@@H]1CN(c2ncc(-c3ccc(-c4ccnc(OC)c4)cc3O)nn2)C[C@H](C)N1]

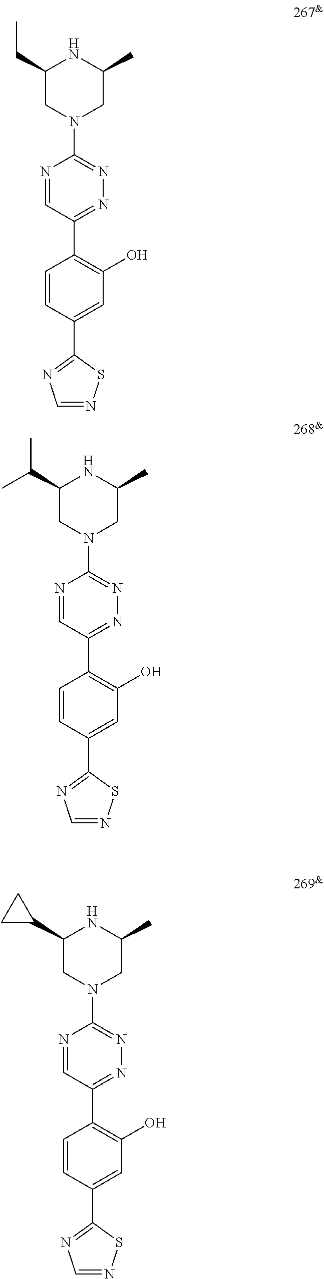

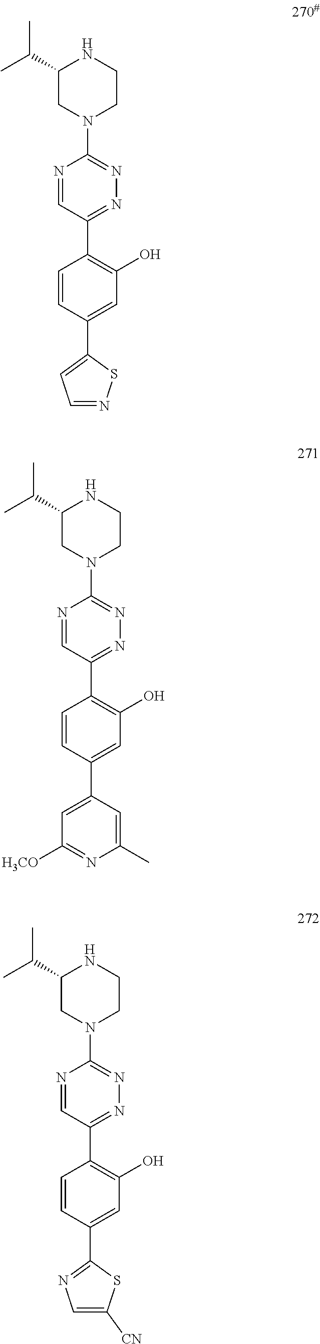

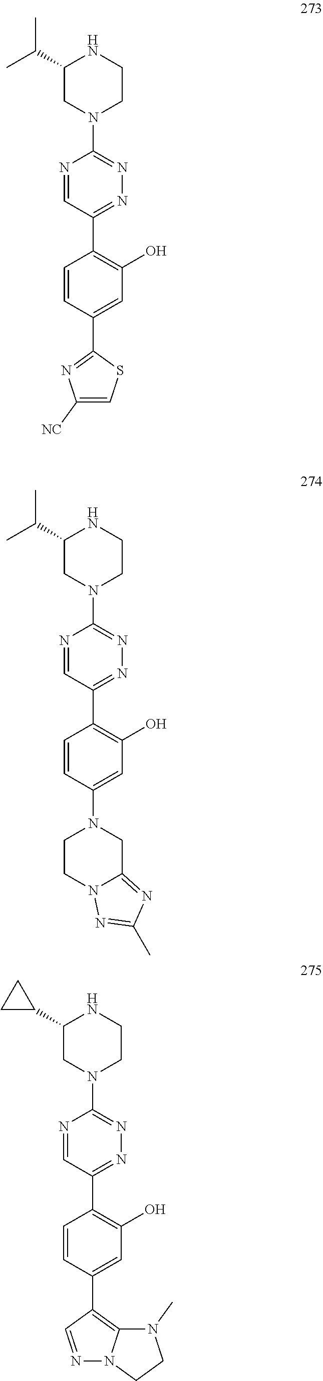 [SMILES: CC(C)[C@H]1CN(c2ncc(-c3ccc(-c4nc(C#N)cs4)cc3O)nn2)CCN1 ; Cc1nc2n(n1)CCN(c1ccc(-c3cnc(N4CCN[C@@H](C(C)C)C4)nn3)c(O)c1)C2 ; CN1CCn2ncc(-c3ccc(-c4cnc(N5CCN[C@@H](C6CC6)C5)nn4)c(O)c3)c21]

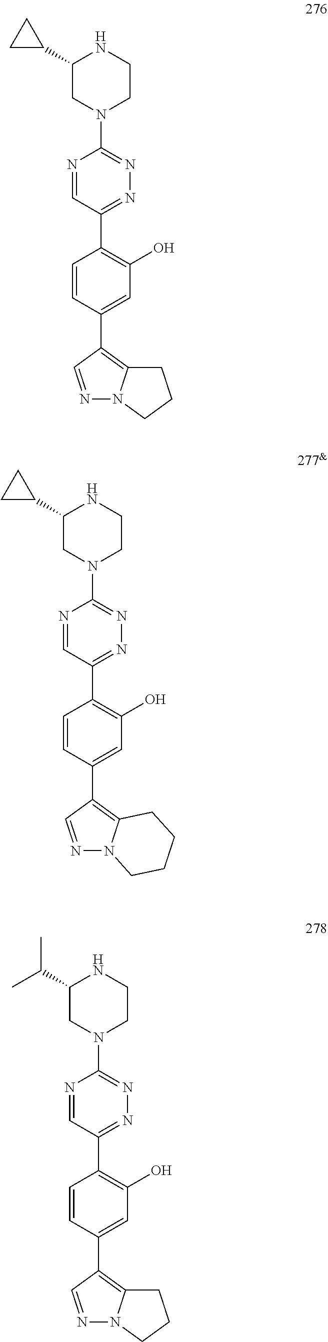 [SMILES: Oc1cc(-c2cnn3c2CCC3)ccc1-c1cnc(N2CCN[C@@H](C3CC3)C2)nn1 ; Oc1cc(-c2cnn3c2CCCC3)ccc1-c1cnc(N2CCN[C@@H](C3CC3)C2)nn1 ; CC(C)[C@H]1CN(c2ncc(-c3ccc(-c4cnn5c4CCC5)cc3O)nn2)CCN1]

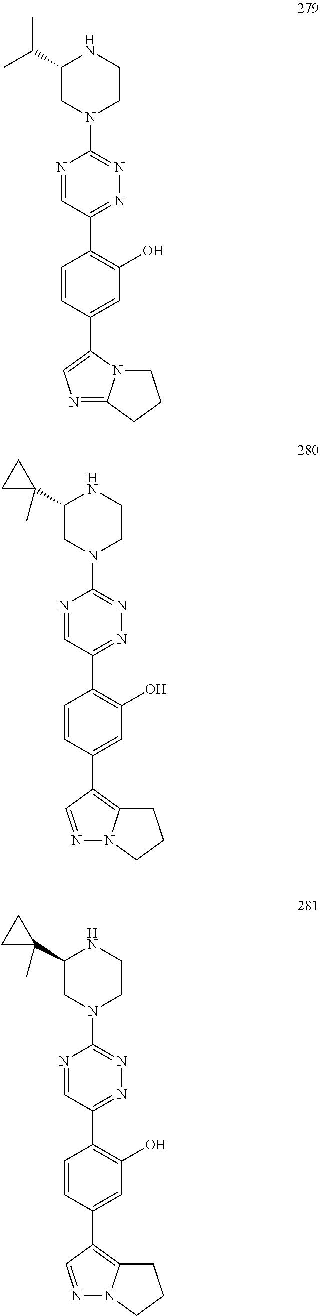 [SMILES: CC(C)[C@H]1CN(c2ncc(-c3ccc(-c4cnc5n4CCC5)cc3O)nn2)CCN1 ; CC1([C@H]2CN(c3ncc(-c4ccc(-c5cnn6c5CCC6)cc4O)nn3)CCN2)CC1 ; CC1([C@@H]2CN(c3ncc(-c4ccc(-c5cnn6c5CCC6)cc4O)nn3)CCN2)CC1]

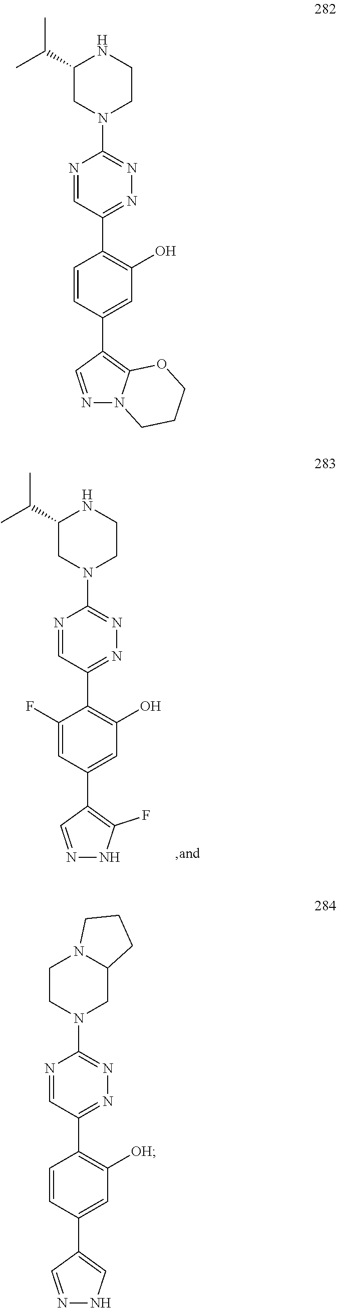 [SMILES: CC(C)[C@H]1CN(c2ncc(-c3ccc(-c4cnn5c4OCCC5)cc3O)nn2)CCN1 ; CC(C)[C@H]1CN(c2ncc(-c3c(O)cc(-c4cn[nH]c4F)cc3F)nn2)CCN1 ; Oc1cc(-c2cn[nH]c2)ccc1-c1cnc(N2CCN3CCCC3C2)nn1]

wherein a form of the compound is selected from the group consisting of a salt, hydrate, ester, solvate, and tautomer form thereof.

An aspect the compound of Formula (I) or a form thereof (wherein compound number (#¹) indicates that the salt form was isolated) includes a compound selected from the group consisting of:

| Cpd | Name |
|---|---|
| 1¹ | 2-{3-[(3S)-3-cyclopropylpiperazin-1-yl]-1,2,4-triazin-6-yl}-5-(3-fluoro-1H-pyrazol-4- |
| | yl)phenol |
| 2¹ | 2-{3-[(3S)-3-cyclopropylpiperazin-1-yl]-1,2,4-triazin-6-yl}-5-{6- |
| | [(²H₃)methyloxy]pyrimidin-4-yl}phenol |
| 3¹ | 5-(3-fluoro-1H-pyrazol-4-yl)-2-{3-[(3S)-3-(propan-2-yl)piperazin-1-yl]-1,2,4-triazin- |
| | 6-yl}phenol |
| 4¹ | 5-(3-fluoro-1H-pyrazol-4-yl)-2-{3-[3-(2-hydroxypropan-2-yl)piperazin-1-yl]-1,2,4- |
| | triazin-6-yl}phenol |
| 5¹ | 2-[3-(3-cyclopropylpiperazin-1-yl)-1,2,4-triazin-6-yl]-5-(1H-pyrazol-4-yl)phenol |
| 6¹ | 2-{3-[3-(1-hydroxycyclopropyl)piperazin-1-yl]-1,2,4-triazin-6-yl}-5-(1H-pyrazol-4- |
| | yl)phenol |
| 7¹ | 2-{3-[(3R)-3-cyclopropylpiperazin-1-yl]-1,2,4-triazin-6-yl}-5-(3-fluoro-1H-pyrazol-4- |
| | yl)phenol |
| 8¹ | 2-{3-[3-(2-hydroxypropan-2-yl)piperazin-1-yl]-1,2,4-triazin-6-yl}-5-{6- |
| | [(²H₃)methyloxy]pyrimidin-4-yl}phenol |
| 9¹ | 2-{3-[(3S)-3-cyclopropylpiperazin-1-yl]-1,2,4-triazin-6-yl}-5-[1-(²H₃)methyl-1H- |
| | pyrazol-4-yl]phenol |
| 10¹ | 5-{6-[(²H₃)methyloxy]pyrimidin-4-yl}-2-{3-[(3S)-3-(propan-2-yl)piperazin-1-yl]- |
| | 1,2,4-triazin-6-yl}phenol |
| 11¹ | 2-{3-[(3S)-3-cyclopropylpiperazin-1-yl]-1,2,4-triazin-6-yl}-5-(2H-1,2,3-triazol-2- |
| | yl)phenol |
| 12¹ | 2-{3-[(3S)-3-cyclopropylpiperazin-1-yl]-1,2,4-triazin-6-yl}-5-(1-methyl-1H-pyrazol- |
| | 4-yl)phenol |
| 13¹ | 2-{3-[3-(2-hydroxypropan-2-yl)piperazin-1-yl]-1,2,4-triazin-6-yl}-5-(1H-pyrazol-4- |
| | yl)phenol |
| 14¹ | 2-{3-[3-(2-hydroxypropan-2-yl)piperazin-1-yl]-1,2,4-triazin-6-yl}-5-(2H-1,2,3- |
| | triazol-2-yl)phenol |
| 15¹ | 5-(3-fluoro-1H-pyrazol-4-yl)-2-{3-[3-(propan-2-yl)piperazin-1-yl]-1,2,4-triazin-6- |
| | yl]phenol |
| 16¹ | 2-{3-[(3S)-3-tert-butylpiperazin-1-yl]-1,2,4-triazin-6-yl}-5-(1H-pyrazol-4-yl)phenol |
| 17¹ | 2-{3-[(3S)-3-tert-butylpiperazin-1-yl]-1,2,4-triazin-6-yl}-5-{6- |
| | [(²H₃)methyloxy]pyrimidin-4-yl}phenol |
| 18¹ | 2-{3-[(3S)-3-tert-butylpiperazin-1-yl]-1,2,4-triazin-6-yl}-5-(2H-1,2,3-triazol-2- |
| | yl)phenol |
| 19¹ | 2-{3-[3-(2-hydroxypropan-2-yl)piperazin-1-yl]-1,2,4-triazin-6-yl}-5-[1-(²H₃)methyl- |
| | 1H-pyrazol-4-yl]phenol |
| 20 | 2-{3-[(3S)-3-(propan-2-yl)piperazin-1-yl]-1,2,4-triazin-6-yl}-5-(1H-pyrazol-4- |
| | yl)phenol |
| 21¹ | 2-{3-[(3S)-3-tert-butylpiperazin-1-yl]-1,2,4-triazin-6-yl]-5-(1-methyl-1H-pyrazol-4- |
| | yl)phenol |
| 22 | 2-{3-[3-(propan-2-yl)piperazin-1-yl]-1,2,4-triazin-6-yl}-5-(1H-pyrazol-4-yl)phenol |
| 23 | 2-{3-[(3S)-3-ethylpiperazin-1-yl]-1,2,4-triazin-6-yl}-5-(1H-pyrazol-4-yl)phenol |
| 24¹ | 2-[3-(3-ethylpiperazin-1-yl)-1,2,4-triazin-6-yl]-5-(1H-pyrazol-4-yl)phenol |
| 25¹ | 5-(3-fluoro-1H-pyrazol-4-yl)-2-{3-[(3R)-3-(2-hydroxypropan-2-yl)piperazin-1-yl]- |
| | 1,2,4-triazin-6-yl]phenol |
| 26¹ | 2-{3-[3-(2-hydroxypropan-2-yl)-4-methylpiperazin-1-yl]-1,2,4-triazin-6-yl}-5-{6- |
| | [(²H₃)methyloxy]pyrimidin-4-yl}phenol |
| 27¹ | 2-[3-(3-cyclopropyl-4-methylpiperazin-1-yl)-1,2,4-triazin-6-yl]-5-(1H-pyrazol-4- |
| | yl)phenol |
| 28¹ | 3-fluoro-5-(6-methoxypyrimidin-4-yl)-2-{3-[(3S)-3-(propan-2-yl)piperazin-1-yl]- |
| | 1,2,4-triazin-6-yl}phenol |
| 29¹ | 2-{3-[3-(1-methoxycyclopropyl)piperazin-1-yl]-1,2,4-triazin-6-yl}-5-(1H-pyrazol-4- |
| | yl)phenol |
| 30 | 2-[3-(3-cyclobutylpiperazin-1-yl)-1,2,4-triazin-6-yl]-5-(1H-pyrazol-4-yl)phenol |
| 31¹ | 2-[3-(3-propylpiperazin-1-yl)-1,2,4-triazin-6-yl]-5-(1H-pyrazol-4-yl)phenol |
| 32¹ | 2-[3-(3-cyclopropylpiperazin-1-yl)-1,2,4-triazin-6-yl]-3-fluoro-5-(5-fluoro-1H- |
| | pyrazol-4-yl)phenol |
| 33¹ | 2-{3-[3-(butan-2-yl)piperazin-1-yl]-1,2,4-triazin-6-yl}-5-(1H-pyrazol-4-yl)phenol |
| 34 | 2-{3-[4-methyl-3-(propan-2-yl)piperazin-1-yl]-1,2,4-triazin-6-yl}-5-(1H-pyrazol-4- |
| | yl)phenol |
| 35¹ | 5-(1-methyl-1H-pyrazol-4-yl)-2-{3-[3-(propan-2-yl)piperazin-1-yl]-1,2,4-triazin-6- |
| | yl}phenol |
| 36¹ | 2-{3-[3-(2,2-dilluorocyclopropyl)piperazin-1-yl]-1,2,4-triazin-6-yl}-5-(1H-pyrazol-4- |
| | yl)phenol |
| 37¹ | 2-{3-[(3S)-3-propylpiperazin-1-yl]-1,2,4-triazin-6-yl}-5-(1H-pyrazol-4-yl)phenol |
| 38¹ | 2-[3-(3-ethenylpiperazin-1-yl)-1,2,4-triazin-6-yl]-5-(1H-pyrazol-4-yl)phenol |
| 39¹ | 2-[3-(3-ethylpiperazin-1-yl)-1,2,4-triazin-6-yl]-5-[1-(²H₃)methyl-1H-pyrazol-4- |
| | yl]phenol |
| 40 | 2-{3-[(3R)-3-(propan-2-yl)piperazin-1-yl]-1,2,4-triazin-6-yl}-5-(1H-pyrazol-4- |
| | yl)phenol |
| 41¹ | 5-[1-(²H₃)methyl-1H-pyrazol-4-yl]-2-{3-[3-(propan-2-yl)piperazin-1-yl]-1,2,4-triazin- |
| | 6-yl}phenol |
| 42 | 2-[3-(3-methylpiperazin-1-yl)-1,2,4-triazin-6-yl]-5-(1H-pyrazol-4-yl)phenol |
| 43¹ | 2-[3-(6,9-diazaspiro[4.5]decan-9-yl)-1,2,4-triazin-6-yl]-5-(1H-pyrazol-4-yl)phenol |
| 44¹ | 5-(2-methylpyridin-4-yl)-2-{3-[3-(propan-2-yl)piperazin-1-yl]-1,2,4-triazin-6- |
| | yl}phenol |
| 45¹ | 2-{3-[(3S)-3-(hydroxymethyl)piperazin-1-yl]-1,2,4-triazin-6-yl}-5-(2H-1,2,3-triazol- |
| | 2-yl)phenol |
| 46 | 2-{3-[3-(2-methylpropyl)piperazin-1-yl]-1,2,4-triazin-6-yl}-5-(1H-pyrazol-4- |
| | yl)phenol |
| 47¹ | 5-[1-(Hz)methyl-1H-pyrazol-4-yl]-2-{3-[(3S)-3-(propan-2-yl)piperazin-1-yl]-1,2,4- |
| | triazin-6-yl}phenol |
| 48 | 2-{3-[(3R)-3-ethylpiperazin-1-yl]-1,2,4-triazin-6-yl}-5-(1H-pyrazol-4-yl)phenol |
| 49¹ | 2-{3-[(3R,5S)-3,5-dimethylpiperazin-1-yl]-1,2,4-triazin-6-yl}-5-[1-(²H₃)methyl-1H- |
| | pyrazol-4-yl]phenol |
| 50¹ | 2-[3-(5,8-diazaspiro[3.5]nonan-8-yl)-1,2,4-triazin-6-yl]-5-(1H-pyrazol-4-yl)phenol |
| 51¹ | 5-[1-(²H₃)methyl-1H-pyrazol-4-yl]-2-{3-[(3R,5S)-3,4,5-trimethylpiperazin-1-yl]- |
| | 1,2,4-triazin-6-yl}phenol |
| 52¹ | 5-(2H-1,2,3-triazol-2-yl)-2-{3-[(3R,5S)-3,4,5-trimethylpiperazin-1-yl]-1,2,4-triazin-6- |
| | yl}phenol |
| 53 | 2-{3-[(3R)-3-(methoxymethyl)piperazin-1-yl]-1,2,4-triazin-6-yl}-5-(1H-pyrazol-4- |
| | yl)phenol |
| 54¹ | 2-{3-[(3R,5S)-3,5-dimethylpiperazin-1-yl]-1,2,4-triazin-6-yl}-5-(2H-1,2,3-triazol-2- |
| | yl)phenol |
| 55¹ | 2-[3-(4,7-diazaspiro[2.5]octan-7-yl)-1,2,4-triazin-6-yl]-5-(1H-pyrazol-4-yl)phenol |
| 56¹ | 2-[3-(3,3-dimethylpiperazin-1-yl)-1,2,4-triazin-6-yl]-5-(1H-pyrazol-4-yl)phenol |
| 57¹ | 2-[3-(4,7-diazaspiro[2.5]octan-7-yl)-1,2,4-triazin-6-yl]-5-(3-fluoro-1H-pyrazol-4- |
| | yl)phenol |
| 58¹ | 2-[3-(8-methyl-3,8-diazabicyclo[3.2.1]octan-3-yl)-1,2,4-triazin-6-yl]-5-[l- |
| | (²H₃)methyl-1H-pyrazol-4-yl]phenol |
| 59¹ | (7R,8aS)-2-{6-[2-hydroxy-4-(1H-pyrazol-4-yl)phenyl]-1,2,4-triazin-3- |
| | yl}octahydropyrrolo[1,2-a]pyrazin-7-ol |
| 60 | 2-{3-[4-(propan-2-yl)piperazin-1-yl]-1,2,4-triazin-6-yl}-5-(1H-pyrazol-4-yl)phenol |
| 61¹ | 2-[3-(3-phenylpiperazin-1-yl)-1,2,4-triazin-6-yl]-5-(1H-pyrazol-4-yl)phenol |
| 62¹ | 5-(1H-pyrazol-4-yl)-2-{3-[3-(pyridin-4-yl)piperazin-1-yl]-1,2,4-triazin-6-yl}phenol |
| 63 | 2-[3-(4-cyclopropylpiperazin-1-yl)-1,2,4-triazin-6-yl]-5-(1H-pyrazol-4-yl)phenol |
| 64 | 2-[3-(hexahydropyrazino[2,l-c][1,4]oxazin-8(1H)-yl)-1,2,4-triazin-6-yl]-5-(1H- |
| | pyrazol-4-yl)phenol |
| 65¹ | 5-(7-fluoro-2-methyl-2H-indazol-5-yl)-2-{3-[3-(hydroxymethyl)piperazin-1-yl]-1,2,4- |
| | triazin-6-yl}phenol |
| 66¹ | 2-{3-[(3R,5S)-3,5-dimethylpiperazin-1-yl]-1,2,4-triazin-6-yl]-5-(8-fluoro-2- |
| | methylimidazo[1,2-a]pyridin-6-yl)phenol |
| 67¹ | 5-(7-fluoro-2-methyl-2H-indazol-5-yl)-2-[3-(3-methylpiperazin-1-yl)-1,2,4-triazin-6- |
| | yl]phenol |
| 68¹ | 2-[3-(3-ethylpiperazin-1-yl)-1,2,4-triazin-6-yl]-5-(8-fluoro-2-methylimidazo[1,2- |
| | a]pyridin-6-yl)phenol |
| 69¹ | 5-(2,8-dimethylimidazo[1,2-b]pyridazin-6-yl)-2-{3-[(3R,5S)-3,5-dimethylpiperazin-1- |
| | yl]-1,2,4-triazin-6-yl}phenol |
| 70¹ | 5-(8-methoxy-2-methyl[1,2,4]triazolo[1,5-b]pyridazin-6-yl)-2-{3-[(3S)-3-(propan-2- |
| | yl)piperazin-1-yl]-1,2,4-triazin-6-yl}phenol |
| 71¹ | 5-(7-fluoro-2-methyl-2H-indazol-5-yl)-2-{3-[3-(propan-2-yl)piperazin-1-yl]-1,2,4- |
| | triazin-6-yl}phenol |
| 72 | 2-{3-[(3R,5S)-3,5-dimethylpiperazin-1-yl]-1,2,4-triazin-6-yl]-5-(2-methyl-2H- |
| | indazol-5-yl)phenol |
| 73¹ | 5-(8-fluoro-2-methylimidazo[1,2-a]pyridin-6-yl)-2-[3-(4-methylpiperazin-1-yl)-1,2,4- |
| | triazin-6-yl]phenol |
| 74¹ | 2-[3-(3-ethylpiperazin-1-yl)-1,2,4-triazin-6-yl]-5-(7-fluoro-2-methyl-2H-indazol-5- |
| | yl)phenol |
| 75 | 2-{3-[(3R,5S)-3,5-dimethylpiperazin-1-yl]-1,2,4-triazin-6-yl}-5-(2- |
| | methylimidazo[1,2-b]pyridazin-6-yl)phenol |
| 76 | 2-{3-[(3R,5R)-3,5-dimethylpiperazin-1-yl]-1,2,4-triazin-6-yl}-5-(7-fluoro-2-methyl- |
| | 2H-indazol-5-yl)phenol |
| 77¹ | 2-[3-(4-ethylpiperazin-1-yl)-1,2,4-triazin-6-yl]-5-(8-fluoro-2-methylimidazo[1,2- |
| | a]pyridin-6-yl)phenol |
| 78¹ | 5-(2,8-dimethyl[1,2,4]triazolo[1,5-a]pyrazin-6-yl)-2-{3-[(3S)-3-(propan-2- |
| | yl)piperazin-1-yl]-1,2,4-triazin-6-yl}phenol |
| 79 | 2-{3-[(3R,5S)-3,5-dimethylpiperazin-1-yl]-1,2,4-triazin-6-yl}-5-(7-fluoro-2-methyl- |
| | 2H-indazol-5-yl)phenol |
| 80¹ | 5-(4-{3-[(3R,5S)-3,5-dimethylpiperazin-1-yl]-1,2,4-triazin-6-yl}-3-hydroxyphenyl)-2- |
| | methyl-2H-indazole-7-carbonitrile |
| 81¹ | 5-(7-fluoro-2-methyl-2H-indazol-5-yl)-2-{3-[(3S)-3-(propan-2-yl)piperazin-1-yl]- |
| | 1,2,4-triazin-6-yl}phenol |
| 82 | 5-(2-methylimidazo[1,2-b]pyridazin-6-yl)-2-[3-(4-methylpiperazin-1-yl)-1,2,4-triazin- |
| | 6-yl]phenol |
| 83¹ | 2-{3-[(3R,5S)-3,5-dimethylpiperazin-1-yl]-1,2,4-triazin-6-yl}-5-(2,8- |
| | dimethyl[1,2,4]triazolo[1,5-b]pyridazin-6-yl)phenol |
| 84¹ | 2-{3-[(3S)-3-cyclopropylpiperazin-1-yl]-1,2,4-triazin-6-yl]-5-(6,8-dimethyl-7H- |
| | purin-2-yl)phenol |
| 85¹ | 5-(2-methyl[1,2,4]triazolo[1,5-a]pyridin-6-yl)-2-{3-[(3S)-3-(propan-2-yl)piperazin-1- |
| | yl]-1,2,4-triazin-6-yl}phenol |
| 86¹ | 2-{3-[(3R,5S)-3,5-dimethylpiperazin-1-yl]-1,2,4-triazin-6-yl}-5-(2- |
| | methylimidazo[1,2-a]pyrazin-6-yl)phenol |
| 87 | 6-(4-{3-[(3R,5S)-3,5-dimethylpiperazin-1-yl]-1,2,4-triazin-6-yl}-3-hydroxyphenyl)-2- |
| | methylimidazo[1,2-a]pyridine-8-carbonitrile |
| 88¹ | 5-(2-methyl-2H-indazol-5-yl)-2-{3-[(3S)-3-(propan-2-yl)piperazin-1-yl]-1,2,4-triazin- |
| | 6-yl}phenol |
| 89¹ | 2-{3-[(3R)-4-ethyl-3-methylpiperazin-1-yl]-1,2,4-triazin-6-yl}-5-(8-fluoro-2- |
| | methylimidazo[1,2-a]pyridin-6-yl)phenol |
| 90¹ | 2-{3-[(3R,5S)-3,5-dimethylpiperazin-1-yl]-1,2,4-triazin-6-yl}-5-(8-methoxy-2- |
| | methyl[1,2,4]triazolo[1,5-b]pyridazin-6-yl)phenol |
| 91¹ | 5-(2-methyl[1,2,4]triazolo[1,5-a]pyrazin-6-yl)-2-{3-[(3S)-3-(propan-2-yl)piperazin-1- |
| | yl]-1,2,4-triazin-6-yl}phenol |
| 92¹ | 5-(2,8-dimethyl[1,2,4]triazolo[1,5-b]pyridazin-6-yl)-2-{3-[(3S)-3-(propan-2- |
| | yl)piperazin-1-yl]-1,2,4-triazin-6-yl}phenol |
| 93¹ | 5-(8-methoxy-2-methyl[1,2,4]triazolo[1,5-a]pyrazin-6-yl)-2-{3-[(3S)-3-(propan-2- |
| | yl)piperazin-1-yl]-1,2,4-triazin-6-yl}phenol |
| 94¹ | 5-(8-fluoro-2-methylimidazo[1,2-a]pyridin-6-yl)-2-{3-[(3R,5S)-3,4,5- |
| | trimethylpiperazin-1-yl]-1,2,4-triazin-6-yl}phenol |
| 95¹ | 5-(7-fluoro-2-methyl-2H-indazol-5-yl)-2-[3-(hexahydropyrrolo[1,2-a]pyrazin-2(1H)- |
| | yl)-1,2,4-triazin-6-yl]phenol |
| 96¹ | 5-(2,8-dimethylimidazo[1,2-a]pyrazin-6-yl)-2-{3-[(3S)-3-(propan-2-yl)piperazin-1- |
| | yl]-1,2,4-triazin-6-yl]phenol |
| 97 | 2-{3-[(3R,5S)-3,5-dimethylpiperazin-1-yl]-1,2,4-triazin-6-yl}-5-(2-methyl-2H- |
| | pyrazolo[3,4-b]pyridin-5-yl)phenol |
| 98¹ | 5-(2,8-dimethylimidazo[1,2-b]pyridazin-6-yl)-2-{3-[(3R)-3-methylpiperazin-1-yl]- |
| | 1,2,4-triazin-6-yl]phenol |
| 99 | 5-(7-fluoro-2-methyl-2H-indazol-5-yl)-2-{3-[(8aS)-hexahydropyrrolo[1,2-a]pyrazin- |
| | 2(1H)-yl]-1,2,4-triazin-6-yl]phenol |
| 100¹ | 2-{3-[(3R,5S)-3,5-dimethylpiperazin-1-yl]-1,2,4-triazin-6-yl}-5-(2- |
| | methyl[1,2,4]triazolo[1,5-b]pyridazin-6-yl)phenol |
| 101¹ | 2-{3-[(3R,5S)-3,5-dimethylpiperazin-1-yl]-1,2,4-triazin-6-yl}-5-(2,8- |
| | dimethyl[1,2,4]triazolo[1,5-a]pyrazin-6-yl)phenol |
| 102¹ | 5-(imidazo[1,2-b]pyridazin-6-yl)-2-{3-[(3S)-3-(propan-2-yl)piperazin-1-yl]-1,2,4- |
| | triazin-6-yl}phenol |
| 103¹ | 5-(2,8-dimethylimidazo[1,2-a]pyrazin-6-yl)-2-{3-[(3R,5S)-3,5-dimethylpiperazin-1- |
| | yl]-1,2,4-triazin-6-yl}phenol |
| 104¹ | 5-(2,8-dimethylimidazo[1,2-b]pyridazin-6-yl)-2-[3-(4-methylpiperazin-1-yl)-1,2,4- |
| | triazin-6-yl]phenol |
| 105¹ | 5-(7-fluoro-2-methyl-2H-indazol-5-yl)-2-{3-[(8aR)-hexahydropyrrolo[1,2-a]pyrazin- |
| | 2(1H)-yl]-1,2,4-triazin-6-yl}phenol |
| 106¹ | 2-{3-[(3R,5S)-3,5-dimethylpiperazin-1-yl]-1,2,4-triazin-6-yl}-5-(2- |
| | methyl[1,2,4]triazolo[1,5-a]pyrazin-6-yl)phenol |
| 107¹ | 2-{3-[(3R)-3,4-dimethylpiperazin-1-yl]-1,2,4-triazin-6-yl}-5-(8-fluoro-2- |
| | methylimidazo[1,2-a]pyridin-6-yl)phenol |
| 108¹ | 6-(3-hydroxy-4-{3-[(3R,5S)-3,4,5-trimethylpiperazin-1-yl]-1,2,4-triazin-6-yl]phenyl)- |
| | 2-methylimidazo[1,2-b]pyridazine-8-carbonitrile |
| 109¹ | 5-(8-cyclopropyl-2-methyl[1,2,4]triazolo[1,5-a]pyrazin-6-yl)-2-{3-[(3R,5S)-3,5- |
| | dimethylpiperazin-1-yl]-1,2,4-triazin-6-yl}phenol |
| 110 | 2-[3-(4-methylpiperazin-1-yl)-1,2,4-triazin-6-yl]-5-(2-methyl-2H-pyrazolo[3,4- |
| | b]pyridin-5-yl)phenol |
| 111¹ | 5-(2,8-dimethylimidazo[1,2-b]pyridazin-6-yl)-2-{3-[(3R,5S)-3,4,5-trimethylpiperazin- |
| | 1-yl]-1,2,4-triazin-6-yl}phenol |
| 112¹ | 2-{3-[(3R,5S)-3,5-dimethylpiperazin-1-yl]-1,2,4-triazin-6-yl}-5-(2- |
| | methyl[1,2,4]triazolo[1,5-a]pyrimidin-6-yl)phenol |
| 113¹ | 5-(imidazo[1,2-a]pyrazin-6-yl)-2-{3-[(3S)-3-(propan-2-yl)piperazin-1-yl]-1,2,4- |
| | triazin-6-yl}phenol |
| 114¹ | 5-(imidazo[1,2-a]pyridin-6-yl)-2-{3-[(3S)-3-(propan-2-yl)piperazin-1-yl]-1,2,4- |
| | triazin-6-yl}phenol |
| 115¹ | 2-{3-[(3S)-3-(propan-2-yl)piperazin-1-yl]-1,2,4-triazin-6-yl}-5-([1,2,4]triazolo[4,3- |
| | a]pyridin-6-yl)phenol |
| 116¹ | 2-{3-[(3R,5S)-3,5-dimethylpiperazin-1-yl]-1,2,4-triazin-6-yl}-5-(4,6- |
| | dimethyl[1,3]thiazolo[5,4-c]pyridin-2-yl)phenol |
| 117¹ | 2-{3-[(3R,5S)-3,5-dimethylpiperazin-1-yl]-1,2,4-triazin-6-yl}-5-(5,7- |
| | dimethyl[1,2,4]triazolo[1,5-a]pyrimidin-2-yl)phenol |
| 118¹ | 2-{3-[(3R,5S)-3,5-dimethylpiperazin-1-yl]-1,2,4-triazin-6-yl)-5-[2- |
| | (trifluoromethyl)imidazo[1,2-b]pyridazin-6-yl]phenol |
| 119¹ | 2-{3-[(3R,5S)-3,5-dimethylpiperazin-1-yl]-1,2,4-triazin-6-yl}-5-(6- |
| | methyl[1,3]thiazolo[4,5-b]pyrazin-2-yl)phenol |
| 120¹ | 2-{3-[(3R,5S)-3,5-dimethylpiperazin-1-yl]-1,2,4-triazin-6-yl}-5-(5-methylfuro[3,2- |
| | b]pyridin-2-yl)phenol |
| 121¹ | 5-(7-methoxy-2-methyl-2H-indazol-5-yl)-2-[3-(4-methylpiperazin-1-yl)-1,2,4-triazin- |
| | 6-yl]pyridin-3-ol |
| 122¹ | 5-(8-fluoro-2-methylimidazo[1,2-a]pyridin-6-yl)-2-[3-(4-methylpiperazin-1-yl)-1,2,4- |
| | triazin-6-yl]pyridin-3-ol |
| 123¹ | 2-{3-[(3S)-3-ethylpiperazin-1-yl]-1,2,4-triazin-6-yl}-5-(3-fluoro-1H-pyrazol-4- |
| | yl)phenol |
| 124¹ | 2-[3-(4-methylpiperazin-1-yl)-1,2,4-triazin-6-yl]-5-(1H-pyrazol-4-yl)pyridin-3-ol |
| 125¹ | 2-{3-[(3S)-3-tert-butylpiperazin-1-yl]-1,2,4-triazin-6-yl}-5-(3-fluoro-1H-pyrazol-4- |
| | yl)phenol |
| 126¹ | 5-(2,8-dimethylimidazo[1,2-a]pyridin-6-yl)-2-[3-(4-methylpiperazin-1-yl)-1,2,4- |
| | triazin-6-yl]pyridin-3-ol |
| 127¹ | 3-methyl-2-{3-[(3S)-3-(propan-2-yl)piperazin-1-yl]-1,2,4-triazin-6-yl}-5-(1H-pyrazol- |
| | 4-yl)phenol |
| 128¹ | 2-{3-[(3S)-3-(propan-2-yl)piperazin-1-yl]-1,2,4-triazin-6-yl}-5-([1,2,4]triazolo[1,5- |
| | a]pyrazin-6-yl)phenol |
| 129¹ | 2-{3-[(3S)-3-tert-butylpiperazin-1-yl]-1,2,4-triazin-6-yl}-5-([1,2,4]triazolo[1,5- |
| | a]pyrazin-6-yl)phenol |
| 130¹ | 2-{3-[(3S)-3-cyclopropylpiperazin-1-yl]-1,2,4-triazin-6-yl}-5-([1,2,4]triazolo[1,5- |
| | a]pyrazin-6-yl)phenol |
| 131¹ | 5-(2,8-dimethylimidazo[1,2-a]pyridin-6-yl)-2-{3-[(3S)-3-(propan-2-yl)piperazin-1- |
| | yl]-1,2,4-triazin-6-yl}phenol |
| 132¹ | 2-{3-[(3S)-3-ethylpiperazin-1-yl]-1,2,4-triazin-6-yl}-5-(2H-1,2,3-triazol-2-yl)phenol |
| 133¹ | 2-{3-[(3S)-3-ethylpiperazin-1-yl]-1,2,4-triazin-6-yl}-5-{6- |
| | [(²H₃)methyloxy]pyrimidin-4-yl}phenol |
| 134¹ | 2-{3-[3-(1-methylcyclopropyl)piperazin-1-yl]-1,2,4-triazin-6-yl}-5-(1H-pyrazol-4- |
| | yl)phenol |
| 135¹ | 2-[3-(3,8-diazabicyclo[3.2.1]octan-3-yl)-1,2,4-triazin-6-yl]-5-(1H-pyrazol-4-yl)phenol |
| 136¹ | 2-{3-[(3R)-3-cyclopropylpiperazin-1-yl]-1,2,4-triazin-6-yl}-5-(1H-pyrazol-4- |
| | yl)phenol |
| 137¹ | 2-{3-[(3S)-3-cyclopropylpiperazin-1-yl]-1,2,4-triazin-6-yl}-5-(1H-pyrazol-4- |
| | yl)phenol |
| 138¹ | 5-(8-ethyl-2-methylimidazo[1,2-a]pyridin-6-yl)-2-{3-[(3S)-3-(propan-2-yl)piperazin- |
| | 1-yl]-1,2,4-triazin-6-yl}phenol |
| 139¹ | 5-[2-methyl-8-(trilluoromethyl)imidazo[1,2-a]pyridin-6-yl]-2-{3-[(3S)-3-(propan-2- |
| | yl)piperazin-1-yl]-1,2,4-triazin-6-yl}phenol |
| 140¹ | 5-(2,7-dimethyl-2H-indazol-5-yl)-2-{3-[(3S)-3-(propan-2-yl)piperazin-1-yl]-1,2,4- |
| | triazin-6-yl}phenol |
| 141¹ | 5-(2-methylimidazo[1,2-a]pyrazin-6-yl)-2-{3-[(3S)-3-(propan-2-yl)piperazin-1-yl]- |
| | 1,2,4-triazin-6-yl}phenol |
| 142 | 5-(1H-imidazol-1-yl)-2-{3-[(3S)-3-(propan-2-yl)piperazin-1-yl]-1,2,4-triazin-6- |
| | yl}phenol |
| 143 | 5-(6-methylpyrazin-2-yl)-2-{3-[(3S)-3-(propan-2-yl)piperazin-1-yl]-1,2,4-triazin-6- |
| | yl}phenol |
| 144 | 2-{3-[(3S)-3-(propan-2-yl)piperazin-1-yl]-1,2,4-triazin-6-yl}-5-(pyrazin-2-yl)phenol |
| 145 | 5-(5-methylpyrazin-2-yl)-2-{3-[(3S)-3-(propan-2-yl)piperazin-1-yl]-1,2,4-triazin-6- |
| | yl}phenol |
| 146¹ | 5-(1H-pyrazol-4-yl)-2-{3-[3-(2,2,2-trifluoroethyl)piperazin-1-yl]-1,2,4-triazin-6- |
| | yl}phenol |
| 147¹ | 5-(2-methyl[1,2,4]triazolo[1,5-a]pyridin-7-yl)-2-{3-[(3S)-3-(propan-2-yl)piperazin-1- |
| | yl]-1,2,4-triazin-6-yl}phenol |
| 148¹ | 2-{3-[rac-(3S,5R)-3-ethyl-5-methylpiperazin-1-yl]-1,2,4-triazin-6-yl}-5-(1H-pyrazol- |
| | 4-yl)phenol |
| 149¹ | 5-(6-methylpyrimidin-4-yl)-2-{3-[(3RS)-3-(propan-2-yl)piperazin-1-yl]-1,2,4-triazin- |
| | 6-yl}phenol |
| 150 | 5-(6-ethylpyrimidin-4-yl)-2-{3-[(3S)-3-(propan-2-yl)piperazin-1-yl]-1,2,4-triazin-6- |
| | yl}phenol |
| 151 | 2-{3-[(3S)-3-(propan-2-yl)piperazin-1-yl]-1,2,4-triazin-6-yl}-5-(pyrimidin-2- |
| | yl)phenol |
| 152¹ | 4-fluoro-5-[1-(²H₃)methyl-1H-pyrazol-4-yl]-2-{3-[(3S)-3-(propan-2-yl)piperazin-1- |
| | yl]-1,2,4-triazin-6-yl}phenol |
| 153¹ | 2-{3-[(8aS)-hexahydropyrrolo[1,2-a]pyrazin-2(1H)-yl]-1,2,4-triazin-6-yl}-5-(2H- |
| | 1,2,3-triazol-2-yl)phenol |
| 154¹ | 2-{3-[(3S)-3-(propan-2-yl)piperazin-1-yl]-1,2,4-triazin-6-yl}-5-(2H-1,2,3-triazol-2- |
| | yl)phenol |
| 155¹ | 5-(5-methyl-1H-pyrazolo[4,3-b]pyridin-1-yl)-2-{3-[(3S)-3-(propan-2-yl)piperazin-1- |
| | yl]-1,2,4-triazin-6-yl}phenol |
| 156¹ | 2-{3-[(3S)-3-tert-butylpiperazin-1-yl]-1,2,4-triazin-6-yl}-4-fluoro-5-[1-(²H₃)methyl- |
| | 1H-pyrazol-4-yl]phenol |
| 157¹ | 5-(7-fluoro-2-methyl-2H-indazol-5-yl)-2-{3-[(3S)-3-(propan-2-yl)piperazin-1-yl]- |
| | 1,2,4-triazin-6-yl}pyridin-3-ol |
| 158¹ | 4-fluoro-2-{3-[(3S)-3-(propan-2-yl)piperazin-1-yl]-1,2,4-triazin-6-yl}-5-(1H-pyrazol- |
| | 4-yl)phenol |
| 159¹ | 5-(5-methyl-1H-pyrrolo[3,2-b]pyridin-1-yl)-2-{3-[(3S)-3-(propan-2-yl)piperazin-1- |
| | yl]-1,2,4-triazin-6-yl}phenol |
| 160 | 5-(6-methylpyridin-3-yl)-2-{3-[(3S)-3-(propan-2-yl)piperazin-1-yl]-1,2,4-triazin-6- |
| | yl]phenol |
| 161 | 2-{3-[(3S)-3-(propan-2-yl)piperazin-1-yl]-1,2,4-triazin-6-yl}-5-(pyridin-4-yl)phenol |
| 162¹ | 2-{3-[(3S)-3-(propan-2-yl)piperazin-1-yl]-1,2,4-triazin-6-yl}-5-(1H-pyrazolo[3,4- |
| | d]pyrimidin-1-yl)phenol |
| 163¹ | 5-(3-chloro-1H-pyrazol-4-yl)-2-{3-[(3S)-3-cyclopropylpiperazin-1-yl]-1,2,4-triazin-6- |
| | yl}phenol |
| 164¹ | 2-{3-[(3S)-3-tert-butylpiperazin-1-yl]-1,2,4-triazin-6-yl}-4-fluoro-5-(1H-pyrazol-4- |
| | yl)phenol |
| 165¹ | 2-{3-[(3S)-3-methylpiperazin-1-yl]-1,2,4-triazin-6-yl}-5-(1H-pyrazol-4-yl)phenol |
| 166¹ | 2-{3-[(3R)-3-methylpiperazin-1-yl]-1,2,4-triazin-6-yl}-5-(1H-pyrazol-4-yl)phenol |
| 167 | 2-{3-[(3R,5S)-3,5-dimethylpiperazin-1-yl]-1,2,4-triazin-6-yl}-5-(2- |
| | methyl[1,2,4]triazolo[1,5-a]pyrazin-6-yl)phenol |
| 168¹ | 2-{3-[(3S)-3-cyclopropylpiperazin-1-yl]-1,2,4-triazin-6-yl}-5-(2-methyl-2H- |
| | [1,2,3]triazolo[4,5-b]pyridin-6-yl)phenol |
| 169¹ | 2-{3-[(3S)-3-cyclopropylpiperazin-1-yl]-1,2,4-triazin-6-yl}-5-(1-methyl-1H- |
| | [1,2,3]triazolo[4,5-b]pyridin-6-yl)phenol |
| 170 | 2-{3-[(3S)-3-cyclopropylpiperazin-1-yl]-1,2,4-triazin-6-yl}-5-(1,2,4-thiadiazol-5- |
| | yl)phenol |
| 171¹ | 2-{3-[(3S)-3-cyclopropylpiperazin-1-yl]-1,2,4-triazin-6-yl}-5-(1-methyl-1H- |
| | [1,2,3]triazolo[4,5-b]pyridin-5-yl)phenol |
| 172¹ | 2-{3-[(3S)-3-cyclopropylpiperazin-1-yl]-1,2,4-triazin-6-yl}-5-(2-methyl-2H- |
| | [1,2,3]triazolo[4,5-c]pyridin-6-yl)phenol |
| 173¹ | 2-{3-[(3S)-3-cyclopropylpiperazin-1-yl]-1,2,4-triazin-6-yl}-5-(3-methyl-3H- |
| | [1,2,3]triazolo[4,5-b]pyridin-5-yl)phenol |
| 174¹ | 2-{3-[(3S)-3-cyclopropylpiperazin-1-yl]-1,2,4-triazin-6-yl}-5-(1-methyl-1H- |
| | [1,2,3]triazolo[4,5-c]pyridin-6-yl)phenol |
| 175¹ | 2-{3-[(3S)-3-(propan-2-yl)piperazin-1-yl]-1,2,4-triazin-6-yl}-5-(2H-1,2,3-triazol-2- |
| | yl)pyridin-3-ol |
| 176¹ | 2-{3-[(2S,5S)-2,5-dimethylpiperazin-1-yl]-1,2,4-triazin-6-yl}-5-(1H-pyrazol-4- |
| | yl)phenol |
| 177¹ | 2-{3-[(3S)-3-cyclopropylpiperazin-1-yl]-1,2,4-triazin-6-yl}-5-(pyridin-4-yl)phenol |
| 178¹ | 2-{3-[(3S)-3-cyclopropylpiperazin-1-yl]-1,2,4-triazin-6-yl}-5-(3-fluoropyridin-4- |
| | yl)phenol |
| 179¹ | 4-{3-[(3S)-3-cyclopropylpiperazin-1-yl]-1,2,4-triazin-6-yl}-4‘-(methylamino)[1,1‘- |
| | biphenyl]-3-ol |
| 180¹ | 2-{3-[(3S)-3-cyclopropylpiperazin-1-yl]-1,2,4-triazin-6-yl}-5-(2-methyl-2H- |
| | [1,2,3]triazolo[4,5-b]pyridin-6-yl)pyridin-3-ol |
| 181¹ | 2-{3-[(3S)-3-cyclopropylpiperazin-1-yl]-1,2,4-triazin-6-yl}-5-(7-fluoro-2-methyl-2H- |
| | indazol-5-yl)pyridin-3-ol |
| 182¹ | 2-{3-[(3S)-3-cyclopropylpiperazin-1-yl]-1,2,4-triazin-6-yl}-5-(2H-1,2,3-triazol-2- |
| | yl)pyridin-3-ol |
| 183¹ | 2-{3-[(3R)-3-cyclopropylpiperazin-1-yl]-1,2,4-triazin-6-yl}-5-(4-methyl-2H-1,2,3- |
| | triazol-2-yl)phenol |
| 184¹ | 5-(4-methyl-2H-1,2,3-triazol-2-yl)-2-{3-[(3S)-3-(propan-2-yl)piperazin-1-yl]-1,2,4- |
| | triazin-6-yl}phenol |
| 185 | 2-{3-[(3S)-3-(propan-2-yl)piperazin-1-yl]-1,2,4-triazin-6-yl}-5-(1,3-thiazol-2- |
| | yl)phenol |
| 186 | 5-[4-(difluoromethyl)-1,3-thiazol-2-yl]-2-{3-[(3S)-3-(propan-2-yl)piperazin-1-yl]- |
| | 1,2,4-triazin-6-yl}phenol |
| 187¹ | 2-{3-[4-methyl-3-(oxetan-3-yl)piperazin-1-yl]-1,2,4-triazin-6-yl}-5-(1H-pyrazol-4- |
| | yl)phenol |
| 188 | 5-(4-chloro-1,3-thiazol-2-yl)-2-{3-[(3S)-3-(propan-2-yl)piperazin-1-yl]-1,2,4-triazin- |
| | 6-yl}phenol |
| 189 | 5-(5-chloro-1,3-thiazol-2-yl)-2-{3-[(3S)-3-(propan-2-yl)piperazin-1-yl]-1,2,4-triazin- |
| | 6-yl}phenol |
| 190¹ | 2-{3-[(2R,5S)-2,5-dimethylpiperazin-1-yl]-1,2,4-triazin-6-yl}-5-(1H-pyrazol-4- |
| | yl)phenol or |
| | 2-{3-[(2S,5R)-2,5-dimethylpiperazin-1-yl]-1,2,4-triazin-6-yl}-5-(1H-pyrazol-4- |
| | yl)phenol |
| 191¹ | 2-{3-[(3S)-3-(propan-2-yl)piperazin-1-yl]-1,2,4-triazin-6-yl}-5-([1,3]thiazolo[5,4- |
| | b]pyridin-2-yl)phenol |
| 192 | 2-{3-[(3S)-3-cyclopropylpiperazin-1-yl]-1,2,4-triazin-6-yl}-5-(pyrimidin-4-yl)phenol |
| 193¹ | 2-{3-[(3S)-3-cyclopropylpiperazin-1-yl]-1,2,4-triazin-6-yl}-5-(3- |
| | methyl[1,2,3]triazolo[1,5-a]pyridin-6-yl)phenol |
| 194¹ | 2-{3-[(3S)-3-cyclopropylpiperazin-1-yl]-1,2,4-triazin-6-yl}-5-(1-methylimidazo[1,5- |
| | a]pyridin-6-yl)phenol |
| 195¹ | 2-{3-[(3S)-3-cyclopropylpiperazin-1-yl]-1,2,4-triazin-6-yl}-5-(3-methylimidazo[1,5- |
| | a]pyridin-7-yl)phenol |
| 196 | 5-(5-fluoro-1,3-thiazol-2-yl)-2-{3-[(3S)-3-(propan-2-yl)piperazin-1-yl]-1,2,4-triazin- |
| | 6-yl}phenol |
| 197 | 2-{3-[(3R,5S)-3,5-dimethylpiperazin-1-yl]-1,2,4-triazin-6-yl}-5-(2-methyl-2H- |
| | [1,2,3]triazolo[4,5-b]pyridin-6-yl)phenol |
| 198 | 2-{3-[(3R,5S)-3,5-dimethylpiperazin-1-yl]-1,2,4-triazin-6-yl}-5-(2-methyl-2H- |
| | [1,2,3]triazolo[4,5-c]pyridin-6-yl)phenol |
| 199 | 5-(5-cyclopropyl-1,3,4-oxadiazol-2-yl)-2-{3-[(3S)-3-(propan-2-yl)piperazin-1-yl]- |
| | 1,2,4-triazin-6-yl}phenol |
| 200 | 5-(4-methoxy-1,3-thiazol-2-yl)-2-{3-[(3S)-3-(propan-2-yl)piperazin-1-yl]-1,2,4- |
| | triazin-6-yl}phenol |
| 201 | 2-{3-[rac-(3R,5S)-3,5-dimethylpiperazin-1-yl]-1,2,4-triazin-6-yl}-5-(2-methyl-2H- |
| | [1,2,3]triazolo[4,5-b]pyridin-5-yl)phenol |
| 202¹ | 2-{3-[(3S)-3-cyclopropylpiperazin-1-yl]-1,2,4-triazin-6-yl}-5-(2-methyl-2H- |
| | [1,2,3]triazolo[4,5-b]pyridin-5-yl)phenol |
| 203 | 2-[3-(octahydro-2H-pyrido[1,2-a]pyrazin-2-yl)-1,2,4-triazin-6-yl]-5-(1H-pyrazol-4- |
| | yl)phenol |
| 204 | 5-(5-methoxy-1,3,4-thiadiazol-2-yl)-2-{3-[(3S)-3-(propan-2-yl)piperazin-1-yl]-1,2,4- |
| | triazin-6-yl}phenol |
| 205 | 5-[5-(difluoromethyl)-1,3,4-thiadiazol-2-yl]-2-{3-[(3S)-3-(propan-2-yl)piperazin-1- |
| | yl]-1,2,4-triazin-6-yl}phenol |
| 206 | 2-{3-[rac-(3R,5S)-3,5-dimethylpiperazin-1-yl]-1,2,4-triazin-6-yl}-5-(3- |
| | methyl[1,2,3]triazolo[1,5-a]pyridin-6-yl)phenol |
| 207 | 5-(2,6-dimethoxypyrimidin-4-yl)-2-{3-[(3S)-3-(propan-2-yl)piperazin-1-yl]-1,2,4- |
| | triazin-6-yl}phenol |
| 208¹ | 2-{3-[(3S)-3-cyclopropylpiperazin-1-yl]-1,2,4-triazin-6-yl}-5-(1-ethyl-1H-pyrazol-4- |
| | yl)phenol |
| 209 | 2-{3-[(3RS)-3-cyclopropylpiperazin-1-yl]-1,2,4-triazin-6-yl}-5-(2-fluoropyridin-4- |
| | yl)phenol |
| 210 | 5-[6-(azetidin-1-yl)pyrimidin-4-yl]-2-{3-[(3RS)-3-cyclopropylpiperazin-1-yl]-1,2,4- |
| | triazin-6-yl}phenol |
| 211¹ | 2-{3-[(3S)-3-tert-butylpiperazin-1-yl]-1,2,4-triazin-6-yl}-5-(2-methyl-2H- |
| | [1,2,3]triazolo[4,5-b]pyridin-6-yl)phenol |
| 212¹ | 5-(2-methyl-2H-[1,2,3]triazolo[4,5-b]pyridin-6-yl)-2-{3-[(3S)-3-(propan-2- |
| | yl)piperazin-1-yl]-1,2,4-triazin-6-yl}phenol |
| 213¹ | 2-{3-[(3S)-3-cyclopropylpiperazin-1-yl]-1,2,4-triazin-6-yl]-5-(1,2,4-thiadiazol-3- |
| | yl)phenol |
| 214 | 2-{3-[(3S,5R)-3-ethyl-5-methylpiperazin-1-yl]-1,2,4-triazin-6-yl]-5-(2-methyl-2H- |
| | [1,2,3]triazolo[4,5-b]pyridin-6-yl)phenol |
| 215 | 2-{3-[(3R,5S)-3-cyclopropyl-5-methylpiperazin-1-yl]-1,2,4-triazin-6-yl}-5-(5-fluoro- |
| | 1H-pyrazol-4-yl)phenol |
| 216 | 2-{3-[(3S,5R)-3-methyl-5-(propan-2-yl)piperazin-1-yl]-1,2,4-triazin-6-yl]-5-(1H- |
| | pyrazol-4-yl)phenol |
| 217 | 5-(5-fluoro-1H-pyrazol-4-yl)-2-{3-[(3S,5R)-3-methyl-5-(propan-2-yl)piperazin-1-yl]- |
| | 1,2,4-triazin-6-yl]phenol |
| 218 | 2-{3-[(3R,5S)-3-ethyl-5-methylpiperazin-1-yl]-1,2,4-triazin-6-yl}-5-(5-fluoro-1H- |
| | pyrazol-4-yl)phenol |
| 219 | 2-{3-[(3S,5R)-3-cyclopropyl-5-methylpiperazin-1-yl]-1,2,4-triazin-6-yl}-5-(2,6- |
| | dimethoxypyrimidin-4-yl)phenol |
| 220 | 2-{3-[(3R,5S)-3-cyclopropyl-5-methylpiperazin-1-yl]-1,2,4-triazin-6-yl}-5-(2,6- |
| | dimethoxypyrimidin-4-yl)phenol |
| 221 | 2-{3-[(3S,5R)-3-cyclopropyl-5-methylpiperazin-1-yl]-1,2,4-triazin-6-yl]-5-(5-fluoro- |
| | 1H-pyrazol-4-yl)phenol |
| 222 | 5-(5-fluoro-1H-pyrazol-4-yl)-2-{3-[(3R,5S)-3-methyl-5-(propan-2-yl)piperazin-1-yl]- |
| | 1,2,4-triazin-6-yl]phenol |
| 223 | 2-{3-[(3S,5R)-3-ethyl-5-methylpiperazin-1-yl]-1,2,4-triazin-6-yl]-5-(5-fluoro-1H- |
| | pyrazol-4-yl)phenol |
| 224 | 2-{3-[(3R,5S)-3-cyclopropyl-5-methylpiperazin-1-yl]-1,2,4-triazin-6-yl]-5-(1H- |
| | pyrazol-4-yl)phenol |
| 225 | 2-{3-[(3S,5R)-3-cyclopropyl-5-methylpiperazin-1-yl]-1,2,4-triazin-6-yl}-5-(1H- |
| | pyrazol-4-yl)phenol |
| 226 | 2-{3-[(3R,5S)-3-cyclopropyl-5-methylpiperazin-1-yl]-1,2,4-triazin-6-yl}-5-(6- |
| | methoxypyrimidin-4-yl)phenol |
| 227 | 5-(6-methoxypyrimidin-4-yl)-2-{3-[(3S,5R)-3-methyl-5-(propan-2-yl)piperazin-1-yl]- |
| | 1,2,4-triazin-6-yl}phenol |
| 228 | 2-{3-[(3R,5S)-3-ethyl-5-methylpiperazin-1-yl]-1,2,4-triazin-6-yl}-5-(6- |
| | methoxypyrimidin-4-yl)phenol |
| 229 | 2-{3-[(3R,5S)-3-methyl-5-(propan-2-yl)piperazin-1-yl]-1,2,4-triazin-6-yl}-5-(1H- |
| | pyrazol-4-yl)phenol |
| 230 | 5-(6-methoxypyrimidin-4-yl)-2-{3-[(3R,5S)-3-methyl-5-(propan-2-yl)piperazin-1-yl]- |
| | 1,2,4-triazin-6-yl}phenol |
| 231¹ | 2-{3-[(3S,5R)-3-cyclobutyl-5-methylpiperazin-1-yl]-1,2,4-triazin-6-yl}-5-(1H- |
| | pyrazol-4-yl)phenol |
| 232¹ | 2-{3-[(3S)-3-cyclopropylpiperazin-1-yl]-1,2,4-triazin-6-yl}-5-(5-methyl-1,3-oxazol-2- |
| | yl)phenol |
| 233¹ | 2-{3-[(3S)-3-cyclopropylpiperazin-1-yl]-1,2,4-triazin-6-yl}-5-(1,3-oxazol-2-yl)phenol |
| 234 | 2-{3-[(3S,5R)-3-ethyl-5-methylpiperazin-1-yl]-1,2,4-triazin-6-yl}-5-(6- |
| | methoxypyrimidin-4-yl)phenol |
| 235 | 2-{3-[(3S,5R)-3-cyclopropyl-5-methylpiperazin-1-yl]-1,2,4-triazin-6-yl}-5-(6- |
| | methoxypyrimidin-4-yl)phenol |
| 236¹ | 2-{3-[(3S,5R)-3-cyclobutyl-5-methylpiperazin-1-yl]-1,2,4-triazin-6-yl}-5-(3-fluoro- |
| | 1H-pyrazol-4-yl)phenol |
| 237¹ | 2-{3-[(3S,5R)-3-cyclobutyl-5-methylpiperazin-1-yl]-1,2,4-triazin-6-yl}-5-(2H-1,2,3- |
| | triazol-2-yl)phenol |
| 238¹ | 2-{3-[(3S,5R)-3-cyclobutyl-5-methylpiperazin-1-yl]-1,2,4-triazin-6-yl}-5-[l- |
| | (²H₃)methyl-1H-pyrazol-4-yl]phenol |
| 239¹ | 2-{3-[(3S)-3-(propan-2-yl)piperazin-1-yl]-1,2,4-triazin-6-yl}-5-(1,2,4-thiadiazol-3- |
| | yl)phenol |
| 240¹ | 2-{3-[(3S,5R)-3-cyclobutyl-5-methylpiperazin-1-yl]-1,2,4-triazin-6-yl}-5-(1-methyl- |
| | 1H-pyrazol-3-yl)phenol |
| 241¹ | 2-{3-[(3S)-3-(1-methylcyclopropyl)piperazin-1-yl]-1,2,4-triazin-6-yl}-5-(2-methyl- |
| | 2H-[1,2,3]triazolo[4,5-b]pyridin-6-yl)phenol |
| 242¹ | 2-{3-[(3R)-3-(1-methylcyclopropyl)piperazin-1-yl]-1,2,4-triazin-6-yl}-5-(2-methyl- |
| | 2H-[1,2,3]triazolo[4,5-b]pyridin-6-yl)phenol |
| 243¹ | 2-{3-[(3S)-3-(propan-2-yl)piperazin-1-yl]-1,2,4-triazin-6-yl}-5-(pyrazolo[1,5- |
| | a]pyrimidin-3-yl)phenol |
| 244 | 2-{3-[(3S)-3-(propan-2-yl)piperazin-1-yl]-1,2,4-triazin-6-yl}-5-(pyrazolo[1,5- |
| | a]pyridin-3-yl)phenol |
| 245¹ | 2-{3-[(3S)-3-cyclopropylpiperazin-1-yl]-1,2,4-triazin-6-yl}-5-(5-methyl-1,2,4- |
| | thiadiazol-3-yl)phenol |
| 246¹ | 2-{3-[(3S)-3-cyclopropylpiperazin-1-yl]-1,2,4-triazin-6-yl}-5-(2-methyl-1,3-thiazol-4- |
| | yl)phenol |
| 247¹ | 2-{3-[(3S,5R)-3-tert-butyl-5-methylpiperazin-1-yl]-1,2,4-triazin-6-yl]-5-(1H-pyrazol- |
| | 4-yl)phenol |
| 248¹ | 2-{3-[(3S,5R)-3-tert-butyl-5-methylpiperazin-1-yl]-1,2,4-triazin-6-yl]-5-(3-fluoro- |
| | 1H-pyrazol-4-yl)phenol |
| 249¹ | 2-{3-[(3S,5R)-3-ethenyl-5-methylpiperazin-1-yl]-1,2,4-triazin-6-yl]-5-(1H-pyrazol-4- |
| | yl)phenol |
| 250¹ | 2-{3-[(3S)-3-(propan-2-yl)piperazin-1-yl]-1,2,4-triazin-6-yl]-5-(1,2-thiazol-4- |
| | yl)phenol |
| 251¹ | 2-{3-[(3S)-3-cyclopropylpiperazin-1-yl]-1,2,4-triazin-6-yl]-5-(2-methoxypyridin-4- |
| | yl)phenol |
| 252¹ | 2-{3-[(3S)-3-cyclopropylpiperazin-1-yl]-1,2,4-triazin-6-yl}-5-(1,2-thiazol-3-yl)phenol |
| 253 | 5-(4-methyl-1,2-thiazol-5-yl)-2-{3-[(3S)-3-(propan-2-yl)piperazin-1-yl]-1,2,4-triazin- |
| | 6-yl}phenol |
| 254¹ | 2-{3-[(3S,5R)-3-tert-butyl-5-methylpiperazin-1-yl]-1,2,4-triazin-6-yl}-5-(1,2,4- |
| | thiadiazol-3-yl)phenol |
| 255¹ | 2-{3-[(3S,5R)-3-tert-butyl-5-methylpiperazin-1-yl]-1,2,4-triazin-6-yl}-5-(6- |
| | methoxypyrimidin-4-yl)phenol |
| 256 | 2-{3-[(3S,5R)-3-cyclopropyl-5-methylpiperazin-1-yl]-1,2,4-triazin-6-yl}-5-(2- |
| | methoxypyridin-4-yl)phenol |
| 257 | 5-(2-methoxypyridin-4-yl)-2-{3-[(3R,5S)-3-methyl-5-(propan-2-yl)piperazin-1-yl]- |
| | 1,2,4-triazin-6-yl}phenol |
| 258 | 2-{3-[(3S)-3-tert-butylpiperazin-1-yl]-1,2,4-triazin-6-yl}-5-(1,2,4-thiadiazol-3- |
| | yl)phenol |
| 259 | 5-(1-methyl-1H-1,2,4-triazol-3-yl)-2-{3-[(3S)-3-(propan-2-yl)piperazin-1-yl]-1,2,4- |
| | triazin-6-yl}phenol |
| 260¹ | 5-(1-methyl-1H-1,2,3-triazol-4-yl)-2-{3-[(3S)-3-(propan-2-yl)piperazin-1-yl]-1,2,4- |
| | triazin-6-yl}phenol |
| 261¹ | 5-(1-methyl-1H-1,2,3-triazol-5-yl)-2-{3-[(3S)-3-(propan-2-yl)piperazin-1-yl]-1,2,4- |
| | triazin-6-yl}phenol |
| 262¹ | 5-(2-methyl-2H-1,2,3-triazol-4-yl)-2-{3-[(3S)-3-(propan-2-yl)piperazin-1-yl]-1,2,4- |
| | triazin-6-yl}phenol |
| 263¹ | 5-(2,1,3-benzothiadiazol-5-yl)-2-{3-[(3S)-3-(propan-2-yl)piperazin-1-yl]-1,2,4-triazin- |
| | 6-yl]phenol |
| 264¹ | 2-{3-[(3S)-3-(propan-2-yl)piperazin-1-yl]-1,2,4-triazin-6-yl}-5- |
| | ([1,2,5]thiadiazolo[3,4-b]pyridin-6-yl)phenol |
| 265¹ | 2-{3-[(3S)-3-(propan-2-yl)piperazin-1-yl]-1,2,4-triazin-6-yl}-5-(1,2,5-thiadiazol-3- |
| | yl)phenol |
| 266 | 2-{3-[(3S,5R)-3-ethyl-5-methylpiperazin-1-yl]-1,2,4-triazin-6-yl}-5-(2- |
| | methoxypyridin-4-yl)phenol or |
| | 2-{3-[(3R,5S)-3-ethyl-5-methylpiperazin-1-yl]-1,2,4-triazin-6-yl}-5-(2- |
| | methoxypyridin-4-yl)phenol |
| 267 | 2-{3-[(3S,5R)-3-ethyl-5-methylpiperazin-1-yl]-1,2,4-triazin-6-yl}-5-(1,2,4-thiadiazol- |
| | 5-yl)phenol or |
| | 2-{3-[(3R,5S)-3-ethyl-5-methylpiperazin-1-yl]-1,2,4-triazin-6-yl}-5-(1,2,4-thiadiazol- |
| | 5-yl)phenol |
| 268 | 2-{3-[(3R,5S)-3-methyl-5-(propan-2-yl)piperazin-1-yl]-1,2,4-triazin-6-yl}-5-(1,2,4- |
| | thiadiazol-5-yl)phenol or |
| | 2-{3-[(3S,5R)-3-methyl-5-(propan-2-yl)piperazin-1-yl]-1,2,4-triazin-6-yl}-5-(1,2,4- |
| | thiadiazol-5-yl)phenol |
| 269 | 2-{3-[(3S,5R)-3-cyclopropyl-5-methylpiperazin-1-yl]-1,2,4-triazin-6-yl}-5-(1,2,4- |
| | thiadiazol-5-yl)phenol or |
| | 2-{3-[(3R,5S)-3-cyclopropyl-5-methylpiperazin-1-yl]-1,2,4-triazin-6-yl}-5-(1,2,4- |
| | thiadiazol-5-yl)phenol |
| 270¹ | 2-{3-[(3S)-3-(propan-2-yl)piperazin-1-yl]-1,2,4-triazin-6-yl}-5-(1,2-thiazol-5- |
| | yl)phenol |
| 271¹ | 5-(2-methoxy-6-methylpyridin-4-yl)-2-{3-[(3S)-3-(propan-2-yl)piperazin-1-yl]-1,2,4- |
| | triazin-6-yl}phenol |
| 272¹ | 2-(3-hydroxy-4-{3-[(3S)-3-(propan-2-yl)piperazin-1-yl]-1,2,4-triazin-6-yl}phenyl)- |
| | 1,3-thiazole-5-carbonitrile |
| 273¹ | 2-(3-hydroxy-4-{3-[(3S)-3-(propan-2-yl)piperazin-1-yl]-1,2,4-triazin-6-yl}phenyl)- |
| | 1,3-thiazole-4-carbonitrile |
| 274¹ | 5-(2-methyl-5,6-dihydro[1,2,4]triazolo[1,5-a]pyrazin-7(8H)-yl)-2-{3-[(3S)-3-(propan- |
| | 2-yl)piperazin-1-yl]-1,2,4-triazin-6-yl}phenol |
| 275¹ | 2-{3-[(3S)-3-cyclopropylpiperazin-1-yl]-1,2,4-triazin-6-yl}-5-(1-methyl-2,3-dihydro- |
| | IH-imidazo[1,2-b]pyrazol-7-yl)phenol |
| 276¹ | 2-{3-[(3S)-3-cyclopropylpiperazin-1-yl]-1,2,4-triazin-6-yl}-5-(5,6-dihydro-4H- |
| | pyrrolo[1,2-b]pyrazol-3-yl)phenol |
| 277¹ | 2-{3-[(3S)-3-cyclopropylpiperazin-1-yl]-1,2,4-triazin-6-yl}-5-(4,5,6,7- |
| | tetrahydropyrazolo[1,5-a]pyridin-3-yl)phenol or |
| | 2-{3-[(3R)-3-cyclopropylpiperazin-1-yl]-1,2,4-triazin-6-yl}-5-(4,5,6,7- |
| | tetrahydropyrazolo[1,5-a]pyridin-3-yl)phenol |
| 278 | 5-(5,6-dihydro-4H-pyrrolo[1,2-b]pyrazol-3-yl)-2-{3-[(3S)-3-(propan-2-yl)piperazin-1- |
| | yl]-1,2,4-triazin-6-yl}phenol |
| 279 | 5-(6,7-dihydro-5H-pyrrolo[1,2-a]imidazol-3-yl)-2-{3-[(3S)-3-(propan-2-yl)piperazin- |
| | 1-yl]-1,2,4-triazin-6-yl}phenol |
| 280 | 5-(5,6-dihydro-4H-pyrrolo[1,2-b]pyrazol-3-yl)-2-{3-[(3S)-3-(1- |
| | methylcyclopropyl)piperazin-1-yl]-1,2,4-triazin-6-yl}phenol |
| 281 | 5-(5,6-dihydro-4H-pyrrolo[1,2-b]pyrazol-3-yl)-2-{3-[(3R)-3-(1- |
| | methylcyclopropyl)piperazin-1-yl]-1,2,4-triazin-6-yl}phenol |
| 282 | 5-(6,7-dihydro-5H-pyrazolo[5,1-b][1,3]oxazin-3-yl)-2-{3-[(3S)-3-(propan-2- |
| | yl)piperazin-1-yl]-1,2,4-triazin-6-yl}phenol, and |
| 283¹ | 3-fluoro-5-(5-fluoro-1H-pyrazol-4-yl)-2-{3-[(3S)-3-(propan-2-yl)piperazin-1-yl]- |
| | 1,2,4-triazin-6-yl}phenol; |

wherein a form of the compound is selected from the group consisting of a salt, hydrate, solvate, and tautomer form thereof.

Another aspect of the compound of Formula (I) or a form thereof is a compound salt selected from the group consisting of:

| Cpd | Name |
|---|---|
| 1 | 2-{3-[(3S)-3-cyclopropylpiperazin-1-yl]-1,2,4-triazin-6-yl}-5-(3-fluoro-1H-pyrazol-4- |
| | yl)phenol dihydrochloride |
| 2 | 2-{3-[(3S)-3-cyclopropylpiperazin-1-yl]-1,2,4-triazin-6-yl}-5-{6- |
| | [(²H₃)methyloxy]pyrimidin-4-yl}phenol dihydrochloride |
| 3 | 5-(3-fluoro-1H-pyrazol-4-yl)-2-{3-[(3S)-3-(propan-2-yl)piperazin-1-yl]-1,2,4-triazin- |
| | 6-yl}phenol dihydrochloride |
| 4 | 5-(3-fluoro-1H-pyrazol-4-yl)-2-{3-[3-(2-hydroxypropan-2-yl)piperazin-1-yl]-1,2,4- |
| | triazin-6-yl}phenol dihydrochloride |
| 5 | 2-[3-(3-cyclopropylpiperazin-1-yl)-1,2,4-triazin-6-yl]-5-(1H-pyrazol-4-yl)phenol |
| | dihydrochloride |
| 6 | 2-{3-[3-(1-hydroxycyclopropyl)piperazin-1-yl]-1,2,4-triazin-6-yl}-5-(1H-pyrazol-4- |
| | yl)phenol diformate |
| 7 | 2-{3-[(3R)-3-cyclopropylpiperazin-1-yl]-1,2,4-triazin-6-yl}-5-(3-fluoro-1H-pyrazol-4- |
| | yl)phenol dihydrochloride |
| 8 | 2-{3-[3-(2-hydroxypropan-2-yl)piperazin-1-yl]-1,2,4-triazin-6-yl}-5-{6- |
| | [(²H₃)methyloxy]pyrimidin-4-yl}phenol dihydrochloride |
| 9 | 2-{3-[(3S)-3-cyclopropylpiperazin-1-yl]-1,2,4-triazin-6-yl]-5-[1-(²H₃)methyl-1H- |
| | pyrazol-4-yl]phenol dihydrochloride |
| 10 | 5-{6-[(²H₃)methyloxy]pyrimidin-4-yl}-2-{3-[(3S)-3-(propan-2-yl)piperazin-1-yl]- |
| | 1,2,4-triazin-6-yl}phenol dihydrochloride |
| 11 | 2-{3-[(3S)-3-cyclopropylpiperazin-1-yl]-1,2,4-triazin-6-yl]-5-(2H-1,2,3-triazol-2- |
| | yl)phenol dihydrochloride |
| 12 | 2-{3-[(3S)-3-cyclopropylpiperazin-1-yl]-1,2,4-triazin-6-yl}-5-(1-methyl-1H-pyrazol- |
| | 4-yl)phenol dihydrochloride |
| 13 | 2-{3-[3-(2-hydroxypropan-2-yl)piperazin-1-yl]-1,2,4-triazin-6-yl}-5-(1H-pyrazol-4- |
| | yl)phenol dihydrochloride |
| 14 | 2-{3-[3-(2-hydroxypropan-2-yl)piperazin-1-yl]-1,2,4-triazin-6-yl}-5-(2H-1,2,3- |
| | triazol-2-yl)phenol dihydrochloride |
| 15 | 5-(3-fluoro-1H-pyrazol-4-yl)-2-{3-[3-(propan-2-yl)piperazin-1-yl]-1,2,4-triazin-6- |
| | yl]phenol dihydrochloride |
| 16 | 2-{3-[(3S)-3-tert-butylpiperazin-1-yl]-1,2,4-triazin-6-yl}-5-(1H-pyrazol-4-yl)phenol |
| | dihydrochloride |
| 17 | 2-{3-[(3S)-3-tert-butylpiperazin-1-yl]-1,2,4-triazin-6-yl}-5-{6- |
| | [(²H₃)methyloxy]pyrimidin-4-yl}phenol dihydrochloride |
| 18 | 2-{3-[(3S)-3-tert-butylpiperazin-1-yl]-1,2,4-triazin-6-yl}-5-(2H-1,2,3-triazol-2- |
| | yl)phenol dihydrochloride |
| 19 | 2-{3-[3-(2-hydroxypropan-2-yl)piperazin-1-yl]-1,2,4-triazin-6-yl}-5-[1-(²H₃)methyl- |
| | 1H-pyrazol-4-yl]phenol dihydrochloride |
| 21 | 2-{3-[(3S)-3-tert-butylpiperazin-1-yl]-1,2,4-triazin-6-yl}-5-(1-methyl-1H-pyrazol-4- |
| | yl)phenol dihydrochloride |
| 24 | 2-[3-(3-ethylpiperazin-1-yl)-1,2,4-triazin-6-yl]-5-(1H-pyrazol-4-yl)phenol |
| | dihydrochloride |
| 25 | 5-(3-fluoro-1H-pyrazol-4-yl)-2-{3-[(3R)-3-(2-hydroxypropan-2-yl)piperazin-1-yl]- |
| | 1,2,4-triazin-6-yl}phenol dihydrochloride |
| 26 | 2-{3-[3-(2-hydroxypropan-2-yl)-4-methylpiperazin-1-yl]-1,2,4-triazin-6-yl}-5-{6- |
| | [(²H₃)methyloxy]pyrimidin-4-yl}phenol dihydrochloride |
| 27 | 2-[3-(3-cyclopropyl-4-methylpiperazin-1-yl)-1,2,4-triazin-6-yl]-5-(1H-pyrazol-4- |
| | yl)phenol dihydrochloride |
| 28 | 3-fluoro-5-(6-methoxypyrimidin-4-yl)-2-{3-[(3S)-3-(propan-2-yl)piperazin-1-yl]- |
| | 1,2,4-triazin-6-yl}phenol formate |
| 29 | 2-{3-[3-(1-methoxycyclopropyl)piperazin-1-yl]-1,2,4-triazin-6-yl}-5-(1H-pyrazol-4- |
| | yl)phenol diformate |
| 31 | 2-[3-(3-propylpiperazin-1-yl)-1,2,4-triazin-6-yl]-5-(1H-pyrazol-4-yl)phenol |
| | dihydrochloride |
| 32 | 2-[3-(3-cyclopropylpiperazin-1-yl)-1,2,4-triazin-6-yl]-3-fluoro-5-(5-fluoro-1H- |
| | pyrazol-4-yl)phenol formate |
| 33 | 2-{3-[3-(butan-2-yl)piperazin-1-yl]-1,2,4-triazin-6-yl}-5-(1H-pyrazol-4-yl)phenol |
| | dihydrochloride |
| 35 | 5-(1-methyl-1H-pyrazol-4-yl)-2-{3-[3-(propan-2-yl)piperazin-1-yl]-1,2,4-triazin-6- |
| | yl}phenol dihydrochloride |
| 36 | 2-{3-[3-(2,2-difluorocyclopropyl)piperazin-1-yl]-1,2,4-triazin-6-yl}-5-(1H-pyrazol-4- |
| | yl)phenol dihydrochloride |
| 38 | 2-[3-(3-ethenylpiperazin-1-yl)-1,2,4-triazin-6-yl]-5-(1H-pyrazol-4-yl)phenol |
| | dihydrochloride |
| 39 | 2-[3-(3-ethylpiperazin-1-yl)-1,2,4-triazin-6-yl]-5-[1-(²H₃)methyl-1H-pyrazol-4- |
| | yl]phenol dihydrochloride |
| 41 | 5-[1-(²H₃)methyl-1H-pyrazol-4-yl]-2-{3-[3-(propan-2-yl)piperazin-1-yl]-1,2,4-triazin- |
| | 6-yl}phenol dihydrochloride |
| 43 | 2-[3-(6,9-diazaspiro[4.5]decan-9-yl)-1,2,4-triazin-6-yl]-5-(1H-pyrazol-4-yl)phenol |
| | dihydrochloride |
| 44 | 5-(2-methylpyridin-4-yl)-2-{3-[3-(propan-2-yl)piperazin-1-yl]-1,2,4-triazin-6- |
| | yl]phenol dihydrochloride |
| 45 | 2-{3-[(3S)-3-(hydroxymethyl)piperazin-1-yl]-1,2,4-triazin-6-yl}-5-(2H-1,2,3-triazol- |
| | 2-yl)phenol dihydrochloride |
| 47 | 5-[1-(²H₃)methyl-1H-pyrazol-4-yl]-2-{3-[(3S)-3-(propan-2-yl)piperazin-1-yl]-1,2,4- |
| | triazin-6-yl} phenol dihydrochloride |
| 49 | 2-{3-[(3R,5S)-3,5-dimethylpiperazin-1-yl]-1,2,4-triazin-6-yl}-5-[1-(²H₃)methyl-1H- |
| | pyrazol-4-yl]phenol dihydrochloride |
| 50 | 2-[3-(5,8-diazaspiro[3.5]nonan-8-yl)-1,2,4-triazin-6-yl]-5-(1H-pyrazol-4-yl)phenol |
| | dihydrochloride |
| 51 | 5-[1-(²H₃)methyl-1H-pyrazol-4-yl]-2-{3-[(3R,5S)-3,4,5-trimethylpiperazin-1-yl]- |
| | 1,2,4-triazin-6-yl}phenol dihydrochloride |
| 52 | 5-(2H-1,2,3-triazol-2-yl)-2-{3-[(3R,5S)-3,4,5-trimethylpiperazin-1-yl]-1,2,4-triazin-6- |
| | yl}phenol dihydrochloride |
| 54 | 2-{3-[(3R,5S)-3,5-dimethylpiperazin-1-yl]-1,2,4-triazin-6-yl}-5-(2H-1,2,3-triazol-2- |
| | yl)phenol dihydrochloride |
| 55 | 2-[3-(4,7-diazaspiro[2.5]octan-7-yl)-1,2,4-triazin-6-yl]-5-(1H-pyrazol-4-yl)phenol |
| | dihydrochloride |
| 56 | 2-[3-(3,3-dimethylpiperazin-1-yl)-1,2,4-triazin-6-yl]-5-(1H-pyrazol-4-yl)phenol |
| | dihydrochloride |
| 57 | 2-[3-(4,7-diazaspiro[2.5]octan-7-yl)-1,2,4-triazin-6-yl]-5-(3-fluoro-1H-pyrazol-4- |
| | yl)phenol dihydrochloride |
| 58 | 2-[3-(8-methyl-3,8-diazabicyclo[3.2.1]octan-3-yl)-1,2,4-triazin-6-yl]-5-[1- |
| | (²H₃)methyl-1H-pyrazol-4-yl]phenol dihydrochloride |
| 59 | (7R,8aS)-2-{6-[2-hydroxy-4-(1H-pyrazol-4-yl)phenyl]-1,2,4-triazin-3- |
| | yl}octahydropyrrolo[1,2-a]pyrazin-7-ol dihydrochloride |
| 61 | 2-[3-(3-phenylpiperazin-1-yl)-1,2,4-triazin-6-yl]-5-(1H-pyrazol-4-yl)phenol |
| | dihydrochloride |
| 62 | 5-(1H-pyrazol-4-yl)-2-{3-[3-(pyridin-4-yl)piperazin-1-yl]-1,2,4-triazin-6-yl}phenol |
| | dihydrochloride |
| 65 | 5-(7-fluoro-2-methyl-2H-indazol-5-yl)-2-{3-[3-(hydroxymethyl)piperazin-1-yl]-1,2,4- |
| | triazin-6-yl}phenol dihydrochloride |
| 66 | 2-{3-[(3R,5S)-3,5-dimethylpiperazin-1-yl]-1,2,4-triazin-6-yl}-5-(8-fluoro-2- |
| | methylimidazo[1,2-a]pyridin-6-yl)phenol dihydrochloride |
| 67 | 5-(7-fluoro-2-methyl-2H-indazol-5-yl)-2-[3-(3-methylpiperazin-1-yl)-1,2,4-triazin-6- |
| | yl]phenol dihydrochloride |
| 68 | 2-[3-(3-ethylpiperazin-1-yl)-1,2,4-triazin-6-yl]-5-(8-fluoro-2-methylimidazo[1,2-a] |
| | pyridin-6-yl)phenol dihydrochloride |
| 69 | 5-(2,8-dimethylimidazo[1,2-b]pyridazin-6-yl)-2-{3-[(3R,5S)-3,5-dimethylpiperazin-1- |
| | yl]-1,2,4-triazin-6-yl}phenol dihydrochloride |
| 70 | 5-(8-methoxy-2-methyl[1,2,4]triazolo[1,5-b]pyridazin-6-yl)-2-{3-[(3S)-3-(propan-2- |
| | yl)piperazin-1-yl]-1,2,4-triazin-6-yl}phenol dihydrochloride |
| 71 | 5-(7-fluoro-2-methyl-2H-indazol-5-yl)-2-{3-[3-(propan-2-yl)piperazin-1-yl]-1,2,4- |
| | triazin-6-yl}phenol dihydrochloride |
| 73 | 5-(8-fluoro-2-methylimidazo[1,2-a]pyridin-6-yl)-2-[3-(4-methylpiperazin-1-yl)-1,2,4- |
| | triazin-6-yl]phenol dihydrochloride |
| 74 | 2-[3-(3-ethylpiperazin-1-yl)-1,2,4-triazin-6-yl]-5-(7-fluoro-2-methyl-2H-indazol-5- |
| | yl)phenol dihydrochloride |
| 77 | 2-[3-(4-ethylpiperazin-1-yl)-1,2,4-triazin-6-yl]-5-(8-fluoro-2-methylimidazo[1,2- |
| | a]pyridin-6-yl)phenol dihydrochloride |
| 78 | 5-(2,8-dimethyl[1,2,4]triazolo[1,5-a]pyrazin-6-yl)-2-{3-[(3S)-3-(propan-2- |
| | yl)piperazin-1-yl]-1,2,4-triazin-6-yl}phenol dihydrochloride |
| 80 | 5-(4-{3-[(3R,5S)-3,5-dimethylpiperazin-1-yl]-1,2,4-triazin-6-yl}-3-hydroxyphenyl)-2- |
| | methyl-2H-indazole-7-carbonitrile dihydrochloride |
| 81 | 5-(7-fluoro-2-methyl-2H-indazol-5-yl)-2-{3-[(3S)-3-(propan-2-yl)piperazin-1-yl]- |
| | 1,2,4-triazin-6-yl}phenol dihydrochloride |
| 83 | 2-{3-[(3R,5S)-3,5-dimethylpiperazin-1-yl]-1,2,4-triazin-6-yl}-5-(2,8- |
| | dimethyl[1,2,4]triazolo[1,5-b]pyridazin-6-yl)phenol dihydrochloride |
| 84 | 2-{3-[(3S)-3-cyclopropylpiperazin-1-yl]-1,2,4-triazin-6-yl}-5-(6,8-dimethyl-7H-purin- |
| | 2-yl)phenol diformate |
| 85 | 5-(2-methyl[1,2,4]triazolo[1,5-a]pyridin-6-yl)-2-{3-[(3S)-3-(propan-2-yl)piperazin-1- |
| | yl]-1,2,4-triazin-6-yl}phenol dihydrochloride |
| 86 | 2-{3-[(3R,5S)-3,5-dimethylpiperazin-1-yl]-1,2,4-triazin-6-yl}-5-(2- |
| | methylimidazo[1,2-a]pyrazin-6-yl)phenol dihydrochloride |
| 88 | 5-(2-methyl-2H-indazol-5-yl)-2-{3-[(3S)-3-(propan-2-yl)piperazin-1-yl]-1,2,4-triazin- |
| | 6-yl}phenol dihydrochloride |
| 89 | 2-{3-[(3R)-4-ethyl-3-methylpiperazin-1-yl]-1,2,4-triazin-6-yl}-5-(8-fluoro-2- |
| | methylimidazo[1,2-a]pyridin-6-yl)phenol dihydrochloride |
| 90 | 2-{3-[(3R,5S)-3,5-dimethylpiperazin-1-yl]-1,2,4-triazin-6-yl}-5-(8-methoxy-2- |
| | methyl[1,2,4]triazolo[1,5-b]pyridazin-6-yl)phenol dihydrochloride |
| 91 | 5-(2-methyl[1,2,4]triazolo[1,5-a]pyrazin-6-yl)-2-{3-[(3S)-3-(propan-2-yl)piperazin-1- |
| | yl]-1,2,4-triazin-6-yl}phenol dihydrochloride |
| 92 | 5-(2,8-dimethyl[1,2,4]triazolo[1,5-b]pyridazin-6-yl)-2-{3-[(3S)-3-(propan-2- |
| | yl)piperazin-1-yl]-1,2,4-triazin-6-yl}phenol dihydrochloride |
| 93 | 5-(8-methoxy-2-methyl[1,2,4]triazolo[1,5-a]pyrazin-6-yl)-2-{3-[(3S)-3-(propan-2- |
| | yl)piperazin-1-yl]-1,2,4-triazin-6-yl}phenol dihydrochloride |
| 94 | 5-(8-fluoro-2-methylimidazo[1,2-a]pyridin-6-yl)-2-{3-[(3R,5S)-3,4,5- |
| | trimethylpiperazin-1-yl]-1,2,4-triazin-6-yl}phenol dihydrochloride |
| 95 | 5-(7-fluoro-2-methyl-2H-indazol-5-yl)-2-[3-(hexahydropyrrolo[1,2-a]pyrazin-2(1H)- |
| | yl)-1,2,4-triazin-6-yl]phenol dihydrochloride |
| 96 | 5-(2,8-dimethylimidazo[1,2-a]pyrazin-6-yl)-2-{3-[(3S)-3-(propan-2-yl)piperazin-1- |
| | yl]-1,2,4-triazin-6-yl}phenol dihydrochloride |
| 98 | 5-(2,8-dimethylimidazo[1,2-b]pyridazin-6-yl)-2-{3-[(3R)-3-methylpiperazin-1-yl]- |
| | 1,2,4-triazin-6-yl}phenol dihydrochloride |
| 100 | 2-{3-[(3R,5S)-3,5-dimethylpiperazin-1-yl]-1,2,4-triazin-6-yl}-5-(2- |
| | methyl[1,2,4]triazolo[1,5-b]pyridazin-6-yl)phenol dihydrochloride |
| 101 | 2-{3-[(3R,5S)-3,5-dimethylpiperazin-1-yl]-1,2,4-triazin-6-yl}-5-(2,8- |
| | dimethyl[1,2,4]triazolo[1,5-a]pyrazin-6-yl)phenol dihydrochloride |
| 102 | 5-(imidazo[1,2-b]pyridazin-6-yl)-2-{3-[(3S)-3-(propan-2-yl)piperazin-1-yl]-1,2,4- |
| | triazin-6-yl}phenol dihydrochloride |
| 103 | 5-(2,8-dimethylimidazo[1,2-a]pyrazin-6-yl)-2-{3-[(3R,5S)-3,5-dimethylpiperazin-1- |
| | yl]-1,2,4-triazin-6-yl}phenol dihydrochloride |
| 104 | 5-(2,8-dimethylimidazo[1,2-b]pyridazin-6-yl)-2-[3-(4-methylpiperazin-1-yl)-1,2,4- |
| | triazin-6-yl]phenol dihydrochloride |
| 105 | 5-(7-fluoro-2-methyl-2H-indazol-5-yl)-2-{3-[(8aR)-hexahydropyrrolo[1,2-a]pyrazin- |
| | 2(1H)-yl]-1,2,4-triazin-6-yl}phenol dihydrochloride |
| 106 | 2-{3-[(3R,5S)-3,5-dimethylpiperazin-1-yl]-1,2,4-triazin-6-yl}-5-(2- |
| | methyl[1,2,4]triazolo[l,5-a]pyrazin-6-yl)phenol dihydrochloride |
| 107 | 2-{3-[(3R)-3,4-dimethylpiperazin-1-yl]-1,2,4-triazin-6-yl]-5-(8-fluoro-2- |
| | methylimidazo[1,2-a]pyridin-6-yl)phenol dihydrochloride |
| 108 | 6-(3-hydroxy-4-{3-[(3R,5S)-3,4,5-trimethylpiperazin-1-yl]-1,2,4-triazin-6-yl}phenyl)- |
| | 2-methylimidazo[1,2-b]pyridazine-8-carbonitrile dihydrochloride |
| 109 | 5-(8-cyclopropyl-2-methyl[1,2,4]triazolo[l,5-a]pyrazin-6-yl)-2-{3-[(3R,5S)-3,5- |
| | dimethylpiperazin-1-yl]-1,2,4-triazin-6-yl]phenol dihydrochloride |
| 111 | 5-(2,8-dimethylimidazo[1,2-b]pyridazin-6-yl)-2-{3-[(3R,5S)-3,4,5-trimethylpiperazin- |
| | 1-yl]-1,2,4-triazin-6-yl]phenol dihydrochloride |
| 112 | 2-{3-[(3R,5S)-3,5-dimethylpiperazin-1-yl]-1,2,4-triazin-6-yl}-5-(2- |
| | methyl[1,2,4]triazolo[l,5-a]pyrimidin-6-yl)phenol dihydrochloride |
| 113 | 5-(imidazo[1,2-a]pyrazin-6-yl)-2-{3-[(3S)-3-(propan-2-yl)piperazin-1-yl]-1,2,4- |
| | triazin-6-yl}phenol dihydrochloride |
| 114 | 5-(imidazo[1,2-a]pyridin-6-yl)-2-{3-[(3S)-3-(propan-2-yl)piperazin-1-yl]-1,2,4- |
| | triazin-6-yl}phenol dihydrochloride |
| 115 | 2-{3-[(3S)-3-(propan-2-yl)piperazin-1-yl]-1,2,4-triazin-6-yl}-5-([1,2,4]triazolo[4,3- |
| | a]pyridin-6-yl)phenol dihydrochloride |
| 116 | 2-{3-[(3R,5S)-3,5-dimethylpiperazin-1-yl]-1,2,4-triazin-6-yl}-5-(4,6- |
| | dimethyl[1,3]thiazolo[5,4-c]pyridin-2-yl)phenol dihydrochloride |
| 117 | 2-{3-[(3R,5S)-3,5-dimethylpiperazin-1-yl]-1,2,4-triazin-6-yl}-5-(5,7- |
| | dimethyl[1,2,4]triazolo[1,5-a]pyrimidin-2-yl)phenol dihydrochloride |
| 118 | 2-{3-[(3R,5S)-3,5-dimethylpiperazin-1-yl]-1,2,4-triazin-6-yl}-5-[2- |
| | (trifluoromethyl)imidazo[1,2-b]pyridazin-6-yl]phenol dihydrochloride |
| 119 | 2-{3-[(3R,5S)-3,5-dimethylpiperazin-1-yl]-1,2,4-triazin-6-yl}-5-(6- |
| | methyl[l,3]thiazolo[4,5-b]pyrazin-2-yl)phenol dihydrochloride |
| 120 | 2-{3-[(3R,5S)-3,5-dimethylpiperazin-1-yl]-1,2,4-triazin-6-yl}-5-(5-methylfuro[3,2- |
| | b]pyridin-2-yl)phenol dihydrochloride |
| 121 | 5-(7-methoxy-2-methyl-2H-indazol-5-yl)-2-[3-(4-methylpiperazin-1-yl)-1,2,4-triazin- |
| | 6-yl]pyridin-3-ol hydrochloride |
| 122 | 5-(8-fluoro-2-methylimidazo[1,2-a]pyridin-6-yl)-2-[3-(4-methylpiperazin-1-yl)-1,2,4- |
| | triazin-6-yl]pyridin-3-ol hydrochloride |
| 123 | 2-{3-[(3S)-3-ethylpiperazin-1-yl]-1,2,4-triazin-6-yl}-5-(3-fluoro-1H-pyrazol-4- |
| | yl)phenol dihydrochloride |
| 124 | 2-[3-(4-methylpiperazin-1-yl)-1,2,4-triazin-6-yl]-5-(1H-pyrazol-4-yl)pyridin-3-ol |
| | hydrochloride |
| 125 | 2-{3-[(3S)-3-tert-butylpiperazin-1-yl]-1,2,4-triazin-6-yl}-5-(3-fluoro-1H-pyrazol-4- |
| | yl)phenol dihydrochloride |
| 126 | 5-(2,8-dimethylimidazo[1,2-a]pyridin-6-yl)-2-[3-(4-methylpiperazin-1-yl)-1,2,4- |
| | triazin-6-yl]pyridin-3-ol hydrochloride |
| 127 | 3-methyl-2-{3-[(3S)-3-(propan-2-yl)piperazin-1-yl]-1,2,4-triazin-6-yl}-5-(1H-pyrazol- |
| | 4-yl)phenol formate |
| 128 | 2-{3-[(3S)-3-(propan-2-yl)piperazin-1-yl]-1,2,4-triazin-6-yl}-5-([1,2,4]triazolo[1,5- |
| | a]pyrazin-6-yl)phenol dihydrochloride |
| 129 | 2-{3-[(3S)-3-tert-butylpiperazin-1-yl]-1,2,4-triazin-6-yl}-5-([1,2,4]triazolo[1,5- |
| | a]pyrazin-6-yl)phenol dihydrochloride |
| 130 | 2-{3-[(3S)-3-cyclopropylpiperazin-1-yl]-1,2,4-triazin-6-yl}-5-([1,2,4]triazolo[1,5- |
| | a]pyrazin-6-yl)phenol dihydrochloride |
| 131 | 5-(2,8-dimethylimidazo[1,2-a]pyridin-6-yl)-2-{3-[(3S)-3-(propan-2-yl)piperazin-1- |
| | yl]-1,2,4-triazin-6-yl}phenol dihydrochloride |
| 132 | 2-{3-[(3S)-3-ethylpiperazin-1-yl]-1,2,4-triazin-6-yl}-5-(2H-1,2,3-triazol-2-yl)phenol |
| | dihydrochloride |
| 133 | 2-{3-[(3S)-3-ethylpiperazin-1-yl]-1,2,4-triazin-6-yl}-5-{6-[(²H₃)methyloxy]pyrimidin- |
| | 4-yl}phenol dihydrochloride |
| 134 | 2-{3-[3-(1-methylcyclopropyl)piperazin-1-yl]-1,2,4-triazin-6-yl}-5-(1H-pyrazol-4- |
| | yl)phenol formate |
| 135 | 2-[3-(3,8-diazabicyclo[3.2.1]octan-3-yl)-1,2,4-triazin-6-yl]-5-(1H-pyrazol-4-yl)phenol |
| | diformate |
| 136 | 2-{3-[(3R)-3-cyclopropylpiperazin-1-yl]-1,2,4-triazin-6-yl}-5-(1H-pyrazol-4- |
| | yl)phenol dihydrochloride |
| 137 | 2-{3-[(3S)-3-cyclopropylpiperazin-1-yl]-1,2,4-triazin-6-yl}-5-(1H-pyrazol-4- |
| | yl)phenol dihydrochloride |
| 138 | 5-(8-ethyl-2-methylimidazo[1,2-a]pyridin-6-yl)-2-{3-[(3S)-3-(propan-2-yl)piperazin- |
| | 1-yl]-1,2,4-triazin-6-yl}phenol dihydrochloride |
| 139 | 5-[2-methyl-8-(trifluoromethyl)imidazo[1,2-a]pyridin-6-yl]-2-{3-[(3S)-3-(propan-2- |
| | yl)piperazin-1-yl]-1,2,4-triazin-6-yl}phenol dihydrochloride |
| 140 | 5-(2,7-dimethyl-2H-indazol-5-yl)-2-{3-[(3S)-3-(propan-2-yl)piperazin-1-yl]-1,2,4- |
| | triazin-6-yl}phenol dihydrochloride |
| 141 | 5-(2-methylimidazo[1,2-a]pyrazin-6-yl)-2-{3-[(3S)-3-(propan-2-yl)piperazin-1-yl]- |
| | 1,2,4-triazin-6-yl}phenol dihydrochloride |
| 146 | 5-(1H-pyrazol-4-yl)-2-{3-[3-(2,2,2-trifluoroethyl)piperazin-1-yl]-1,2,4-triazin-6- |
| | yl]phenol diformate |
| 147 | 5-(2-methyl[1,2,4]triazolo[l,5-a]pyridin-7-yl)-2-{3-[(3S)-3-(propan-2-yl)piperazin-1- |
| | yl]-1,2,4-triazin-6-yl}phenol dihydrochloride |
| 148 | 2-{3-[rac-(3S,5R)-3-ethyl-5-methylpiperazin-1-yl]-1,2,4-triazin-6-yl}-5-(1H-pyrazol- |
| | 4-yl)phenol dihydrochloride |
| 149 | 5-(6-methylpyrimidin-4-yl)-2-{3-[(3RS)-3-(propan-2-yl)piperazin-1-yl]-1,2,4-triazin- |
| | 6-yl}phenol dihydrochloride |
| 152 | 4-fluoro-5-[1-(²H₃)methyl-1H-pyrazol-4-yl]-2-{3-[(3S)-3-(propan-2-yl)piperazin-1- |
| | yl]-1,2,4-triazin-6-yl}phenol formate |
| 153 | 2-{3-[(8aS)-hexahydropyrrolo[1,2-a]pyrazin-2(1H)-yl]-1,2,4-triazin-6-yl}-5-(2H- |
| | 1,2,3-triazol-2-yl)phenol dihydrochloride |
| 154 | 2-{3-[(3S)-3-(propan-2-yl)piperazin-1-yl]-1,2,4-triazin-6-yl}-5-(2H-1,2,3-triazol-2- |
| | yl)phenol dihydrochloride |
| 155 | 5-(5-methyl-1H-pyrazolo[4,3-b]pyridin-1-yl)-2-{3-[(3S)-3-(propan-2-yl)piperazin-1- |
| | yl]-1,2,4-triazin-6-yl}phenol dihydrochloride |
| 156 | 2-{3-[(3S)-3-tert-butylpiperazin-1-yl]-1,2,4-triazin-6-yl}-4-fluoro-5-[1-(²H₃)methyl- |
| | 1H-pyrazol-4-yl]phenol formate |
| 157 | 5-(7-fluoro-2-methyl-2H-indazol-5-yl)-2-{3-[(3S)-3-(propan-2-yl)piperazin-1-yl]- |
| | 1,2,4-triazin-6-yl}pyridin-3-ol hydrochloride |
| 158 | 4-fluoro-2-{3-[(3S)-3-(propan-2-yl)piperazin-1-yl]-1,2,4-triazin-6-yl}-5-(1H-pyrazol- |
| | 4-yl)phenol formate |
| 159 | 5-(5-methyl-1H-pyrrolo[3,2-b]pyridin-1-yl)-2-{3-[(3S)-3-(propan-2-yl)piperazin-1- |
| | yl]-1,2,4-triazin-6-yl}phenol formate |
| 162 | 2-{3-[(3S)-3-(propan-2-yl)piperazin-1-yl]-1,2,4-triazin-6-yl}-5-(1H-pyrazolo[3,4- |
| | d]pyrimidin-1-yl)phenol dihydrochloride |
| 163 | 5-(3-chloro-1H-pyrazol-4-yl)-2-{3-[(3S)-3-cyclopropylpiperazin-1-yl]-1,2,4-triazin-6- |
| | yl}phenol dihydrochloride |
| 164 | 2-{3-[(3S)-3-tert-butylpiperazin-1-yl]-1,2,4-triazin-6-yl}-4-fluoro-5-(1H-pyrazol-4- |
| | yl)phenol formate |
| 165 | 2-{3-[(3S)-3-methylpiperazin-1-yl]-1,2,4-triazin-6-yl}-5-(1H-pyrazol-4-yl)phenol |
| | dihydrochloride |
| 166 | 2-{3-[(3R)-3-methylpiperazin-1-yl]-1,2,4-triazin-6-yl}-5-(1H-pyrazol-4-yl)phenol |
| | dihydrochloride |
| 168 | 2-{3-[(3S)-3-cyclopropylpiperazin-1-yl]-1,2,4-triazin-6-yl}-5-(2-methyl-2H- |
| | [1,2,3]triazolo[4,5-b]pyridin-6-yl)phenol dihydrochloride |
| 169 | 2-{3-[(3S)-3-cyclopropylpiperazin-1-yl]-1,2,4-triazin-6-yl}-5-(1-methyl-1H- |
| | [1,2,3]triazolo[4,5-b]pyridin-6-yl)phenol dihydrochloride |
| 171 | 2-{3-[(3S)-3-cyclopropylpiperazin-1-yl]-1,2,4-triazin-6-yl}-5-(1-methyl-1H- |
| | [1,2,3]triazolo[4,5-b]pyridin-5-yl)phenol dihydrochloride |
| 172 | 2-{3-[(3S)-3-cyclopropylpiperazin-1-yl]-1,2,4-triazin-6-yl}-5-(2-methyl-2H- |
| | [1,2,3]triazolo[4,5-c]pyridin-6-yl)phenol dihydrochloride |
| 173 | 2-{3-[(3S)-3-cyclopropylpiperazin-1-yl]-1,2,4-triazin-6-yl}-5-(3-methyl-3H- |
| | [1,2,3]triazolo[4,5-b]pyridin-5-yl)phenol dihydrochloride |
| 174 | 2-{3-[(3S)-3-cyclopropylpiperazin-1-yl]-1,2,4-triazin-6-yl}-5-(1-methyl-1H- |
| | [1,2,3]triazolo[4,5-c]pyridin-6-yl)phenol dihydrochloride |
| 175 | 2-{3-[(3S)-3-(propan-2-yl)piperazin-1-yl]-1,2,4-triazin-6-yl}-5-(2H-1,2,3-triazol-2- |
| | yl)pyridin-3-ol dihydrochloride |
| 176 | 2-{3-[(2S,5S)-2,5-dimethylpiperazin-1-yl]-1,2,4-triazin-6-yl}-5-(1H-pyrazol-4- |
| | yl)phenol dihydrochloride |
| 177 | 2-{3-[(3S)-3-cyclopropylpiperazin-1-yl]-1,2,4-triazin-6-yl}-5-(pyridin-4-yl)phenol |
| | dihydrochloride |
| 178 | 2-{3-[(3S)-3-cyclopropylpiperazin-1-yl]-1,2,4-triazin-6-yl}-5-(3-fluoropyridin-4- |
| | yl)phenol dihydrochloride |
| 179 | 4-{3-[(3S)-3-cyclopropylpiperazin-1-yl]-1,2,4-triazin-6-yl}-4′-(methylamino) |
| | [1,1′-biphenyl]-3-ol dihydrochloride |
| 180 | 2-{3-[(3S)-3-cyclopropylpiperazin-1-yl]-1,2,4-triazin-6-yl}-5-(2-methyl-2H- |
| | [1,2,3]triazolo[4,5-b]pyridin-6-yl)pyridin-3-ol trifluoroacetate |
| 181 | 2-{3-[(3S)-3-cyclopropylpiperazin-1-yl]-1,2,4-triazin-6-yl}-5-(7-fluoro-2-methyl-2H- |
| | indazol-5-yl)pyridin-3-ol hydrochloride |
| 182 | 2-{3-[(3S)-3-cyclopropylpiperazin-1-yl]-1,2,4-triazin-6-yl]-5-(2H-1,2,3-triazol-2- |
| | yl)pyridin-3-ol trifluoroacetate |
| 183 | 2-{3-[(3R)-3-cyclopropylpiperazin-1-yl]-1,2,4-triazin-6-yl}-5-(4-methyl-2H-1,2,3- |
| | triazol-2-yl)phenol dihydrochloride |
| 184 | 5-(4-methyl-2H-1,2,3-triazol-2-yl)-2-{3-[(3S)-3-(propan-2-yl)piperazin-1-yl]-1,2,4- |
| | triazin-6-yl}phenol dihydrochloride |
| 187 | 2-{3-[4-methyl-3-(oxetan-3-yl)piperazin-1-yl]-1,2,4-triazin-6-yl}-5-(1H-pyrazol-4- |
| | yl)phenol formate |
| 190 | 2-{3-[(2R,5S)-2,5-dimethylpiperazin-1-yl]-1,2,4-triazin-6-yl}-5-(1H-pyrazol-4- |
| | yl)phenol or enantiomer trifluoroacetate |
| 191 | 2-{3-[(3S)-3-(propan-2-yl)piperazin-1-yl]-1,2,4-triazin-6-yl}-5-(1,3]thiazolo[5,4- |
| | b]pyridin-2-yl)phenol trifluoroacetate |
| 193 | 2-{3-[(3S)-3-cyclopropylpiperazin-1-yl]-1,2,4-triazin-6-yl}-5-(3- |
| | methyl[1,2,3]triazolo[l,5-a]pyridin-6-yl)phenol formate |
| 194 | 2-{3-[(3S)-3-cyclopropylpiperazin-1-yl]-1,2,4-triazin-6-yl}-5-(1-methylimidazo[1,5- |
| | a]pyridin-6-yl)phenol formate |
| 195 | 2-{3-[(3S)-3-cyclopropylpiperazin-1-yl]-1,2,4-triazin-6-yl]-5-(3-methylimidazo[1,5- |
| | a]pyridin-7-yl)phenol formate |
| 202 | 2-{3-[(3S)-3-cyclopropylpiperazin-1-yl]-1,2,4-triazin-6-yl}-5-(2-methyl-2H- |
| | [1,2,3]triazolo[4,5-b]pyridin-5-yl)phenol dihydrochloride |
| 208 | 2-{3-[(3S)-3-cyclopropylpiperazin-1-yl]-1,2,4-triazin-6-yl}-5-(1-ethyl-1H-pyrazol-4- |
| | yl)phenol dihydrochloride |
| 211 | 2-{3-[(3S)-3-tert-butylpiperazin-1-yl]-1,2,4-triazin-6-yl}-5-(2-methyl-2H- |
| | [1,2,3]triazolo[4,5-b]pyridin-6-yl)phenol dihydrochloride |
| 212 | 5-(2-methyl-2H-[1,2,3]triazolo[4,5-b]pyridin-6-yl)-2-{3-[(3S)-3-(propan-2- |
| | yl)piperazin-1-yl]-1,2,4-triazin-6-yl}phenol dihydrochloride |
| 213 | 2-{3-[(3S)-3-cyclopropylpiperazin-1-yl]-1,2,4-triazin-6-yl]-5-(1,2,4-thiadiazol-3- |
| | yl)phenol trifluoroacetate |
| 231 | 2-{3-[(3S,5R)-3-cyclobutyl-5-methylpiperazin-1-yl]-1,2,4-triazin-6-yl}-5-(1H- |
| | pyrazol-4-yl)phenol dihydrochloride |
| 232 | 2-{3-[(3S)-3-cyclopropylpiperazin-1-yl]-1,2,4-triazin-6-yl}-5-(5-methyl-1,3-oxazol-2- |
| | yl)phenol trifluoroacetate |
| 233 | 2-{3-[(3S)-3-cyclopropylpiperazin-1-yl]-1,2,4-triazin-6-yl}-5-(1,3-oxazol-2-yl)phenol |
| | trifluoroacetate |
| 236 | 2-{3-[(3S,5R)-3-cyclobutyl-5-methylpiperazin-1-yl]-1,2,4-triazin-6-yl}-5-(3-fluoro- |
| | 1H-pyrazol-4-yl)phenol dihydrochloride |
| 237 | 2-{3-[(3S,5R)-3-cyclobutyl-5-methylpiperazin-1-yl]-1,2,4-triazin-6-yl}-5-(2H-1,2,3- |
| | triazol-2-yl)phenol dihydrochloride |
| 238 | 2-{3-[(3S,5R)-3-cyclobutyl-5-methylpiperazin-1-yl]-1,2,4-triazin-6-yl}-5-[1- |
| | (²H₃)methyl-1H-pyrazol-4-yl]phenol hydrochloride |
| 239 | 2-{3-[(3S)-3-(propan-2-yl)piperazin-1-yl]-1,2,4-triazin-6-yl}-5-(1,2,4-thiadiazol-3- |
| | yl)phenol trifluoroacetate |
| 240 | 2-{3-[(3S,5R)-3-cyclobutyl-5-methylpiperazin-1-yl]-1,2,4-triazin-6-yl]-5-(1-methyl- |
| | 1H-pyrazol-3-yl)phenol hydrochloride |
| 241 | 2-{3-[(3S)-3-(1-methylcyclopropyl)piperazin-1-yl]-1,2,4-triazin-6-yl}-5-(2-methyl- |
| | 2H-[1,2,3]triazolo[4,5-b]pyridin-6-yl)phenol hydrochloride |
| 242 | 2-{3-[(3R)-3-(1-methylcyclopropyl)piperazin-1-yl]-1,2,4-triazin-6-yl}-5-(2-methyl- |
| | 2H-[1,2,3]triazolo[4,5-b]pyridin-6-yl)phenol hydrochloride |
| 243 | 2-{3-[(3S)-3-(propan-2-yl)piperazin-1-yl]-1,2,4-triazin-6-yl}-5-(pyrazolo[1,5- |
| | a]pyrimidin-3-yl)phenol trifluoroacetate |
| 245 | 2-{3-[(3S)-3-cyclopropylpiperazin-1-yl]-1,2,4-triazin-6-yl}-5-(5-methyl-1,2,4- |
| | thiadiazol-3-yl)phenol trifluoroacetate |
| 246 | 2-{3-[(3S)-3-cyclopropylpiperazin-1-yl]-1,2,4-triazin-6-yl}-5-(2-methyl-1,3-thiazol-4- |
| | yl)phenol trifluoroacetate |
| 247 | 2-{3-[(3S,5R)-3-tert-butyl-5-methylpiperazin-1-yl]-1,2,4-triazin-6-yl}-5-(1H-pyrazol- |
| | 4-yl)phenol dihydrochloride |
| 248 | 2-{3-[(3S,5R)-3-tert-butyl-5-methylpiperazin-1-yl]-1,2,4-triazin-6-yl}-5-(3-fluoro-1H- |
| | pyrazol-4-yl)phenol dihydrochloride |
| 249 | 2-{3-[(3S,5R)-3-ethenyl-5-methylpiperazin-1-yl]-1,2,4-triazin-6-yl]-5-(1H-pyrazol-4- |
| | yl)phenol dihydrochloride |
| 250 | 2-{3-[(3S)-3-(propan-2-yl)piperazin-1-yl]-1,2,4-triazin-6-yl}-5-(1,2-thiazol-4- |
| | yl)phenol hydrochloride |
| 251 | 2-{3-[(3S)-3-cyclopropylpiperazin-1-yl]-1,2,4-triazin-6-yl}-5-(2-methoxypyridin-4- |
| | yl)phenol trifluoroacetate |
| 252 | 2-{3-[(3S)-3-cyclopropylpiperazin-1-yl]-1,2,4-triazin-6-yl}-5-(1,2-thiazol-3-yl)phenol |
| | trifluoroacetate |
| 254 | 2-{3-[(3S,5R)-3-tert-butyl-5-methylpiperazin-1-yl]-1,2,4-triazin-6-yl}-5-(1,2,4- |
| | thiadiazol-3-yl)phenol hydrochloride |
| 255 | 2-{3-[(3S,5R)-3-tert-butyl-5-methylpiperazin-1-yl]-1,2,4-triazin-6-yl}-5-(6- |
| | methoxypyrimidin-4-yl)phenol hydrochloride |
| 260 | 5-(1-methyl-1H-1,2,3-triazol-4-yl)-2-{3-[(3S)-3-(propan-2-yl)piperazin-1-yl]-1,2,4- |
| | triazin-6-yl}phenol dihydrochloride |
| 261 | 5-(1-methyl-1H-1,2,3-triazol-5-yl)-2-{3-[(3S)-3-(propan-2-yl)piperazin-1-yl]-1,2,4- |
| | triazin-6-yl}phenol dihydrochloride |
| 262 | 5-(2-methyl-2H-1,2,3-triazol-4-yl)-2-{3-[(3S)-3-(propan-2-yl)piperazin-1-yl]-1,2,4- |
| | triazin-6-yl}phenol dihydrochloride |
| 263 | 5-(2,1,3-benzothiadiazol-5-yl)-2-{3-[(3S)-3-(propan-2-yl)piperazin-1-yl]-1,2,4-triazin- |
| | 6-yl}phenol hydrochloride |
| 264 | 2-{3-[(3S)-3-(propan-2-yl)piperazin-1-yl]-1,2,4-triazin-6-yl}-5- |
| | ([1,2,5]thiadiazolo[3,4-b]pyridin-6-yl)phenol dihydrochloride |
| 265 | 2-{3-[(3S)-3-(propan-2-yl)piperazin-1-yl]-1,2,4-triazin-6-yl}-5-(1,2,5-thiadiazol-3- |
| | yl)phenol trifluoroacetate |
| 270 | 2-{3-[(3S)-3-(propan-2-yl)piperazin-1-yl]-1,2,4-triazin-6-yl}-5-(1,2-thiazol-5- |
| | yl)phenol dihydrochloride |
| 271 | 5-(2-methoxy-6-methylpyridin-4-yl)-2-{3-[(3S)-3-(propan-2-yl)piperazin-1-yl]-1,2,4- |
| | triazin-6-yl}phenol dihydrochloride |
| 272 | 2-(3-hydroxy-4-{3-[(3S)-3-(propan-2-yl)piperazin-1-yl]-1,2,4-triazin-6-yl}phenyl)- |
| | l,3-thiazole-5-carbonitrile hydrochloride |
| 273 | 2-(3-hydroxy-4-{3-[(3S)-3-(propan-2-yl)piperazin-1-yl]-1,2,4-triazin-6-yl}phenyl)- |
| | 1,3-thiazole-4-carbonitrile hydrochloride |
| 274 | 5-(2-methyl-5,6-dihydro[1,2,4]triazolo[1,5-a]pyrazin-7(8H)-yl)-2-{3-[(3S)-3-(propan- |
| | 2-yl)piperazin-1-yl]-1,2,4-triazin-6-yl]phenol formate |
| 275 | 2-{3-[(3S)-3-cyclopropylpiperazin-1-yl]-1,2,4-triazin-6-yl]-5-(1-methyl-2,3-dihydro- |
| | 1H-imidazo[1,2-b]pyrazol-7-yl)phenol dihydrochloride |
| 276 | 2-{3-[(3S)-3-cyclopropylpiperazin-1-yl]-1,2,4-triazin-6-yl]-5-(5,6-dihydro-4H- |
| | pyrrolo[1,2-b]pyrazol-3-yl)phenol dihydrochloride |
| 277 | 2-{3-[(3S)-3-cyclopropylpiperazin-1-yl]-1,2,4-triazin-6-yl}-5-(4,5,6,7- |
| | tetrahydropyrazolo[1,5-a]pyridin-3-yl)phenol dihydrochloride or |
| | 2-{3-[(3R)-3-cyclopropylpiperazin-1-yl]-1,2,4-triazin-6-yl}-5-(4,5,6,7- |
| | tetrahydropyrazolo[1,5-a]pyridin-3-yl)phenol or enantiomer dihydrochloride |
| 283 | 3-fluoro-5-(5-fluoro-1H-pyrazol-4-yl)-2-{3-[(3S)-3-(propan-2-yl)piperazin-1-yl]- |
| | 1,2,4-triazin-6-yl]phenol formate, and |
| 284 | 2-[3-(hexahydropyrrolo[1,2-a]pyrazin-2(1H)-yl)-1,2,4-triazin-6-yl]-5- |
| | (1H-pyrazol-4-yl)phenol; |

wherein a form of the compound is selected from the group consisting of a hydrate, solvate, and tautomer form thereof.

### Chemical Definitions

The chemical terms used above and throughout the description herein, unless specifically defined otherwise, shall be understood by one of ordinary skill in the art to have the following indicated meanings.

As used herein, the term “C₁₋₄₄alkyl” generally refers to saturated hydrocarbon radicals having from one to four carbon atoms in a straight or branched chain configuration, including, but not limited to, methyl, ethyl, n-propyl (also referred to as propyl or propanyl), isopropyl, n-butyl (also referred to as butyl or butanyl), isobutyl, sec-butyl, tert-butyl and the like. A C₁₋₄₄alkyl radical is optionally substituted with substituent species as described herein where allowed by available valences.

As used herein, the term “C₂₋₄alkenyl” generally refers to partially unsaturated hydrocarbon radicals having from two to four carbon atoms in a straight or branched chain configuration and one or more carbon-carbon double bonds therein, including, but not limited to, ethenyl (also referred to as vinyl), allyl, propenyl and the like. A C₂₋₄alkenyl radical is optionally substituted with substituent species as described herein where allowed by available valences.

As used herein, the term “C₂₋₈alkynyl” generally refers to partially unsaturated hydrocarbon radicals having from two to eight carbon atoms in a straight or branched chain configuration and one or more carbon-carbon triple bonds therein, including, but not limited to, ethynyl, propynyl, butynyl and the like. In certain aspects, C₂₋₈alkynyl includes, but is not limited to, C₂₋₆alkynyl, C₂₋₄alkynyl and the like. A C₂₋₈alkynyl radical is optionally substituted with substituent species as described herein where allowed by available valences.

As used herein, the term “C₁₋₄alkoxy” generally refers to saturated hydrocarbon radicals having from one to four carbon atoms in a straight or branched chain configuration of the formula: —O—C₁₋₄alkyl, including, but not limited to, methoxy, ethoxy, n-propoxy, isopropoxy, n-butoxy, isobutoxy, sec-butoxy, tert-butoxy and the like. A C₁₋₄₄alkoxy radical is optionally substituted with substituent species as described herein where allowed by available valences.

As used herein, the term “C₃₋₆cycloalkyl” generally refers to a saturated or partially unsaturated monocyclic, bicyclic or polycyclic hydrocarbon radical, including, but not limited to, cyclopropyl, cyclobutyl, cyclopentyl, and cyclohexyl. A C₃₋₆cycloalkyl radical is optionally substituted with substituent species as described herein where allowed by available valences.

As used herein, the term “aryl” generally refers to a monocyclic, bicyclic or polycyclic aromatic carbon atom ring structure radical, including, but not limited to, phenyl, naphthyl, anthracenyl, fluorenyl, azulenyl, phenanthrenyl and the like. An aryl radical is optionally substituted with substituent species as described herein where allowed by available valences.

As used herein, the term “heteroaryl” generally refers to a monocyclic, bicyclic or polycyclic aromatic carbon atom ring structure radical in which one or more carbon atom ring members have been replaced, where allowed by structural stability, with one or more heteroatoms, such as an O, S or N atom, including, but not limited to, furanyl, thienyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl, benzofuranyl, benzothienyl, benzoimidazolyl, 1,3-benzothiazolyl, 1,3-benzoxazolyl, purinyl, quinolinyl, isoquinolinyl, quinazolinyl, quinoxalinyl, 1,3-diazinyl, 1,2-diazinyl, 1,2-diazolyl, 1,4-diazanaphthalenyl, acridinyl, furo[3,2-b]pyridinyl, furo[3,2-c]pyridinyl, furo[2,3-c]pyridinyl, 6H-thieno[2,3-b]pyrrolyl, thieno[3,2-c]pyridinyl, thieno[2,3-d]pyrimidinyl, 1H-pyrrolo[2,3-b]pyridinyl, 1H-pyrrolo[2,3-c]pyridinyl, 1H-pyrrolo[3,2-b]pyridinyl, pyrrolo[1,2-a]pyrazinyl, pyrrolo[1,2-b]pyridazinyl, pyrazolo[1,5-a]pyridinyl, pyrazolo[1,5-a]pyrazinyl, imidazo[1,2-a]pyridinyl, 3H-imidazo[4,5-b]pyridinyl, imidazo[1,2-a]pyrimidinyl, imidazo[1,2-c]pyrimidinyl, imidazo[1,2-b]pyridazinyl, imidazo[1,2-a]pyrazinyl, imidazo[2,1-b][1,3]thiazolyl, imidazo[2,1-b][1,3,4]thiadiazolyl, [1,2,4]triazolo[1,5-a]pyridinyl, [1,2,4]triazolo[4,3-a]pyridinyl and the like. A heteroaryl radical is optionally substituted on a carbon or nitrogen atom ring member with substituent species as described herein where allowed by available valences.

In certain aspects, the nomenclature for a heteroaryl radical may differ, such as in non-limiting examples where furanyl may also be referred to as furyl, thiophenyl may also be referred to as thienyl, pyridinyl may also be referred to as pyridyl, benzothiophenyl may also be referred to as benzothienyl and 1,3-benzoxazolyl may also be referred to as 1,3-benzooxazolyl.

In certain other aspects, the term for a heteroaryl radical may also include other regioisomers, such as in non-limiting examples where the term pyrrolyl may also include 2H-pyrrolyl, 3H-pyrrolyl and the like, the term pyrazolyl may also include 1H-pyrazolyl and the like, the term imidazolyl may also include 1H-imidazolyl and the like, the term triazolyl may also include 1H-1,2,3-triazolyl and the like, the term oxadiazolyl may also include 1,2,4-oxadiazolyl, 1,3,4-oxadiazolyl and the like, the term tetrazolyl may also include 1H-tetrazolyl, 2H-tetrazolyl and the like, the term indolyl may also include 1H-indolyl and the like, the term indazolyl may also include 1H-indazolyl, 2H-indazolyl and the like, the term benzoimidazolyl may also include 1H-benzoimidazolyl and the term purinyl may also include 9H-purinyl and the like.

As used herein, the term “heterocyclyl” generally refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including, but not limited to, oxiranyl, oxetanyl, azetidinyl, tetrahydrofuranyl, pyrrolinyl, pyrrolidinyl, pyrazolinyl, pyrazolidinyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, pyranyl, dihydro-2H-pyranyl, thiopyranyl, 1,3-dioxanyl, 1,2,5,6-tetrahydropyridinyl, 1,2,3,6-tetrahydropyridinyl, piperidinyl, piperazinyl, morpholinyl, thiomorpholinyl, 1,4-diazepanyl, 1,3-benzodioxolyl, 1,4-benzodioxanyl, 2,3-dihydro-1,4-benzodioxinyl, hexahydropyrrolo[3,4-b]pyrrol-(1H)-yl, (3aS,6aS)-hexahydropyrrolo[3,4-b]pyrrol-(1H)-yl, (3aR,6aR)-hexahydropyrrolo[3,4-b]pyrrol-(1H)-yl, hexahydropyrrolo[3,4-b]pyrrol-(2H)-yl, (3aS,6aS)-hexahydropyrrolo[3,4-b]pyrrol-(2H)-yl, (3aR,6aR)-hexahydropyrrolo[3,4-b]pyrrol-(2H)-yl, hexahydropyrrolo[3,4-c]pyrrol-(1H)-yl, (3aR,6aS)-hexahydropyrrolo[3,4-c]pyrrol-(1H)-yl, (3aR,6aR)-hexahydropyrrolo[3,4-c]pyrrol-(1H)-yl, octahydro-5H-pyrrolo[3,2-c]pyridinyl, octahydro-6H-pyrrolo[3,4-b]pyridinyl, (4aR,7aR)-octahydro-6H-pyrrolo[3,4-b]pyridinyl, (4aS,7aS)-octahydro-6H-pyrrolo[3,4-b]pyridinyl, hexahydropyrrolo[1,2-a]pyrazin-(1H)-yl, (7R,8aS)-hexahydropyrrolo[1,2-a]pyrazin-(1H)-yl, (8aS)-hexahydropyrrolo[1,2-a]pyrazin-(1H)-yl, (8aR)-hexahydropyrrolo[1,2-a]pyrazin-(1H)-yl, (8aS)-octahydropyrrolo[1,2-a]pyrazin-(1H)-yl, (8aR)-octahydropyrrolo[1,2-a]pyrazin-(1H)-yl, hexahydropyrrolo[1,2-a]pyrazin-(2H)-one, octahydro-2H-pyrido[1,2-a]pyrazinyl, 3-azabicyclo[3.1.0]hexyl, (1R,5S)-3-azabicyclo[3.1.0]hexyl, 8-azabicyclo[3.2.1]octyl, (1R,5S)-8-azabicyclo[3.2.1]octyl, 8-azabicyclo[3.2.1]oct-2-enyl, (1R,5S)-8-azabicyclo[3.2.1]oct-2-enyl, 9-azabicyclo[3.3.1]nonyl, (1R,5S)-9-azabicyclo[3.3.1]nonyl, 2,5-diazabicyclo[2.2.1]heptyl, (1S,4S)-2,5-diazabicyclo[2.2.1]heptyl, 2,5-diazabicyclo[2.2.2]octyl, 3,8-diazabicyclo[3.2.1]octyl, (1R,5S)-3,8-diazabicyclo[3.2.1]octyl, 1,4-diazabicyclo[3.2.2]nonyl, azaspiro[3.3]heptyl, 2,6-diazaspiro[3.3]heptyl, 2,6-diazaspiro[3.4]octyl, 2,7-diazaspiro[3.5]nonyl, 5,8-diazaspiro[3.5]nonyl, 2,7-diazaspiro[4.4]nonyl, 6,9-diazaspiro[4.5]decyl, 7-azadispiro[5.1.5⁸.3⁶]hexadecanyl and the like. A heterocyclyl radical is optionally substituted on a carbon or nitrogen atom ring member with substituent species as described herein where allowed by available valences.

In certain aspects, the nomenclature for a heterocyclyl radical may differ, such as in non-limiting examples where 1,3-benzodioxolyl may also be referred to as benzo[d][1,3]dioxolyl and 2,3-dihydro-1,4-benzodioxinyl may also be referred to as 2,3-dihydrobenzo[b][1,4]dioxinyl.

As used herein, the term “C₁₋₄alkoxy-C₁₋₄alkyl” refers to a radical of the formula: —C₁₋₄alkyl-O—C₁₋₄alkyl.

As used herein, the term “C₁₋₄alkyl-amino” refers to a radical of the formula: —NH—C₁₋₄alkyl.

As used herein, the term “(C₁₋₄alkyl)₂-amino” refers to a radical of the formula: —N(C₁₋₄alkyl)₂.

As used herein, the term “C₁₋₄alkyl-carbonyl” refers to a radical of the formula: —C(O)—C₁₋₄alkyl.

As used herein, the term “C₁₋₄alkyl-carbonyl-amino” refers to a radical of the formula: —NH—C(O)—C₁₋₄alkyl.

As used herein, the term “C₁₋₄alkyl-thio” refers to a radical of the formula: —S—C₁₋₄alkyl.

As used herein, the term “amino-C₁₋₄alkyl” refers to a radical of the formula: —C₁₋₄alkyl-NH₂.

As used herein, the term “deutero-C₁₋₄alkyl,” refers to a radical of the formula: —C₁₋₄alkyl-deutero, wherein C₁₋₄alkyl is partially or completely substituted with one or more deuterium atoms where allowed by available valences.

As used herein, the term “halo” or “halogen” generally refers to a halogen atom radical, including fluoro, chloro, bromo and iodo.

As used herein, the term “halo-C₁₋₄alkoxy” refers to a radical of the formula: —O—C₁₋₄alkyl-halo, wherein C₁₋₄alkyl is partially or completely substituted with one or more halogen atoms where allowed by available valences.

As used herein, the term “halo-C₁₋₄alkyl” refers to a radical of the formula: —C₁₋₄alkyl-halo, wherein C₁₋₄alkyl is partially or completely substituted with one or more halogen atoms where allowed by available valences.

As used herein, the term “halo-C₁₋₄alkyl-amino” refers to a radical of the formula: —NH—C₁₋₄alkyl-halo.

As used herein, the term “hydroxy” refers to a radical of the formula: —OH.

As used herein, the term “hydroxy-C₁₋₄alkyl” refers to a radical of the formula: —C₁₋₄alkyl-OH, wherein C₁₋₄alkyl is partially or completely substituted with one or more hydroxy radicals where allowed by available valences.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

For the purposes of this description, where one or more substituent variables for a compound of Formula (I) or a form thereof encompass functionalities incorporated into a compound of Formula (I), each functionality appearing at any location within the disclosed compound may be independently selected, and as appropriate, independently and/or optionally substituted.

As used herein, the terms “independently selected,” or “each selected” refer to functional variables in a substituent list that may occur more than once on the structure of Formula (I), the pattern of substitution at each occurrence is independent of the pattern at any other occurrence. Further, the use of a generic substituent variable on any formula or structure for a compound described herein is understood to include the replacement of the generic substituent with species substituents that are included within the particular genus, e.g., aryl may be replaced with phenyl or naphthalenyl and the like, and that the resulting compound is to be included within the scope of the compounds described herein.

As used herein, the terms “each instance of” or “in each instance, when present,” when used preceding a phrase such as “ . . . C₃₋₁₄cycloalkyl, C₃₋₁₄cycloalkyl-C₁₋₄alkyl, aryl, aryl-C₁₋₄alkyl, heteroaryl, heteroaryl-C₁₋₄alkyl, heterocyclyl and heterocyclyl-C₁₋₄alkyl,” are intended to refer to the C₃₋₁₄cycloalkyl, aryl, heteroaryl and heterocyclyl ring systems when each are present either alone or as a substituent.

As used herein, the term “optionally substituted” means optional substitution with the specified substituent variables, groups, radicals or moieties.

### Compound Forms

As used herein, the term “form” means a compound of Formula (I) having a form selected from the group consisting of a free acid, free base, hydrate, solvate, ester, stereoisomer, and tautomer form thereof.

In certain aspects described herein, the form of the compound of Formula (I) is a free acid, free base or salt thereof.

In certain aspects described herein, the form of the compound of Formula (I)) is a salt thereof.

In certain aspects described herein, the form of the compound of Formula (I) is a tautomer thereof.

In certain aspects described herein, the form of the compound of Formula (I) is a pharmaceutically acceptable form.

In certain aspects described herein, the compound of Formula (I) or a form thereof is isolated for use.

As used herein, the term “isolated” means the physical state of a compound of Formula (I) or a form thereof after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “protected” means that a functional group in a compound of Formula (I) or a form thereof is in a form modified to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, methoxymethanol, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters. In certain instances, the protecting group may also be a polymer resin, such as a Wang resin or a 2-chlorotrityl-chloride resin. Protecting groups may be added or removed in accordance with standard techniques, which are well-known to those skilled in the art and as described herein.

One or more compounds described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

As used herein, the term “hydrate” means a solvate wherein the solvent molecule is water.

The compounds of Formula (I) can form salts, which are intended to be included within the scope of this description. Reference to a compound of Formula (I) a form thereof herein is understood to include reference to salt forms thereof, unless otherwise indicated. The term “salt(s)”, as employed herein, denotes acidic salts formed with inorganic and/or organic acids, as well as basic salts formed with inorganic and/or organic bases. In addition, when a compound of Formula (I)) or a form thereof contains both a basic moiety, such as, without limitation an amine moiety, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds described herein that are safe and effective (i.e., non-toxic, physiologically acceptable) for use in mammals and that possess biological activity, although other salts are also useful. Salts of the compounds of the Formula (I) may be formed, for example, by reacting a compound of Formula (I) or a form thereof with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. Particular aspects of acid addition salts include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate salts and the like. Certain particular aspects of acid addition salts include chloride or dichloride.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium and zinc salts.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formula (I) and forms thereof, may further exist in a tautomeric form. All such tautomeric forms are contemplated and intended to be included within the scope of the compounds of Formula (I) or a form thereof as described herein.

The compounds of Formula (I) or a form thereof may contain asymmetric or chiral centers, and, therefore, exist in different stereoisomeric forms. The present description is intended to include all stereoisomeric forms of the compounds of Formula (I) as well as mixtures thereof, including racemic mixtures.

The compounds described herein may include one or more chiral centers, and as such may exist as racemic mixtures (R/S) or as substantially pure enantiomers and diastereomers. The compounds may also exist as substantially pure (R) or (S) enantiomers (when one chiral center is present). In one particular aspect, the compounds described herein are (S) isomers and may exist as enantiomerically pure compositions substantially comprising only the (S) isomer. In another particular aspect, the compounds described herein are (R) isomers and may exist as enantiomerically pure compositions substantially comprising only the (R) isomer. As one of skill in the art will recognize, when more than one chiral center is present, the compounds described herein may also exist as a (R,R), (R,S), (S,R) or (S,S) isomer, as defined by IUPAC Nomenclature Recommendations.

As used herein, the term “chiral” refers to a carbon atom bonded to four nonidentical substituents. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al. Angew. Chem. Inter. Edit. 1966, 5, 385; errata 511).

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formula (I)) or a form thereof is a substantially pure (S) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formula (I) or a form thereof is a substantially pure (R) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, a “racemate” is any mixture of isometric forms that are not “enantiomerically pure”, including mixtures such as, without limitation, in a ratio of about 50/50, about 60/40, about 70/30, or about 80/20.

In addition, the present description embraces all geometric and positional isomers. For example, if a compound of Formula (I) or a form thereof incorporates a double bond or a fused ring, both the cis- and trans-forms, as well as mixtures, are embraced within the scope of the description. Diastereomeric mixtures can be separated into their individual diastereomers on the basis of their physical chemical differences by methods well known to those skilled in the art, such as, for example, by chromatography and/or fractional crystallization. Enantiomers can be separated by use of chiral HPLC column or other chromatographic methods known to those skilled in the art. Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this description.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this description, as are positional isomers (such as, for example, 4-pyridinyl and 3-pyridinyl). Individual stereoisomers of the compounds described herein may, for example, be substantially free of other isomers, or may be present in a racemic mixture, as described supra.

The use of the terms “salt”, “solvate” and the like, is intended to equally apply to the salt and solvate of enantiomers, stereoisomers, rotamers, tautomers, positional isomers, or racemates of the instant compounds.

### Compound Uses

In accordance with the intended scope of the present description, aspects of the present description include compounds that have been identified and have been demonstrated to be useful in selectively preventing, treating or ameliorating HD and have been provided for use for preventing, treating or ameliorating HD.

An aspect of the present description includes a method for preventing, treating or ameliorating HD in a subject in need thereof comprising, administering to the subject an effective amount of a compound of Formula (I) or a form thereof.

An aspect of the present description includes a method for treating or ameliorating HD in a subject in need thereof comprising, administering to the subject an effective amount of a compound of Formula (I) or a form thereof.

An aspect of the present description includes a method for preventing HD in a subject in need thereof comprising, administering to the subject an effective amount of a compound of Formula (I) or a form thereof.

An aspect of the present description includes a method for treating HD in a subject in need thereof comprising, administering to the subject an effective amount of a compound of Formula (I) or a form thereof.

An aspect of the present description includes a method for ameliorating HD in a subject in need thereof comprising, administering to the subject an effective amount of a compound of Formula (I) or a form thereof.

Another aspect of the present description includes a method for treating or ameliorating HD in a subject in need thereof comprising, administering to the subject an effective amount of a compound salt of Formula (I) or a form thereof.

Another aspect of the present description includes a method for treating or ameliorating HD in a subject in need thereof comprising, administering to the subject an effective amount of a compound or compound salt of Formula (I) or a form thereof, in a combination product, or as a combination therapy, with one or more therapeutic agents.

An aspect of the present description includes a method for use of a compound of Formula (I) or a form or composition thereof for treating or ameliorating HD in a subject in need thereof comprising, administering to the subject an effective amount of the compound of Formula (I) or a form or composition thereof.

Another aspect of the present description includes a method for use of a compound salt of Formula (I) or a form or composition thereof for treating or ameliorating HD in a subject in need thereof comprising, administering to the subject an effective amount of the compound salt of Formula (I) or a form thereof.

An aspect of the present description includes a use for a compound of Formula (I) or a form thereof for treating or ameliorating HD in a subject in need thereof comprising, administering to the subject an effective amount of the compound of Formula (I) or a form thereof.

Another aspect of the present description includes a use for a compound salt of Formula (I) or a form thereof for treating or ameliorating HD in a subject in need thereof comprising, administering to the subject an effective amount of the compound salt of Formula (I) or a form thereof.

An aspect of the present description includes a use for a compound of Formula (I) or a form thereof in the manufacture of a medicament for treating or ameliorating HD in a subject in need thereof comprising, administering to the subject an effective amount of the medicament.

Another aspect of the present description includes a use for a compound salt of Formula (I)) or a form thereof in the manufacture of a medicament for treating or ameliorating HD in a subject in need thereof comprising, administering to the subject an effective amount of the medicament.

An aspect of the present description includes in vitro or in vivo use of the compound of Formula (I) or a form thereof having activity toward HD.

An aspect of the present description includes a use of the compound of Formula (I) or a form thereof in a combination therapy to provide additive or synergistic activity, thus enabling the development of a combination product for treating or ameliorating HD.

Another aspect of the present description includes a combination therapy comprising compounds described herein in combination with one or more known drugs or one or more known therapies may be used to treat HD regardless of whether HD is responsive to the known drug.

An aspect of the present description includes a use for a compound of Formula (I) or a form thereof in a combination product with one or more therapeutic agents for treating or ameliorating HD in a subject in need thereof comprising, administering to the subject an effective amount of the compound of Formula (I) or a form thereof in combination with an effective amount of the one or more agents.

Another aspect of the present description includes a use for a compound salt of Formula (I) or a form thereof in a combination product with one or more therapeutic agents for treating or ameliorating HD in a subject in need thereof comprising, administering to the subject an effective amount of the compound salt of Formula (I) or a form thereof in combination with an effective amount of the one or more agents.

In an aspect of a use or method provided herein, compounds of Formula (I) or a form thereof used in combination with one or more additional agents can be administered to a subject or contacted with a subject or patient cell(s) prior to, concurrently with, or subsequent to administering to the subject or patient or contacting the cell with an additional agent(s). A compound(s) of Formula (I) or a form thereof and an additional agent(s) can be administered to a subject or contacted with a cell in single composition or different compositions. In a specific aspect, a compound(s) of Formula (I) or a form thereof is used in combination with gene therapy to inhibit HTT expression (using, e.g., viral delivery vectors) or the administration of another small molecule HTT inhibitor. In another specific aspect, a compound(s) of Formula (I) or a form thereof are used in combination with cell replacement using differentiated non-mutant HTT stem cells. In another specific aspect, a compound(s) of Formula (I) or a form thereof are used in combination with cell replacement using differentiated HTT stem cells.

In one aspect, provided herein is the use of compounds of Formula (I) or a form thereof in combination with supportive standard of care therapies, including palliative care.

In one respect, for each of such aspects, the subject is treatment naive. In another respect, for each of such aspects, the subject is not treatment naive.

As used herein, the term “preventing” refers to keeping a disease, disorder or condition from occurring in a subject that may be predisposed to the disease, disorder and/or condition but has not yet been diagnosed as having the disease, disorder and/or condition.

As used herein, the term “treating” refers to inhibiting the progression of a disease, disorder or condition in a subject already exhibiting the symptoms of the disease, disorder and/or condition, i.e., arresting the development of a disease, disorder and/or condition that has already affected the subject.

As used herein, the term “ameliorating” refers to relieving the symptoms of a disease, disorder or condition in a subject already exhibiting the symptoms of the disease, disorder and/or condition, i.e., causing regression of the disease, disorder and/or condition that has already affected the subject.

As used herein, the term “subject” refers to an animal or any living organism having sensation and the power of voluntary movement, and which requires oxygen and organic food. Nonlimiting examples include members of the human, primate, equine, porcine, bovine, murine, rattus, canine and feline specie. In certain aspects, the subject is a mammal or a warm-blooded vertebrate animal. In other aspects, the subject is a human. As used herein, the term “patient” may be used interchangeably with “subject” and “human”.

As used herein, the terms “effective amount” or “therapeutically effective amount” mean an amount of compound of Formula (I) or a form, composition or medicament thereof that achieves a target plasma concentration that is effective in treating or ameliorating HD as described herein and thus producing the desired therapeutic, ameliorative, inhibitory or preventative effect in a subject in need thereof. In one aspect, the effective amount may be the amount required to treat HD in a subject or patient, more specifically, in a human.

In another aspect, the concentration-biological effect relationships observed with regard to a compound of Formula (I) or a form thereof indicate a target plasma concentration ranging from approximately 0.001 μg/mL to approximately 50 μg/mL, from approximately 0.01 μg/mL to approximately 20 μg/mL, from approximately 0.05 μg/mL to approximately 10 μg/mL, or from approximately 0.1 μg/mL to approximately 5 μg/mL. To achieve such plasma concentrations, the compounds described herein may be administered at doses that vary, such as, for example, without limitation, from 1.0 ng to 10,000 mg.

In one aspect, the dose administered to achieve an effective target plasma concentration may be administered based upon subject or patient specific factors, wherein the doses administered on a weight basis may be in the range of from about 0.001 mg/kg/day to about 3500 mg/kg/day, or about 0.001 mg/kg/day to about 3000 mg/kg/day, or about 0.001 mg/kg/day to about 2500 mg/kg/day, or about 0.001 mg/kg/day to about 2000 mg/kg/day, or about 0.001 mg/kg/day to about 1500 mg/kg/day, or about 0.001 mg/kg/day to about 1000 mg/kg/day, or about 0.001 mg/kg/day to about 500 mg/kg/day, or about 0.001 mg/kg/day to about 250 mg/kg/day, or about 0.001 mg/kg/day to about 200 mg/kg/day, or about 0.001 mg/kg/day to about 150 mg/kg/day, or about 0.001 mg/kg/day to about 100 mg/kg/day, or about 0.001 mg/kg/day to about 75 mg/kg/day, or about 0.001 mg/kg/day to about 50 mg/kg/day, or about 0.001 mg/kg/day to about 25 mg/kg/day, or about 0.001 mg/kg/day to about 10 mg/kg/day, or about 0.001 mg/kg/day to about 5 mg/kg/day, or about 0.001 mg/kg/day to about 1 mg/kg/day, or about 0.001 mg/kg/day to about 0.5 mg/kg/day, or about 0.001 mg/kg/day to about 0.1 mg/kg/day, or from about 0.01 mg/kg/day to about 3500 mg/kg/day, or about 0.01 mg/kg/day to about 3000 mg/kg/day, or about 0.01 mg/kg/day to about 2500 mg/kg/day, or about 0.01 mg/kg/day to about 2000 mg/kg/day, or about 0.01 mg/kg/day to about 1500 mg/kg/day, or about 0.01 mg/kg/day to about 1000 mg/kg/day, or about 0.01 mg/kg/day to about 500 mg/kg/day, or about 0.01 mg/kg/day to about 250 mg/kg/day, or about 0.01 mg/kg/day to about 200 mg/kg/day, or about 0.01 mg/kg/day to about 150 mg/kg/day, or about 0.01 mg/kg/day to about 100 mg/kg/day, or about 0.01 mg/kg/day to about 75 mg/kg/day, or about 0.01 mg/kg/day to about 50 mg/kg/day, or about 0.01 mg/kg/day to about 25 mg/kg/day, or about 0.01 mg/kg/day to about 10 mg/kg/day, or about 0.01 mg/kg/day to about 5 mg/kg/day, or about 0.01 mg/kg/day to about 1 mg/kg/day, or about 0.01 mg/kg/day to about 0.5 mg/kg/day, or about 0.01 mg/kg/day to about 0.1 mg/kg/day, or from about 0.1 mg/kg/day to about 3500 mg/kg/day, or about 0.1 mg/kg/day to about 3000 mg/kg/day, or about 0.1 mg/kg/day to about 2500 mg/kg/day, or about 0.1 mg/kg/day to about 2000 mg/kg/day, or about 0.1 mg/kg/day to about 1500 mg/kg/day, or about 0.1 mg/kg/day to about 1000 mg/kg/day, or about 0.1 mg/kg/day to about 500 mg/kg/day, or about 0.1 mg/kg/day to about 250 mg/kg/day, or about 0.1 mg/kg/day to about 200 mg/kg/day, or about 0.1 mg/kg/day to about 150 mg/kg/day, or about 0.1 mg/kg/day to about 100 mg/kg/day, or about 0.1 mg/kg/day to about 75 mg/kg/day, or about 0.1 mg/kg/day to about 50 mg/kg/day, or about 0.1 mg/kg/day to about 25 mg/kg/day, or about 0.1 mg/kg/day to about 10 mg/kg/day, or about 0.1 mg/kg/day to about 5 mg/kg/day, or about 0.1 mg/kg/day to about 1 mg/kg/day, or about 0.1 mg/kg/day to about 0.5 mg/kg/day.

Effective amounts for a given subject may be determined by routine experimentation that is within the skill and judgment of a clinician or a practitioner skilled in the art in light of factors related to the subject. Dosage and administration may be adjusted to provide sufficient levels of the active agent(s) or to maintain the desired effect. Factors which may be taken into account include genetic screening, severity of the disease state, status of disease progression, general health of the subject, ethnicity, age, weight, gender, diet, time of day and frequency of administration, drug combination(s), reaction sensitivities, experience with other therapies, and tolerance/response to therapy.

The dose administered to achieve an effective target plasma concentration may be orally administered once (once in approximately a 24 hour period; i.e., “q.d.”), twice (once in approximately a 12 hour period; i.e., “b.i.d.” or “q.12h”), thrice (once in approximately an 8 hour period; i.e., “t.i.d.” or “q.8h”), or four times (once in approximately a 6 hour period; i.e., “q.d.s.”, “q.i.d.” or “q.6h”) daily.

In certain aspects, the dose administered to achieve an effective target plasma concentration may also be administered in a single, divided, or continuous dose for a patient or subject having a weight in a range of between about 40 to about 200 kg (which dose may be adjusted for patients or subjects above or below this range, particularly children under 40 kg). The typical adult subject is expected to have a median weight in a range of about 70 kg. Long-acting pharmaceutical compositions may be administered every 2, 3 or 4 days, once every week, or once every two weeks depending on half-life and clearance rate of the particular formulation.

The compounds and compositions described herein may be administered to the subject via any drug delivery route known in the art. Nonlimiting examples include oral, ocular, rectal, buccal, topical, nasal, sublingual, transdermal, subcutaneous, intramuscular, intravenous (bolus and infusion), intracerebral, and pulmonary routes of administration.

In another aspect, the dose administered may be adjusted based upon a dosage form described herein formulated for delivery at about 0.02, 0.025, 0.03, 0.05, 0.06, 0.075, 0.08, 0.09, 0.10, 0.20, 0.25, 0.30, 0.50, 0.60, 0.75, 0.80, 0.90, 1.0, 1.10, 1.20, 1.25, 1.50, 1.75, 2.0, 3.0, 5.0, 10, 20, 30, 40, 50, 100, 150, 200, 250, 300, 400, 500, 1000, 1500, 2000, 2500, 3000 or 4000 mg/day.

For any compound, the effective amount can be estimated initially either in cell culture assays or in relevant animal models, such as a mouse, guinea pig, chimpanzee, marmoset or tamarin animal model. Relevant animal models may also be used to determine the appropriate concentration range and route of administration. Such information can then be used to determine useful doses and routes for administration in humans. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is therapeutic index, and can be expressed as the ratio, LD₅₀/ED₅₀. In certain aspects, the effective amount is such that a large therapeutic index is achieved. In further particular aspects, the dosage is within a range of circulating concentrations that include an ED₅₀ with little or no toxicity. The dosage may vary within this range depending upon the dosage form employed, sensitivity of the patient, and the route of administration.

In one aspect, provided herein are methods for modulating the amount of HTT (huntingtin protein), comprising contacting a human cell with a compound of Formula (I) or a form thereof. In a specific aspect, provided herein are methods for modulating the amount of HTT, comprising contacting a human cell with a compound of Formula (I)) or a form thereof that modulates the expression of HTT. The human cell can be contacted with a compound of Formula (I)) or a form thereof in vitro, or in vivo, e.g., in a non-human animal or in a human. In a specific aspect, the human cell is from or in a human. In another specific aspect, the human cell is from or in a human with HD. In another specific aspect, the human cell is from or in a human with HD, caused by a CAG repeat in the Htt gene, resulting in a loss of HTT expression and/or function. In another aspect, the human cell is from a human with HD. In another aspect, the human cell is in a human with HD. In one aspect, the compound is a form of the compound of Formula (I).

In a specific aspect, provided herein is a method for enhancing the inhibition of mutant HTT transcribed from the Htt gene, comprising contacting a human cell with a compound of Formula (I) or a form thereof. The human cell can be contacted with a compound of Formula (I) or a form thereof in vitro, or in vivo, e.g., in a non-human animal or in a human. In a specific aspect, the human cell is from or in a human. In another specific aspect, the human cell is from or in a human with HD. In another specific aspect, the human cell is from or in a human with HD, caused by a CAG repeat in the Htt gene, resulting in a loss of wild-type “normal” HTT expression and/or function. In another aspect, the human cell is from a human with HD. In another aspect, the human cell is in a human with HD. In one aspect, the compound is a form of the compound of Formula (I).

In another aspect, provided herein is a method for modulating the inhibition of mutant HTT transcribed from the Htt gene, comprising administering to a non-human animal model for HD a compound of Formula (I) or a form thereof. In a specific aspect, provided herein is a method for modulating the inhibition of mutant HTT transcribed from the Htt gene, comprising administering to a non-human animal model for HD a compound of Formula (I) or a form thereof. In a specific aspect, the compound is a form of the compound of Formula (I).

In another aspect, provided herein is a method for decreasing the amount of mutant HTT, comprising contacting a human cell with a compound of Formula (I) or a form thereof. In a specific aspect, provided herein is a method for decreasing the amount of mutant HTT, comprising contacting a human cell with a compound of Formula (I) that inhibits the transcription of mutant HTT (huntingtin mRNA) from the Htt gene. In another specific aspect, provided herein is a method for decreasing the amount of HTT, comprising contacting a human cell with a compound of Formula (I) that inhibits the expression of mutant HTT transcribed from the Htt gene. The human cell can be contacted with a compound of Formula (I) or a form thereof in vitro, or in vivo, e.g., in a non-human animal or in a human. In a specific aspect, the human cell is from or in a human. In another specific aspect, the human cell is from or in a human with HD. In another specific aspect, the human cell is from or in a human with HD, caused by a CAG repeat in the Htt gene, resulting in a loss of HTT expression and/or function. In another aspect, the human cell is from a human with HD. In another aspect, the human cell is in a human with HD. In one aspect, the compound is a form of the compound of Formula (I)).

In certain aspects, treating or ameliorating HD with a compound of Formula (I) or a form thereof (alone or in combination with an additional agent) has a therapeutic effect and/or beneficial effect. In a specific aspect, treating HD with a compound of Formula (I) or a form thereof (alone or in combination with an additional agent) results in one, two or more of the following effects: (i) reduces or ameliorates the severity of HD; (ii) delays onset of HD; (iii) inhibits the progression of HD; (iv) reduces hospitalization of a subject; (v) reduces hospitalization length for a subject; (vi) increases the survival of a subject; (vii) improves the quality of life for a subject; (viii) reduces the number of symptoms associated with HD; (ix) reduces or ameliorates the severity of a symptom(s) associated with HD; (x) reduces the duration of a symptom associated with HD; (xi) prevents the recurrence of a symptom associated with HD; (xii) inhibits the development or onset of a symptom of HD; and/or (xiii) inhibits of the progression of a symptom associated with HD.

### Metabolites

Another aspect included within the scope of the present description are the use of in vivo metabolic products of the compounds described herein. Such products may result, for example, from the oxidation, reduction, hydrolysis, amidation, esterification and the like of the administered compound, primarily due to enzymatic processes. Accordingly, the description includes the use of compounds produced by a process comprising contacting a compound described herein with a mammalian tissue or a mammal for a period of time sufficient to yield a metabolic product thereof.

Such products typically are identified by preparing a radio-labeled isotopologue (e.g., ¹⁴C or ³H) of a compound described herein, administering the radio-labeled compound in a detectable dose (e.g., greater than about 0.5 mg/kg) to a mammal such as a rat, mouse, guinea pig, dog, monkey or human, allowing sufficient time for metabolism to occur (typically about 30 seconds to about 30 hours), and identifying the metabolic conversion products from urine, bile, blood or other biological samples. The conversion products are easily isolated since they are “radiolabeled” by virtue of being isotopically-enriched (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds described herein even if they possess no biological activity of their own.

### Pharmaceutical Compositions

In accordance with the intended scope of the present description, aspects of the present description include compounds that have been identified and have been demonstrated to be useful in selectively preventing, treating or ameliorating HD and have been provided for use as one or more pharmaceutical compositions for preventing, treating or ameliorating HD.

An aspect of the present description includes a use for a compound of Formula (I) or a form thereof in the preparation of a pharmaceutical composition for treating or ameliorating HD in a subject in need thereof comprising, administering to the subject an effective amount of the compound of Formula (I) or a form thereof in admixture with one or more pharmaceutically acceptable excipients.

An aspect of the present description includes a use for a pharmaceutical composition of the compound of Formula (I) or a form thereof in the preparation of a kit for treating or ameliorating HD in a subject in need thereof comprising, the pharmaceutical composition of the compound of Formula (I) or a form thereof and instructions for administering the pharmaceutical composition.

As used herein, the term “composition” means a product comprising the specified ingredients in the specified amounts, as well as any product which results, directly or indirectly, from combination of the specified ingredients in the specified amounts.

The pharmaceutical composition may be formulated to achieve a physiologically compatible pH, ranging from about pH 3 to about pH 11. In certain aspects, the pharmaceutical composition is formulated to achieve a pH of from about pH 3 to about pH 7.

In other aspects, the pharmaceutical composition is formulated to achieve a pH of from about pH 5 to about pH 8.

The term “pharmaceutically acceptable excipient” refers to an excipient for administration of a pharmaceutical agent, such as the compounds described herein. The term refers to any pharmaceutical excipient that may be administered without undue toxicity. Pharmaceutically acceptable excipients may be determined in part by the particular composition being administered, as well as by the particular mode of administration and/or dosage form. Nonlimiting examples of pharmaceutically acceptable excipients include carriers, solvents, stabilizers, adjuvants, diluents, etc. Accordingly, there exists a wide variety of suitable formulations of pharmaceutical compositions for the instant compounds described herein (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive antibodies. Other exemplary excipients include antioxidants such as ascorbic acid; chelating agents such as EDTA; carbohydrates such as dextrin, hydroxyalkylcellulose, hydroxyalkylmethylcellulose (e.g., hydroxypropylmethylcellulose, also known as HPMC), stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions described herein may be formulated in any form suitable for the intended use described herein. Suitable formulations for oral administration include solids, liquid solutions, emulsions and suspensions, while suitable inhalable formulations for pulmonary administration include liquids and powders. Alternative formulations include syrups, creams, ointments, tablets, and lyophilized solids which can be reconstituted with a physiologically compatible solvent prior to administration.

When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents, and preserving agents, in order to provide a palatable preparation.

Pharmaceutically acceptable excipients suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid, or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate, or kaolin, or as soft gelatin capsules wherein the active ingredient is mixed with non-aqueous or oil medium, such as glycerin, propylene glycol, polyethylene glycol, peanut oil, liquid paraffin, or olive oil.

In other aspects, pharmaceutical compositions described herein may be formulated as suspensions comprising a compound of Formula (I) or a form thereof in admixture with one or more pharmaceutically acceptable excipients suitable for the manufacture of a suspension. In yet other aspects, pharmaceutical compositions described herein may be formulated as dispersible powders and granules suitable for preparation of a suspension by the addition of one or more excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions described herein may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions described herein may be in the form of a sterile injectable preparation, such as a sterile injectable aqueous emulsion or oleaginous suspension. Such emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propanediol. The sterile injectable preparation may also be prepared as a lyophilized powder. Among the acceptable vehicles and solvents that may be employed are water, Ringer's solution and isotonic sodium chloride solution. In addition, sterile fixed oils may be employed as a solvent or suspending medium. For this purpose any bland fixed oil may be employed including synthetic mono- or di-glycerides. In addition, fatty acids such as oleic acid may likewise be used in the preparation of injectables.

The compounds described herein may be substantially insoluble in water and sparingly soluble in most pharmaceutically acceptable protic solvents and vegetable oils, but generally soluble in medium-chain fatty acids (e.g., caprylic and capric acids) or triglycerides and in propylene glycol esters of medium-chain fatty acids. Thus, contemplated in the description are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In certain aspects, the compound described herein is formulated for oral administration in a lipid-based composition suitable for low solubility compounds. Lipid-based formulations can generally enhance the oral bioavailability of such compounds. As such, pharmaceutical compositions described herein may comprise a effective amount of a compound of Formula (I) or a form thereof, together with at least one pharmaceutically acceptable excipient selected from medium chain fatty acids or propylene glycol esters thereof (e.g., propylene glycol esters of edible fatty acids such as caprylic and capric fatty acids) and pharmaceutically acceptable surfactants, such as polysorbate 20 or 80 (also referred to as Tween® 20 or Tween® 80, respectively) or polyoxyl 40 hydrogenated castor oil.

In other aspects, the bioavailability of low solubility compounds may be enhanced using particle size optimization techniques including the preparation of nanoparticles or nanosuspensions using techniques known to those skilled in the art. The compound forms present in such preparations include amorphous, partially amorphous, partially crystalline or crystalline forms.

In alternative aspects, the pharmaceutical composition may further comprise one or more aqueous solubility enhancer(s), such as a cyclodextrin. Nonlimiting examples of cyclodextrin include hydroxypropyl, hydroxyethyl, glucosyl, maltosyl and maltotriosyl derivatives of α-, β-, and γ-cyclodextrin, and hydroxypropyl-β-cyclodextrin (HPBC). In certain aspects, the pharmaceutical composition further comprises HPBC in a range of from about 0.1% to about 20%, from about 1% to about 15%, or from about 2.5% to about 10%. The amount of solubility enhancer employed may depend on the amount of the compound in the composition.

### Preparation of Compounds

### General Synthetic Methods

As disclosed herein, general methods for preparing the compounds of Formula (I) or a form thereof as described herein are available via standard, well-known synthetic methodology. Many of the starting materials are commercially available or, when not available, can be prepared using the routes described below using techniques known to those skilled in the art. The synthetic schemes provided herein comprise multiple reaction steps, each of which is intended to stand on its own and can be carried out with or without any preceding or succeeding step(s). In other words, each of the individual reaction steps of the synthetic schemes provided herein in isolation is contemplated.

Compounds of Formula GS1-8, wherein A, B, X, R_{W}, and n are as defined for Formula (I) may be prepared as described in General Scheme 1 below.

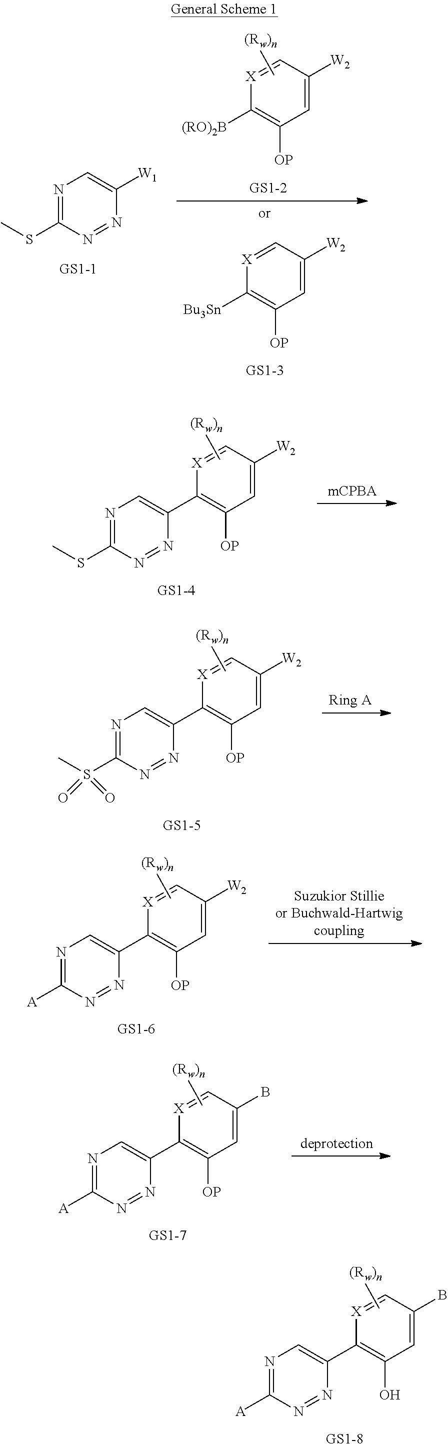

Compound GS1-1 (where W₁ is bromine, chlorine and the like) is converted to Compound GS1-4 by a Suzuki coupling with an aryl-boronic acid (or pinacol boronic ester) GS1-2 (where W₂ is bromine, chlorine and the like; R₁ is hydrogen, fluorine, chlorine, hydroxy, methoxy, aryl or heteroaryl; and P is a protecting group such as MOM and the like) in the presence of a catalyst (such as Pd(dppf)Cl₂ and the like) and base (such as aqueous K₂CO₃ and the like) in a suitable solvent (such as 1,4-dioxane and the like). Alternatively, Compound GS1-1 is converted to Compound GS1-4 by a Stille coupling with an aryl-stanane GS1-3 (where W₂ is bromine, chlorine and the like; and P is a protecting group such as MOM and the like) in the presence of a catalyst (such as Pd(PPh₃)₂Cl₂ and the like) and additive (such as CuI and the like) in a suitable solvent (such as 1,4-dioxane and the like). Compound GS1-4 is converted to Compound GS1-5 by treatment with an oxidizing agent (such as mCPBA or oxone and the like) in a suitable solvent (such as dichloromethane and the like). Compound GS1-5 is converted to Compound GS1-6 by a nucleophilic substitution with a piperazine Ring A in the presence of a suitable base (such as Et₃N and the like) in a suitable solvent (such as DMF and the like). Compound GS1-6 is converted to Compound GS1-7 by a Suzuki coupling with an aryl- or heteroaryl-boronic acid (or pinacol boronic ester) in the presence of a catalyst (such as Pd(dppf)Cl₂ and the like) and a base (such as aqueous K₂CO₃ and the like) in a suitable solvent (such as 1,4-dioxane and the like). Alternatively, Compound A7 is converted to Compound GS1-7 by a Stille coupling with an aryl- or heteroaryl-stannane in the presence of a catalyst (such as Pd₂(dba)₃ and the like), a ligand (such as X-Phos and the like) and a base (such as CsF and the like) in a suitable solvent (such as 1,4-dioxane and the like). Alternatively, Compound GS1-6 is converted to Compound GS1-7 by treatment with pinacolatodiboron and a base (such as KOAc and the like) in the presence of a catalyst (such as Pd(dppf)Cl₂ and the like) in an appropriate solvent (such as 1,4-dioxane and the like), followed by addition of an aryl- or heteroaryl-halide. Alternatively, Compound GS1-6 is converted to Compound GS1-7 by a Buchwald-Hartwig coupling with a heteroaryl or amine in the presence of a catalyst (such as Pd₂(dba)₃ and the like), a ligand (such as tBuX-Phos and the like) and a base (such as K₃PO₄ and the like) in a suitable solvent (such as 1,4-dioxane and the like). Compound GS1-7 is converted to Compound GS1-8 upon treatment with conditions appropriate to the removal of the protecting groups (such as HCl in dioxane for a MOM protecting group) in a suitable solvent (such as dioxane and the like).

### SPECIFIC SYNTHETIC EXAMPLES

To describe in more detail and assist in understanding, the following non-limiting examples are offered to more fully illustrate the scope of compounds described herein and are not to be construed as specifically limiting the scope thereof. Such variations of the compounds described herein that may be now known or later developed, which would be within the purview of one skilled in the art to ascertain, are considered to fall within the scope of the compounds as described herein and hereinafter claimed. These examples illustrate the preparation of certain compounds. Those of skill in the art will understand that the techniques described in these examples represent techniques, as described by those of ordinary skill in the art, that function well in synthetic practice, and as such constitute preferred modes for the practice thereof. However, it should be appreciated that those of skill in the art should, in light of the present disclosure, appreciate that many changes can be made in the specific methods that are disclosed and still obtain a like or similar result without departing from the spirit and scope of the present description.

Other than in the following examples of the embodied compounds, unless indicated to the contrary, all numbers expressing quantities of ingredients, reaction conditions, experimental data, and so forth used in the specification and claims are to be understood as being modified by the term “about.” Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and rounding techniques used by those of skill in the art.

While the numerical ranges and parameters setting forth the broad scope of the present description are approximations, the numerical values set forth in the examples set forth below are reported as precisely as possible. Any numerical value, however, inherently contains certain errors necessarily resulting from the standard deviation found in their respective testing measurements.

The starting materials used in the examples provided are commercially available or can be prepared according to methods known to one skilled in the art or can be prepared by the procedures disclosed herein. Structural confirmation was preformed using analytical techniques known to those skilled in the art such as ¹H or ¹³C nuclear magnetic resonance spectroscopy or mass spectrometry.

Compounds of Formula (I) were synthesized using procedures similar to those described in International Application Publication No. WO/2019/191229 A1, filed on Mar. 27, 2019, and claiming priority to U.S. Provisional Application 62/648,699 filed on Mar. 27, 2018, by substituting the appropriate starting materials, reagents and reaction conditions.

### Compound Examples

As used above, and throughout the present description, the following abbreviations, unless otherwise indicated, shall be understood to have the following meanings:

| Abbreviation | Meaning |
|---|---|
| Δ | heating (chemistry) or deletion (biology) |
| AcOH or HOAc | acetic acid |
| Ar | argon |
| ACN or CH₃CN | acetonitrile |
| aq. | aqueous |
| atm | atmosphere(s) |
| BBr₃ | boron tribromide |
| B₂pin₂ | bis(pinacolato)diboron |
| Boc | tert-butoxy-carbonyl |
| Boc₂O | di-tert-butyl dicarbonate |
| t-Bu | tert-butyl |
| t-BuOK or KOtBu | postassium tert-butoxide |
| BuOH or n-BuOH | n-butanol |
| t-BuXPhos | 2-di-tert-butylphosphino-2′,4′,6′- |
| | triisopropylbiphenyl |
| ° C. | degrees Centigrade |
| Celite ® or Celite | diatomaceous earth |
| mCPBA | meta-chloroperoxybenzoic acid |
| CuI | copper(I) iodide |
| d/h/hr/hrs/min/s | day(d)/hour(h, hr or hrs)/minute(min)/second(s) |
| DCM or CH₂Cl₂ | dichloromethane |
| DIPEA | N, N-diisopropylethylamine |
| DMAP | 4-dimethylaminopyridine |
| DMF | dimethylformamide |
| DMSO | dimethylsulfoxide |
| EtOAc | ethyl acetate |
| EtOH | ethanol |
| Et₂O | diethyl ether |
| equiv | equivalents |
| H₂ | hydrogen |
| HBr | hydrobromic acid |
| HC1 | hydrochloric acid |
| H₂SO₄ | sulfuric acid |
| K₂CO₃ | potassium carbonate |
| K₃PO₄ | tripotassium phosphate |
| KOAc | potassium acetate |
| KOH | potassium hydroxide |
| LC/MS, LCMS | liquid chromatographic mass spectroscopy |
| or LC-MS | |
| LiOt-Bu | lithium tert-butoxide |
| LiOH | lithium hydroxide |
| MeOH | methanol |
| MeSO₃H | methanesulfonic acid |
| MgSO₄ | magnesium sulfate |
| mL | milliliter |
| MOM | methoxymethyl |
| MS | mass spectroscopy |
| NBS | N-bromosuccinimide |
| NEt₃ | triethylamine |
| NH₄CI | ammonium chloride |
| NH₄OAc | ammonium acetate |
| Na₂CO₃ | sodium carbonate |
| NaH | sodium hydride |
| NaHCO₃ | sodium bicarbonate |
| NaOH | sodium hydroxide |
| Na₂SO₄ | sodium sulfate |
| N₂ | nitrogen |
| NH₄Cl | ammoniuim chloride |
| NMP | N-methylpyrrolidone |
| NMR | nuclear magnetic resonance |
| Pd | palladium |
| Pd/C | palladium on carbon |
| PdCl₂(PPh₃)₂ | bis(triphenylphosphine)palladium(II) dichloride |
| Pd₂(dba)3 | tris(dibenzylideneacetone)dipalladium(0) |
| Pd(dppf)Cl₂or | [1,1′-bis(diphenylphosphino) |
| Pd(dppf) | ferrocene]dichloropalladium(II), |
| Cl₂—CH₂Cl₂ | complex with dichloromethane |
| PhMe | toluene |
| Psi | pounds per square inch pressure |
| QPhos | 1,2,3,4,5-pentaphenyl-1′-(di-tert- |
| | butylphosphino)ferrocene |
| Rt or rt | room temperature |
| S-Phos, SPhos | 2-dicyclohexylphosphino-2′, |
| or Sphos | 6′-dimethoxybiphenyl |
| S-Phos G₂ | chloro(2-dicyclohexylphosphino-2′,6′-dimethoxy- |
| | 1,1′-biphenyl)(2′-amino-1,1′-biphenyl-2-yl) |
| | palladium(II) |
| TBAF | tetrabutylammonium fluoride |
| TBS | tert-butyldimethylsilyl |
| TEA, Et₃N or NEt₃ | triethylamine |
| Tf | trifluoromethane sulfonyl or triflate |
| TFA | trifluoroacetic acid |
| THF | tetrahydrofuran |
| THP | tetrahydropyranyl |
| TIPS | tiisopropylsilane |
| TLC | thin layer chromatography |
| UPLC | Ulta performance liquid chromatography |
| XPhos Pd G4 | ligand for classic cross-coupling reactions |
| | (CAS Number 1599466-81-5) |

### Example 1

### Preparation of Compound 213

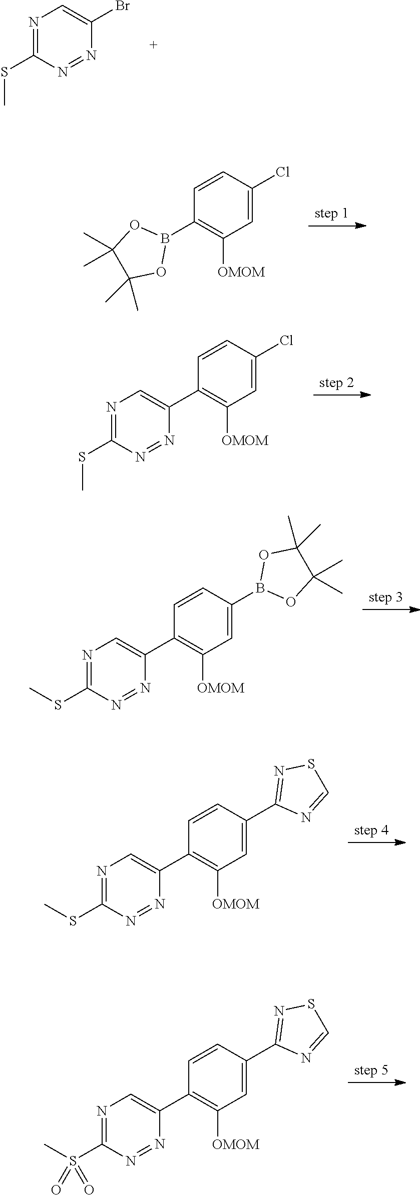

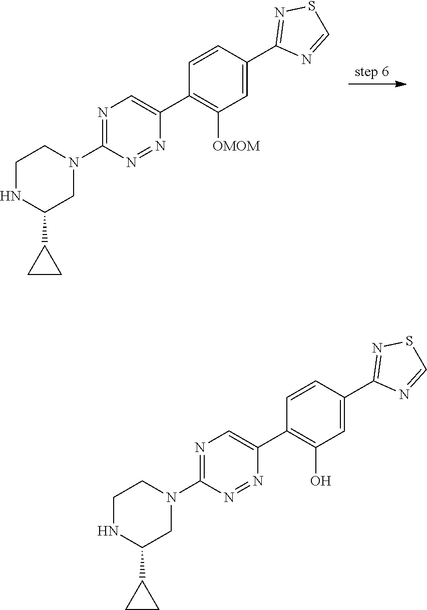

Step 1: To a dry round bottom flask were added: 6-bromo-3-methylsulfanyl-1,2,4-triazine (7.0 g, 34.0 mmol), 2-[4-chloro-2-(methoxymethoxy)phenyl]-4,4,5,5-tetramethyl-1,3,2-dioxaborolane (10 g, 33.5 mmol), K₂CO₃ (9.5 g, 68 mmol), PddppfCl₂ (3.8 g, 5.1 mmol). The mixture was degassed with argon for 10 min, then dioxane (100 mL) and water (20 mL) were added and the reaction was heated at 90° C. for 16 h (overnight). Reaction was cooled down to rt, partitioned between EtOAc and water, organic parts were dried over Na₂SO₄, concentrated, purified by silica-gel column chromatography eluting with a gradient 0-20% EtOAc in pentanes. Provided 6-[4-chloro-2-(methoxymethoxy)phenyl]-3-methylsulfanyl-1,2,4-triazine (4.5 g, 44% yield) as yellow solid. MS m/z 298.1, 300.1 [M+H]⁺.

Step 2: A suspension of 6-[4-chloro-2-(methoxymethoxy)phenyl]-3-methylsulfanyl-1,2,4-triazine (350 mg, 1.18 mmol) was added 4,4,5,5-tetramethyl-2-(4,4,5,5-tetramethyl-1,3,2-dioxaborolan-2-yl)-1,3,2-dioxaborolane (750 mg, 2.9 mmol), KOAc (575 mg, 5.86 mmol), X Phos Pd G4 (175 mg, 0.20 mmol) in dry dioxane (5 mL) was sparged with argon for 10 minutes, then heated to 90° C. under argon atmosphere for 2 h, after which complete conversion to 6-(2-(methoxymethoxy)-4-(4,4,5,5-tetramethyl-1,3,2-dioxaborolan-2-yl)phenyl)-3-(methylthio)-1,2,4-triazine was observed. The reaction mixture was then cooled to room temperature and used directly in the next step. MS m/z 390.4 [M+H]⁺.

Step 3: To the mixture from Step 2 above were added: 3-bromo-1,2,4-thiadiazole (0.15 g, 0.91 mmol), (2-dicyclohexylphosphino-2′,4′,6′-triisopropyl-1,1′-biphenyl)[2-(2′-amino-1,1′-biphenyl)]palladium(II) methanesulfonate (0.16 g, 0.181 mmol) and potassium carbonate (0.38 g, 2.72 mmol). The reaction was degassed via bubbling of nitrogen for 5 min and continued under nitrogen. 1,4-Dioxane (3 mL) and water (3 mL) were added to the mixture and the reaction was stirred at 90° C. for 2 h. The reaction was cooled to room temperature, diluted with water and extracted with EtOAc. Combined organic layers were dried over MgSO₄, filtered and concentrated under reduced pressure. The residue was purified by column chromatography eluting with a gradient (0-100%) EtOAc/hexanes to afford 3-[3-(methoxymethoxy)-4-(3-methylsulfanyl-1,2,4-triazin-6-yl)phenyl]-1,2,4-thiadiazole (0.2 g, 60% yield). MS m/z 348.2 [M+H]⁺.

Step 4: To a solution of 3-[3-(methoxymethoxy)-4-(3-methylsulfanyl-1,2,4-triazin-6-yl)phenyl]-1,2,4-thiadiazole (0.2 g, 0.6 mmol) in CH₂Cl₂ (5 mL) was added 3-chloroperoxybenzoic acid (0.2 g, 1.0 mmol) portion-wise. The reaction mixture was stirred at room temperature for 16 h. Solvent was removed under reduced pressure, the reminder was purified by column chromatography eluting with a gradient (0-10%) CH₂Cl₂/MeOH to afford 3-[3-(methoxymethoxy)-4-(3-methylsulfonyl-1,2,4-triazin-6-yl)phenyl]-1,2,4-thiadiazole (0.1 g, 50% yield); MS m/z 380.3 [M+H]⁺.

Step 5: To a solution of 3-[3-(methoxymethoxy)-4-(3-methylsulfonyl-1,2,4-triazin-6-yl)phenyl]-1,2,4-thiadiazole (0.1 g, 0.3 mmol) in ACN (3 mL) were added (2S)-2-cyclopropylpiperazine (0.07 g, 0.6 mmol) and N,N-diisopropylethylamine (0.2 mL, 1 mmol). The reaction mixture was stirred at 80° C. for 30 min. Solvent was removed under reduced pressure, the reminder was purified by column chromatography eluting with a gradient (0-20%) CH₂Cl₂/MeOH to afford 3-[4-[3-[(3S)-3-cyclopropylpiperazin-1-yl]-1,2,4-triazin-6-yl]-3-(methoxymethoxy)phenyl]-1,2,4-thiadiazole (0.07 g, 60% yield). MS m/z 426.5 [M+H]⁺.

Step 6: To a solution of 3-[4-[3-[(3S)-3-cyclopropylpiperazin-1-yl]-1,2,4-triazin-6-yl]-3-(methoxymethoxy)phenyl]-1,2,4-thiadiazole (0.07 g, 0.2 mmol) in CH₂Cl₂ (1 mL) and 2 drops of MeOH was added HCl (4 mol/L) in 1,4-dioxane (0.1 mL, 0.4 mmol). The reaction was stirred for 1 h until UPLC showed complete consumption of the starting material. The solvents were removed under reduced pressure, the reminder was purified by prep-HPLC eluting with a gradient 5-40% ACN in water (containing 0.1% formic acid) to provide 2-[3-[(3S)-3-cyclopropylpiperazin-1-yl]-1,2,4-triazin-6-yl]-5-(1,2,4-thiadiazol-3-yl)phenol (55 mg, 90% yield). MS m/z 382.2 [M+H]⁺; ¹H NMR (500 MHz, DMSO-d₆) δ: 10.99 (s, 1H), 10.39 (s, 1H), 9.16 (s, 1H), 9.10 (s, 1H), 8.00 (d, J=8.1 Hz, 1H), 7.95 (d, J=1.7 Hz, 1H), 7.87 (dd, J=8.1, 1.6 Hz, 1H), 4.83-4.67 (m, 2H), 3.59-3.35 (m, 3H), 3.18-3.07 (m, 1H), 2.77-2.63 (m, 1H), 1.04 (tt, J=8.4, 3.9 Hz, 1H), 0.67 (ddp, J=17.3, 8.5, 4.2 Hz, 2H), 0.56 (dq, J=9.9, 4.7 Hz, 1H), 0.45 (dq, J=8.8, 4.5 Hz, 1H).

Using the procedure described for Example 1, above, additional compounds described herein may be prepared by substituting the appropriate starting material, suitable reagents and reaction conditions, obtaining compounds such as those selected from:

| Cpd | Data |
|---|---|
| 1 | MS m/z 382.4 [M + H]⁺; ¹H NMR (500 MHz, methanol-d₄) δ: 9.05 (s, 1H), |
| | 7.98 (s, 1H), 7.83 (d, J = 8.7 Hz, 1H), 7.18-7.23 (m, 2H), 4.80 (br d, J = 12.4 |
| | Hz, 1H), 4.64 (d, J = 13.7 Hz, 1H), 3.12-3.22 (m, 2H), 3.01 (dd, J = 12.9, 10.9 |
| | Hz, 1H), 2.79-2.89 (m, 1H), 1.94-2.03 (m, 1H), 0.82-0.95 (m, 1H), 0.57-0.67 |
| | (m, 2H), 0.31-0.43 (m, 2H); 3Hs not observed (2 NHs and OH). |
| 2 | MS m/z 409.5 [M + H]⁺; ¹H NMR (500 MHz, methanol-d₄) δ: 9.10 (s, 1H), |
| | 8.81 (s, 1H), 7.98 (d, J = 8.2 Hz, 1H), 7.70 (s, 1H), 7.68 (br d, J = 8.2 Hz, 1H), |
| | 7.33 (s, 1H), 4.83 (br d, J = 13.3 Hz, 1H), 4.72 (d, J = 13.4 Hz, 1H), 3.12-3.24 |
| | (m, 2H), 3.02 (dd, J = 12.8, 10.7 Hz, 1H), 2.83 (td, J = 12.2, 2.5 Hz, 1H), 1.98 |
| | (td, J = 9.2, 2.7 Hz, 1H), 0.83-0.95 (m, 1H), 0.57-0.67 (m, 2H), 0.30-0.43 (m, |
| | 2H); 2Hs not observed (NH and OH). |
| 3 | MS m/z 384.1 [M + H]⁺; ¹H NMR (500 MHz, methanol-d₄) δ: 8.93 (s, 1H), |
| | 7.86 (s, 1H), 7.72 (d, J = 8.6 Hz, 1H), 7.11-7.07 (m, 2H), 4.73-4.52 (m, |
| | 2H), 3.09-2.95 (m, 2H), 2.77-2.65 (m, 2H), 2.34 (ddd, J = 10.4, 7.0, 3.0 |
| | Hz, 1H), 1.62 (h, J = 6.9 Hz, 1H), 0.95 (dd, J = 6.9, 1.9 Hz, 6H); 3Hs not |
| | observed (2 NHs and OH). |
| 4 | MS m/z 400.4 [M + H]⁺; ¹H NMR (500 MHz, methanol-d₄) δ: 9.07 (s, 1H), |
| | 7.98 (d, J = 1.8 Hz, 1H), 7.85 (d, J = 8.5 Hz, 1H), 7.16-7.26 (m, 2H), 4.91-4.95 |
| | (m, 1H), 4.75 (d, J = 13.3 Hz, 1H), 3.20 (d, J = 13.0 Hz, 1H), 3.08 (td, J = 12.4, |
| | 2.5 Hz, 1H), 2.81-2.94 (m, 2H), 2.66 (br d, J = 10.1 Hz, 1H), 1.31 (d, J = 12.4 |
| | Hz, 6H); 4Hs not observed (2 NHs and 2OHs). |
| 5 | MS m/z 364.3 [M + H]⁺; ¹H NMR (500 MHz, DMSO-d₆) δ: 9.79 (d, J = 10.5 |
| | Hz, 1H), 9.43 (d, J = 10.8 Hz, 1H), 9.14 (s, 1H), 8.10 (s, 2H), 7.87 (d, J = 8.0 |
| | Hz, 1H), 7.21-7.27 (m, 2H), 4.61-4.80 (m, 2H), 3.45-3.60 (m, 2H), 3.41 (dt, J = |
| | 12.8, 2.9 Hz, 1H), 2.99-3.11 (m, 1H), 2.61-2.72 (m, 1H), 1.10 (ddt, J = |
| | 13.0, 8.0, 4.4 Hz, 1H), 0.56-0.73 (m, 3H), 0.38-0.47 (m, 1H); 1H not observed. |
| 6 | MS m/z 380.5 [M + H]⁺; ¹H NMR (500 MHz, methanol-d₄) δ: 9.08-9.15 (s, |
| | 1H), 8.31-8.43 (s, 2H), 8.01 (s, 2H), 7.78-7.87 (m, 1H), 7.21-7.25 (m, 1H), |
| | 7.18-7.21 (m, 1H), 4.97-5.09 (m, 2H), 3.47-3.59 (m, 2H), 3.37-3.46 (m, 1H), |
| | 3.23-3.31 (m, 1H), 2.87-2.96 (m, 1H), 0.92-1.05 (m, 2H), 0.76-0.88 (m, 2H); |
| | 2Hs not observed (NH and OH). |
| 7 | MS m/z 382.4 [M + H]⁺; ¹H NMR (500 MHz, methanol-d₄) δ: 9.06 (br s, 1H), |
| | 7.98 (br s, 1H), 7.84 (d, J = 7.8 Hz, 1H), 7.21 (br s, 2H), 4.80 (br d, J = 12.5 Hz, |
| | 1H), 4.69 (d, J = 12.5 Hz, 1H), 3.11-3.23 (m, 2H), 3.00 (br dd, J = 10.6, 9.8 Hz, |
| | 1H), 2.73-2.90 (m, 1H), 1.87-2.09 (m, 1H), 0.78-0.97 (m, 1H), 0.55-0.71 (m, |
| | 2H), 0.27-0.48 (m, 2H); 3Hs not observed (2 NHs and OH). |
| 8 | MS m/z 427.4 [M + H]⁺; ¹H NMR (500 MHz, methanol-d₄) δ: 9.12 (s, 1H), |
| | 8.81 (s, 1H), 7.95-8.08 (m, 1H), 7.63-7.78 (m, 2H), 7.34 (s, 1H), 4.95-5.07 |
| | (m, 1H), 4.03-4.14 (m, 1H), 3.12-3.21 (m, 2H), 2.91-3.08 (m, 2H), 2.71-2.89 |
| | (m, 1H), 1.34 (br d, J = 13.9 Hz, 6H); 3Hs not observed (NH and 2OHs). |
| 9 | MS m/z 381.5 [M + H]⁺; ¹H NMR (500 MHz, DMSO-d₆) δ: 9.66 (d, J = 10.5 |
| | Hz, 1H), 9.36 (d, J = 10.8 Hz, 1H), 9.12 (s, 1H), 8.17 (s, 1H), 7.83-7.89 (m, |
| | 2H), 7.15-7.22 (m, 2H), 4.60-4.82 (m, 2H), 3.38-3.57 (m, 3H), 3.07 (q, J = |
| | 11.3 Hz, 1H), 2.63-2.71 (m, 1H), 1.05-1.15 (m, 1H), 0.55-0.73 (m, 3H), |
| | 0.39-0.47 (m, 1H). |
| 10 | MS m/z 411.5 [M + H]⁺; ¹H NMR (500 MHz, methanol-d₄) δ: 9.10 (br s, 1H), |
| | 8.81 (s, 1H), 7.98 (br s, 1H), 7.61-7.77 (m, 2H), 7.33 (s, 1H), 4.64-4.78 (m, |
| | 2H), 3.12-3.22 (m, 2H), 2.81-2.95 (m, 2H), 2.38-2.57 (m, 1H), 1.72-1.82 (m, |
| | 1H), 0.95-1.18 (m, 6H); 2Hs not observed (NH and OH). |
| 12 | MS m/z 378.5 [M + H]⁺; ¹H NMR (500 MHz, methanol-d₄) δ: 9.05 (s, 1H), |
| | 8.01 (s, 1H), 7.86 (s, 1H), 7.81 (d, J = 8.1 Hz, 1H), 7.19 (br d, J = 8.4 Hz, 1H), |
| | 7.16 (s, 1H), 4.79 (d, J = 13.3 Hz, 1H), 4.68 (br d, J = 13.3 Hz, 1H), 3.95 (s, |
| | 3H), 3.08-3.21 (m, 2H), 3.00 (dd, J = 13.5, 12.1 Hz, 1H), 2.82 (td, J = 12.8, 2.5 |
| | Hz, 1H), 1.96 (br td, J = 8.5, 1.8 Hz, 1H), 0.81-0.96 (m, 1H), 0.54-0.70 (m, |
| | 2H), 0.30-0.42 (m, 2H); 2Hs not observed (NH and OH). |
| 13 | MS m/z 382.3 [M + H]⁺; ¹H NMR (500 MHz, methanol-d₄) δ: 8.47-8.40 (m, |
| | 1H), 8.35 (br s, 1H), 7.70 (d, J = 8.1 Hz, 1H), 7.38-7.19 (m, 2H), 6.03 (br s, |
| | 1H), 5.20-4.98 (m, 1H), 4.39-4.25 (m, 1H), 3.78-3.38 (m, 5H), 1.48-1.37 |
| | (m, 6H); 4Hs not observed (2 NHs and 2OHs). |
| 15 | MS m/z 384.1 [M + H]⁺; ¹H NMR (500 MHz, methanol-d₄) δ: 8.97 (s, 1H), |
| | 7.91-7.85 (m, 1H), 7.74-7.70 (m, 1H), 7.12-7.02 (m, 2H), 4.84 (d, J = |
| | 13.0 Hz, 1H), 4.74-4.69 (m, 1H), 3.30-3.25 (m, 1H), 3.20-3.16 (m, 1H), |
| | 2.98 (td, J = 12.9, 9.6 Hz, 2H), 2.77 (ddd, J = 11.2, 7.5, 3.3 Hz, 1H), 1.80 (h, |
| | J = 6.9 Hz, 1H), 1.02 (dd, J = 6.7, 1.9 Hz, 6H); 3Hs not observed (2 NHs and OH). |
| 16 | MS m/z 380.3 [M + H]⁺; ¹H NMR (500 MHz, methanol-d₄) δ: 9.13 (s, 1H), 8.07 |
| | (s, 2H), 7.84 (d, J = 8.1 Hz, 1H), 7.26 (d, J = 8.5 Hz, 1H), 7.22 (s, 1H), 5.13 (br |
| | d, J = 14.2 Hz, 1H), 5.00 (d, J = 14.3 Hz, 1H), 3.57 (d, J = 12.8 Hz, 1H), 3.45 (td, |
| | J = 13.5, 3.1 Hz, 1H), 3.23-3.33 (m, 2H), 3.18 (dd, J = 11.3, 2.1 Hz, 1H), 1.19 (s, |
| | 9H); 3Hs not observed (2 NHs and OH). |
| 17 | MS m/z 425.6 [M + H]⁺; ¹H NMR (500 MHz, methanol-d₄) δ: 9.07 (s, 1H), |
| | 8.79 (s, 1H), 7.95 (d, J = 8.2 Hz, 1H), 7.67 (s, 1H), 7.64 (d, J = 8.2 Hz, 1H), |
| | 7.29 (s, 1H), 4.91 (br d, J = 13.0 Hz, 1H), 4.76 (br d, J = 13.0 Hz, 1H), 3.19 (br |
| | d, J = 12.1 Hz, 1H), 3.08 (td, J = 12.7, 2.9 Hz, 1H), 2.78-2.92 (m, 2H), 2.46 (br |
| | d, J = 9.3 Hz, 1H), 1.06 (s, 9H); 2Hs not observed (NH and OH). |
| 19 | MS m/z 399.5 [M + H]⁺; ¹H NMR (500 MHz, methanol-d₄) δ: 9.07 (s, 1H), |
| | 8.02 (s, 1H), 7.86 (s, 1H), 7.83 (d, J = 7.9 Hz, 1H), 7.20 (dd, J = 8.4, 1.4 Hz, |
| | 1H), 7.17 (d, J = 1.4 Hz, 1H), 4.93 (br d, J = 12.1 Hz, 1H), 4.78 (d, J = 13.2 Hz, |
| | 1H), 3.24 (d, J = 13.1 Hz, 1H), 3.11 (td, J = 12.1, 3.5 Hz, 1H), 2.92 (t, J = 12.1 |
| | Hz, 2H), 2.73 (br d, J = 11.4 Hz, 1H), 1.34 (s, 3H), 1.31 (s, 3H); 3Hs not |
| | observed (NH and 2OHs). |
| 20 | MS m/z 366.2 [M + H]⁺; ¹H NMR (500 MHz, methanol-d₄) δ: 9.01 (s, 1H), |
| | 8.10-7.92 (m, 2H), 7.79 (d, J = 8.1 Hz, 1H), 7.18 (s, 2H), 4.85-4.72 (m, |
| | 1H), 4.69-4.61 (m, 1H), 3.17-3.06 (m, 2H), 2.88-2.77 (m, 2H), 2.48-2.42 |
| | (m, 1H), 1.73 (qd, J = 6.8, 13.7 Hz, 1H), 1.06 (dd, J = 3.4, 6.8 Hz, 6H); 3Hs |
| | not observed (2 NHs and OH). |
| 21 | MS m/z 394.5 [M + H]⁺; ¹H NMR (500 MHz, methanol-d₄) δ: 9.05 (s, 1H), |
| | 8.02 (s, 1H), 7.86 (s, 1H), 7.82 (d, J = 8.2 Hz, 1H), 7.19 (d, J = 8.2 Hz, 1H), |
| | 7.17 (s, 1H), 4.89-4.93 (m, 1H), 4.73 (d, J = 12.8 Hz, 1H), 3.96 (s, 3H), 3.18 |
| | (br d, J = 12.7 Hz, 1H), 3.07 (td, J = 12.7, 2.5 Hz, 1H), 2.85 (dd, J = 12.7, 10.2 |
| | Hz, 2H), 2.45 (br d, J = 10.2 Hz, 1H), 1.06 (s, 9H); 2Hs not observed (NH and OH). |
| 22 | MS m/z 366.6 [M + H]⁺; ¹H NMR (500 MHz, methanol-d₄) δ: 9.04 (s, 1H), |
| | 8.01 (br s, 2H), 7.81 (d, J = 8.2 Hz, 1H), 7.26-7.15 (m, 2H), 4.81 (d, J = |
| | 13.0 Hz, 1H), 4.68 (d, J = 13.7 Hz, 1H), 3.19-3.08 (m, 2H), 2.90-2.79 (m, |
| | 2H), 2.52-2.43 (m, 1H), 1.80-1.68 (m, 1H), 1.07 (d, J = 6.7 Hz, 6H); 3Hs |
| | not observed (2 NHs and OH). |
| 23 | MS m/z 352.2 [M + H]⁺; ¹H NMR (500 MHz, methanol-d₄) δ: 9.02 (s, 1H), |
| | 8.06-7.92 (m, 2H), 7.78 (d, J = 8.1 Hz, 1H), 7.21-7.16 (m, 2H), 4.78-4.64 |
| | (m, 2H), 3.15 (s, 2H), 2.94-2.85 (m, 1H), 2.84-2.75 (m, 1H), 2.74-2.65 |
| | (m, 1H), 1.56 (s, 2H), 1.06 (t, J = 7.6 Hz, 3H); 3Hs not observed (2 NHs and OH). |
| 24 | MS m/z 352.2 [M + H]⁺; ¹H NMR (500 MHz, methanol-d₄) δ: 8.61 (s, 2H), |
| | 7.73 (d, J = 8.2 Hz, 1H), 7.43-7.29 (m, 2H), 6.04 (s, 1H), 4.42-4.14 (m, |
| | 2H), 3.82-3.40 (m, 5H), 1.92-1.77 (m, 2H), 1.24-1.10 (m, 3H); 3Hs not |
| | observed (2 NHs and OH). |
| 25 | MS m/z 400.4 [M + H]⁺; ¹H NMR (500 MHz, methanol-d₄) δ: 9.06 (s, 1H), |
| | 7.98 (d, J = 1.4 Hz, 1H), 7.84 (d, J = 8.7 Hz, 1H), 7.16-7.26 (m, 2H), 4.91 (d, |
| | J = 13.4 Hz, 1H), 4.75 (d, J = 13.4 Hz, 1H), 3.21 (d, J = 12.8 Hz, 1H), 3.08 (td, |
| | J = 12.8, 3.1 Hz, 1H), 2.82-2.93 (m, 2H), 2.67 (d, J = 11.0 Hz, 1H), 1.31 (d, |
| | J = 12.8 Hz, 6H); 4Hs not observed (2 NHs and 2OHs). |
| 26 | MS m/z 441.5 [M + H]⁺; ¹H NMR (500 MHz, methanol-d₄) δ: 9.11 (s, 1H), |
| | 8.81 (s, 1H), 7.98 (d, J = 8.2 Hz, 1H), 7.71 (d, J = 2.0 Hz, 1H), 7.68 (dd, J = 8.1, |
| | 2.0 Hz, 1H), 7.34 (s, 1H), 4.61 (dd, J = 13.8, 4.3 Hz, 1H), 4.25-4.38 (m, 1H), |
| | 3.37-3.51 (m, 2H), 3.09-3.26 (m, 1H), 2.74-2.85 (m, 1H), 2.59 (s, 3H), 2.46 |
| | (dd, J = 8.9, 4.3 Hz, 1H), 1.30 (d, J = 6.4 Hz, 6H); 2Hs not observed (2OHs). |
| 27 | MS m/z 378.4 [M + H]⁺; ¹H NMR (500 MHz, DMSO-d₆) δ: 9.14 (d, J = 10.9 |
| | Hz, 1H), 8.96 (s, 1H), 8.09 (s, 2H), 7.87 (dd, J = 10.1, 4.0 Hz, 1H), 7.15-7.37 |
| | (m, 3H), 4.75-4.84 (m, 2H), 3.45-3.64 (m, 3H), 3.08-3.15 (m, 1H), 2.94 (s, |
| | 3H), 2.60-2.73 (m, 1H), 1.08-1.17 (m, 1H), 0.82-0.89 (m, 1H), 0.69-0.80 (m, |
| | 1H), 0.66-0.63 (m, 1H), 0.31-0.39 (m, 1H). |
| 28 | MS m/z 426.5 [M + H]⁺; ¹H NMR (500 MHz, methanol-d₄) δ: 8.82 (s, 2H), |
| | 8.51 (s, 1H), 7.57 (s, 1H), 7.52 (d, J = 12.1 Hz, 1H), 7.35 (s, 1H), 5.00 (d, |
| | J = 13.3 Hz, 1H), 4.81-4.80 (m, 1H), 4.07 (m, 3H), 3.37-3.39 (m, 2H), 3.07- |
| | 3.13 (m, 2H), 2.86-2.90 (m, 1H), 1.89-1.93 (m, 1H), 1.13 (d, J = 6.85 Hz, 6H); |
| | 2Hs not observed (NH and OH), 1H from formic acid salt. |
| 29 | MS m/z 394.5 [M + H]⁺; ¹H NMR (500 MHz, methanol-d₄) δ: 9.11 (s, 1H), |
| | 8.31 (s, 1H), 8.02 (s, 2H), 7.77-7.89 (m, 1H), 7.16-7.28 (m, 1H), 4.95-5.05 |
| | (m, 2H), 3.48-3.56 (m, 1H), 3.35-3.45 (m, 2H), 3.36 (s, 3H), 3.25-3.31 (m, |
| | 2H), 1.10-1.26 (m, 2H), 0.81-0.94 (m, 2H); 3Hs not observed (2 NHs and OH). |
| 30 | MS m/z 378.2 [M + H]⁺; ¹H NMR (500 MHz, methanol-d₄) δ: 9.03 (s, 1H), |
| | 8.07-7.88 (m, 2H), 7.79 (d, J = 8.1 Hz, 1H), 7.22-7.16 (m, 2H), 4.72-4.63 |
| | (m, 2H), 3.24-3.13 (m, 2H), 2.97-2.87 (m, 1H), 2.85-2.72 (m, 2H), |
| | 2.47-2.37 (m, 1H), 2.19-2.09 (m, 2H), 2.05-1.84 (m, 4H); 3Hs not observed |
| | (2 NHs and OH). |
| 31 | MS m/z 366.3 [M + H]⁺; ¹H NMR (500 MHz, DMSO-d₆) δ: 9.13 (s, 1H), 8.14 |
| | (s, 2H), 7.87 (d, J = 8.5 Hz, 1H), 7.27-7.23 (m, 2H), 4.80-4.65 (m, 2H), |
| | 3.60-3.49 (m, 1H), 3.41-3.25 (m, 3H), 3.16-3.04 (m, 1H), 1.76-1.61 (m, |
| | 2H), 1.53-1.36 (m, 2H), 0.94 (t, J = 7.2 Hz, 3H); 3Hs not observed |
| | (2 NHs and OH). |
| 32 | MS m/z 400.3 [M + H]⁺; [M + H]⁺; ¹H NMR (500 MHz, methanol-d₄) δ: 8.71- |
| | 8.69 (m, 1H), 8.42 (s, 1H), 7.91 (d, J = 1.6 Hz, 1H), 6.97-7.0 (m, 1H), 6.90 (d, |
| | J = 12.4 Hz, 1H), 4.79-4.82 (m, 1H), 4.69-4.72 (m, 1H), 3.26-3.31 (m, 2H), |
| | 3.07-3.16 (m, 1H), 2.89-2.95 (m, 1H), 2.18-2.22 (m, 1H), 0.84-0.87 (m, 1H), |
| | 0.58-0.62 (m, 2H), 0.36-0.39 (m, 2H); 3Hs not observed (2 NHs and OH), 1H |
| | from formic acid salt. |
| 33 | MS m/z 380.2 [M + H]⁺; ¹H NMR (500 MHz, DMSO-d₆) δ: 9.14 (s, 1H), 8.14 |
| | (s, 2H), 7.87 (d, J = 8.5 Hz, 1H), 7.26-7.24 (m, 2H), 4.83-4.72 (m, 2H), |
| | 3.57-3.48 (m, 1H), 3.40-3.32 (m, 1H), 3.31-3.22 (m, 2H), 3.17 (s, 1H), |
| | 1.93-1.84 (m, 1H), 1.62-1.51 (m, 1H), 1.39-1.29 (m, 1H), 1.07-1.03 (m, |
| | 3H), 0.93 (t, J = 7.3 Hz, 3H); 3Hs not observed (2 NHs and OH). |
| 34 | MS m/z 380.3 [M + H]⁺; ¹H NMR (500 MHz, methanol-d₄) δ: 9.04 (s, 1H), |
| | 8.13-7.88 (m, 2H), 7.81 (d, J = 8.1 Hz, 1H), 7.24-7.17 (m, 2H), 4.74-4.61 |
| | (m, 2H), 3.22 (d, J = 2.6 Hz, 1H), 3.05-2.88 (m, 2H), 2.45-2.39 (m, 1H), |
| | 2.39-2.36 (m, 3H), 2.31-2.23 (m, 1H), 2.07-2.00 (m, 1H), 1.10 (d, J = 6.9 |
| | Hz, 3H), 1.02 (d, J = 6.9 Hz, 3H); 2Hs not observed (NH and OH). |
| 35 | MS m/z 380.2 [M + H]⁺; ¹H NMR (500 MHz, methanol-d₄) δ: 8.93 (s, 1H), |
| | 7.90 (s, 1H), 7.74 (s, 1H), 7.69 (d, J = 8.2 Hz, 1H), 7.07 (dd, J = 8.1, 1.8 Hz, |
| | 1H), 7.04 (d, J = 1.7 Hz, 1H), 4.71 (d, J = 12.7 Hz, 1H), 4.58 (d, J = 14.9 Hz, |
| | 1H), 3.83 (s, 3H), 3.09-2.96 (m, 2H), 2.80-2.70 (m, 2H), 2.39 (ddd, J = |
| | 10.4, 7.1, 3.0 Hz, 1H), 1.64 (h, J = 6.9 Hz, 1H), 0.96 (dd, J = 6.8, 2.0 Hz, |
| | 6H); 2Hs not observed (NH and OH). |
| 36 | MS m/z 400.3 [M + H]⁺; ¹H NMR (500 MHz, methanol-d₄) δ: 9.23 (s, 0.5H), |
| | 8.49 (s, 1H), 8.39 (s, 1H), 7.94 (d, J = 8.2 Hz, 0.5H), 7.71 (d, J = 8.2 Hz, 0.5H), |
| | 7.27-7.36 (m, 2H), 6.02 (s, 0.5H), 4.80-4.87 (m, 1H), 4.12-4.33 (m, 1H), |
| | 3.42-3.88 (m, 5H), 2.05-2.23 (m, 1H), 1.85-2.00 (m, 1H), 1.65-1.83 (m, 1H); |
| | 3Hs not observed (2 NHs and OH). |
| 37 | MS m/z 366.3 [M + H]⁺; ¹H NMR (500 MHz, methanol-d₄) δ: 9.13 (s, 1H), |
| | 8.02 (s, 2H), 7.85-7.82 (m, 1H), 7.25-7.19 (m, 2H), 5.00-4.88 (m, 2H), |
| | 3.58-3.49 (m, 2H), 3.37 (s, 1H), 3.32-3.23 (m, 2H), 1.81-1.70 (m, 2H), |
| | 1.65-1.51 (m, 2H), 1.07 (t, J = 7.3 Hz, 3H); 3Hs not observed (2 NHs and OH). |
| 38 | MS m/z 350.2 [M + H]⁺; ¹H NMR (500 MHz, methanol-d₄) δ: 9.11 (s, 0.5 H), |
| | 8.35 (s, 1H), 8.26 (s, 1H), 7.82 (d, J = 8.2 Hz, 0.5H), 7.59 (d, J = 8.2 Hz, 0.5H), |
| | 7.14-7.26 (m, 2H), 5.83-5.95 (m, 1.5H), 5.49-5.69 (m, 2H), 4.80-4.87 (m, |
| | 1H), 3.91-4.09 (m, 2H), 3.31-3.69 (m, 4H); 3Hs not observed (2 NHs and OH). |
| 39 | MS m/z 369.3 [M + H]⁺; ¹H NMR (500 MHz, methanol-d₄) δ: 9.13 (s, 1H), |
| | 8.04 (s, 1H), 7.87 (s, 1H), 7.83 (d, J = 8.2 Hz, 1H), 7.22 (d, J = 7.9 Hz, 1H), |
| | 7.18 (s, 1H), 4.93-5.01 (m, 2H), 3.48-3.61 (m, 2H), 3.24-3.38 (m, 3H), 1.73- |
| | 1.92 (m, 2H), 1.16 (t, J = 7.6 Hz, 3H); 2 Hs not observed (NH and OH). |
| 40 | MS m/z 366.2 [M + H]⁺; ¹H NMR (500 MHz, methanol-d₄) δ: 9.01 (s, 1H), |
| | 8.10-7.92 (m, 2H), 7.79 (d, J = 8.1 Hz, 1H), 7.18 (s, 2H), 4.85-4.72 (m, |
| | 1H), 4.69-4.61 (m, 1H), 3.17-3.06 (m, 2H), 2.88-2.77 (m, 2H), 2.48-2.42 |
| | (m, 1H), 1.73 (qd, J = 6.8, 13.7 Hz, 1H), 1.06 (dd, J = 3.4, 6.8 Hz, 6H); 3Hs |
| | not observed (2 NHs and OH). |
| 41 | MS m/z 383.2 [M + H]⁺; ¹H NMR (500 MHz, methanol-d₄) δ: 9.13 (s, 1H) |
| | 8.01 (s, 1H), 7.87-7.78 (m, 2H) 7.22-7.17 (m, 1H) 7.16 (s, 1H) 5.10-5.01 (m, |
| | 1H) 4.98-4.92 (m, 1H) 3.56-3.12 (m, 4H), 2.25-2.13 (m, 1H), 2.06-1.95 (m, |
| | 1H), 1.17 (d, J = 6.9 Hz, 6H); 2Hs not observed (NH and OH). |
| 42 | MS m/z 338.2 [M + H]⁺; ¹H NMR (500 MHz, methanol-d₄) δ: 9.09 (s, 1H), |
| | 7.96 (s, 2H), 7.81 (d, J = 8.1 Hz, 1H), 7.25-7.14 (m, 2H), 4.95-4.86 (m, |
| | 2H), 3.59-3.42 (m, 3H), 3.31-3.20 (m, 2H), 1.46 (d, J = 6.6 Hz, 3H); 3Hs |
| | not observed (2 NHs and OH). |
| 43 | MS m/z 378.4 [M + H]⁺; ¹H NMR (500 MHz, methanol-d₄) δ: 9.05 (s, 1H), |
| | 7.94-8.13 (m, 2H), 7.82 (d, J = 8.2 Hz, 1H), 7.23 (dd, J = 8.2, 1.8 Hz, 1H), 7.20 |
| | (d, J = 1.5 Hz, 1H), 3.94 (dd, J = 6.8, 5.1 Hz, 2H), 3.79-3.83 (m, 2H), 3.02 (dd, |
| | J = 6.8, 4.6 Hz, 2H), 1.71-1.85 (m, 6H), 1.57-1.65 (m, 2H); 3Hs not observed |
| | (2 NHs and OH). |
| 44 | MS m/z 391.3 [M + H]⁺; ¹H NMR (500 MHz, DMSO-d₆) δ: 9.15 (s, 1H), 8.82 |
| | (d, J = 6.4 Hz, 1H), 8.31 (s, 1H), 8.19-8.15 (m, 1H), 8.09 (d, J = 8.1 Hz, |
| | 1H), 7.66-7.60 (m, 2H), 4.94-4.69 (m, 2H), 3.62-3.50 (m, 1H), 3.44-3.37 |
| | (m, 1H), 3.37-3.26 (m, 1H), 3.16-3.08 (m, 2H), 2.82 (s, 3H), 2.14-2.01 |
| | (m, 1H), 1.08 (t, J = 6.9 Hz, 6H); 2Hs not observed (NH and OH). |
| 46 | MS m/z 380.2 [M + H]⁺; ¹H NMR (500 MHz, methanol-d₄) δ: 9.10 (s, 1H), |
| | 7.95 (s, 2H), 7.81 (d, J = 8.1 Hz, 1H), 7.25-7.14 (m, 2H), 5.02-4.85 (m, |
| | 2H), 3.61-3.47 (m, 2H), 3.43-3.36 (m, 1H), 3.28 (s, 2H), 1.94-1.82 (m, |
| | 1H), 1.72-1.56 (m, 2H), 1.05 (d, J = 6.6 Hz, 6H); 3Hs not observed (2 NHs and OH). |
| 47 | MS m/z 383.5 [M + H]⁺; ¹H NMR (500 MHz, methanol-d₄) δ: 9.06 (s, 1H), |
| | 8.02 (s, 1H), 7.86 (s, 1H), 7.82 (d, J = 8.1 Hz, 1H), 7.19 (d, J = 8.2 Hz, 1H), |
| | 7.16 (s, 1H), 4.83 (d, J = 12.8 Hz, 1H), 4.70 (d, J = 14.0 Hz, 1H), 3.11-3.21 (m, |
| | 2H), 2.79-2.93 (m, 2H), 2.44-2.58 (m, 1H), 1.76 (h, J = 6.9 Hz, 1H), 1.08 (d, |
| | J = 6.9 Hz, 6H); 2Hs not observed (NH and OH). |
| 48 | MS m/z 352.2 [M + H]⁺; ¹H NMR (500 MHz, methanol-d₄) δ: 9.02 (s, 1H), |
| | 8.06-7.92 (m, 2H), 7.78 (d, J = 8.1 Hz, 1H), 7.21-7.16 (m, 2H), 4.78-4.64 |
| | (m, 2H), 3.15 (s, 2H), 2.94-2.85 (m, 1H), 2.84-2.75 (m, 1H), 2.74-2.65 |
| | (m, 1H), 1.56 (s, 2H), 1.06 (t, J = 7.6 Hz, 3H); 3Hs not observed (2 NHs and OH). |
| 49 | MS m/z 369.3 [M + H]⁺; ¹H NMR (500 MHz, methanol-d₄) δ: 9.13 (s, 1H), |
| | 8.01 (s, 1H), 7.85 (s, 1H), 7.84 (d, J = 8.1 Hz, 1H), 7.20 (d, J = 6.6 Hz, 1H), |
| | 7.16 (d, J = 1.5 Hz, 1H), 5.05 (dd, J = 14.3, 2.7 Hz, 2H), 3.43-3.52 (m, 2H), |
| | 3.04 (dd, J = 14.5, 11.6 Hz, 2H), 1.44 (d, J = 6.6 Hz, 6H); 2Hs not observed |
| | (NH and OH). |
| 50 | MS m/z 364.3 [M + H]⁺; ¹H NMR (500 MHz, methanol-d₄) δ: 9.05 (s, 1H), |
| | 8.07 (br s, 1H), 7.93 (br s, 1H), 7.82 (d, J = 8.2 Hz, 1H), 7.22 (d, J = 8.1 Hz, |
| | 1H), 7.19 (s, 1H), 3.93 (s, 2H), 3.85-3.91 (m, 2H), 2.87-2.96 (m, 2H), 2.00- |
| | 2.09 (m, 2H), 1.94 (td, J = 14.2, 7.2 Hz, 4H); 3Hs not observed (2 NHs and OH). |
| 51 | MS m/z 383.5 [M + H]⁺; ¹H NMR (500 MHz, methanol-d₄) δ: 9.03 (s, 1H), |
| | 7.99 (s, 1H), 7.84 (s, 1H), 7.76 (d, J = 7.0 Hz, 1H), 7.09-7.19 (m, 2H), 4.64 (br |
| | d, J = 12.8 Hz, 2H), 2.80-2.91 (m, 2H), 2.30-2.38 (m, 4H), 1.23 (d, J = 6.1 Hz, |
| | 6H); 2Hs not observed (NH and OH). |
| 53 | MS m/z 368.2 [M + H]⁺; ¹H NMR (500 MHz, methanol-d₄) δ: 8.98 (s, 1H), |
| | 7.95 (s, 2H), 7.79-7.73 (m, 1H), 7.18-7.15 (m, 2H), 4.65 (t, J = 14.2 Hz, |
| | 2H), 3.51-3.41 (m, 2H), 3.41-3.40 (m, 3H), 3.19-3.10 (m, 2H), 3.04-2.97 |
| | (m, 1H), 2.96-2.82 (m, 2H); 3Hs not observed (2 NHs and OH). |
| 55 | MS m/z 350.2 [M + H]⁺; ¹H NMR (500 MHz, DMSO-d₆) δ: 9.11 (s, 1H), 8.14 |
| | (s, 2H), 7.87 (d, J = 7.9 Hz, 1H), 7.27-7.22 (m, 2H), 4.23-4.14 (m, 2H), |
| | 4.03 (s, 2H), 3.35-3.29 (m, 1H), 1.19-1.02 (m, 3H), 0.97-0.92 (m, 2H); |
| | 3Hs not observed (2 NHs and OH). |
| 56 | MS m/z 352.3 [M + H]⁺; ¹H NMR (500 MHz, methanol-d₄) δ: 9.09-9.18 (m, |
| | 1H), 8.02-8.08 (m, 2H), 7.85 (d, J = 8.2 Hz, 1H), 7.26 (dd, J = 8.1, 1.7 Hz, 1H), |
| | 7.22 (d, J = 1.5 Hz, 1H), 4.23 (br t, J = 5.3 Hz, 2H), 4.06 (s, 2H), 3.44 (br d, |
| | J = 5.5 Hz, 1H), 3.40-3.47 (m, 1H),1.5O (s, 6H); 3Hs not observed (2 NHs and OH). |
| 57 | MS m/z 368.3 [M + H]⁺; ¹H NMR (500 MHz, methanol-d₄) δ: 9.05 (s, 1H), |
| | 7.98 (s, 1H), 7.85 (d, J = 8.5 Hz, 1H), 7.16-7.26 (m, 2H), 3.97-4.06 (m, 2H), |
| | 3.66-3.72 (m, 1H), 3.02-3.13 (m, 2H), 0.84-0.98 (m, 1H), 0.63-0.78 (m, 4H); |
| | 3Hs not observed (2 NHs and OH). |
| 58 | MS m/z 381.2[M + H]⁺; ¹H NMR (500 MHz, methanol-d₄) δ: 9.03 (s, 1H), |
| | 7.99 (s, 1H), 7.84 (s, 1H), 7.80 (d, J = 8.1 Hz, 1H), 7.17 (br d, J = 8.2 Hz, 1H), |
| | 7.14 (s, 1H), 4.36 (br d, J = 12.5 Hz, 2H), 3.38 (s, 2H), 3.27 (s, 1H), 2.41 (s, |
| | 3H), 2.06-2.18 (m, 3H), 1.70 (br d, J = 7.9 Hz, 2H); 1H not observed (OH). |
| 59 | MS m/z 380.4 [M + H]⁺; ¹H NMR (500 MHz, methanol-d₄) δ: 9.07 (s, 1H), |
| | 8.09 (br s, 1H), 7.95 (br s, 1H), 7.84 (d, J = 8.2 Hz, 1H), 7.24 (dd, J = 8.2, 1.5 |
| | Hz, 1H), 7.21 (d, J = 1.5 Hz, 1H), 4.93 (dd, J = 13.7, 1.5 Hz, 1H), 4.81 (dd, |
| | J = 13.7, 1.8 Hz, 1H), 4.43-4.51 (m, 1H), 3.54 (dd, J = 9.8, 6.7 Hz, 1H), 3.12- |
| | 3.24 (m, 2H), 2.84 (t, J = 11.4 Hz, 1H), 2.39-2.55 (m, 2H), 2.18-2.27 (m, 1H), |
| | 1.82-1.91 (m, 2H); 3Hs not observed (2 NHs and OH). |
| 60 | MS m/z 366.4 [M + H]⁺; ¹H NMR (500 MHz, methanol-d₄) δ: 9.11 (s, 1H), |
| | 8.00-7.94 (m, 2H), 7.85-7.81 (m, 1H), 7.73 (s, 1H), 7.25-7.16 (m, 2H), |
| | 5.05 (d, J = 13.9 Hz, 2H), 3.69-3.52 (m, 5H), 3.28-3.15 (m, 2H), 1.46 (d, J = |
| | 6.6 Hz, 6H); 1H not observed (NH or OH). |
| 61 | MS m/z 400.2 [M + H]⁺; ¹H NMR (500 MHz, DMSO-d₆) δ: 9.16 (s, 1H), 8.13 |
| | (s, 2H), 7.88 (d, J = 7.9 Hz, 1H), 7.73 (d, J = 7.2 Hz, 2H), 7.57-7.45 (m, |
| | 3H), 7.28-7.21 (m, 2H), 4.92-4.79 (m, 2H), 4.57 (br s, 1H), 3.79-3.61 (m, |
| | 2H), 3.54-3.47 (m, 1H), 3.36-3.24 (m, 1H); 3Hs not observed (2 NHs and OH). |
| 62 | MS m/z 401.3 [M + H]⁺; ¹H NMR (500 MHz, DMSO-d₆) δ: 9.17 (s, 1H), 9.15- |
| | 9.00 (m, 2H), 8.49-8.33 (m, 2H), 8.15 (s, 2H), 7.95-7.77 (m, 1H), 7.26 (s, |
| | 2H), 5.02-4.78 (m, 3H), 3.89-3.70 (m, 2H), 3.66-3.55 (m, 1H), 3.36-3.25 |
| | (m, 1H); 3Hs not observed (2 NHs and OH). |
| 63 | MS m/z 364.4 [M + H]⁺; ¹H NMR (500 MHz, DMSO-d₆) δ: 9.07 (s, 1H), 8.28- |
| | 7.91 (m, 2H), 7.85 (d, J = 8.1 Hz, 1H), 7.26-7.17 (m, 2H), 3.81 (br s, 4H), |
| | 2.66 (br s, 4H), 1.69 (br s, 1H), 0.47 (d, J = 5.8 Hz, 2H), 0.39 (d, J = 2.0 Hz, |
| | 2H); 2Hs not observed (NH and OH). |
| 64 | MS m/z 380.4 [M + H]⁺ |
| 96 | MS m/z 394.5 [M + H]⁺; ¹H NMR (500 MHz, methanol-d₄) δ: 9.11 (s, 1H), |
| | 8.31 (s, 1H), 8.02 (s, 2H), 7.77-7.89 (m, 1H), 7.16-7.28 (m, 1H), 4.95-5.05 |
| | (m, 2H), 3.48-3.56 (m, 1H), 3.35-3.45 (m, 2H), 3.36 (s, 3H), 3.25-3.31 (m, |
| | 2H), 1.10-1.26 (m, 2H), 0.81-0.94 (m, 2H); 3Hs not observed (2 NHs and OH). |
| 123 | MS m/z 370.2 [M + H]⁺; ¹H NMR (500 MHz, DMSO-d₆) δ: 9.09 (s, 1H), 8.23- |
| | 8.20 (m, 1H), 7.88 (d, J = 8.1 Hz, 1H), 7.25-7.22 (m, 1H), 7.20 (d, J = 8.2 |
| | Hz, 1H), 4.79-4.63 (m, 2H), 3.60-3.49 (m, 1H), 3.42-3.34 (m, 1H), 3.34- |
| | 3.27 (m, 1H), 3.27-3.18 (m, 1H), 3.16-3.04 (m, 1H), 1.85-1.64 (m, 2H), |
| | 1.03 (t, J = 7.5 Hz, 3H); 3Hs not observed (2 NHs and OH). |
| 125 | MS m/z 398.5 [M + H]⁺; ¹H NMR (500 MHz, methanol-d₄) δ: 9.06 (s, 1H), |
| | 7.98 (s, 1H), 7.85 (br d, J = 8.4 Hz, 1H), 7.18-7.28 (m, 2H), 4.90 (d, J = 13.2 |
| | Hz, 1H), 4.74 (d, J = 13.3 Hz, 1H), 3.18 (d, J = 12.5 Hz, 1H), 3.07 (t, J = 12.5 |
| | Hz, 1H), 2.85 (t, J = 11.6 Hz, 2H), 2.46 (d, J = 11.6 Hz, 1H), 1.06 (s, 9H); 3Hs |
| | not observed (2 NHs and OH). |
| 127 | MS m/z 380.3 [M + H]⁺; ¹H NMR (500 MHz, methanol-d₄) δ: 8.40 (s, 1H), |
| | 8.36 (s, 1H), 7.85 (s, 2H), 6.99 (s, 1H), 6.88 (s, 1H), 4.90 (d, J = 13.2 Hz, 1H), |
| | 4.81-4.80 (m, 1H), 3.28-3.31 (m, 2H), 3.00-3.07 (m, 2H), 2.81-2.84 (m, |
| | 1H), 2.12, (s, 3H), 1.80-1.84 (m, 1H), 1.02 (dd, J = 5.85, 0.85 Hz, 6H); 3Hs |
| | not observed (2 NHs and OH), 1H from formic acid salt. |
| 133 | MS m/z 397.4 [M + H]⁺; ¹H NMR (500 MHz, methanol-d₄) δ: 9.21 (s, 1H), |
| | 9.00 (s, 1H), 8.09 (d, J = 8.1 Hz, 1H), 7.61-7.70 (m, 2H), 7.51 (s, 1H), 4.99 (d, |
| | J = 13.6 Hz, 1H), 4.91 (d, J = 16.0 Hz, 1H), 3.53-3.61 (m, 2H), 3.47 (br s, 1H), |
| | 3.34-3.38 (m, 2H), 1.78-1.86 (m, 2H), 1.18 (t, J = 7.2 Hz, 3H); 2Hs not |
| | observed (NH and OH). |
| 134 | MS m/z 378.2 [M + H]⁺; ¹H NMR (500 MHz, methanol-d₄) δ: 9.09 (s, 1H), |
| | 8.02 (s, 2H), 7.82 (d, J = 8.2 Hz, 1H), 7.23 (dd, J = 8.2, 1.7 Hz, 1H), 7.19 (s, |
| | 1H), 4.99-4.93 (m, 2H), 3.50-3.44 (m, 1H), 3.19 (td, J = 12.5, 3.6 Hz, |
| | 1H), 2.60 (dd, J = 11.6, 3.2 Hz, 1H), 1.23 (s, 3H), 0.76-0.66 (m, 2H), 0.65- |
| | 0.55 (m, 2H); 5Hs not observed (2 NHs and OH; CH₂ signal overlaps with |
| | MeOH signal). |
| 135 | MS m/z 350.3 [M + H]⁺; ¹H NMR (500 MHz, methanol-d₄) δ: 9.10-9.17 (m, |
| | 1H), 8.33 (s, 1H), 8.02 (s, 2H), 7.80-7.90 (m, 1H), 7.19-7.30 (m, 1H), 4.68- |
| | 4.78 (m, 2H), 4.21-4.30 (m, 2H), 3.42-3.53 (m, 2H), 2.13-2.22 (m, 2H), 2.00- |
| | 2.10 (m, 2H); 3Hs not observed (2 NHs and OH). |
| 136 | MS m/z 364.4 [M + H]⁺; ¹H NMR (500 MHz, DMSO-d₆) δ: 9.78 (d, J = 10.5 |
| | Hz, 1H), 9.44 (d, J = 10.8 Hz, 1H), 9.16 (s, 1H), 8.13 (s, 2H), 7.87 (d, J = 8.0 |
| | Hz, 1H), 7.23-7.29 (m, 2H), 4.59-4.72 (m, 2H), 3.45-3.61 (m, 2H), 3.34-3.54 |
| | (m, 1H), 2.99-3.14 (m, 1H), 2.66-2.71 (m, 1H), 1.06-1.12 (m, 1H), 0.55-0.72 |
| | (m, 3H), 0.38-0.46 (m, 1H); 1H not observed (OH or NHs). |
| 137 | MS m/z 364.4 [M + H]⁺; ¹H NMR (500 MHz, DMSO-d₆) δ: 9.76 (d, J = 10.0 |
| | Hz, 1H), 9.42 (d, J = 11.0 Hz, 1H), 9.18 (s, 1H), 8.17 (s, 2H), 7.88 (d, J = 8.0 |
| | Hz, 1H), 7.26-7.31 (m, 2H), 4.57-4.71 (m, 2H), 3.48-3.63 (m, 2H), 3.34-3.59 |
| | (m, 1H), 2.97-3.13 (m, 1H), 2.64-2.71 (m, 1H), 1.09-1.18 (m, 1H), 0.57-0.73 |
| | (m, 3H), 0.37-0.46 (m, 1H); 1H not observed (OH or NHs). |
| 143 | MS m/z 392.3 [M + H]⁺; ¹H NMR (500 MHz, DMSO-d₆) δ: 9.10 (s, 1H), 9.05 |
| | (s, 1H), 8.54 (s, 1H), 7.99 (d, J = 8.1 Hz, 1H), 7.83 (s, 1H), 7.74 (d, J = 8.2 |
| | Hz, 1H), 4.92-4.71 (m, 2H), 3.57-3.48 (m, 1H), 3.43-3.36 (m, 1H), 3.33- |
| | 3.24 (m, 1H), 3.16-3.09 (m, 2H), 2.59 (s, 3H), 2.12-2.01 (m, 1H), 1.11- |
| | 1.06 (m, 6H); 2Hs not observed (NH and OH). |
| 144 | MS m/z 378.3 [M + H]⁺; ¹H NMR (500 MHz, DMSO-d₆) δ: 9.26 (s, 1H), 9.12 |
| | (s, 1H), 8.77-8.74 (m, 1H), 8.65 (d, J = 2.3 Hz, 1H), 8.01 (d, J = 8.2 Hz, |
| | 1H), 7.84-7.82 (m, 1H), 7.76 (dd, J = 1.5, 8.2 Hz, 1H), 4.91-4.73 (m, 2H), |
| | 3.48-3.37 (m, 2H), 3.28 (dd, J = 11.3, 13.9 Hz, 1H), 3.20-3.09 (m, 2H), |
| | 2.09-2.02 (m, 1H), 1.11-1.04 (m, 6H); 2Hs not observed (NH and OH). |
| 145 | MS m/z 392.3 [M + H]⁺; ¹H NMR (500 MHz, DMSO-d₆) δ: 9.11 (s, 2H), 8.64 |
| | (s, 1H), 7.99 (d, J = 8.1 Hz, 1H), 7.81-7.78 (m, 1H), 7.71 (dd, J = 1.4, 8.2 |
| | Hz, 1H), 4.85 (d, J = 13.4 Hz, 1H), 4.81-4.73 (m, 1H), 3.52 (s, 1H), 3.43- |
| | 3.35 (m, 1H), 3.27 (d, J = 14.0 Hz, 1H), 3.17 (s, 2H), 2.55 (s, 3H), 2.11-2.01 |
| | (m, 1H), 1.10-1.04 (m, 6H); 2Hs not observed (NH and OH). |
| 146 | MS m/z 406.1 [M + H]⁺; ¹H NMR (500 MHz, methanol-d₄) δ: 9.02 (s, 1H), |
| | 8.00 (s, 2H), 7.76 (d, J = 8.1 Hz, 1H), 7.17 (d, J = 1.7 Hz, 1H), 7.15 (s, 1H), |
| | 4.90-4.81 (m, 1H), 4.74-4.64 (m, 1H), 3.48-3.41 (m, 1H), 3.19-3.05 |
| | (m, 2H), 2.71-2.51 (m, 2H); 5Hs not observed (2 NHs and OH; CH₂ signal |
| | overlaps with MeOH signal). |
| 148 | MS m/z 366.3 [M + H]⁺; ¹H NMR (500 MHz, DMSO-d₆) δ: 9.14 (s, 1H), 8.14 |
| | (s, 2H), 7.87 (d, J = 8.2 Hz, 1H), 7.30-7.19 (m, 2H), 4.94-4.78 (m, 2H), |
| | 3.42-3.32 (m, 1H), 3.24-3.18 (m, 2H), 3.17-3.10 (m, 1H), 1.89-1.79 (m, |
| | 1H), 1.77-1.66 (m, 1H), 1.39 (d, J = 6.4 Hz, 3H), 1.09-0.98 (m, 3H); 3Hs |
| | not observed (2 NHs and OH). |
| 149 | MS m/z 392.3 [M + H]⁺; ¹H NMR (500 MHz, DMSO-d₆) δ: 9.14 (s, 1H), 9.13- |
| | 9.11 (m, 1H), 8.05-7.99 (m, 2H), 7.91 (s, 1H), 7.79 (d, J = 8.2 Hz, 1H), |
| | 4.90-4.73 (m, 2H), 3.56-3.47 (m, 1H), 3.43-3.37 (m, 1H), 3.28 (dd, J = |
| | 11.2, 13.8 Hz, 1H), 3.17-3.10 (m, 2H), 2.57 (s, 3H), 2.11-1.99 (m, 1H), |
| | 1.11-1.04 (m, 6H); 2Hs not observed (NH and OH). |
| 150 | MS m/z 406.4 [M + H]⁺; ¹H NMR (500 MHz, DMSO-d₆) δ: 9.15 (s, 1H), 9.10 |
| | (s, 1H), 8.01 (d, J = 8.1 Hz, 1H), 7.99-7.97 (m, 1H), 7.90 (s, 1H), 7.82- |
| | 7.78 (m, 1H), 4.86-4.65 (m, 2H), 3.49-3.40 (m, 1H), 3.18-2.98 (m, 2H), |
| | 2.98-2.87 (m, 1H), 2.83 (q, J = 7.6 Hz, 2H), 1.97-1.84 (m, 1H), 1.30 (t, J = |
| | 7.6 Hz, 3H), 1.08-1.01 (m, 7H); 2Hs not observed (NH and OH). |
| 151 | MS m/z 378.2 [M + H]⁺; ¹H NMR (500 MHz, DMSO-d₆) δ: 9.07 (s, 1H), 8.93 |
| | (d, J = 4.9 Hz, 2H), 8.08 (s, 1H), 8.02-7.97 (m, 2H), 7.48 (t, J = 4.8 Hz, 1H), |
| | 4.82-4.62 (m, 2H), 3.48-3.41 (m, 1H), 3.28-3.18 (m, 2H), 3.07-2.88 (m, |
| | 2H), 1.89-1.78 (m, 1H), 1.02 (dd, J = 3.2, 6.7 Hz, 6H); 2Hs not observed |
| | (NH and OH). |
| 152 | MS m/z 401.4 [M + H]⁺; ¹H NMR (500 MHz, methanol-d₄) δ: 9.09 (s, 1H), |
| | 8.52 (br. s, 1H), 8.07 (d, J = 1.45 Hz, 1H), 7.91 (s, 1H), 7.67 (d, J = 12.4 Hz, |
| | 1H), 7.21 (d, J = 6.55 Hz, 1H), 4.96 (d, J = 13.2 Hz, 1H), 4.81-4.83 (m, 1H), |
| | 3.28-3.31 (m, 2H), 3.05-3.11 (m, 2H), 2.84-2.88 (m, 1H), 1.89-1.93 (m, 1H), |
| | 1.12 (d, J = 6.7 Hz, 6H); 2Hs not observed (NH and OH), 1H from formic acid salt. |
| 156 | MS m/z 415.4 [M + H]⁺; ¹H NMR (500 MHz, methanol-d₄) δ: 8.92 (s, 1H), |
| | 8.43 (s, 1H), 7.94 (d, J = 1.45 Hz, 1H), 7.79 (s, 1H), 7.54 (d, J = 12.4 Hz, 1H), |
| | 7.07 (d, J = 6.55 Hz, 1H), 4.83 (d, J = 13.2 Hz, 1H), 4.68 (d, J = 13.5 Hz, 1H), |
| | 3.13-3.16 (m, 1H), 3.05-3.11 (m, 1H), 2.79-2.87 (m, 2H), 2.50-2.52 (m, 1H), |
| | 0.96 (s, 9H); 2Hs not observed (NH and OH), 1H from formic acid salt. |
| 158 | MS m/z 384.3, [M + H]⁺; ¹H NMR (500 MHz, methanol-d₄) δ: 8.97 (s, 1H), |
| | 8.40 (s, 1H), 7.95 (br. s, 2H), 7.58 (d, J = 12.1 Hz, 1H), 7.14 (d, J = 6.55 Hz, |
| | 1H), 4.85 (d, J = 13.2 Hz, 1H), 4.69-4.72 (m, 1H), 3.26-3.30 (m, 1H), 3.15- |
| | 3.18 (m, 1H), 2.92-2.99 (m, 2H), 2.70-2.73 (m, 1H), 1.74-1.79 (m, 1H), 1.00 |
| | (d, J = 6.8 Hz, 6H); 3Hs not observed (2 NHs and OH), 1H from formic acid salt. |
| 160 | MS m/z 391.4 [M + H]⁺; ¹H NMR (500 MHz, methanol-d₄) δ: 9.14 (s, 1H), |
| | 8.74-8.68 (m, 1H), 8.04 (dd, J = 2.3, 8.1 Hz, 1H), 7.95 (d, J = 7.9 Hz, 1H), |
| | 7.43 (d, J = 8.1 Hz, 1H), 7.28-7.25 (m, 2H), 5.07 (br d, J = 13.1 Hz, 1H), |
| | 4.97 (br d, J = 14.3 Hz, 1H), 3.60-3.51 (m, 2H), 3.31-3.22 (m, 2H), 3.13 |
| | (ddd, J = 3.2, 7.4, 10.9 Hz, 1H), 2.62 (s, 3H), 2.16-2.05 (m, 1H), 1.19 (dd, J = |
| | 4.3, 6.8 Hz, 6H); 2Hs not observed (NH and OH). |
| 161 | MS m/z 377.4 [M + H]⁺; ¹H NMR (500 MHz, methanol-d₄) δ: 9.04 (s, 1H), |
| | 8.60 (d, J = 5.6 Hz, 2H), 7.93 (d, J = 8.1 Hz, 1H), 7.73 (d, J = 6.0 Hz, 2H), |
| | 7.36-7.32 (m, 2H), 4.80 (br d, J = 12.8 Hz, 1H), 4.67 (br d, J = 13.7 Hz, |
| | 1H), 3.17-3.06 (m, 2H), 2.88-2.76 (m, 2H), 2.44 (ddd, J = 2.8, 7.2, 10.3 |
| | Hz, 1H), 1.72 (qd, J = 6.8, 13.6 Hz, 1H), 1.06 (d, J = 6.7 Hz, 6H); 2Hs not |
| | observed (NH and OH). |
| 163 | MS m/z 398.1 [M + H]⁺; ¹H NMR (500 MHz, methanol-d₄) δ: 8.79 (s, 1H), |
| | 7.76 (s, 1H), 7.59 (d, J = 8.2 Hz, 1H), 6.92-7.07 (m, 2H), 4.53 (br d, J = 13.1 |
| | Hz, 1H), 4.36 (d, J = 14.6 Hz, 1H), 2.84-2.94 (m, 2H), 2.73 (dd, J = 13.1, 10.7 |
| | Hz, 1H), 2.50-2.60 (m, 1H), 1.65-1.70 (m, 1H), 0.53-0.67 (m, 1H), 0.28-0.37 |
| | (m, 2H), 0.05-0.16 (m, 2H); 3Hs not observed (2 NHs and OH). |
| 164 | MS m/z 398.3 [M + H]⁺; ¹H NMR (500 MHz, methanol-d₄) δ: 8.96 (s, 1H), 8.41 |
| | (s, 1H), 7.95 (br. s, 2H), 7.57 (d, J = 12.1 Hz, 1H), 7.13 (d, J = 6.55 Hz, 1H), 4.89 |
| | (d,J = 13.2Hz, 1H), 4.69-4.72 (m, 1H), 3.13-3.16 (m, 1H), 3.05-3.11 (m, 1H), |
| | 2.86-3.00 (m, 2H), 2.62-2.65 (m, 1H), 0.99 (s, 9H); 3Hs not observed (2 NHs |
| | and OH), 1H from formic acid salt. |
| 165 | MS m/z 338.0 [M + H]⁺; ¹H NMR (500 MHz, methanol-d₄) δ: 9.16 (s, 1H), 8.10 |
| | (s, 2H), 7.87 (d, J = 8.2 Hz, 1H), 7.27 (dd, J = 8.2, 1.8 Hz, 1H), 7.24 (d, J = 1.8 |
| | Hz, 1H), 4.89-4.95 (m, 2H), 4.17-4.28 (m, 2H), 3.40-3.66 (m, 3H), 1.46 (d, |
| | J = 6.0 Hz, 3H); 3 Hs not observed (2 NHs and OH). |
| 166 | MS m/z 338.0 [M + H]⁺; ¹H NMR (500 MHz, methanol-d₄) δ: 9.18 (s, 1H), |
| | 8.14-8.11 (m, 2H), 7.89 (d, J = 8.1 Hz, 1H), 7.29 (br d, J = 7.3 Hz, 1H), 7.25 (br |
| | d, J = 6.7 Hz, 1H), 4.91-4.98 (m, 2H), 4.09-4.36 (m, 2H), 3.42-3.73 (m, 3H), |
| | 1.46 (br d, J = 5.8 Hz, 3H); 3 Hs not observed (2 NHs and OH). |
| 170 | MS m/z 382.0 [M + H]⁺; ¹H NMR (500 MHz, methanol-d₄) δ: 9.14 (s, 1H), |
| | 8.81 (s, 1H), 8.04 (d, J = 8.1 Hz, 1H), 7.62-7.70 (m, 2H), 4.93 (d, J = 13.7 |
| | Hz, 1H), 4.75-4.87 (m, 1H), 3.37 (t, J = 12.3 Hz, 2H), 3.26 (dd, J = 13.8, 10.6 |
| | Hz, 1H), 3.00-3.09 (m, 1H), 2.35 (d, J = 10.8 Hz, 1H), 0.89-1.04 (m, 1H), |
| | 0.68-0.78 (m, 2H), 0.45-0.51 (m, 2H); 2Hs not observed (OH and NH). |
| 177 | MS m/z 375.3 [M + H]⁺; ¹H NMR (500 MHz, methanol-d₄) δ: 9.21 (s, 1H), |
| | 8.92-8.86 (m, 2H), 8.43 (d, J = 6.3 Hz, 2H), 8.17-8.11 (m, 1H), 7.67-7.53 |
| | (m, 2H), 5.06-4.97 (m, 1H), 4.96-4.89 (m, 1H), 3.64-3.49 (m, 3H), 3.29- |
| | 3.21 (m, 1H), 2.76-2.66 (m, 1H), 1.16-1.06 (m, 1H), 0.89-0.76 (m, 2H), |
| | 0.66-0.51 (m, 2H); 2Hs not observed (NH and OH). |
| 178 | MS m/z 393.3 [M + H]⁺; ¹H NMR (500 MHz, DMSO-d₆) δ: 9.09 (s, 1H), 8.79 |
| | (d, J = 2.3 Hz, 1H), 8.59 (d, J = 5.0 Hz, 1H), 8.00 (d, J = 8.1 Hz, 1H), 7.80- |
| | 7.71 (m, 1H), 7.38 (s, 1H), 7.30 (d, J = 8.1 Hz, 1H), 4.80-4.67 (m, 2H), 3.64- |
| | 3.49 (m, 2H), 3.46-3.36 (m, 1H), 3.07 (br d, J = 11.7 Hz, 1H), 2.72-2.61 |
| | (m, 1H), 1.17-1.10 (m, 1H), 0.71-0.59 (m, 3H), 0.46-0.40 (m, 1H); 2Hs |
| | not observed (NH and OH). |
| 179 | MS m/z 403.2 [M + H]⁺; ¹H NMR (500 MHz, DMSO-d₆) δ: 9.11 (s, 1H), 7.92 |
| | (d, J = 8.1 Hz, 1H), 7.70 (br d, J = 8.2 Hz, 2H), 7.34-7.21 (m, 4H), 4.79- |
| | 4.65 (m, 2H), 3.60-3.47 (m, 3H), 3.46-3.40 (m, 1H), 3.07 (br d, J = 9.8 Hz, |
| | 1H), 2.87 (s, 3H), 2.69-2.62 (m, 1H), 0.71-0.59 (m, 3H), 0.47-0.39 (m, |
| | 1H); 2Hs not observed (NH and OH). |
| 185 | MS m/z 383.5 [M + H]⁺; ¹H NMR (500 MHz, methanol-d₄) δ: 9.10 (s, 1H), |
| | 7.99-7.93 (m, 1H), 7.92 (d, J = 3.3 Hz, 1H), 7.66 (d, J = 3.2 Hz, 1H), 7.58 |
| | (d, J = 7.3 Hz, 2H), 4.88-4.91 (m, 1H), 4.75 (d, J = 13.4 Hz, 1H), 3.13-3.24 |
| | (m, 2H), 2.91 (dd, J = 13.5, 10.3 Hz, 2H), 2.56 (t, J = 8.0 Hz, 1H), 1.72-1.83 |
| | (m, 1H), 1.09 (d, J = 6.8 Hz, 6H); 2Hs not observed (OH and NH). |
| 186 | MS m/z 433.5 [M + H]⁺; ¹H NMR (500 MHz, methanol-d₄) δ: 9.09 (s, 1H), |
| | 7.94-7.99 (m, 2H), 7.57-7.63 (m, 2H), 6.77-7.03 (m, 1H), 4.86-4.93 (m, 1H), |
| | 4.74 (d, J = 13.3 Hz, 1H), 3.12-3.23 (m, 2H), 2.87-2.92 (m, 2H), 2.52-2.56 |
| | (m, 1H), 1.77 (h, J = 6.9 Hz, 1H), 1.09 (d, J = 6.8 Hz, 6H); 2Hs not observed |
| | (OH and NH). |
| 187 | MS m/z 394.1 [M + H]⁺; ¹H NMR (500 MHz, DMSO-d₆) δ: 1H NMR (DMSO- |
| | d₆) δ: 12.93 (br s, 1H), 11.31 (br s, 1H), 9.09 (s, 1H), 7.96-8.19 (m, 2H), |
| | 7.82-7.89 (m, 1H), 7.17-7.27 (m, 2H), 4.68 (dd, J = 8.2, 5.8 Hz, 1H), 4.51-4.64 |
| | (m, 3H), 4.36 (d, J = 12.8 Hz, 1H), 4.26 (d, J = 12.5 Hz, 1H), 3.19-3.35 (m, |
| | 2H), 2.88 (dd, J = 13.0, 9.3 Hz, 1H), 2.81 (dt, J = 11.5, 3.1 Hz, 1H), 2.54 (dd, |
| | J = 9.5, 2.7 Hz, 1H), 2.29 (td, J = 11.5, 4.0 Hz, 1H), 2.11 (s, 3H). |
| 188 | MS m/z 417.3, 419.3 [M + H]⁺; ¹H NMR (500 MHz, DMSO-d₆) δ: 11.15 (s, |
| | 1H), 9.00 (s, 1H), 7.97 (d, J = 8.1 Hz, 1H), 7.84 (s, 1H), 7.56 (d, J = 1.8 Hz, |
| | 1H), 7.51 (dd, J = 8.1, 1.8 Hz, 1H), 4.65 (d, J = 12.6 Hz, 1H), 4.56 (d, J = |
| | 12.6 Hz, 1H), 2.97-3.09 (m, 2H), 2.62-2.83 (m, 2H), 2.29-2.42 (m, 1H), 1.66 |
| | (h, J = 6.7 Hz, 1H), 0.97 (d, J = 6.8 Hz, 6H); 1H not observed (OH or NH). |
| 189 | MS m/z 417.3, 419.3 [M + H]⁺; ¹H NMR (500 MHz, DMSO-d₆) δ: 11.22 (s, |
| | 1H), 9.00 (s, 1H), 7.94-8.02 (m, 2H), 7.52 (d, J = 1.8 Hz, 1H), 7.46 (dd, J = |
| | 8.1, 1.8 Hz, 1H), 4.64 (d, J = 12.5 Hz, 1H), 4.55 (d, J = 12.5 Hz, 1H), 2.95- |
| | 3.08 (m, 2H), 2.61-2.81 (m, 2H), 2.33-2.36 (m, 1H), 1.65 (h, J = 6.8 Hz, 1H), |
| | 0.97 (d, J = 6.8 Hz, 6H)); 1H not observed (OH or NH). |
| 192 | MS m/z 376.2 [M + H]⁺; ¹H NMR (500 MHz, methanol-d₄) δ: 9.22-9.19 (m, |
| | 1H), 9.08 (s, 1H), 8.80 (d, J = 5.5 Hz, 1H), 7.99-7.96 (m, 2H), 7.81-7.77 |
| | (m, 1H), 7.75 (dd, J = 1.7, 8.2 Hz, 1H), 4.82-4.79 (m, 1H), 4.79-4.64 (m, |
| | 1H), 3.22-3.12 (m, 2H), 3.02 (dd, J = 10.6, 13.2 Hz, 1H), 2.84 (dt, J = 3.4, |
| | 12.2 Hz, 1H), 2.01-1.93 (m, 1H), 0.94-0.80 (m, 1H), 0.61 (td, J = 4.0, 7.6 |
| | Hz, 2H), 0.41-0.31 (m, 2H); 2Hs not observed (NH and OH). |
| 196 | MS m/z 401.4 [M + H]⁺; ¹H NMR (500 MHz, DMSO-d₆) δ: 11.19 (s, 1H), |
| | 9.00 (s, 1H), 7.95 (d, J = 8.1 Hz, 1H), 7.79 (d, J = 3.1 Hz, 1H), 7.49 (d, J = |
| | 1.8 Hz, 1H), 7.42 (dd, J = 8.1, 1.8 Hz, 1H), 4.64 (d, J = 12.6 Hz, 1H), 4.55 (d, |
| | J = 12.6 Hz, 1H), 2.96-3.08 (m, 2H), 2.65-2.79 (m, 2H), 2.32-2.37 (m, 1H), |
| | 1.65 (h, J = 6.7 Hz, 1H), 0.97 (d, J = 6.8 Hz, 6H); 1H not observed (OH or NH). |
| 199 | MS m/z 408.5 [M + H]⁺; ¹H NMR (500 MHz, DMSO-d₆) δ: 11.20 (s, 1H), |
| | 9.00 (s, 1H), 8.02 (d, J = 8.1 Hz, 1H), 7.57 (d, J = 1.6 Hz, 1H), 7.54 (dd, J = |
| | 8.1, 1.7 Hz, 1H), 4.67 (d, J = 12.5 Hz, 1H), 4.57 (d, J = 12.7 Hz, 1H), 3.00- |
| | 3.10 (m, 2H), 2.69-2.83 (m, 2H), 2.29-2.37 (m, 1H), 1.58-1.73 (m, 1H), 1.07- |
| | 1.31 (m, 5H), 0.98 (d, J = 6.9, 6H); 1H not observed (OH or NH). |
| 200 | MS m/z 413.4 [M + H]⁺; ¹H NMR (500 MHz, DMSO-d₆) δ: 11.11 (s, 1H), |
| | 9.00 (s, 1H), 7.94 (d, J = 8.1 Hz, 1H), 7.52 (d, J = 1.8 Hz, 1H), 7.46 (dd, J = |
| | 8.2, 1.8 Hz, 1H), 6.67 (s, 1H), 4.65 (d, J = 12.7 Hz, 1H), 4.56 (d, J = 12.6 Hz, |
| | 1H), 3.89 (s, 3H), 2.98-3.09 (m, 2H), 2.62-2.83 (m, 2H), 2.37 (s, 1H), 1.66 |
| | (h, J = 6.8 Hz, 1H), 0.98 (d, J = 6.9, 6H); 1H not observed (OH or NH). |
| 203 | MS m/z 378.4 [M + H]⁺; ¹H NMR (500 MHz, methanol-d₄) δ: 9.02 (s, 1H), |
| | 8.09-7.88 (m, 2H), 7.79 (d, J = 8.2 Hz, 1H), 7.23-7.15 (m, 2H), 4.75 (d, J = |
| | 13.4 Hz, 1H), 4.64 (d, J = 13.1 Hz, 1H), 3.23 (dt, J = 3.1, 12.9 Hz, 1H), 2.97- |
| | 2.90 (m, 2H), 2.82 (dd, J = 10.8, 13.3 Hz, 1H), 2.35 (dt, J = 3.2, 12.1 Hz, |
| | 1H), 2.22-2.06 (m, 2H), 1.86 (br d, J = 12.7 Hz, 1H), 1.79-1.64 (m, 3H), |
| | 1.47-1.30 (m, 2H); 2Hs not observed (NH and OH). |
| 204 | MS m/z 414.4 [M + H]⁺; ¹H NMR (500 MHz, DMSO-d₆) δ: 11.15 (s, 1H), |
| | 8.95-9.12 (m, 1H), 7.97 (d, J = 8.2 Hz, 1H), 7.24-7.63 (m, 3H), 4.73 (d, J = |
| | 13.1 Hz, 1H), 4.64 (d, J = 13.0 Hz, 1H), 4.20 (s, 3H), 3.17 (s, 3H), 2.81-3.02 |
| | (m, 2H), 1.76 (s, 1H), 1.00 (d, J = 6.8 Hz, 6H). |
| 205 | MS m/z 434.4 [M + H]⁺; ¹H NMR (500 MHz, DMSO-d₆) δ: 11.33 (s, 1H), |
| | 9.03 (s, 1H), 8.04 (d, J = 8.1 Hz, 1H), 7.51-7.81 (m, 3H), 4.66 (d, J = 12.6 |
| | Hz, 1H), 4.56 (d, J = 12.7 Hz, 1H), 2.97-3.09 (m, 2H), 2.77 (dd, J = 12.6, |
| | 10.5 Hz, 1H), 2.62-2.71 (m, 1H), 2.28-2.41 (m, 1H), 1.65 (h, J = 6.7 Hz, 1H), |
| | 0.97 (d, J = 6.8, 6H); 1H not observed (OH or NH). |
| 207 | MS m/z 438.5 [M + H]⁺; ¹H NMR (500 MHz, DMSO-d₆) δ: 11.21 (s, 1H), |
| | 9.05 (s, 1H), 7.97 (d, J = 8.2 Hz, 1H), 7.81 (d, J = 1.8 Hz, 1H), 7.73 (dd, J = |
| | 8.3, 1.8 Hz, 1H), 7.11 (s, 1H), 4.65 (d, J = 12.6 Hz, 1H), 4.55 (d, J = 12.6 Hz, |
| | 1H), 4.01 (s, 3H), 3.96 (s, 3H), 2.90-3.10 (m, 2H), 2.76 (dd, J = 12.7, 10.4 |
| | Hz, 1H), 2.61-2.72 (m, 1H), 2.33-2.40 (m, 1H), 1.65 (h, J = 6.7 Hz, 1H), 0.97 |
| | (d, J = 6.8 Hz, 6H); 1H not observed (OH or NH). |
| 208 | MS m/z 392.3 [M + H] |
| 209 | MS m/z 393.2 [M + H]⁺; ¹H NMR (500 MHz, methanol-d₄) δ: 9.13 (s, 1H), |
| | 8.28 (d, J = 5.3 Hz, 1H), 7.99 (d, J = 8.1 Hz, 1H), 7.63 (d, J = 5.3 Hz, 1H), |
| | 7.43-7.34 (m, 3H), 5.00-4.78 (m, 1H), 3.43 (d, J = 6.7 Hz, 2H), 3.37-3.29 |
| | (m, 2H), 3.11 (dt, J = 3.5, 12.9 Hz, 1H), 2.45 (dt, J = 3.1, 10.0 Hz, 1H), 1.02 |
| | (tdd, J = 4.5, 8.5, 12.9 Hz, 1H), 0.81-0.71 (m, 2H), 0.58-0.46 (m, 2H); 2Hs |
| | not observed (NH and OH). |
| 210 | MS m/z 431.4 [M + H]⁺; ¹H NMR (500 MHz, methanol-d₄) δ: 9.04 (s, 1H), |
| | 8.46 (s, 1H), 7.89 (d, J = 8.2 Hz, 1H), 7.57-7.51 (m, 2H), 6.68 (s, 1H), 4.79 |
| | (d, J = 12.1 Hz, 1H), 4.68 (d, J = 13.1 Hz, 1H), 4.20 (t, J = 7.6 Hz, 4H), 3.20- |
| | 3.10 (m, 2H), 2.98 (dd, J = 10.9, 12.7 Hz, 1H), 2.85-2.77 (m, 1H), 2.50 |
| | (quin, J = 7.6 Hz, 2H), 1.97-1.90 (m, 1H), 0.86 (dt, J = 4.1, 8.5 Hz, 1H), |
| | 0.64-0.57 (m, 2H), 0.41-0.31 (m, 2H); 2Hs not observed (NH and OH). |
| 215 | MS m/z 396.0 [M + H]⁺; ¹H NMR (400 MHz, methanol-d₄) δ 9.03 (s, 1H), |
| | 7.98 (s, 1H), 7.82 (d, J = 8.8 Hz, 1H), 7.20 (d,J = 6.8 Hz, 2H), 4.83 (d, J = 13.2 |
| | Hz, 1H), 4.70 (d, J = 12.8 Hz, 1H), 2.84 (t, J = 12.0 Hz, 2H), 2.66 (t, J = 12.0, |
| | 1H), 1.94 (t, J = 9.2 Hz, 1H), 1.21 (d, J = 6.4 Hz, 3H), 0.85 (q, J = 3.2 Hz, 1H), |
| | 0.60 (d, J = 8.0 Hz, 2H), 0.36 (q, J = 14.8 Hz, 2H); 3Hs not observed (2 NHs |
| | and OH). |
| 216 | MS m/z: 380.0 [M + H]⁺; ¹H NMR (400 MHz, methanol-d₄) δ: 9.03 (s, 1H), |
| | 8.01(s, 2H), 7.80(d, J = 8.4 Hz, 1H), 7.22-7.18(m, 2H), 4.89-4.84 (m, 1H), |
| | 4.71-4.68 (m, 1H), 2.93-2.81 (m, 1H), 2.69-2.59 (m, 2H), 2.49-2.46 (m, 1H), |
| | 1.73-1.71 (m, 1H), 1.21 (d, J = 6.4 Hz, 3H), 1.07 (d, J = 6.2 Hz, 6H); 3Hs not |
| | observed (2 NHs and OH). |
| 217 | MS m/z: 398.0 [M + H]⁺; ¹H NMR (400 MHz, methanol-d₄) δ 9.03 (s, 1H), |
| | 7.95 (d, J = 2.0 Hz, 1H), 7.80 (d, J = 8.4 Hz, 1H), 7.18 (d, J = 6.8 Hz, 2H), 4.76 |
| | (t, J = 14.4 Hz, 1H), 4.60 (s, 1H), 2.97 (s, 1H), 2.69 (m, 3H), 1.76 (q, J = 6.8 |
| | Hz, 1H), 1.24 (d, J = 6.4 Hz, 3H), 1.08 (t, J = 6.4 Hz, 6H); 3Hs not observed |
| | (2 NHs and OH). |
| 218 | MS m/z: 384.0 [M + H]⁺; ¹H NMR (400 MHz, methanol-d₄) δ 9.03 (s, 1H), |
| | 7.97 (d, J = 1.6 Hz, 1H), 7.82 (d, J = 8.8 Hz, 1H), 7.20 (d,J = 6.4 Hz, 2H), 4.83 |
| | (d, J = 12.8 Hz, 1H), 4.73 (d, J = 12.8 Hz, 1H), 2.91 (m, 1H), 2.63 (q, J = 11.2 |
| | Hz, 3H), 1.55 (m, 2H), 1.20 (d, J = 6.0 Hz, 3H), 1.07 (t, J = 7.6 Hz, 3H); 3Hs |
| | not observed (2 NHs and OH). |
| 219 | MS m/z: 450.1 [M + H]⁺; ¹H NMR (400 MHz, methanol-d₄) δ: 9.08 (s, 1H), |
| | 7.93 (d, J = 7.6 Hz, 1H), 7.71 (s, 1H), 7.66 (d, J = 9.2 Hz, 1H), 6.96 (s, 1H), |
| | 4.75 (d, J = 12.8 Hz, 1H), 4.10 (s, 3H), 4.01 (s, 3H), 2.90-2.84 (m, 2H), 2.68 |
| | (t, J = 11.8 Hz, 1H), 1.98 (t, J = 11.2 Hz, 1H), 1.31 (s, 1H), 1.22 (d, J = 5.2 |
| | Hz, 3H), 0,91-0.86 (m, 1H), 0.61 (d, J = 7.6 Hz, 2H), 0.36 (d, J = 14.8 Hz, |
| | 2H); 2Hs not observed (NH and OH). |
| 220 | MS m/z: 450.1 [M + H]⁺; ¹H NMR (400 MHz, methanol-d₄) δ: 9.08 (s, 1H), |
| | 7.93 (d, J = 7.6 Hz, 1H), 7.71 (s, 1H), 7.66 (d, J = 9.2 Hz, 1H), 6.96 (s, 1H), |
| | 4.75 (d, J = 12.4 Hz, 1H), 4.10 (s, 3H), 4.01 (s, 3H), 2.90-2.84 (m, 2H), 2.68 |
| | (t, J = 12.0 Hz, 1H), 1.98 (t, J = 11.2 Hz, 1H), 1.31 (s, 1H), 1.22 (d, J = 6.4 |
| | Hz, 3H), 0,90-0.83 (m, 1H), 0.61 (d, J = 8.0 Hz, 2H), 0.42-0.33 (m, 2H); 3Hs |
| | not observed (2 NHs and OH). |
| 221 | MS m/z: 396.0 [M + H]⁺; ¹H NMR (400 MHz, methanol-d₄) δ: 7.45 (s, 1H), |
| | 6.40 (d, J = 2.0 Hz, 1H), 6.23 (t, J = 6.8 Hz, 1H), 5.61 (d, J = 6.4 Hz, 2H), 4.82 |
| | (t, J = 1.2 Hz, 1H), 4.67 (d, J = 12.8 Hz, 1H), 2.82 (m, 2H), 2.63 (q, J = 2.4 Hz, |
| | 1H), 1.94 (m, 1H), 1.19 (d, J = 6.4 Hz, 3H), 0.82 (m, 1H), 0.58 (m, 2H), 0.35 |
| | (m, 2H); 3Hs not observed (2 NHs and OH). |
| 222 | MS m/z: 398.0 [M + H]⁺; ¹H NMR (400 MHz, methanol-d₄) δ: 9.03 (s, 1H), |
| | 7.97 (d, J = 2.0 Hz, 1H), 7.82 (d, J = 8.8 Hz, 1H), 7.20 (d,J = 6.8 Hz, 2H), 4.86 |
| | (s, 1H), 4.69 (t, J = 12.0 Hz, 1H), 2.88 (m, 1H), 2.66 (m, 2H), 2.50 (m, 1H), |
| | 1.72 (m, 1H), 1.21 (d, J = 6.4 Hz, 3H), 1.08 (q, J = 0.8 Hz, 6H); 3Hs not |
| | observed (2 NHs and OH). |
| 223 | MS m/z: 384. [M + H]⁺; ¹H NMR (400 MHz, methanol-d₄) δ 9.05 (s, 1H), |
| | 7.98 (s, 1H), 7.83 (d, J = 8.8 Hz, 1H), 7.21 (d,J = 6.8 Hz, 2H), 4.84 (d, J = 13.6 |
| | Hz, 1H), 4.74 (d, J = 13.2 Hz, 1H), 2.91 (s, 1H), 2.63 (q, J = 12.4 Hz, 3H), 1.56 |
| | (s, 2H), 1.21 (d, J = 6.0 Hz, 3H), 1.07 (t, J = 7.2 Hz, 3H); 3Hs not observed (2 |
| | NHs and OH). |
| 224 | MS m/z: 378.1 [M + H]⁺; ¹H NMR (400 MHz, methanol-d₄) δ: 9.04 (s, 1 H), |
| | 8.10-7.89 (m, 2H), 7.80 (d, J = 8.0 Hz, 1 H), 7.18(dd, J = 1.6 Hz, 1H), 7.18(d, |
| | J = 1.6 Hz, 1H), 4.87-4.82 (m, 1H), 4.72-4.66 (m, 1H), 2.86-2.84 (m, 2H), |
| | 2.71-2.58 (m, 1H), 2.02-1.92 (m, 1H), 1.21(d, J = 6.0 Hz, 3H), 0.91-0.80(m, |
| | 1H), 0.60-0.59 (m, 2H), 0.41-0.27 (m, 2H); 3Hs not observed (2 NHs and OH). |
| 225 | MS m/z: 378. [M + H]⁺; ¹H NMR (400 MHz, methanol-d₄) δ: 9.04 (s, 1H), |
| | 8.09-7.91(m, 2H), 7.08(d, J = 8.0 Hz, 1H), 7.21(dd, J = 1.8 Hz, 1H), 7.18(d, J = |
| | 1.6 Hz, 1H), 4.85-4.82 (m, 1H), 4.72-4.68 (m, 1H), 2.84-2.81(m, 2H), 2.72- |
| | 2.62(m, 1H), 1.99-1.92(m, 1H), 1.21(d, J = 6.4 Hz, 3H), 0.90-0.78(m, 1H), |
| | 0.60-0.58 (m, 2H), 0.38-0.31 (m, 2H); 3Hs not observed (2 NHs and OH). |
| 226 | MS m/z: 420.1 [M + H]⁺; ¹H NMR (400 MHz, DMSO-d₆) δ: 11.17 (s, 1H), |
| | 9.04 (s, 1H), 8.87 (s, 1H), 7.96 (d, J = 8.4 Hz, 1H), 7.82 (d, J = 1.6 Hz, 1H), |
| | 7.75 (q, J = 8.0 Hz, 1H), 7.47 (s, 1H), 4.65 (q, J = 11.2 Hz, 2H), 3.98 (s, 3H), |
| | 2.54-2.76 (m, 4H), 1.93 (d, J = 19.6 Hz, 1H), 1.08 (d, J = 6.4 Hz, 3H), 0.76- |
| | 0.79 (m, 1H), 0.44 (d, J = 4.8 Hz, 2H), 0.24-0.31 (m, 2H). |
| 227 | MS m/z: 422.1 [M + H]⁺; ¹H NMR (400 MHz, DMSO-d₆) δ: 11.21 (s, 1H), |
| | 9.05 (s, 1H), 8.89 (d, J = 0.8 Hz, 1H), 7.98 (d, J = 8.0 Hz, 1H), 7.84 (d, J = 1.6 |
| | Hz, 1H), 7.76 (q, J = 8.0 Hz, 1H), 7.49 (d, J = 1.2 Hz, 1H), 4.67 (q, J = 36.0 |
| | Hz, 2H), 4.01 (d, J = 7.6 Hz, 3H), 2.75-2.77 (m, 1H), 2.57-2.66 (m, 2H), 2.42- |
| | 2.46 (m, 2H), 1.64-1.69 (m, 1H), 1.10 (d, J = 6.4 Hz, 3H), 1.00 (d, J = 3.6 Hz, |
| | 3H), 0.98 (d, J = 3.6 Hz, 3H). |
| 228 | MS m/z: 408.2 [M + H]⁺; ¹H NMR (400 MHz, methanol-d₄) δ: 8.97 (s, 1H), |
| | 8.75 (s, 1H), 7.85 (d, J = 8.4 Hz, 1H), 7.57 (t, J = 6.0 Hz, 2H), 7.17 (s, 1H), |
| | 4.75 (q, J = 48.0 Hz, 2H), 4.02 (s, 3H), 2.88-2.93 (m, 1H), 2.57-2.71 (m, 3H), |
| | 1.48-1.55 (m, 2H), 1.18 (d, J = 6.4 Hz, 3H), 1.03 (d, J = 7.6 Hz, 3H); 2Hs not |
| | observed (NH and OH). |
| 229 | MS m/z: 380.0 [M + H]⁺; ¹H NMR (500 MHz, methanol-d₄) δ: 9.06 (s, 1 H), |
| | 8.13-7.92(m, 2H), 7.83(d, J = 8.4 Hz, 1H), 7.23(dd, J = 1.0 Hz, 1H), 7.20 (s, |
| | 1H), 4.87-4.86 (m, 1H), 4.71-4.70 (m, 1H), 2.93-2.82 (m, 1H), 2.69-2.64 (m, |
| | 2H), 2.53-2.48 (m, 1H), 1.77-1.70 (m, 1H), 1.22(d, J = 3.0 Hz, 3H), 1.09- |
| | 1.07(m, 6H); 3Hs not observed (2 NHs and OH). |
| 230 | MS m/z: 422.0 [M + H]⁺; ¹H NMR (400 MHz, methanol-d₄) δ: 9.02 (s, 1H), |
| | 8.80 (s, 1H), 7.90 (d, J = 8.4 HZ, 1H), 7.62 (q, J = 14.8 Hz, 2H), 7.22 (s, 1H), |
| | 4.92 (q, J = 12.4 Hz, 1H), 4.76 (d, J = 11.6 Hz, 1H), 4.07 (s, 3H), 2.94 (s, 1H), |
| | 2.65-2.76 (m, 2H), 2.55-2.56 (m, 1H), 1.73-1.75 (m, 1H), 1.24 (d, J = 6.0 Hz, |
| | 3H), 1.08 (d, J = 6.4 Hz, 6H); 2Hs not observed (NH and OH). |
| 231 | MS m/z 392.3 [M + H]⁺; ¹H NMR (500 MHz, DMSO-d₆) δ: 11.31 (s, 1H), |
| | 9.08 (s, 1H), 8.22 (s, 1H), 7.93 (s, 1H), 7.84 (d, J = 8.0 Hz, 1H), 7.22 (d, J = |
| | 8.0 Hz, 1H), 7.21 (s, 1H), 4.69-4.52 (m, 2H), 4.12-4.08 (m, 1H), 3.17 (d, J = |
| | 5.0 Hz, 3H), 2.08-1.78 (m, 6H), 1.28-1.00 (m, 4H); 2Hs not observed (2 NHs). |
| 232 | MS m/z 379.5 [M + H]⁺; ¹H NMR (500 MHz, DMSO-d₆) δ 11.01 (s, 1H), 9.14 |
| | (s, 1H), 9.07 (s, 1H), 7.96 (d, J = 8.1 Hz, 1H), 7.58 (d, J = 1.7 Hz, 1H), 7.53 |
| | (dd, J = 8.1, 1.6 Hz, 1H), 7.03 (s, 1H), 4.81-4.65 (m, 2H), 3.45 (ddt, J = |
| | 17.6, 14.2, 6.9 Hz, 3H), 3.15-3.07 (m, 1H), 2.73-2.64 (m, 1H), 2.41 (s, |
| | 3H), 1.04 (tp, J = 8.7, 4.7 Hz, 1H), 0.67 (ttd, J = 13.4, 8.7, 4.7 Hz, 2H), 0.56 |
| | (dq, J = 9.9, 4.7 Hz, 1H), 0.44 (dq, J = 8.6, 4.5 Hz, 1H). |
| 233 | MS m/z 365.4 [M + H]⁺; ¹H NMR (500 MHz, DMSO-d₆) δ 9.05 (s, 1H), 8.26 |
| | (s, 1H), 7.98 (d, J = 8.1 Hz, 1H), 7.63 (d, J = 1.7 Hz, 1H), 7.58 (dd, J = 8.1, |
| | 1.6 Hz, 1H), 7.42 (s, 1H), 4.76-4.60 (m, 2H), 3.42-3.21 (m, 3H), 2.96 (td, |
| | J = 12.9, 3.5 Hz, 1H), 2.43 (td, J = 9.9, 3.3 Hz, 1H), 1.01-0.92 (m, 1H), 0.60 |
| | (dtt, J = 13.1, 8.6, 4.0 Hz, 2H), 0.47 (dq, J = 9.6, 4.5 Hz, 1H), 0.41-0.34 (m, |
| | 1H); 2Hs not observed (NH and OH). |
| 234 | MS m/z: 408.1 [M + H]⁺; ¹H NMR (400 MHz, methanol-d₄ and CDCl₃) δ: |
| | 9.05 (s, 1H), 8.81 (d, J = 0.8 Hz, 1H), 7.93 (d, J = 8.4 Hz, 1H), 7.62-7.65 (m, |
| | 2H), 7.24 (d, J = 0.8 Hz, 1H), 4.86 (d, J = 12.8 Hz, 1H), 4.78 (d, J = 13.6 Hz, |
| | 1H), 4.07 (s, 3H), 2.93-2.97 (m, 1H), 2.63-2.75 (m, 3H), 1.53-1.59 (m, 2H), |
| | 1.23 (d, J = 6.4 Hz, 3H), 1.08 (t, J = 7.6 Hz, 3H); 2Hs not observed (NH and OH). |
| 235 | MS m/z: 420.0 [M + H]⁺; ¹H NMR (400 MHz, methanol-d₄ and CDCl₃) δ: |
| | 9.06 (s, 1H), 8.79 (s, 1H), 7.93 (d, J = 8.0 Hz, 1H), 7.61-7.65 (m, 2H), 7.25 (d, |
| | J = 0.8 Hz, 1H), 4.87 (q, J = 16.0 Hz, 2H), 4.06 (s, 3H), 4.92 (t, J = 3.0 Hz, 2H), |
| | 2.68-2.74 (m, 1H), 1.98-2.04 (m, 1H), 1.23 (q, J = 16.0 Hz, 3H), 0.83-0.89 |
| | (m, 1H), 0.60-0.63 (m, 2H), 0.36-0.37 (m, 2H); 2Hs not observed (NH and OH). |
| 236 | MS m/z 410.3 [M + H]⁺; ¹H NMR (500 MHz, methanol-d₄) δ: 9.02 (s, 1H), |
| | 7.97 (s, 1H), 7.82 (d, J = 8.5 Hz, 1H), 7.20 (d, J = 8.5 Hz, 1H), 7.19 (s, 1H), |
| | 4.73-4.69 (m, 2H), 2.95-2.86 (m, 1H), 2.78-2.73 (m, 1H), 2.62 (t, J = 11.5 |
| | Hz, 1H), 2.52 (t, J = 13.0 Hz, 1H), 2.41-2.32 (m, 1H), 2.17-2.10 (m, 2H), |
| | 2.03-1.86 (m, 4H), 1.20 (d, J = 6.0 Hz, 3H); 3Hs not observed (2 NHs and OH). |
| 238 | MS m/z 409.4 [M + H]⁺; ¹H NMR (500 MHz, methanol-d₄) δ: 9.03 (s, 1H), |
| | 7.99 (s, 1H), 7.84 (s, 1H), 7.80 (d, J = 8.5 Hz, 1H), 7.17 (d, J = 8.0 Hz, 1H), |
| | 7.14 (s, 1H), 4.77-4.67 (m, 2H), 2.92-2.86 (m, 1H), 2.76 (t, J = 9.5 Hz, 1H), |
| | 2.63 (t, J = 13.0 Hz, 1H), 2.53 (t, J = 13.0 Hz, 1H), 2.41-2.32 (m, 1H), 2.16- |
| | 2.09 (m, 2H), 2.02-1.85 (m, 4H), 1.20 (d, J = 6.5 Hz, 3H); 2Hs not observed |
| | (NH and OH). |
| 239 | MS m/z 384.5 [M + H]⁺; ¹H NMR (500 MHz, DMSO-d₆) δ 11.00 (s, 1H), |
| | 10.39 (s, 1H), 9.10 (s, 1H), 8.01 (d, J = 8.1 Hz, 1H), 7.95 (s, 1H), 7.88 (d, J = |
| | 1.5 Hz, 1H), 4.83 (dd, J = 38.0, 13.7 Hz, 2H), 3.49-3.38 (m, 2H), 3.21 (tt, J = |
| | 19.0, 11.0 Hz, 3H), 1.98 (h, J = 6.8 Hz, 1H), 1.07 (dd, J = 6.9, 4.3 Hz, 6H); |
| | 1H not observed (NH or OH). |
| 240 | MS m/z 406.4 [M + H]⁺; ¹H NMR (500 MHz, methanol-d₄) δ: 9.06 (s, 1H), |
| | 7.85 (d, J = 8.5 Hz, 1H), 7.63 (s, 1H), 7.40 (d, J = 8.5 Hz, 1H), 7.39 (s, 1H), |
| | 6.66 (s, 1H), 4.75 (d, J = 13.5 Hz, 2H), 3.96 (s, 3H), 2.96-2.90 (m, 1H), 2.82- |
| | 2.76 (m, 1H), 2.66 (t, J = 13.0 Hz, 1H), 2.55 (t, J = 13.0 Hz, 1H), 2.42-2.33 |
| | (m, 1H), 2.18-2.11 (m, 2H), 2.04-1.86 (m, 4H), 1.22 (d, J = 6.5 Hz, 3H); 2Hs |
| | not observed (NH and OH). |
| 245 | MS m/z 396.3 [M + H]⁺; ¹H NMR (500 MHz, DMSO-d₆) δ 10.95 (s, 1H), 9.09 |
| | (s, 1H), 7.98 (d, J = 8.1 Hz, 1H), 7.89 (d, J = 1.5 Hz, 1H), 7.82 (dd, J = 8.2, |
| | 1.6 Hz, 1H), 4.80-4.68 (m, 2H), 3.52-3.32 (m, 4H), 3.11 (dq, J = 14.2, 8.1 |
| | Hz, 1H), 2.80 (s, 3H), 2.70 (t, J = 6.2 Hz, 1H), 1.05 (tp, J = 8.8, 4.8 Hz, 1H), |
| | 0.68 (dtq, J = 17.2, 8.6, 4.6, 4.0 Hz, 2H), 0.56 (dq, J = 9.8, 4.6 Hz, 1H), 0.45 |
| | (dq, J = 8.7, 4.5 Hz, 1H); 1H not observed (NH or OH). |
| 246 | MS m/z 395.3 [M + H]⁺; ¹H NMR (500 MHz, DMSO-d₆) δ 9.11 (s, 1H), 7.98 |
| | (s, 1H), 7.91 (d, J = 8.2 Hz, 1H), 7.63 (d, J = 1.6 Hz, 1H), 7.55 (dd, J = 8.3, |
| | 1.6 Hz, 1H), 4.78-4.66 (m, 2H), 3.45 (td, J = 14.1, 13.3, 6.2 Hz, 3H), 3.11 |
| | (dtd, J = 12.8, 8.8, 4.4 Hz, 1H), 2.74 (s, 3H), 2.71-2.64 (m, 1H), 1.04 (tt, J = |
| | 8.3, 3.9 Hz, 1H), 0.67 (dtq, J = 17.2, 8.6, 4.4 Hz, 2H), 0.56 (dq, J = 10.0, 4.7 |
| | Hz, 1H), 0.44 (dq, J = 8.7, 4.5 Hz, 1H); 2Hs not observed (NH and OH). |
| 247 | MS m/z 394.3 [M + H]⁺; ¹H NMR (500 MHz, methanol-d₄) δ: 9.02 (s, 1H), |
| | 8.10-7.90 (s, 2H), 7.80 (d, J = 8.5 Hz, 1H), 7.21 (d, J = 8.5 Hz, 1H), 7.19 (s, |
| | 1H), 4.86 (d, J = 12.5 Hz, 1H), 4.68 (d, J = 12.5 Hz, 1H), 2.93-2.84 (m, 1H), |
| | 2.76 (t, J = 11.5 Hz, 1H), 2.62 (t, J = 12.5 Hz, 1H), 2.50 (t, J = 13.0 Hz, 1H), |
| | 1.21 (d, J = 6.5 Hz, 3H), 1.06 (s, 9H); 3Hs not observed (2 NHs and OH). |
| 248 | MS m/z 412.5 [M + H]⁺; ¹H NMR (500 MHz, methanol-d₄) δ: 9.00 (s, 1H), |
| | 7.95 (s, 1H), 7.80 (d, J = 9.0 Hz, 1H), 7.18 (d, J = 9.0 Hz, 1H), 7.17 (s, 1H), |
| | 4.68 (d, J = 13.0 Hz, 1H), 3.74 (t, J = 5.5 Hz, 1H), 3.67 (q, J = 5.0 Hz, 2H), |
| | 3.58 (t, J = 5.0 Hz, 1H), 2.94-2.86 (m, 1H), 2.77 (t, J = 12.0 Hz, 1H), 2.63 (t, |
| | J = 12.5 Hz, 1H), 2.52 (dd, J = 10.5, 2.0 Hz, 1H), 1.22 (d, J = 6.5 Hz, 3H), |
| | 1.05 (s, 9H). |
| 249 | MS m/z 364.3 [M + H]⁺; ¹H NMR (500 MHz, methanol-d₄) δ: 9.03 (s, 1H), |
| | 8.10-7.90 (s, 2H), 7.80 (d, J = 8.0 Hz, 1H), 7.21 (d, J = 8.0 Hz, 1H), 7.18 (s, |
| | 1H), 5.94-5.86 (m, 1H), 5.40 (d, J = 17.5 Hz, 1H), 5.28 (d, J = 11.0 Hz, 1H), |
| | 4.77-4.70 (m, 2H), 3.42-3.39 (m, 1H), 2.98-2.91 (m, 1H), 2.77 (t, J = 12.5 |
| | Hz, 1H), 2.65 (t, J = 12.5 Hz, 1H), 1.21 (d, J = 6.5 Hz, 3H); 3Hs not observed |
| | (2 NHs and OH). |
| 250 | MS m/z 383.3 [M + H]⁺; ¹H NMR (500 MHz, DMSO-d₆) δ: 11.23 (br s, 1H), |
| | 9.40 (s, 1H), 9.15 (s, 1H), 9.12 (br s, 1H), 9.06 (s, 1H), 7.96 (d, J = 8.1 Hz, |
| | 1H), 7.44 (dd, J = 8.2, 1.8 Hz, 1H), 7.40 (s, 1H), 4.85 (d, J = 13.8 Hz, 1H), |
| | 4.77 (d, J = 14.1 Hz, 1H), 3.55-3.33 (m, 2H), 3.32-3.06 (m, 3H), 2.12- |
| | 1.88 (m, 1H), 1.07 (app t, J = 6.0 Hz, 6H). |
| 251 | MS m/z 405.3 [M + H]⁺; ¹H NMR (500 MHz, DMSO-d₆) δ 9.12 (s, 1H), 8.27 |
| | (d, J = 5.3 Hz, 1H), 7.97 (d, J = 8.1 Hz, 1H), 7.40 (dd, J = 8.1, 1.8 Hz, 1H), |
| | 7.35 (d, J = 1.8 Hz, 1H), 7.30 (dd, J = 5.3, 1.6 Hz, 1H), 7.09 (d, J = 1.5 Hz, |
| | 1H), 4.81-4.65 (m, 2H), 3.52-3.38 (m, 3H), 3.18 (s, 3H), 3.15-3.05 (m, |
| | 1H), 2.75-2.63 (m, 1H), 1.04 (tq, J = 8.7, 4.3 Hz, 1H), 0.68 (dddd, J = 15.3, |
| | 12.6, 8.2, 4.4 Hz, 2H), 0.56 (dq, J = 9.8, 4.6 Hz, 1H), 0.44 (dq, J = 8.5, 4.5 |
| | Hz, 1H); 2Hs not observed (NH and OH). |
| 252 | MS m/z 381.2 [M + H]⁺; ¹H NMR (500 MHz, DMSO-d₆) δ 11.04 (s, 1H), 9.19 |
| | (d, J = 4.7 Hz, 1H), 9.12 (s, 1H), 8.00-7.93 (m, 2H), 7.71 (d, J = 1.6 Hz, |
| | 1H), 7.65 (dd, J = 8.2, 1.7 Hz, 1H), 4.81-4.65 (m, 2H), 3.51-3.40 (m, 3H), |
| | 3.16-3.04 (m, 1H), 2.70 (d, J = 9.9 Hz, 1H), 1.04 (ddt, J = 13.0, 8.6, 4.5 Hz, |
| | 1H), 0.67 (dtt, J = 21.3, 8.6, 4.2 Hz, 2H), 0.56 (dq, J = 9.8, 4.6 Hz, 1H), 0.45 |
| | (dq, J = 8.4, 4.5 Hz, 1H); 1H not observed (NH or OH). |
| 253 | MS m/z 397.3 [M + H]⁺; ¹H NMR (500 MHz, DMSO-d₆) δ: 11.18 (br s, 1H), |
| | 9.01 (s, 1H), 8.48 (s, 1H), 7.97 (d, J = 7.9 Hz, 1H), 7.18-7.12 (m, 2H), 4.65 |
| | (d, J = 12.6 Hz, 1H), 4.55 (d, J = 12.7 Hz, 1H), 3.16-2.96 (m, 2H), 2.75 (dd, |
| | J = 12.6, 10.5 Hz, 1H), 2.68 (td, J = 11.6, 3.3 Hz, 1H), 2.40 (s, 3H), 2.37-2.33 |
| | (m, 1H), 1.65 (q, J = 6.7 Hz, 1H), 0.97 (dd, J = 6.8, 2.2 Hz, 6H); 1H not |
| | observed (NH or OH). |
| 254 | MS m/z 344.3 [M + H]⁺; ¹H NMR (500 MHz, DMSO-d₆) δ: 9.37 (s, 1H), 8.27 |
| | (s, 1H), 7.15 (d, J = 8.5 Hz, 1H), 7.12 (s, 1H), 7.12 (d, J = 8.5 Hz, 1H), 4.09 |
| | (d, J = 13.0 Hz, 1H), 3.91 (d, J = 13.0 Hz, 1H), 2.93 (t, J = 5.0 Hz, 1H), 2.87- |
| | 2.84 (m, 1H), 2.77 (t, J = 5.0 Hz, 1H), 2.11-2.04 (m, 1H), 1.98 (t, J = 12.5 |
| | Hz, 1H), 1.83 (t, J = 13.0 Hz, 1H), 1.70 (d, J = 11.0 Hz, 1H), 0.39 (d, J = 4.0 |
| | Hz, 2H), 0.25 (s, 9H). |
| 255 | MS m/z 344.3 [M + H]⁺; ¹H NMR (500 MHz, DMSO-d₆) δ: 9.19 (s, 1H), 9.09 |
| | (s, 1H), 8.87 (s, 1H), 8.49 (s, 1H), 7.97 (d, J = 8.0 Hz, 1H), 7.88 (s, 1H), 7.75 |
| | (d, J = 8.0 Hz, 1H), 7.45 (s, 1H), 4.95 (d, J = 13.5 Hz, 1H), 4.80 (d, J = 13.5 |
| | Hz, 1H), 3.98 (s, 3H), 3.40-3.25 (m, 3H), 3.17-3.09 (m, 1H), 1.50 (d, J = 6.0 |
| | Hz, 3H), 1.12 (s, 9H). |
| 256 | MS m/z: 419.0 [M + H]⁺; ¹H NMR (400 MHz, DMSO-d₆) δ: 11.25 (m, 1H), |
| | 9.03 (s, 1H), 8.24 (d, J = 5.2 Hz, 1H), 7.94 (d,J = 8.4 Hz, 1H), 7.33 (m, 3H), |
| | 7.08 (d, J = 0.8 Hz, 1H), 4.63 (q, J = 34.4 Hz, 2H), 3.90 (s, 3H), 2.74 (q, J = 1.6 |
| | Hz, 2H), 2.55 (d, J = 12.4 Hz, 1H), 1.93 (m, 1H), 1.07 (d, J = 6.4 Hz, 3H), 0.77 |
| | (m, 1H), 0.45 (m, 2H), 0.27 (m, 3H). |
| 257 | MS m/z: 421.0 [M + H]⁺; ¹H NMR (500 MHz, DMSO-d₆) δ 11.28 (s, 1H), |
| | 9.03 (s, 1H), 8.24 (d, J = 5.2 Hz, 1H), 7.95 (d, J = 8.4 Hz, 1H), 7.33 (m, 3H), |
| | 7.08 (d, J = 0.8 Hz, 1H), 4.64 (q, J = 25.6 Hz, 2H), 3.87 (s, 3H), 2.74 (m, 1H), |
| | 2.59 (q, J = 12.4 Hz, 1H), 2.43 (m, 2H), 1.64 (m, 1H), 1.07 (t, J = 6.4 Hz, 3H), |
| | 0.97 (m, 6H); 1 H not observed. |
| 258 | MS m/z 398.3 [M + H]⁺; ¹H NMR (500 MHz, methanol-d₄) δ: 10.21 (s, 1H), |
| | 9.20 (s, 1H), 8.02 (d, J = 8.2 Hz, 1H), 7.98 (s, 1H), 7.97 (br s, 1H), 5.22 (br d, |
| | J = 11.9 Hz, 1H), 5.09 (d, J = 14.0 Hz, 1H), 3.57 (d, J = 11.9 Hz, 1H), 3.35-3.48 |
| | (m, 2H), 3.19-3.29 (m, 2H), 1.25 (s, 9H); 2 Hs not observed (NH and OH). |
| 259 | MS m/z 381.4 [M + H]⁺; ¹H NMR (500 MHz, CDCl₃) δ: 11.35 (br s, 1H), 9.45 |
| | (br s, 1H), 9.13 (s, 1H), 8.62 (s, 1H), 7.98 (d, J = 8.1 Hz, 1H), 7.74 (s, 1H), |
| | 7.72-7.60 (m, 1H), 4.91 (d, J = 13.9 Hz, 1H), 4.83 (d, J = 14.2 Hz, 1H), |
| | 4.00 (s, 3H), 3.58-3.52 (m, 2H), 3.50-3.42 (m, 2H), 3.35-3.32 (m, 1H), 2.10 |
| | (q, J = 6.8 Hz, 1H), 1.13 (t, J = 6.3 Hz, 6H). |
| 260 | MS m/z 381.4 [M + H]⁺; ¹H NMR (500 MHz, DMSO-d₆) δ: 11.03 (br s, 1H), |
| | 9.27 (br s, 1H), 9.15 (br s, 1H), 8.56 (s, 1H), 7.92 (d, J = 8.0 Hz, 1H), 7.54 (s, |
| | 1H), 7.42 (d, J = 8.1 Hz, 1H), 4.85 (d, J = 13.9 Hz, 1H), 4.77 (d, J = 14.1 Hz, |
| | 1H), 4.11 (s, 3H), 3.54-3.34 (m, 2H), 3.28-3.23 (m, 1H), 3.15 (d, J = 10.4 |
| | Hz, 2H), 2.14-1.94 (m, 1H), 1.08 (t, J = 6.1 Hz, 6H). |
| 261 | MS m/z 381.3 [M + H]⁺; ¹H NMR (500 MHz, DMSO-d₆) δ: 11.17 (br s, 1H), |
| | 9.21-9.36 (m, 1H), 9.10 (s, 1H), 7.99 (d, J = 8.3 Hz, 1H), 7.97 (s, 1H), 7.25- |
| | 7.23 (m, 2H), 4.86 (d, J = 13.9 Hz, 1H), 4.78 (d, J = 14.5 Hz, 1H), 4.13 (s, |
| | 3H), 3.55-3.36 (m, 3H), 3.30-3.25 (m 1H), 3.17-3.15 (m, 1H), 2.04 (h, J = |
| | 6.8 Hz, 1H), 1.08 (t, J = 6.5 Hz, 6H). |
| 262 | MS m/z 381.3 [M + H]⁺; ¹H NMR (500 MHz, DMSO-d₆) δ: 11.17 (br s, 1H), |
| | 9.36 (br s, 1H), 9.10 (s, 1H), 8.30-7.74 (m, 2H), 7.25-7.23 (m, J = 7.9 Hz, |
| | 2H), 4.86 (d, J = 13.9 Hz, 1H), 4.78 (d, J = 14.5 Hz, 1H), 4.13 (s, 3H), 3.49 |
| | (t, J = 13.0 Hz, 1H), 3.42-3.39 (m, 2H), 3.29-3.25 (m, 1H), 3.15-3.13 (m, |
| | 1H), 2.04 (q, J = 6.8 Hz, 1H), 1.08 (t, J = 6.5 Hz, 6H). |
| 265 | MS m/z 384.3 [M + H]⁺; ¹H NMR (500 MHz, DMSO-d₆) δ: 11.21 (br s, 1H), |
| | 9.47 (s, 1H), 9.12 (s, 1H), 8.74 (br s, 1H), 8.03 (d, J = 8.4 Hz, 1H), 7.77-7.77 |
| | (m, 2H), 4.83 (d, J = 13.7 Hz, 1H), 4.75 (d, J = 13.9 Hz, 1H), 3.35-3.32 (m, |
| | 2H), 3.18-3.10 (m, 3H), 1.94-1.92 (m, 1H), 1.05 (dd, J = 7.0, 3.9 Hz, 6H). |
| 266 | MS m/z: 407.1 [M + H]⁺; ¹H NMR (400 MHz, methanol-d₄) δ: 9.07 (s, 1H), |
| | 8.18 (d, J = 6.0 Hz, 1H), 7.94 (d, J = 8.0 Hz, 1H), 7.30-7.33 (m, 2H), 7.26 (q, |
| | J = 8.0 Hz, 1H), 7.07 (d, J = 0.8 Hz, 1H), 4.83-4.89 (m, 1H), 4.74 (d, J = 11.6 |
| | Hz, 1H), 3.96 (s, 3H), 2.85-2.89 (m, 1H), 2.58-2.67 (m, 3H), 1.52-1.57 (m, |
| | 2H), 1.19 (d, J = 6.4 Hz, 3H), 1.06 (t, J = 7.4 Hz, 3H); 2Hs not observed |
| | (NH and OH). |
| 267 | MS m/z: 384.0 [M + H]⁺; ¹H NMR (400 MHz, DMSO-d₆) δ: 9.00 (d, J = 9.6 |
| | Hz, 2H), 8.02 (d, J = 8.0 Hz, 1H), 7.62-7.66 (m, 2H), 4.66 (q, J = 24.0 Hz, 2H), |
| | 2.75 (d, J = 6.8 Hz, 1H), 2.52-2.57 (m, 2H), 1.38-1.45 (m, 2H), 1.23 (s, 1H), |
| | 1.07 (d, J = 6.0 Hz, 3H), 0.95 (t, J = 7.4 Hz, 3H); 2Hs not observed (NH and OH). |
| 268 | MS m/z: 397.9 [M + H]⁺; ¹H NMR (400 MHz, DMSO-d₆) δ: 11.28 (s, 1H), |
| | 8.99 (d, J = 9.6 Hz, 2H), 8.02 (d, J = 8.0 Hz, 1H), 7.64 (t, J = 12.6 Hz, 2H), 4.67 |
| | (q, J = 7.8 Hz, 2H), 2.75 (s, 1H), 2.56-2.65 (m, 2H), 2.41-2.49 (m, 1H), 1.64- |
| | 1.66 (m, 1H), 1.23(s, 1H), 1.08 (d, J = 6.0 Hz, 3H), 0.97 (t, J = 5.2 Hz, 6H). |
| 269 | MS m/z: 396.1 [M + H]⁺; ¹H NMR (400 MHz, DMSO-d₆) δ: 11.24 (s, 1H), |
| | 9.04 (s, 1H), 8.99 (s, 1H), 8.02 (d, J = 8.0 Hz, 1H), 7.65 (d, J = 16.4 Hz, 2H), |
| | 4.70 (d, J = 24.0 Hz, 2H), 2.85 (s, 2H), 1.22 (s, 2H), 1.14 (s, 3H), 0.81 (s, 1H), |
| | 0.50 (s, 2H), 0.31 (d, J = 19.6 Hz, 2H); 1 H not observed (NH or OH). |
| 270 | MS m/z 383.1 [M + H]⁺; ¹H NMR (500 MHz, DMSO-d₆) δ: 9.59 (s, 1H), 9.34 |
| | (s, 1H), 9.08 (s, 1H), 8.62 (s, 1H), 7.96 (d, J = 8.5 Hz, 1H), 7.80 (s, 1H), 7.37 |
| | (s, 2H), 4.84 (d, J = 13.9 Hz, 1H), 4.76 (d, J = 14.1 Hz, 1H), 3.51 (t, J = 12.6 |
| | Hz, 1H), 3.39 (d, J = 12.8 Hz, 1H), 3.28 (dd, J = 13.8, 11.0 Hz, 1H), 3.17- |
| | 3.09 (m, 2H), 2.06 (h, J = 6.8 Hz, 1H), 1.07 (d, J = 6.8 Hz, 6H). |
| 271 | MS m/z 421.2 [M + H]⁺; ¹H NMR (500 MHz, DMSO-d₆) δ: 9.72 (s, 1H), 9.43 |
| | (s, 1H), 9.12 (s, 1H), 7.99 (d, J = 8.0 Hz, 1H), 7.46-7.40 (m, 2H), 7.31 (s, |
| | 1H), 7.06 (s, 1H), 4.85-4.76 (m, 2H), 3.98 (s, 3H), 3.59-3.50 (m, 1H), |
| | 3.43-3.35 (m, 1H), 3.30 (dd, J = 14.0, 11.1 Hz, 1H), 3.13 (d, J = 10.6 Hz, |
| | 2H), 2.52 (s, 3H), 2.08 (h, J = 6.9 Hz, 1H), 1.08 (d, J = 7.0 Hz, 6H). |
| 272 | MS m/z 408.3 [M + H]⁺; ¹H NMR (500 MHz, DMSO-d₆) δ: 9.25 (s, 1H), 8.69 |
| | (s, 1H), 7.91 (d, J = 7.9 Hz, 1H), 7.38 (s, 1H), 7.26 (br d, J = 7.9 Hz, 1H), 4.62 |
| | (d, J = 12.2 Hz, 1H), 4.52 (br d, J = 11.9 Hz, 1H), 2.91-3.04 (m, 2H), 2.71 (dd, |
| | J = 12.5, 10.5 Hz, 1H), 2.64 (td, J = 11.6, 3.6 Hz, 1H), 2.31 (ddd, J = 10.0, 6.7, |
| | 3.4 Hz, 1H), 1.63 (dq, J = 13.4, 6.7 Hz, 1H), 0.96 (dd, J = 6.7, 1.2 Hz, 6H); 2 Hs |
| | not observed (NH and OH). |
| 273 | MS m/z 408.4 [M + H]⁺; ¹H NMR (500 MHz, DMSO-d₆) δ: 9.10 (s, 1H), 8.72 |
| | (s, 1H), 7.98 (d, J = 7.6 Hz, 1H), 7.68 (s, 1H), 7.62 (d, J = 8.5 Hz, 1H), 4.84 (d, |
| | J = 13.4 Hz, 1H), 4.76 (d, J = 14.2 Hz, 1H), 3.49 (t, J = 12.4 Hz, 1H), 3.39 (d, |
| | J = 12.5 Hz, 1H), 3.26 (dd, J = 13.1, 11.0 Hz, 1H), 3.06-3.18 (m, 2H), 2.04 (dq, |
| | J = 14.0, 6.7 Hz, 1H), 1.08 (t, J = 6.7 Hz, 6H); 2 Hs not observed (NH and OH). |
| 275 | MS m/z 419.5 [M + H]⁺; ¹H NMR (500 MHz, methanol-d₄) δ: 8.75 (s, 1H), |
| | 7.53 (d, J = 7.9 Hz, 1H), 7.28 (s, 1H), 6.72 (dd, J = 8.2, 1.5 Hz, 1H), 6.70 (s, |
| | 1H), 4.53 (d, J = 12.8 Hz, 1H), 4.42 (br d, J = 12.8 Hz, 1H), 3.93 (dd, J = 8.5, 8.0 |
| | Hz, 2H), 3.64 (dd, J = 8.5, 8.0 Hz, 2H), 2.86-2.99 (m, 2H), 2.76 (dd, J = 12.8, |
| | 10.7 Hz, 1H), 2.64 (s, 3H), 2.59 (td, J = 12.5, 3.2 Hz, 1H), 1.74 (td, J = 11.0, 3.7 |
| | Hz, 1H), 0.56-0.72 (m, 1H), 0.35-0.44 (m, 2H), 0.02-0.20 (m, 2H); 2 Hs not |
| | observed (NH and OH). |
| 276 | MS m/z 404.4 [M + H]⁺; ¹H NMR (500 MHz, methanol-d₄) δ: 9.11 (s, 1H), |
| | 7.93 (s, 1H), 7.82 (d, J = 8.2 Hz, 1H), 7.15 (d, J = 8.2 Hz, 1H), 7.11 (s, 1H), |
| | 4.95 (dd, J = 14.5, 2.5 Hz, 1H), 4.86-4.89 (m, 1H), 4.14-4.23 (m, 2H), 3.61- |
| | 3.77 (m, 1H), 3.52-3.59 (m, 1H), 3.48 (dd, J = 14.0, 10.7 Hz, 1H), 3.14-3.22 |
| | (m, 3H), 2.72-2.81 (m, 2H), 2.67 (td, J = 10.2, 3.4 Hz, 1H), 1.05-1.15 (m, 1H), |
| | 0.75-0.88 (m, 2H), 0.51-0.69 (m, 2H); 2 Hs not observed (NH and OH). |
| 277 | MS m/z 418.5 [M + H]⁺; ¹H NMR (500 MHz, methanol-d₄) δ: 9.13 (s, 1H), |
| | 7.86 (d, J = 8.2 Hz, 1H), 7.74 (s, 1H), 7.08 (d, J = 1.7 Hz, 2H), 4.92-5.03 (m, |
| | 1H), 4.83-4.86 (m, 1H), 4.19 (t, J = 6.1 Hz, 2H), 3.36-3.56 (m, 3H), 3.12-3.26 |
| | (m, 1H), 3.04 (t, J = 6.3 Hz, 2H), 2.52-2.69 (m, 1H), 2.06-2.19 (m, 2H), 1.93- |
| | 2.00 (m, 2H), 0.97-1.16 (m, 1H), 0.76-0.92 (m, 2H), 0.38-0.65 (m, 2H); 2Hs |
| | not observed (NH and OH). |
| 278 | MS m/z 406.5 [M + H]⁺; ¹H NMR (500 MHz, DMSO-d₆) δ: 11.22 (s, 1H), |
| | 9.04 (s, 1H), 7.90 (s, 1H), 7.84 (d, J = 8.0 Hz, 1H), 7.08-7.15 (m, 2H), 4.63 |
| | (d, J = 12.7 Hz, 1H), 4.53 (d, J = 12.6 Hz, 1H), 4.11 (t, J = 7.3 Hz, 2H), 2.91- |
| | 3.19 (m, 4H), 2.60-2.81 (m, 4H), 2.37 (s, 1H), 1.63-1.68 (m, 1H), 0.98 (d, J = |
| | 6.9 Hz, 6H); 1H not observed (OH or NH). |
| 279 | MS m/z 406.4 [M + H]⁺; ¹H NMR (500 MHz, DMSO-d₆) δ: 11.23 (s, 1H), |
| | 9.03 (s, 1H), 7.87 (d, J = 8.0 Hz, 1H), 7.30 (s, 1H), 7.16 (d, J = 8.6 Hz, 2H), |
| | 4.63 (d, J = 12.7 Hz, 1H), 4.54 (d, J = 12.7 Hz, 1H), 4.22 (t, J = 7.0 Hz, 2H), |
| | 2.89-3.13(m, 2H), 2.67-2.89 (m, 4H), 2.55-2.65 (m, 2H), 2.35-2.43 (m, 1H), |
| | 1.66 (h, J = 6.8 Hz, 1H), 0.98 (d, J = 6.9 Hz, 6H); 1H not observed (OH or NH). |
| 280 | MS m/z 418.1 [M + H]⁺; ¹H NMR (400 MHz, CDCl₃) δ: 12.31 (s, 1H), 8.81 |
| | (s, 1H), 7.85 (s, 1H), 7.61 (d, J = 8.3 Hz, 1H), 7.14 (d, J = 1.6 Hz, 1H), 7.07 |
| | (dd, J = 8.2, 1.7 Hz, 1H), 4.74-4.65 (m, 2H), 4.19 (t, J = 7.3 Hz, 2H), 3.22- |
| | 3.17 (m, 1H), 3.14 (t, J = 12 Hz, 2H), 3.08 (td, J = 11.7, 3.1 Hz, 1H), 3.00 (t, |
| | J = 12 Hz, 1H), 2.85 (td, J = 11.7, 3.0 Hz, 1H), 2.76-2.67 (m, 2H), 1.95 (dd, |
| | J = 10.9, 2.8 Hz, 1H), 1.25 (s, 1H), 1.12 (s, 3H), 0.52-0.45 (m, 1H), 0.42-0.32 |
| | (m, 3H). |
| 281 | MS m/z 418.1 [M + H]⁺; ¹H NMR (400 MHz, CDCl₃) δ: 12.31 (s, 1H), 8.81 |
| | (s, 1H), 7.85 (s, 1H), 7.61 (d, J = 8.3 Hz, 1H), 7.14 (d, J = 1.6 Hz, 1H), 7.07 |
| | (dd, J = 8.2, 1.7 Hz, 1H), 4.74-4.65 (m, 2H), 4.19 (t, J = 7.3 Hz, 2H), 3.22- |
| | 3.17 (m, 1H), 3.14 (t, J = 12 Hz, 2H), 3.08 (td, J = 11.7, 3.1 Hz, 1H), 3.00 (t, |
| | J = 12 Hz, 1H), 2.85 (td, J = 11.7, 3.0 Hz, 1H), 2.76-2.67 (m, 2H), 1.95 (dd, |
| | J = 10.9, 2.8 Hz, 1H), 1.25 (s, 1H), 1.12 (s, 3H), 0.52-0.45 (m, 1H), 0.42-0.32 |
| | (m, 3H). |
| 282 | MS m/z 422.4 [M + H]⁺; ¹H NMR (500 MHz, DMSO-d₆) δ: 11.01 (s, 1H), |
| | 9.09 (s, 1H), 7.83 (d, J = 8.2 Hz, 1H), 7.77 (s, 1H), 7.24 (d, J = 1.8 Hz, 1H), |
| | 7.20 (dd, J = 8.3, 1.6 Hz, 1H), 4.80 (d, J = 13.6 Hz, 1H), 4.68-4.75 (m, 1H), |
| | 4.46 (t, J = 5.2 Hz, 2H), 4.15 (t, J = 6.1 Hz, 2H), 3.38 (s, 2H), 2.93-3.20 (m, |
| | 3H), 2.23-2.28 (m, 2H), 1.80-2.01 (m, 1H), 1.05 (d, J = 6.8 Hz, 6H); 1H not |
| | observed (OH or NH). |
| 283 | MS m/z 402.2 [M + H]⁺; ¹H NMR (500 MHz, methanol-d₄) δ: 8.71 (s, 1H), |
| | 8.36 (s, 1H), 7.91 (d, J = 1.6 Hz, 1H), 6.96 (s, 1 H), 6.90 (d, J = 12.4 Hz, 1H), |
| | 4.89 (d, J = 13.3 Hz, 1H), 4.81-4.80 (m, 1H), 3.26-3.31 (m, 2H), 2.99-3.06 |
| | (m, 2H), 2.80-2.84 (m, 1H), 1.80-1.83 (m, 1H), 1.02 (d, J = 6.8 Hz, 6H); 3Hs |
| | not observed (2 NHs and OH), 1H from formic acid salt. |
| 284 | MS m/z 364.3 [M + H]⁺ |

### Example 2

### Preparation of Compound 100

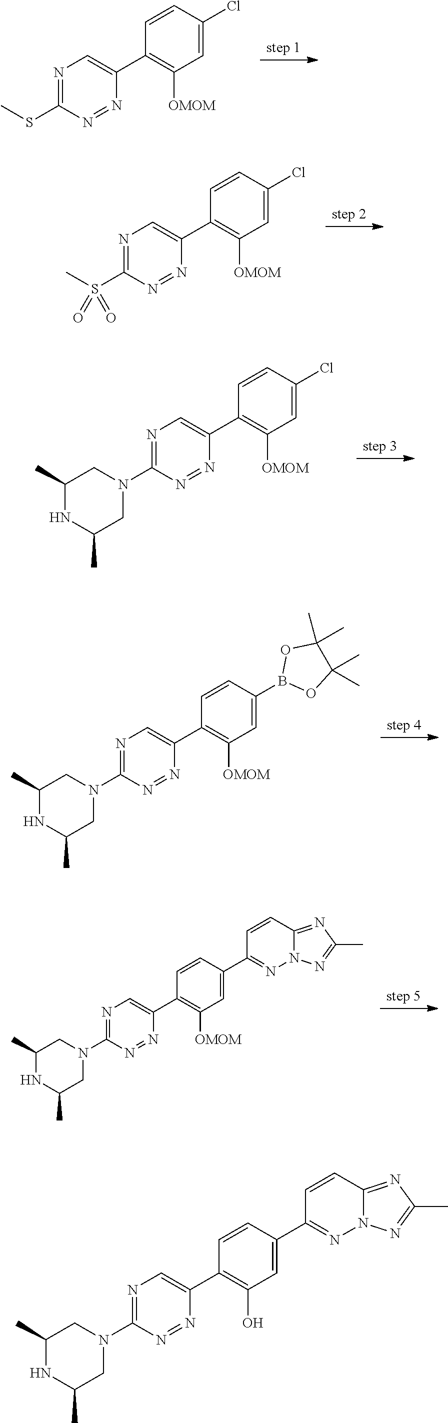

Step 1: To a stirred solution of 6-(4-chloro-2-(methoxymethoxy)phenyl)-3-(methylthio)-1,2,4-triazine (2.0 g, 6.7 mmol) in CH₂Cl₂ (35 mL) was added mCPBA (4.6 g, 20 mmol) portionwise and the reaction was allowed to stir at rt for 5 h. It was then quenched with saturated aqueous NaHCO₃. Organic layers were dried over MgSO₄ and concentrated. The residue was purified by silica gel column chromatography eluting with a gradient EtOAc/hexanes (0-100% EtOAc) to afford 6-(4-chloro-2-(methoxymethoxy)phenyl)-3-(methylsulfonyl)-1,2,4-triazine (1.75 g, 79% yield) as a tan solid. ¹H NMR (500 MHz, CDCl₃) δ: 9.40 (s, 1H), 8.06 (d, J=8.5 Hz, 1H), 7.40 (d, J=1.2 Hz, 1H), 7.27 (dd, J=8.5, 1.2 Hz, 1H), 5.32 (s, 2H), 3.56 (s, 3H), 3.52 (s, 3H).

Step 2: To a stirred solution of 6-(4-chloro-2-(methoxymethoxy)phenyl)-3-(methylsulfonyl)-1,2,4-triazine (900 mg, 2.73 mmol) in ACN (10 mL) were added cis-2,6-dimethylpiperazine (400 mg, 3.5 mmol) and DIPEA (1.0 mL, 5.73 mmol). The reaction mixture was heated at 50° C. for 1 h until UPLC showed complete conversion to the desired product. Solvent was removed under reduced pressure, the residue was purified by silica gel column chromatography eluting with a gradient CH₂Cl₂/MeOH (0-20% MeOH) to afford 6-(4-chloro-2-(methoxymethoxy)phenyl)-3-(cis-3,5-dimethylpiperazin-1-yl)-1,2,4-triazine (993 mg, 70.5% yield) as a yellowish solid. MS m/z 364.2, 366.2 [M+H]⁺.

Step 3: A suspension of 6-(4-chloro-2-(methoxymethoxy)phenyl)-3-(cis-3,5-dimethylpiperazin-1-yl)-1,2,4-triazine (650 mg, 1.79 mmol), 4,4,5,5-tetramethyl-2-(4,4,5,5-tetramethyl-1,3,2-dioxaborolan-2-yl)-1,3,2-dioxaborolane (780 mg, 2.68 mmol), KOAc (525 mg, 5.35 mmol), X Phos Pd G4 (80 mg, 0.09 mmol) in dry dioxane (12 mL) was sparged with argon for 10 minutes, then heated to 90° C. under argon atmosphere for 2 h, after which complete conversion to 3-(cis-3,5-dimethylpiperazin-1-yl)-6-(2-(methoxymethoxy)-4-(4,4,5,5-tetramethyl-1,3,2-dioxaborolan-2-yl)phenyl)-1,2,4-triazine was observed. The reaction mixture was then cooled to room temperature, concentrated and purified by silica gel column chromatography eluting with a gradient CH₂Cl₂/MeOH (0-15% MeOH) to afford 3-(cis-3,5-dimethylpiperazin-1-yl)-6-(2-(methoxymethoxy)-4-(4,4,5,5-tetramethyl-1,3,2-dioxaborolan-2-yl)phenyl)-1,2,4-triazine (800 mg, 98% yield) as a brown crystalline solid. MS m/z 456.5 [M+H]⁺.

Step 4: A mixture of 3-(cis-3,5-dimethylpiperazin-1-yl)-6-(2-(methoxymethoxy)-4-(4,4,5,5-tetramethyl-1,3,2-dioxaborolan-2-yl)phenyl)-1,2,4-triazine (60 mg, 0.13 mmol), 6-bromo-2-methyl-[1,2,4]triazolo[1,5-b]pyridazine (42.0 mg, 0.20 mmol), [1,1′-Bis(diphenylphosphino)ferrocene]dichloropalladium(II) (9.5 mg, 0.013 mmol), and aqueous potassium carbonate (0.2 mL, 2 M) in dioxane (1 mL) was degassed with argon for 10 minutes, then heated to 90° C. for 1 h. The mixture was cooled to room temperature and purified directly by silica gel column chromatography, eluting with a gradient 0-20% methanol in dichloromethane, to afford 6-[4-[3-[cis-3,5-dimethylpiperazin-1-yl]-1,2,4-triazin-6-yl]-3-(methoxymethoxy)phenyl]-2-methyl-[1,2,4]triazolo[1,5-b]pyridazine (50 mg, 83% yield). MS m/z 462.4 [M+H]⁺.

Step 5: 6-[4-[3-[cis-3,5-dimethylpiperazin-1-yl]-1,2,4-triazin-6-yl]-3-(methoxymethoxy)phenyl]-2-methyl-[1,2,4]triazolo[1,5-b]pyridazine (50 mg, 0.11 mmol) was dissolved in methanol (2 mL) and HCl (4 mol/L) in 1,4-dioxane (0.2 mL, 0.8 mmol) was added. The reaction was stirred at room temperature for 2 h, concentrated and purified by silica gel column chromatography, eluting with a gradient CH₂Cl₂/MeOH (10-30% MeOH) to afford 2-[3-[cis-3,5-dimethylpiperazin-1-yl]-1,2,4-triazin-6-yl]-5-(2-methyl-[1,2,4]triazolo[1,5-b]pyridazin-6-yl)phenol;dihydrochloride as a yellow solid (30 mg, 47% yield). MS m/z 418.4 [M+H]⁺; ¹H NMR (500 MHz, DMSO-d₆) δ: 9.90 (s, 1H), 9.57 (s, 1H), 9.11 (s, 1H), 8.41 (d, J=9.5 Hz, 1H), 8.18 (d, J=9.0 Hz, 1H), 8.04 (d, J=8.5 Hz, 1H), 7.80 (s, 1H), 7.71 (d, J=8.0 Hz, 1H), 4.85 (d, J=14.0 Hz, 2H), 3.35-3.38 (m, 2H), 3.19-3.23 (m, 2H), 3.17 (s, 3H), 1.38 (d, J=6.0 Hz, 6H).

Using the procedure described for Example 2, above, additional compounds described herein may be prepared by substituting the appropriate starting material, suitable reagents and reaction conditions, obtaining compounds such as those selected from:

| Cpd | Data |
|---|---|
| 54 | MS m/z 444.5 [M + H]⁺; ¹H NMR (500 MHz, methanol-d₄) δ: |
| | 9.19 (s, 1H), 8.31 (br s, 1H), 8.07-8.09 (m, 1H), 7.95-7.97 (m, |
| | 1H), 3.53-3.57 (m, 4H), 3.44-3.53 (m, 2H), 3.19-3.26 (m, 1H), |
| | 2.65-2.68 (m, 6H), 1.06-1.11 (m, 1H), 0.81-0.85 (m, 2H), 0.57- |
| | 0.60 (m, 2H); 3 Hs not observed (2 NHs and OH). |
| 65 | MS m/z 436.4 [M + H]⁺; ¹H NMR (500 MHz, methanol-d₄) δ: |
| | 8.45 (s, 1H), 7.86 (s, 1H), 7.73 (d, J = 8.2 Hz, 1H), 7.39 (d, J = |
| | 12.7 Hz, 1H), 7.32 (d, J = 8.4 Hz, 1H), 7.27 (s, 1H), 6.05 (s, |
| | 1H), 4.41-4.20 (m, 5H), 4.01-3.90 (m, 1H), 3.76-3.59 (m, 4H), |
| | 1.40 (t, J = 6.8 Hz, 2H); 3Hs not observed (NH and 2 OHs). |
| 66 | MS m/z 434.5 [M + H]⁺; ¹H NMR (500 MHz, methanol-d₄) δ: |
| | 9.19 (s, 1H), 8.92 (s, 1H), 8.01-8.11 (m, 3H), 7.34-7.43 (m, |
| | 2H), 5.08 (br d, J = 14.0 Hz, 2H), 3.45-3.55 (m, 2H), 3.13 (dd, |
| | J = 14.2, 11.7 Hz, 2H), 2.61 (s, 3H), 1.48 (d, J = 6.4 Hz, 6H); |
| | 2Hs not observed (NH and OH). |
| 67 | MS m/z 420.4 [M + H]⁺; ¹H NMR (500 MHz, methanol-d₄) δ: |
| | 8.49-8.44 (m, 1H), 7.88 (s, 1H), 7.74 (d, J = 8.2 Hz, 1H), 7.41 |
| | (d, J = 12.5 Hz, 1H), 7.33 (d, J = 8.4 Hz, 1H), 7.29 (s, 1H), |
| | 6.05 (s, 1H), 4.30 (s, 3H), 4.34-4.3 (m, 2H), 4.25-4.19 (m, 1H), |
| | 3.85-3.39 (m, 4H), 1.47-1.51 (m, 3H); 2Hs not observed (NH |
| | and OH). |
| 68 | MS m/z 434.4 [M + H]⁺; ¹H NMR (500 MHz, methanol-d₄) δ: |
| | 9.08 (s, 1H), 8.58 (s, 1H), 7.93 (d, J = 7.9 Hz, 1H), 7.78 (s, |
| | 1H), 7.42 (d, J = 11.9 Hz, 1H), 7.29 (d, J = 7.6 Hz, 1H), 7.26 |
| | (s, 1H), 4.76 (q, J = 13.5 Hz, 2H), 3.12-3.24 (m, 2H), 2.88 (td, |
| | J = 12.5, 3.9 Hz, 1H), 2.82 (dd, J = 13.0, 10.5 Hz, 1H), 2.63- |
| | 2.73 (m, 1H), 2.43 (s, 3H), 1.57 (quin, J = 7.5 Hz, 2H), 1.07 (t, |
| | J = 7.5 Hz, 3H); 2Hs not observed (NH and OH). |
| 69 | MS m/z 431.5 [M + H]⁺; ¹H NMR (500 MHz, methanol-d₄) δ: |
| | 9.06 (s, 1H), 7.92 (d, J = 8.5 Hz, 1H), 7.90 (s, 1H), 7.57-7.63 |
| | (m, 2H), 7.53 (s, 1H), 4.77 (d, J = 12.6 Hz, 2H), 2.86-3.05 (m, |
| | 2H), 2.71 (t, J = 12.6 Hz, 2H), 2.67 (s, 3H), 2.49 (s, 3H), 1.24 |
| | (d, J = 6.4 Hz, 6H); 2Hs not observed (NH and OH). |
| 70 | MS m/z 462.4 [M + H]⁺; ¹H NMR (500 MHz, methanol-d₄) δ: |
| | 9.11 (s, 1H), 8.01 (br d, J = 8.9 Hz, 1H), 7.72 (s, 1H), 7.71 (s, |
| | 1H), 7.49 (s, 1H), 4.84-4.86 (m, 1H), 4.74 (d, J = 13.4 Hz, 1H), |
| | 4.28 (s, 3H), 3.12-3.23 (m, 2H), 2.79-2.95 (m, 2H), 2.62 (s, |
| | 3H), 2.50-2.56 (m, 1H), 1.77 (td, J = 13.7, 7.0 Hz, 1H), 1.09 (d, |
| | J = 6.7 Hz, 6H); 2 Hs not observed (NH and OH). |
| 71 | MS m/z 448.4 [M + H]⁺; ¹H NMR (500 MHz, methanol-d₄) δ: |
| | 8.43 (s, 1H), 7.86 (s, 1H), 7.75 (d, J = 8.4 Hz, 1H), 7.29 (s, |
| | 3H), 6.06 (s, 1H), 4.40-4.18 (m, 5H), 3.84-3.42 (m, 5H), 2.19- |
| | 2.04 (m, 1H), 1.28-1.14 (m, 6H); 2Hs not observed (NH and |
| | OH). |
| 72 | MS m/z 416.6 [M + H]⁺; ¹H NMR (500 MHz, methanol-d₄) δ: |
| | 9.07 (s, 1H), 8.20 (s, 1H), 7.95 (s, 1H), 7.87 (d, J = 7.9 Hz, |
| | 1H), 7.69 (d, J = 9.0 Hz, 1H), 7.66-7.62 (m, 1H), 7.31-7.27 (m, |
| | 2H), 4.87 (br d, J = 13.6 Hz, 2H), 4.24 (s, 3H), 3.16 (br s, 2H), |
| | 2.83 (t, J = 12.5 Hz, 2H), 1.32 (d, J = 6.4 Hz, 6H); 2Hs not |
| | observed (NH and OH). |
| 73 | MS m/z 420.4 [M + H]⁺; ¹H NMR (500 MHz, methanol-d₄) δ: |
| | 9.21 (s, 1H), 8.91 (s, 1H), 8.07 (s, 1H), 7.97-8.03 (m, 2H), |
| | 7.35-7.42 (m, 2H), 4.95-5.11 (m, 2H), 3.31-3.76 (m, 6H), 3.02 |
| | (s, 3H), 2.66 (s, 3H); 1H not observed (OH). |
| 74 | MS m/z 434.4 [M + H]⁺; ¹H NMR (500 MHz, methanol-d₄) δ: |
| | 8.45 (s, 1H), 7.88 (s, 1H), 7.75 (d, J = 8.2 Hz, 1H), 7.41 (d, J = |
| | 12.7 Hz, 1H), 7.34 (d, J = 8.2 Hz, 1H), 7.29 (s, 1H), 6.06 (s, |
| | 1H), 4.34-4.20 (m, 5H), 3.83-3.41 (m, 5H), 1.94-1.76 (m, 2H), |
| | 1.24-1.10 (m, 3H); 2Hs not observed (NH and OH). |
| 75 | MS m/z 417.2 [M + H]⁺; ¹H NMR (500 MHz, methanol-d₄) δ: |
| | 9.07 (s, 1H), 7.93 (s, 3H), 7.69 (d, J = 9.5 Hz, 1H), 7.65-7.60 |
| | (m, 2H), 4.78 (d, J = 13.1 Hz, 2H), 2.96 (br s, 2H), 2.67 (t, J = |
| | 12.1 Hz, 2H), 2.50 (s, 3H), 1.22 (d, J = 6.4 Hz, 6H); 2Hs not |
| | observed (NH and OH). |
| 76 | MS m/z 434.4 [M + H]⁺; ¹H NMR (500 MHz, methanol-d₄) δ: |
| | 8.42 (s, 1H), 7.86 (s, 1H), 7.74 (d, J = 7.3 Hz, 1H), 7.42-7.23 |
| | (m, 3H), 6.05 (d, J = 4.3 Hz, 1H), 4.31 (s, 3H), 4.17-3.91 (m, |
| | 4H), 3.83-3.70 (m, 2H), 1.52-1.44 (m, 3H), 1.42-1.37 (m, 3H); |
| | 2Hs not observed (NH and OH). |
| 77 | MS m/z 434.4 [M + H]⁺ |
| 78 | MS m/z 446.4 [M + H]⁺; ¹H NMR (500 MHz, DMSO-d₆) δ: |
| | 9.44 (s, 1H), 9.33-9.41 (m, 1H), 9.17-9.23 (m, 1H), 9.13 (s, |
| | 1H), 7.98 (d, J = 8.2 Hz, 1H), 7.84 (s, 1H), 7.74 (d, J = 8.2 Hz, |
| | 1H), 4.85 (d, J = 13.5 Hz, 1H), 4.78 (d, J = 13.5 Hz, 1H), 3.45- |
| | 3.55 (m, 1H), 3.41 (br d, J = 12.5 Hz, 1H), 3.27 (dd, J = 14.3, |
| | 10.7 Hz, 1H), 3.10-3.21 (m, 2H), 2.86 (s, 3H), 2.58 (s, 3H), |
| | 2.04 (sxt, J = 6.4 Hz, 1H), 1.08 (t, J = 6.1Hz, 6H). |
| 79 | MS m/z 434.5 [M + H]⁺; ¹H NMR (500 MHz, methanol-d₄) δ: |
| | 8.43-8.40 (m, 1H), 7.86 (s, 1H), 7.74 (d, J = 8.1 Hz, 1H), 7.41- |
| | 7.30 (m, 2H), 7.29 (s, 1H), 6.06 (s, 1H), 4.40-4.32 (m, 1H), |
| | 4.28 (s, 3H), 4.26-4.18 (m, 1H), 3.73-3.59 (m, 2H), 3.52-3.39 |
| | (m, 2H), 1.54-1.45 (m, 6H); 2 Hs not observed (NH and OH). |
| 80 | MS m/z 441.5 [M + H]⁺; ¹H NMR (500 MHz, methanol-d₄) δ: |
| | 8.53-8.49 (m, 1H), 8.31 (s, 1H), 8.09 (s, 1H), 7.73 (d, J = 8.4 |
| | Hz, 1H), 7.31 (d, J = 8.2 Hz, 1H), 7.27-7.24 (m, 1H), 6.05 (s, |
| | 1H), 4.41-4.34 (m, 1H), 4.31 (s, 3H), 4.29-4.21 (m, 1H), 3.73- |
| | 3.63 (m, 2H), 3.54-3.38 (m, 2H), 1.48-1.50 (m, 6H); 2 Hs not |
| | observed (NH and OH). |
| 81 | MS m/z 448.2 [M + H]⁺; ¹H NMR (500 MHz, methanol-d₄) δ: |
| | 9.05 (s, 1H), 8.31 (d, J = 2.4 Hz, 1H), 7.86 (d, J = 8.1 Hz, 1H), |
| | 7.77 (s, 1H), 7.35-7.23 (m, 3H), 4.76-4.66 (m, 1H), 4.26 (s, |
| | 3H), 3.23-3.09 (m, 2H), 2.94-2.81 (m, 2H), 2.57-2.49 (m, 1H), |
| | 1.81-1.71 (m, 1H), 1.08 (d, J = 6.9 Hz, 6H); 3Hs not observed |
| | (NH, OH and 1 CH overlapped with solvent peak). |
| 82 | MS m/z 403.3 [M + H]⁺; ¹H NMR (500 MHz, methanol-d₄) δ: |
| | 7.81 (s, 1H), 7.65 (d, J = 2.1 Hz, 1H), 7.15 (d, J = 2.3 Hz, 1H), |
| | 7.05 (s, 1H), 6.66 (d, J = 8.7 Hz, 1H), 6.05-6.02 (m, 2H), 3.04 |
| | (s, 3H), 2.70-2.80 (m, 4H), 1.35-1.46 (m, 4H), 1.18 (s, 3H); 1H |
| | not observed (OH). |
| 83 | MS m/z 432.5 [M + H]⁺; ¹H NMR (500 MHz, DMSO-d₆) δ: |
| | 9.49 (d, J = 10.7 Hz, 1H), 9.05-9.08 (m, 2H), 8.02 (s, 1H), 7.98 |
| | (d, J = 8.2 Hz, 1H), 7.70 (d, J = 1.8 Hz, 1H), 7.64 (dd, J = 8.2, |
| | 1.8 Hz, 1H), 4.81 (d, J = 13.9 Hz, 2H), 3.35 (s, 2H), 2.96-3.13 |
| | (m, 2H), 2.61 (s, 3H), 2.50 (s, 3H), 1.30 (d, J = 6.4 Hz, 6H). |
| 85 | MS m/z 431.4 [M + H]⁺; ¹H NMR (500 MHz, DMSO-d₆) δ: |
| | 11.32 (br s, 1H), 9.22 (s, 1H), 9.15 (s, 1H), 9.07 (br s, 1H), |
| | 7.95-8.05 (m, 2H), 7.84 (d, J = 9.2 Hz, 1H), 7.42 (br dd, J = |
| | 7.9, 1.3 Hz, 1H), 7.38 (s, 1H), 4.86 (br d, J = 13.7 Hz, 1H), |
| | 4.79 (br d, J = 14.0 Hz, 1H), 3.35-3.51 (m, 2H), 3.22-3.30 (m, |
| | 2H), 3.17 (s, 3H), 3.09-3.16 (m, 1H), 1.97-2.13 (m, 1H), 1.07 |
| | (dd, J = 7.3, 4.5 Hz, 6H). |
| 86 | MS m/z 417.4 [M + H]⁺; ¹H NMR (500 MHz, DMSO-d₆) δ: |
| | 9.63 (d, J = 10.1 Hz, 1H), 9.40 (d, J = 1.5 Hz, 1H), 9.29 (s, |
| | 1H), 9.24 (d, J = 9.5 Hz, 1H), 9.13 (s, 1H), 8.12 (s, 1H), 8.02 |
| | (d, J = 8.2 Hz, 1H), 7.78 (d, J = 1.7 Hz, 1H), 7.65 (dd, J = 8.2, |
| | 1.8 Hz, 1H), 4.86 (d, J = 13.7 Hz, 2H), 3.35-3.43 (m, 1H), |
| | 3.05-3.19 (m, 3H), 2.53 (s, 3H), 1.37 (d, J = 6.4 Hz, 6H). |
| 87 | MS m/z 441.2 [M + H]⁺; ¹H NMR (500 MHz, DMSO-d₆) δ: |
| | 9.16 (s, 1H), 9.09-9.06 (m, 1H), 8.27-8.22 (m, 1H), 8.01-7.95 |
| | (m, 1H), 7.90-7.85 (m, 1H), 7.36-7.30 (m, 2H), 4.68-4.55 (m, |
| | 2H), 3.60-3.57 (m, 2H), 2.86-2.73 (m, 2H), 2.41 (s, 3H), 1.11- |
| | 1.04 (m, 6H); 2Hs not observed (NH and OH). |
| 88 | MS m/z 430.5 [M + H]⁺; ¹H NMR (500 MHz, DMSO-d₆) δ: |
| | 9.71 (br s, 1H), 9.42 (br s, 1H), 9.12 (s, 1H), 8.43 (s, 1H), 8.00 |
| | (s, 1H), 7.94 (d, J = 8.2 Hz, 1H), 7.70 (d, J = 8.5 Hz, 1H), 7.57 |
| | (dd, J = 8.9, 1.8 Hz, 1H), 7.29-7.37 (m, 2H), 4.85 (br d, J = |
| | 13.4 Hz, 1H), 4.77 (br d, J = 14.0 Hz, 1H), 3.53 (td, J = 12.5, |
| | 1.2 Hz, 1H), 3.40 (br d, J = 12.5 Hz, 1H), 3.29 (dd, J = 13.7, |
| | 11.3 Hz, 1H), 3.07-3.19 (m, 2H), 2.45 (s, 3H), 2.08 (dq, J = |
| | 13.6, 6.8 Hz, 1H), 1.08 (t, J = 6.8 Hz, 6H). |
| 89 | MS m/z 448.4 [M + H]⁺; ¹H NMR (500 MHz, methanol-d₄) δ: |
| | 9.06 (s, 1H), 8.57 (s, 1H), 7.92 (br d, J = 7.9 Hz, 1H), 7.78 (d, |
| | J = 2.0 Hz, 1H), 7.41 (d, J = 11.6 Hz, 1H), 7.28 (br d, J = 7.9 |
| | Hz, 1H), 7.25 (s, 1H), 4.48 (br d, J = 13.4 Hz, 1H), 4.43 (br d, |
| | J = 12.8 Hz, 1H), 3.44-3.57 (m, 1H), 3.22 (dd, J = 14.0, 8.5 Hz, |
| | 1H), 3.08 (dt, J = 12.2, 3.0 Hz, 1H), 2.98 (dquin, J = 13.7, 6.7 |
| | Hz, 1H), 2.62-2.72 (m, 1H), 2.54-2.61 (m, 1H), 2.47-2.53 (m, |
| | 1H), 2.46 (s, 3H), 1.21 (d, J = 6.1 Hz, 3H), 1.15 (t, J = 7.2 Hz, |
| | 3H); 1 H not observed (OH). |
| 90 | MS m/z 448.5 [M + H]⁺; ¹H NMR (500 MHz, DMSO-d₆) δ: |
| | 9.61 (d, J = 10.4 Hz, 1H), 9.22 (d, J = 10.8 Hz, 1H), 9.13 (s, |
| | 1H), 8.04 (d, J = 8.1 Hz, 1H), 7.73-7.82 (m, 2H), 7.55 (s, 1H), |
| | 4.87 (d, J = 13.9 Hz, 2H), 4.22 (s, 3H), 3.37-3.42 (m, 2H), |
| | 3.11-3.17 (m, 2H), 2.52 (s, 3H), 1.36 (d, J = 6.5 Hz, 6H). |
| 91 | MS m/z 432.4 [M + H]⁺; ¹H NMR (500 MHz, methanol-d₄) δ: |
| | 9.29 (s, 1H), 9.25 (s, 1H), 9.19 (s, 1H), 8.00 (d, J = 7.9 Hz, |
| | 1H), 7.76 (s, 1H), 7.71 (br d, J = 8.2 Hz, 1H), 5.10 (d, J = 13.7 |
| | Hz, 1H), 4.99 (d, J = 14.0 Hz, 1H), 3.44-3.61 (m, 2H), 3.25- |
| | 3.32 (m, 2H), 3.14-3.22 (m, 1H), 2.67 (s, 3H), 2.07 (spt, J = 6.8 |
| | Hz, 1H), 1.20 (d, J = 6.8 Hz, 6H); 2Hs not observed (NH and |
| | OH). |
| 92 | MS m/z 446.4 [M + H]⁺; ¹H NMR (500 MHz, methanol-d₄) δ: |
| | 9.16 (s, 1H), 8.02 (br d, J = 7.6 Hz, 1H), 7.99 (s, 1H), 7.67- |
| | 7.73 (m, 2H), 5.03 (d, J = 13.7 Hz, 1H), 4.92 (d, J = 13.7 Hz, |
| | 1H), 3.37-3.49 (m, 2H), 3.11-3.27 (m, 2H), 2.95-3.03 (m, 1H), |
| | 2.74 (s, 3H), 2.64 (s, 3H), 1.88-2.02 (m, 1H), 1.16 (d, J = 6.4 |
| | Hz, 6H); 2 Hs not observed (NH and OH). |
| 93 | MS m/z 462.4 [M + H]⁺; ¹H NMR (500 MHz, methanol-d₄) δ: |
| | 9.17 (s, 1H), 8.83 (s, 1H), 7.96 (br d, J = 8.5 Hz, 1H), 7.74 (s, |
| | 1H), 7.68 (d, J = 8.2 Hz, 1H), 5.09 (d, J = 13.7 Hz, 1H), 4.98 |
| | (d, J = 14.0 Hz, 1H), 4.27-4.36 (m, 2H), 3.70 (s, 3H), 3.43-3.62 |
| | (m, 1H), 3.24-3.32 (m, 1H), 3.05-3.20 (m, 1H), 2.62 (s, 3H), |
| | 2.02-2.16 (m, 1H), 1.19 (d, J = 6.8 Hz, 6H); 2 Hs not observed |
| | (NH and OH). |
| 94 | MS m/z 448.4 [M + H]⁺; ¹H NMR (500 MHz, DMSO-d₆) δ: |
| | 11.10 (br s, 1H), 9.16 (s, 1H), 9.14 (s, 1H), 8.20 (br s, 1H), |
| | 8.17 (s, 1H), 8.04 (d, J = 8.2 Hz, 1H), 7.36-7.44 (m, J = 2.1 Hz, |
| | 2H), 4.83 (d, J = 10.7 Hz, 2H), 3.43 (s, 3H), 3.32-3.40 (m, 2H), |
| | 2.82 (d, J = 4.6 Hz, 2H), 2.53 (s, 3H), 1.47 (d, J = 5.5 Hz, 6H). |
| 95 | MS m/z 446.4 [M + H]⁺ |
| 96 | MS m/z 445.4 [M + H]⁺; ¹H NMR (500 MHz, methanol-d₄) δ: |
| | 9.20 (s, 1H), 9.19 (s, 1H), 8.18 (s, 1H), 8.02 (d, J = 8.2 Hz, |
| | 1H), 7.76 (br s, 1H), 7.71 (br d, J = 8.2 Hz, 1H), 5.10 (d, J = |
| | 14.6 Hz, 1H), 4.99 (d, J = 14.6 Hz, 1H), 3.50-3.63 (m, 2H), |
| | 3.27-3.31 (m, 2H), 3.18-3.27 (m, 1H), 2.99 (s, 3H), 2.66 (s, |
| | 3H), 2.10 (qd, J = 14.3, 6.7 Hz, 1H), 1.20 (dd, J = 6.7, 2.7 Hz, |
| | 6H); 2Hs not observed (NH and OH). |
| 97 | MS m/z 417.4 [M + H]⁺ |
| 98 | MS m/z 417.4 [M + H]⁺; ¹H NMR (500 MHz, DMSO-d₆) δ: |
| | 11.07 (s, 1H), 9.27 (br s, 1H), 9.11 (s, 1H), 8.07 (s, 1H), 8.01 |
| | (d, J = 7.9 Hz, 1H), 7.71 (s, 1H), 7.64 (br d, J = 8.2 Hz, 1H), |
| | 7.62 (s, 1H), 4.69-4.80 (m, 2H), 3.40-3.52 (m, 2H), 3.22-3.30 |
| | (m, 2H), 3.16 (td, J = 12.8, 3.9 Hz, 1H), 2.62 (s, 3H), 2.42 (s, |
| | 3H), 1.33 (d, J = 6.5 Hz, 3H). |
| 99 | MS m/z 446.2 [M + H]⁺; ¹H NMR (500 MHz, DMSO-d₆) δ: |
| | 9.08 (s, 1H), 8.55 (d, J = 2.4 Hz, 1H), 7.94 (d, J = 8.1 Hz, 1H), |
| | 7.87 (s, 1H), 7.42-7.36 (m, 1H), 7.33 (d, J = 8.2 Hz, 1H), 7.31- |
| | 7.29 (m, 1H), 4.83 (d, J = 2.6 Hz, 2H), 4.23 (s, 3H), 3.22-2.99 |
| | (m, 3H), 2.80 (br s, 1H), 2.26-2.04 (m, 2H), 2.03-1.82 (m, 2H), |
| | 1.82-1.64 (m, 2H), 1.53-1.35 (m, 1H); 1H not observed (OH). |
| 101 | MS m/z 432.5 [M + H]⁺; ¹H NMR (500 MHz, DMSO-d₆) δ: |
| | 9.68 (d, J = 10.4 Hz, 1H), 9.43 (s, 1H), 9.30 (d, J = 10.8 Hz, |
| | 1H), 9.12 (s, 1H), 7.98 (d, J = 8.2 Hz, 1H), 7.84 (d, J = 1.7 Hz, |
| | 1H), 7.73 (dd, J = 8.2, 1.7 Hz, 1H), 4.82-4.89 (m, 2H), 3.38- |
| | 3.42 (m, 2H), 3.12-3.19 (m, 2H), 2.86 (s, 3H), 2.58 (s, 3H), |
| | 1.37 (d, J = 6.5 Hz, 6H). |
| 102 | MS m/z 417.5 [M + H]⁺; ¹H NMR (500 MHz, DMSO-d₆) δ: |
| | 11.23 (br s, 1H), 9.36 (br s, 1H), 9.12 (s, 1H), 8.63 (s, 1H), |
| | 8.46 (br d, J = 9.5 Hz, 1H), 8.19 (s, 1H), 8.15 (br d, J = 9.5 Hz, |
| | 1H), 8.07 (d, J = 8.2 Hz, 1H), 7.80 (d, J = 1.2 Hz, 1H), 7.73 |
| | (dd, J = 8.5, 1.8 Hz, 1H), 4.86 (br d, J = 13.7 Hz, 1H), 4.78 (d, |
| | J = 14.3 Hz, 1H), 3.49-3.59 (m, 1H), 3.41 (d, J = 12.5 Hz, 1H), |
| | 3.30 (dd, J = 14.0, 11.3 Hz, 1H), 3.07-3.17 (m, 2H), 2.07 (dq, |
| | J = 13.4, 6.4 Hz, 1H), 1.08 (t, J = 6.4 Hz, 6H). |
| 103 | MS m/z 431.5 [M + H]⁺ |
| 104 | MS m/z 417.4 [M + H]⁺; ¹H NMR (500 MHz, methanol-d₄) δ: |
| | 9.15-9.25 (m, 1H), 8.08 (br s, 1H), 8.02 (d, J = 7.0 Hz, 1H), |
| | 7.86 (br s, 1H), 7.63-7.76 (m, 2H), 3.40-3.82 (m, 4H), 3.37 (s, |
| | 3H), 3.01 (s, 3H), 2.70-2.76 (m, 4H), 2.56 (s, 3H), 1 H not |
| | obsrved (OH) |
| 105 | MS m/z 446.2 [M + H]⁺; ¹H NMR (500 MHz, DMSO-d₆) δ: |
| | 9.08 (s, 1H), 8.55 (d, J = 2.4 Hz, 1H), 7.94 (d, J = 8.1 Hz, 1H), |
| | 7.87 (s, 1H), 7.42-7.36 (m, 1H), 7.33 (d, J = 8.2 Hz, 1H), 7.31- |
| | 7.29 (m, 1H), 4.83 (d, J = 2.6 Hz, 2H), 4.23 (s, 3H), 3.22-2.99 |
| | (m, 3H), 2.80 (br s, 1H), 2.26-2.04 (m, 2H), 2.03-1.82 (m, 2H), |
| | 1.82-1.64 (m, 2H), 1.53-1.35 (m, 1H); 1H not observed (OH). |
| 106 | MS m/z 418.5 [M + H]⁺; ¹H NMR (500 MHz, DMSO-d₆) δ: |
| | 9.59-9.63 (m, 2H), 9.38 (d, J = 1.4 Hz, 1H), 9.18-9.24 (m, 1H), |
| | 9.14 (s, 1H), 8.00 (d, J = 8.2 Hz, 1H), 7.85 (d, J = 1.7 Hz, 1H), |
| | 7.76 (dd, J = 8.2, 1.8 Hz, 1H), 4.86 (d, J = 13.7 Hz, 2H), 3.39- |
| | 3.41 (m, 2H), 3.13 (dd, J = 14.1, 11.5 Hz, 2H), 2.60 (s, 3H), |
| | 1.36 (d, J = 6.4 Hz, 6H). |
| 107 | MS m/z 434.4 [M + H]⁺; ¹H NMR (500 MHz, DMSO-d₆) δ: |
| | 8.72-8.78 (m, 1H), 8.20 (s, 1H), 7.94-8.02 (m, 1H), 7.86 (d, J = |
| | 7.3 Hz, 1H), 7.78 (s, 1H), 7.55 (t, J = 7.6 Hz, 1H), 7.39 (d, J = |
| | 12.5 Hz, 1H), 4.50 (dd, J = 27.2, 13.4 Hz, 2H), 3.21-3.27 (m, |
| | 1H), 2.80-2.91 (m, 2H), 2.51 (s, 3H), 2.26 (s, 3H), 2.21 (td, J = |
| | 11.6, 3.4 Hz, 1H), 2.09-2.16 (m, 1H), 1.10 (d, J = 6.1 Hz, 3H); |
| | 1H not observed (OH). |
| 108 | MS m/z 456.4 [M + H]⁺; ¹H NMR (500 MHz, methanol-d₄) δ: |
| | 9.16 (br s, 1H), 8.19 (s, 1H), 8.11 (s, 1H), 8.01 (br d, J = 7.0 |
| | Hz, 1H), 7.57-7.70 (m, 2H), 5.02 (d, J = 8.9 Hz, 2H), 3.43-3.53 |
| | (m, 4H), 2.99 (s, 3H), 2.56 (s, 3H), 1.58 (d, J = 6.0 Hz, 6H); |
| | 1H not observed (OH). |
| 109 | MS m/z 458.5 [M + H]⁺; ¹H NMR (500 MHz, DMSO-d₆) δ: |
| | 9.60 (d, J = 10.6 Hz, 1H), 9.34 (s, 1H), 9.21 (d, J = 10.5 Hz, |
| | 1H), 9.12 (s, 1H), 7.96 (d, J = 8.2 Hz, 1H), 7.80 (d, J = 1.8 Hz, |
| | 1H), 7.70 (dd, J = 8.2, 1.7 Hz, 1H), 4.82-4.89 (m, 2H), 3.40 (s, |
| | 2H), 3.13 (dd, J = 14.1, 11.5 Hz, 2H), 2.81 (td, J = 8.2, 4.2 Hz, |
| | 1H), 2.58 (s, 3H), 1.41-1.32 (m, 8H), 1.30-1.25 (m, 2H). |
| 110 | MS m/z 403.3 [M + H]⁺; ¹H NMR (500 MHz, methanol-d₄) δ: |
| | 9.09 (s, 1H), 7.97-7.91 (m, 3H), 7.68 (d, J = 9.5 Hz, 1H), 7.65- |
| | 7.61 (m, 2H), 4.03 (br s, 4H), 2.69 (br s, 4H), 2.52-2.49 (m, |
| | 3H), 2.46 (s, 3H); 1H not observed (OH). |
| 111 | MS m/z 445.4 [M + H]⁺; ¹H NMR (500 MHz, methanol-d₄) δ: |
| | 9.23 (s, 1H), 8.33 (s, 1H), 8.26 (s, 1H), 8.11 (d, J = 7.9 Hz, |
| | 1H), 7.68-7.84 (m, 2H), 5.08 (d, J = 13.4 Hz, 2H), 3.44-3.57 |
| | (m, 2H), 3.03 (s, 3H), 2.76-2.87 (m, 2H), 2.68 (s, 3H), 2.05 (s, |
| | 3H), 1.57 (d, J = 6.0 Hz, 6H); 1 H not observed (OH). |
| 112 | MS m/z 418.5 [M + H]⁺; ¹H NMR (500 MHz, DMSO-d₆) δ: |
| | 9.68 (d, J = 2.4 Hz, 1H), 9.63 (d, J = 10.3 Hz, 1H), 9.21-9.29 |
| | (m, 1H), 9.18 (d, J = 2.4 Hz, 1H), 9.16 (s, 1H), 8.03 (d, J = 8.1 |
| | Hz, 1H), 7.47 (dd, J = 8.1, 1.9 Hz, 1H), 7.44 (d, J = 1.9 Hz, |
| | 1H), 4.86 (d, J = 13.8 Hz, 2H), 3.41 (s, 2H), 3.09-3.19 (m, 2H), |
| | 2.55 (s, 3H), 1.36 (d, J = 6.4 Hz, 6H). |
| 113 | MS m/z 417.5 [M + H]⁺; ¹H NMR (500 MHz, methanol-d₄) δ: |
| | 9.28 (s, 1H), 9.23 (s, 1H), 9.21 (s, 1H), 8.26 (s, 1H), 8.06 (s, |
| | 1H), 8.03 (d, J = 8.2 Hz, 1H), 7.76 (s, 1H), 7.71 (d, J = 7.6 Hz, |
| | 1H), 5.11 (d, J = 13.7 Hz, 1H), 5.01 (d, J = 14.0 Hz, 1H), 3.58 |
| | (d, J = 12.5 Hz, 1H), 3.45-3.53 (m, 1H), 3.27-3.32 (m, 2H), |
| | 3.17-3.24 (m, 1H), 2.06 (dq, J = 13.4, 6.8 Hz, 1H), 1.20 (d, J = |
| | 6.8 Hz, 6H); 2Hs not observed (NH and OH). |
| 114 | MS m/z 416.5 [M + H]⁺; ¹H NMR (500 MHz, methanol-d₄) δ: |
| | 9.19 (s, 1H), 9.08 (s, 1H), 8.19 (s, 1H), 8.10 (dd, J = 9.5, 0.9 |
| | Hz, 1H), 8.03 (d, J = 8.2 Hz, 1H), 7.93 (d, J = 1.2 Hz, 1H), |
| | 7.91 (d, J = 9.5 Hz, 1H), 7.39 (dd, J = 8.2, 1.2 Hz, 1H), 7.37 (s, |
| | 1H), 5.09 (br d, J = 13.4 Hz, 1H), 4.98 (d, J = 14.9 Hz, 1H), |
| | 3.50-3.62 (m, 2H), 3.27-3.32 (m, 2H), 3.17-3.26 (m, 1H), 2.10 |
| | (qd, J = 13.7, 6.9 Hz, 1H), 1.20 (dd, J = 6.9, 3.8 Hz, 6H); 2 Hs |
| | not observed (NH and OH). |
| 115 | MS m/z 448.4 [M + H]⁺; ¹H NMR (500 MHz, methanol-d₄) δ: |
| | 9.23 (s, 1H), 9.05 (s, 1H), 8.81 (s, 1H), 7.92 (d, J = 7.9 Hz, |
| | 1H), 7.82 (s, 2H), 7.29 (s, 1H), 7.26 (s, 1H), 4.82 (d, J = 12.5 |
| | Hz, 1H), 4.69 (d, J = 14.3 Hz, 1H), 3.09-3.20 (m, 2H), 2.80- |
| | 2.91 (m, 2H), 2.44-2.54 (m, 1H), 1.75 (dq, J = 13.7, 7.0 Hz, |
| | 1H), 1.07 (dd, J = 6.7, 3.4 Hz, 6H); 2Hs not observed (NH and |
| | OH). |
| 116 | MS m/z 448.4 [M + H]⁺; ¹H NMR (500 MHz, DMSO-d₆) δ: |
| | 9.58 (s, 1H), 9.22 (s, 1H), 9.03 (s, 1H), 8.18 (s, 1H), 8.03 (d, |
| | J = 8.1 Hz, 1H), 7.86 (d, J = 1.8 Hz, 1H), 7.76 (dd, J = 8.2, 1.8 |
| | Hz, 1H), 4.81 (d, J = 13.9 Hz, 2H), 3.34 (s, 2H), 3.09-3.03 (m, |
| | 2H), 2.95 (s, 3H), 2.72 (s, 3H), 1.30 (d, J = 6.5 Hz, 6H). |
| 117 | MS m/z 432.2 [M + H]⁺; ¹H NMR (500 MHz, DMSO-d₆) δ: |
| | 9.90 (s, 1H), 9.58 (s, 1H), 8.96-9.14 (m, 1H), 7.90-8.03 (m, |
| | 1H), 7.80-7.86 (m, 1H), 7.21 (s, 1H), 6.93-7.05 (m, 1H), 4.85 |
| | (d, J = 14.0 Hz, 2H), 3.35-3.38 (m, 2H), 3.19-3.23 (m, 2H), |
| | 2.90 (s, 3H), 2,61 (s, 3H), 1.38 (t, J = 5.9 Hz, 6H). |
| 118 | MS m/z 471.4 [M + H]⁺ |
| 119 | MS m/z 435.4 [M + H]⁺; ¹H NMR (500 MHz, DMSO-d₆) δ: |
| | 11.21 (s, 1H), 9.49 (s, 1H), 9.10 (s, 1H), 8.73 (s, 1H), 8.07 (d, |
| | J = 8.1 Hz, 1H), 7.84 (d, J = 1.8 Hz, 1H), 7.76 (dd, J = 8.2, 1.8 |
| | Hz, 1H), 4.89 (d, J = 13.8 Hz, 2H), 3.42 (s, 2H), 3.11 (dd, J = |
| | 14.1, 11.5 Hz, 2H), 2.69 (s, 3H), 1.36 (d, J = 6.5 Hz, 6H). |
| 120 | MS m/z 417.4 [M + H]⁺; ¹H NMR (500 MHz, DMSO-d₆) δ: |
| | 9.70 (d, J = 10.1 ,Hz, 1H), 9.34 (d, J = 11.1 Hz, 1H), 9.13 (s, |
| | 1H), 8.47 (d, J = 8.3 Hz, 1H), 8.06 (d, J = 8.2 Hz, 1H), 7.78 (s, |
| | 1H), 7.74 (dd, J = 8.2, 1.7 Hz, 1H), 7.70 (d, J = 1.8 Hz, 1H), |
| | 7.56 (d, J = 8.6 Hz, 1H), 4.86 (d, J = 13.8 Hz, 2H), 3.37-3.42 |
| | (m, 2H), 3.11-3.20 (m, 2H), 2.74 (s, 3H), 1.37 (d, J = 6.5 Hz, |
| | 6H). |
| 128 | MS m/z 418.5 [M + H]⁺; ¹H NMR (500 MHz, DMSO-d₆) δ: |
| | 9.75 (d, J = 1.5 Hz, 1H), 9.54 (d, J = 1.4 Hz, 1H), 9.45 (d, J = |
| | 10.6 Hz, 1H), 9.25 (d, J = 10.9 Hz, 1H), 9.15 (s, 1H), 8.80 (s, |
| | 1H), 8.01 (d, J = 8.2 Hz, 1H), 7.88 (d, J = 1.7 Hz, 1H), 7.79 |
| | (dd, J = 8.2, 1.8 Hz, 1H), 4.72-4.92 (m, 2H), 3.51 (td, J = 13.2, |
| | 3.1 Hz, 1H), 3.38-3.45 (m, 1H), 3.28 (dd, J = 14.1, 11.1 Hz, |
| | 1H), 3.14-3.17 (m, 2H), 2.05 (h, J = 6.8 Hz, 1H), 1.08 (dd, J = |
| | 6.9, 5.5 Hz, 6H). |
| 129 | MS m/z 432.5 [M + H]⁺; ¹H NMR (500 MHz, DMSO-d₆) δ: |
| | 9.75 (d, J = 1.4 Hz, 1H), 9.54 (d, J = 1.4 Hz, 1H), 9.34 (d, J = |
| | 10.7 Hz, 1H), 9.15 (s, 1H), 8.93 (d, J = 11.2 Hz, 1H), 8.80 (s, |
| | 1H), 8.01 (d, J = 8.2 Hz, 1H), 7.88 (d, J = 1.7 Hz, 1H), 7.79 |
| | (dd, J = 8.2, 1.8 Hz, 1H), 4.96 (d, J = 13.5 Hz, 1H), 4.84 (d, J = |
| | 14.0 Hz, 1H), 3.45-3.55 (m, 1H), 3.42 (d, J = 12.3 Hz, 1H), |
| | 3.17-3.29 (m, 3H), 1.11 (s, 9H). |
| 130 | MS m/z 416.5 [M + H]⁺; ¹H NMR (500 MHz, DMSO-d₆) δ: |
| | 9.73-9.81 (m, 2H), 9.54 (d, J = 1.4 Hz, 1H), 9.43 (d, J = 10.4 |
| | Hz, 1H), 9.15 (s, 1H), 8.80 (s, 1H), 8.00 (d, J = 8.2 Hz, 1H), |
| | 7.88 (d, J = 1.7 Hz, 1H), 7.79 (dd, J = 8.2, 1.8 Hz, 1H), 4.67- |
| | 4.87 (m, 2H), 3.47-3.61 (m, 2H), 3.39-3.46 (m, 1H), 3.07 (dt, |
| | J = 15.8, 8.5 Hz, 1H), 2.60-2.75 (m, 1H), 1.06-1.17 (m, 1H), |
| | 0.73-0.57 (m, 3H), 0.41-0.46 (m, 1H). |
| 131 | MS m/z 444.4 [M + H]⁺; ¹H NMR (500 MHz, DMSO-d₆) δ: |
| | 9.54 (br s, 1H), 9.32 (br s, 1H), 9.16 (s, 1H), 9.14 (s, 1H), 8.12 |
| | (s, 2H), 8.04 (d, J = 8.2 Hz, 1H), 7.40 (br s, 1H), 7.38 (s, 1H), |
| | 4.85 (d, J = 14.0 Hz, 1H), 4.77 (d, J = 12.8 Hz, 1H), 3.48-3.57 |
| | (m, 1H), 3.41 (d, J = 13.7 Hz, 1H), 3.24-3.34 (m, 1H), 3.07- |
| | 3.20 (m, 2H), 2.68 (s, 3H), 2.55 (s, 3H), 2.06 (dq, J = 13.7, 6.8 |
| | Hz, 1H), 1.08 (t, J = 6.8 Hz, 6H). |
| 138 | MS m/z 458.5 [M + H]⁺; ¹H NMR (500 MHz, methanol-d₄) δ: |
| | 9.11 (s, 1H), 8.54 (s, 1H), 7.92 (d, J = 8.2 Hz, 1H), 7.66 (s, |
| | 1H), 7.41 (s, 1H), 7.29 (br d, J = 8.2 Hz, 1H), 7.25 (s, 1H), |
| | 3.25-3.50 (m, 4H), 3.11-3.18 (m, 2H), 3.03 (q, J = 7.4 Hz, 2H), |
| | 2.95 (br t, J = 7.5 Hz, 1H), 2.46 (s, 3H), 1.97 (dq, J = 14.0, 6.7 |
| | Hz, 1H), 1.42 (t, J = 7.4 Hz, 3H), 1.15 (dd, J = 6.7, 2.7 Hz, |
| | 6H); 2Hs not observed (NH and OH). |
| 139 | MS m/z 498.4 [M + H]⁺; ¹H NMR (500 MHz, methanol-d₄) δ: |
| | 9.13 (s, 1H), 9.07 (s, 1H), 8.16 (s, 1H), 7.98 (s, 1H), 7.96 (s, |
| | 1H), 7.36 (dd, J = 8.2, 1.8 Hz, 1H), 7.32 (s, 1H), 5.06 (d, J = |
| | 14.0 Hz, 1H), 4.96 (d, J = 14.0 Hz, 1H), 3.43-3.64 (m, 2H), |
| | 3.28-3.32 (m, 1H), 3.27 (d, J = 4.0 Hz, 1H), 3.18 (ddd, J = |
| | 10.4, 7.9, 3.7 Hz, 1H), 2.54 (s, 3H), 2.09 (dq, J = 13.7, 6.7 Hz, |
| | 1H), 1.19 (dd, J = 6.7, 3.7 Hz, 6H); 2 Hs not observed (NH and |
| | OH). |
| 140 | MS m/z 444.4 [M + H]⁺; ¹H NMR (500 MHz, methanol-d₄) δ: |
| | 9.02 (s, 1H), 8.22 (s, 1H), 7.84 (d, J = 8.2 Hz, 1H), 7.78 (s, |
| | 1H), 7.39 (s, 1H), 7.26 (dd, J = 8.2, 1.2 Hz, 1H), 7.24 (s, 1H), |
| | 4.77 (br d, J = 12.2 Hz, 1H), 4.65 (d, J = 13.7 Hz, 1H), 4.24 (s, |
| | 3H), 3.09-3.17 (m, 2H), 2.73-2.89 (m, 2H), 2.64 (s, 3H), 2.43 |
| | (ddd, J = 11.3, 6.8, 2.8 Hz, 1H), 1.72 (dq, J = 13.7, 6.8 Hz, |
| | 1H), 1.05 (dd, J = 6.8, 3.7 Hz, 6H); 2 Hs not observed (NH and |
| | OH). |
| 141 | MS m/z 431.5 [M + H]⁺; ¹H NMR (500 MHz, methanol-d₄) δ: |
| | 9.41 (s, 1H), 9.36 (s, 1H), 9.21 (s, 1H), 8.23 (s, 1H), 8.04 (d, |
| | J = 8.2 Hz, 1H), 7.77 (s, 1H), 7.74 (br d, J = 8.2 Hz, 1H), 5.09 |
| | (d, J = 13.7 Hz, 1H), 4.99 (d, J = 15.0 Hz, 1H), 3.35-3.64 (m, |
| | 4H), 3.16-3.25 (m, 1H), 2.68 (s, 3H), 2.02-2.18 (m, 1H), 1.20 |
| | (dd, J = 6.7, 3.1 Hz, 6H); 2 Hs not observed (NH and OH). |
| 147 | MS m/z 431.3 [M + H]⁺; ¹H NMR (500 MHz, methanol-d₄) δ: |
| | 9.08-9.18 (m, 1H), 8.31-8.37 (m, 1H), 8.00-8.06 (m, 1H), 7.87- |
| | 7.95 (m, 1H), 7.51-7.58 (m, 2H), 6.06-6.14 (m, 1H), 4.97-5.14 |
| | (m, 1H), 4.19-4.41 (m, 2H), 3.69-3.82 (m, 1H), 3.54-3.65 (m, |
| | 1H), 3.36-3.53 (m, 2H), 2.79 (s, 3H), 2.04-2.18 (m, 1H), 1.17- |
| | 1.24 (m, 6H); 2Hs not observed (NH and OH). |
| 167 | MS m/z 418.4 [M + H]⁺; ¹H NMR (500 MHz, DMSO-d₆) δ: |
| | 11.13 (s, 1H), 9.63 (d, J = 1.4 Hz, 1H), 9.38 (d, J = 1.4 Hz, |
| | 1H), 9.20 (d, J = 10.0 Hz, 1H), 9.15 (s, 1H), 8.00 (d, J = 8.2 |
| | Hz, 1H), 7.84 (d, J = 1.8 Hz, 1H), 7.76 (dd, J = 8.2, 1.8 Hz, |
| | 1H), 4.90 (d, J = 13.9 Hz, 2H), 3.45 (s, 2H), 3.03 (dd, J = 14.2, |
| | 11.4 Hz, 2H), 2.60 (s, 3H), 1.33 (d, J = 6.4 Hz, 6H). |
| 168 | MS m/z 430.4 [M + H]⁺; ¹H NMR (500 MHz, DMSO-d₆) δ: |
| | 11.34 (s, 1H), 9.12 (d, J = 2.0 Hz, 1H), 9.10 (s, 1H), 8.68 (d, |
| | J = 2.0 Hz, 1H), 8.00 (d, J = 2.0 Hz, 1H), 7.45 (d, J = 8.0 Hz, |
| | 1H), 7.42 (s, 1H), 4.67 (d, J = 12.5 Hz, 1H), 4.58 (s, 3H), 3.16 |
| | (t, J = 10.5 Hz, 2H), 3.06 (t, J = 11.5 Hz, 1H), 2.78 (t, J = 8.5 |
| | Hz, 1H), 2.13 (t, J = 8.5 Hz, 1H), 0.93-0.84 (m, 1H), 0.56-0.47 |
| | (m, 2H), 0.42-0.35 (m, 1H), 0.32-0.28 (m, 1H). |
| 169 | MS m/z 430.4 [M + H]⁺; ¹H NMR (500 MHz, DMSO-d₆) δ: |
| | 11.27 (s, 1H), 9.47 (s, 1H), 9.28 (s, 1H), 9.16 (s, 1H), 9.04 (d, |
| | J = 2.0 Hz, 1H), 8.70 (d, J = 2.0 Hz, 1H), 8.04 (d, J = 8.0 Hz, |
| | 1H), 7.49 (d, J = 9.0 Hz, 1H), 7.47 (s, 1H), 4.76 (d, J = 13.5 |
| | Hz, 1H), 4.70 (d, J = 14.5 Hz, 1H), 4.41 (s, 3H), 3.55-3.43 (m, |
| | 1H), 3.12-3.07 (m, 2H), 2.71-2.63 (m, 1H), 1.11-1.05 (m, 1H), |
| | 0.71-0.64 (m, 1H), 0.60-0.56 (m, 1H), 0.46-0.41 (m, 1H), 0.35- |
| | 0.27 (m, 1H). |
| 171 | MS m/z 430.4 [M + H]⁺; ¹H NMR (500 MHz, DMSO-d₆) δ: |
| | 11.04 (s, 1H), 9.89 (s, 1H), 9.49 (s, 1H), 9.12 (s, 1H), 8.50 (d, |
| | J = 8.5 Hz, 1H), 8.23 (d, J = 8.5 Hz, 1H), 8.01 (d, J = 8.0 Hz, |
| | 1H), 7.94 (d, J = 1.5 Hz, 1H), 7.81 (dd, J = 8.0, 1.5 Hz, 1H), |
| | 4.76 (d, J = 14.0 Hz, 1H), 4.70 (d, J = 14.0 Hz, 1H), 4.38 (s, |
| | 3H), 3.60-3.49 (m, 2H), 3.42 (d, J = 10.5 Hz, 1H), 3.10-3.04 |
| | (m, 1H), 2.67-2.63 (m, 1H), 1.14-1.07 (m, 1H), 0.68-0.58 (m, |
| | 3H), 0.45-0.41 (m, 1H); 1H is from HCl salt. |
| 172 | MS m/z 430.4 [M + H]⁺; ¹H NMR (500 MHz, DMSO-d₆) δ: |
| | 11.13 (s, 1H), 9.57 (s, 1H), 9.08 (s, 1H), 8.47 (d, J = 1.5 Hz, |
| | 1H), 7.97 (d, J = 8.5 Hz, 1H), 7.88 (d, J = 1.5 Hz, 1H), 7.79 |
| | (dd, J = 8.5, 2.0 Hz, 1H), 4.66 (d, J = 12.5 Hz, 1H), 4.61 (s, |
| | 3H), 4.57 (d, J = 12.5 Hz, 1H), 3.14 (t, J = 9.0 Hz, 2H), 3.02 (t, |
| | J = 11.5 Hz, 1H), 2.75 (t, J = 11.5 Hz, 1H), 2.09 (t, J = 8.0 Hz, |
| | 1H), 0.90-0.82 (m, 1H), 0.54-0.46 (m, 2H), 0.38-0.34 (m, 1H), |
| | 0.31-0.27 (m, 1H); 1H not observed (NH or OH). |
| 173 | MS m/z 430.4 [M + H]⁺; ¹H NMR (500 MHz, DMSO-d₆) δ: |
| | 11.13 (s, 1H), 10.04 (s, 1H), 9.59 (s, 1H), 9.11 (s, 1H), 8.63 (d, |
| | J = 9.0 Hz, 1H), 8.08 (d, J = 9.0 Hz, 1H), 7.95 (s, 1H), 7.84 (d, |
| | J = 10.0 Hz, 1H), 4.76 (d, J = 13.0 Hz, 1H), 4.69 (d, J = 13.0 |
| | Hz, 1H), 4.36 (s, 3H), 3.62-3.51 (m, 2H), 3.40 (d, J = 12.5 Hz, |
| | 1H), 3.08-3.01 (m, 1H), 2.67-2.61 (m, 1H), 1.17-1.09 (m, 1H), |
| | 0.69-0.64 (m, 3H), 0.44-0.40 (m, 1H). |
| 174 | MS m/z 430.4 [M + H]⁺; ¹H NMR (500 MHz, DMSO-d₆) δ: |
| | 11.08 (s, 1H), 9.80 (s, 1H), 9.53 (s, 1H), 9.43 (s, 1H), 9.15 (s, |
| | 1H), 8.53 (s, 1H), 8.02 (d, J = 8.5 Hz, 1H), 7.97 (d, J = 1.5 Hz, |
| | 1H), 7.83 (dd, J = 8.5, 1.5 Hz, 1H), 4.76 (d, J = 16.0 Hz, 1H), |
| | 4.69 (d, J = 13.5 Hz, 1H), 4.40 (s, 3H), 3.58-3.41 (m, 3H), |
| | 3.09-3.03 (m, 1H), 2.70-2.63 (m, 1H), 1.14-1.07 (m, 1H), 0.72- |
| | 0.58 (m, 3H), 0.45-0.41 (m, 1H); 1H from HCl salt. |
| 176 | MS m/z 352.3 [M + H]⁺; ¹H NMR (500 MHz, methanol-d₄) δ: |
| | 7.70 (s, 1H), 6.65 (br s, 2H), 6.46 (d, J = 8.2 Hz, 1H), 5.87 (dd, |
| | J = 8.1, 1.8 Hz, 1H), 5.84 (d, J = 1.7 Hz, 1H), 3.59 (t, J = 6.0 |
| | Hz, 1H), 3.25 (dd, J = 12.8, 2.6 Hz, 1H), 1.84-1.66 (m, 2H), |
| | 1.61-1.48 (m, 2H), 0.01 (d, J = 6.9 Hz, 3H), 0.10 (d, J = 6.0 Hz, |
| | 3H); 3Hs not observed (2 NHs and OH). |
| 190 | MS m/z 352.3 [M + H]⁺; ¹H NMR (500 MHz, DMSO-d₆) 5 |
| | 9.54 (br s, 1H), 9.11 (s, 1H), 8.09 (s, 2H), 7.86 (d, J = 8.0 Hz, |
| | 1H), 7.32 (s, 1H), 7.27-7.19 (m, 2H), 7.12 (s, 1H), 5.05 (t, J = |
| | 7.2 Hz, 1H), 4.50 (d, J = 14.3 Hz, 1H), 3.77 (s, 1H), 3.59 (dd, |
| | J = 14.5, 3.7 Hz,1H), 3.4-3.42 (m, 1H), 3.22-3.11 (m, 1H), 1.39 |
| | (d, J = 6.9 Hz, 3H), 1.32 (d, J = 6.8 Hz, 3H). |
| 191 | MS m/z 434.5 [M + H]⁺; ¹H NMR (500 MHz, DMSO-d₆) δ: |
| | 11.14 (s, 1H), 9.11 (s, 1H), 8.76-8.99 (m, 1H), 8.66 (dd, J = |
| | 4.6, 1.5 Hz, 1H), 8.49 (dd, J = 8.2, 1.5 Hz, 1H), 8.06 (d, J = 8.1 |
| | Hz, 1H), 7.80 (d, J = 1.8 Hz, 1H), 7.73 (dd, J = 8.1, 1.8 Hz, |
| | 1H), 7.65 (dd, J = 8.2, 4.6 Hz, 1H), 4.88 (d, J = 13.5 Hz, 1H), |
| | 4.80 (d, J = 14.0 Hz, 1H), 3.38-3.48 (m, 2H), 3.16-3.28 (m, |
| | 3H), 1.98 (h, J = 6.8 Hz, 1H), 1.07 (d, J = 6.9 Hz, 6H). |
| 193 | MS m/z 429.4 [M + H]⁺; ¹H NMR (DMSO-d6) δ: 11.28-11.58 |
| | (m, 1H), 9.33 (s, 1H), 9.10 (s, 1H), 8.21 (s, 1H), 8.02 (d, J = |
| | 9.5 Hz, 1H), 7.99 (d, J = 8.2 Hz, 1H), 7.68 (dd, J = 9.5, 1.2 Hz, |
| | 1H), 7.46 (dd, J = 8.1, 1.7 Hz, 1H), 7.41 (d, J = 1.5 Hz, 1H), |
| | 4.64 (br d, J = 12.2 Hz, 1H), 4.55 (br d, J = 11.3 Hz, 1H), 3.00- |
| | 3.13 (m, 2H), 2.90 (br dd, J = 12.7, 10.5 Hz, 1H), 2.62-2.71 (m, |
| | 1H), 2.58 (s, 3H), 1.90-1.98 (m, 1H), 0.78-0.85 (m, 1H), 0.42- |
| | 0.51 (m, 2H),0.23-0.34 (m, 2H). |
| 194 | MS m/z 428.4 [M + H]⁺; ¹H NMR (500 MHz, DMSO-d₆) δ: |
| | 11.14-11.41 (m, 1H), 9.09 (s, 1H), 8.63 (s, 1H), 8.31 (s, 1H), |
| | 8.16 (s, 1H), 7.96 (d, J = 7.9 Hz, 1H), 7.61 (d, J = 9.5 Hz, 1H), |
| | 7.30 (dd, J = 8.1, 1.7 Hz, 1H), 7.27 (d, J = 1.5 Hz, 1H), 7.01 |
| | (dd, J = 9.5, 1.2 Hz, 1H), 4.67 (br d, J = 12.2 Hz, 1H), 4.58 (br |
| | d, J = 12.8 Hz, 1H), 3.10-3.21 (m, 2H), 2.97-3.09 (m, 1H), |
| | 2.71-2.83 (m, 1H), 2.44 (s, 3H), 2.12 (br t, J = 8.4 Hz, 1H), |
| | 0.81-0.94 (m, 1H), 0.47-0.60 (m, 2H), 0.26-0.41 (m, 2H). |
| 195 | MS m/z 428.4 [M + H]⁺; ¹H NMR (500 MHz, DMSO-d₆) δ: |
| | 9.08 (s, 1H), 8.20 (s, 2H), 8.17 (d, J = 7.6 Hz, 1H), 7.95 (d, J = |
| | 8.2 Hz, 1H), 7.88 (s, 1H), 7.31-7.41 (m, 3H), 7.04 (dd, J = 7.5, |
| | 1.7 Hz, 1H), 4.64 (br d, J = 12.2 Hz, 1H), 4.49-4.59 (m, 1H), |
| | 3.04-3.11 (m, 1H), 2.92 (br dd, J = 12.7, 10.5 Hz, 1H), 2.64- |
| | 2.73 (m, 2H), 2.63 (s, 3H), 1.92-2.02 (m, 1H), 0.78-0.89 (m, |
| | 1H), 0.42-0.53 (m, 2H), 0.23-0.36 (m, 2H). |
| 197 | MS m/z 418.4 [M + H]⁺; ¹H NMR (500 MHz, DMSO-d₆) δ: |
| | 9.13 (d, J = 2.1 Hz, 1H), 9.08 (s, 1H), 8.69 (d, J = 2.1 Hz, 1H), |
| | 8.01 (d, J = 8.2 Hz, 1H), 7.45 (dd, J = 1.7, 8.1 Hz, 1H), 7.41 (d, |
| | J = 1.8 Hz, 1H), 4.59 (s, 5H), 2.78 (ddd, J = 3.1, 6.5, 10.1 Hz, |
| | 2H), 2.56-2.52 (m, 2H), 1.07 (d, J = 6.1 Hz, 6H); 2Hs not |
| | observed (NH and OH). |
| 198 | MS m/z 418.4 [M + H]⁺; ¹H NMR (500 MHz, DMSO-d₆) δ: |
| | 9.57 (s, 1H), 9.06 (s, 1H), 8.47 (s, 1H), 7.97 (d, J = 8.2 Hz, |
| | 1H), 7.90-7.87 (m, 1H), 7.80-7.77 (m, 1H), 4.65-4.57 (m, 5H), |
| | 2.78 (dd, J = 3.1, 6.3 Hz, 2H), 2.58-2.52 (m, 2H), 1.11-1.03 (m, |
| | 6H); 2Hs not observed (NH and OH). |
| 201 | MS m/z 418.2 [M + H]⁺; ¹H NMR (500 MHz, DMSO-d₆) δ: |
| | 9.06 (s, 1H), 8.53 (d, J = 9.0 Hz, 1H), 8.12 (d, J = 8.9 Hz, 1H), |
| | 8.01 (d, J = 8.2 Hz, 1H), 7.90-7.86 (m, 1H), 7.82-7.77 (m, 1H), |
| | 4.70-4.60 (m, 5H), 2.91-2.78 (m, 2H), 2.63-2.54 (m, 2H), 1.12- |
| | 1.07 (m, 6H); 2Hs not observed (NH and OH). |
| 202 | MS m/z 430.4 [M + H]⁺; ¹H NMR (500 MHz, DMSO-d₆) δ: |
| | 11.15 (s, 1H), 9.06 (s, 1H), 8.52 (d, J = 9.0 Hz, 1H), 8.11 (d, |
| | J = 9.0 Hz, 1H), 8.00 (d, J = 8.5 Hz, 1H), 7.87 (s, 1H), 7.78 (d, |
| | J = 9.5 Hz,1H), 4.65 (d, J = 9.5 Hz, 1H), 4.56 (s, 3H), 4.55 (d, |
| | J = 12.0 Hz, 1H), 4.15-4.03 (m, 1H), 3.07 (t, J = 11.5 Hz, 2H), |
| | 2.92 (t, J = 11.0 Hz, 1H), 2.71-2.65 (m, 1H), 1.97 (t, J = 8.5 |
| | Hz, 1H), 0.85-0.78 (m, 1H), 0.51-0.43 (m, 2H), 0.32-0.24 (m, |
| | 2H). |
| 206 | MS m/z 417.2 [M + H]⁺; ¹H NMR (500 MHz, DMSO-d₆) δ: |
| | 9.32 (s, 1H), 9.08 (s, 1H), 8.03-7.97 (m, 2H), 7.68 (d, J = 9.3 |
| | Hz, 1H), 7.45 (dd, J = 1.6, 8.2 Hz, 1H), 7.39 (d, J = 1.5 Hz, |
| | 1H), 4.60 (d, J = 11.9 Hz, 2H), 2.77 (ddd, J = 3.1, 6.4, 10.0 Hz, |
| | 2H), 2.58 (s, 3H), 1.09-1.03 (m, 7H); 3Hs not observed (NH, |
| | OH and 1 CH overlapped with solvent peak). |
| 211 | MS m/z 446.4 [M + H]⁺; ¹H NMR (500 MHz, DMSO-d₆) δ: |
| | 11.22 (s, 1H), 9.33 (d, J = 9.0 Hz, 1H), 9.15 (s, 1H), 9.12 (s, |
| | 1H), 8.92 (d, J = 9.0 Hz, 1H), 8.68 (s, 1H), 8.01 (d, J = 8.0 Hz, |
| | 1H), 7.46 (d, J = 8.0 Hz, 1H), 7.45 (s, 1H), 4.95 (d, J = 13.5 |
| | Hz, 1H), 4.83 (d, J = 14.0 Hz, 1H), 4.58 (s, 3H), 3.49 (t, J = |
| | 12.5 Hz, 2H), 3.27-3.18 (m, 3H), 1.10 (s, 9H); 1H from HCl |
| | salt. |
| 212 | MS m/z 432.3 [M + H]⁺; ¹H NMR (500 MHz, DMSO-d₆) δ: |
| | 11.42 (s, 1H), 9.14 (s, 1H), 9.08 (s, 1H), 8.67 (d, J = 2.0 Hz, |
| | 1H), 8.00 (d, J = 2.0 Hz, 1H), 7.47 (d, J = 7.5 Hz, 1H), 7.42 (s, |
| | 1H), 4.91-4.48 (m 2H), 4.61 (s, 3H), 3.54-3.46 (m, 3H), 3.11 (t, |
| | J = 11.5 Hz, 1H), 2.70 (t, J = 9.0 Hz, 1H), 2.44-2.35 (m, 1H), |
| | 1.09-1.02 (m, 1H), 0.99 (d, J = 6.0 Hz, 6H). |
| 214 | MS m/z 432.4 [M + H]⁺; ¹H NMR (500 MHz, DMSO-d₆) δ: |
| | 11.27 (s, 1H), 9.61 (s, 1H), 9.49 (s, 1H), 9.14 (s, 1H), 9.13 (d, |
| | J = 8.5 Hz, 1H), 8.68 (s, 1H), 8.01 (d, J = 8.5 Hz, 1H), 4.93 (d, |
| | J = 12.5 Hz, 1H), 4.85 (d, J = 12.5 Hz, 1H), 4.58 (s, 3H), 3.39- |
| | 3.37 (m, 1H), 3.25-3.13 (m, 4H), 1.87-1.79 (m, 1H), 1.75-1.68 |
| | (m, 1H), 1.39 (d, J = 6.0 Hz, 3H), 1.04 (d, J = 7.5 Hz, 3H). |
| 241 | MS m/z 444.0 [M + H]⁺; ¹H NMR (400 MHz, CDCl3) 5 : |
| | 12.46 (s, 1H), 9.10 (d, J = 2.2 Hz, 1H), 8.87 (s, 1H), 8.38 (d, |
| | J = 2.2 Hz, 1H), 7.77 (d, J = 8.3 Hz, 1H), 7.39 (d, J = 1.9 Hz, |
| | 1H), 7.27-7.24 (m, 1H), 4.78-4.70 (m, 2H), 4.60 (s, 3H), 3.23- |
| | 3.18 (m, 1H), 3.11 (td, J = 11.7, 3.1 Hz, 1H), 3.03 (t, J = 12, |
| | 1H), 2.87 (td, J = 11.7, 3.1 Hz, 1H), 1.96 (dd, J = 10.8, 2.9 Hz, |
| | 1H), 1.13 (s, 3H), 0.53-0.46 (m, 1H), 0.43-0.32 (m, 3H); 1H |
| | not observed (NH or OH). |
| 242 | MS m/z 444.0 [M + H]⁺; ¹H NMR (400 MHz, CDCl₃) δ: 12.46 |
| | (s, 1H), 9.10 (d, J = 2.2 Hz, 1H), 8.87 (s, 1H), 8.38 (d, J = 2.2 |
| | Hz, 1H), 7.77 (d, J = 8.3 Hz, 1H), 7.39 (d, J = 1.9 Hz, 1H), |
| | 7.27-7.24 (m, 1H), 4.78-4.70 (m, 2H), 4.60 (s, 3H), 3.23-3.18 |
| | (m, 1H), 3.11 (td, J = 11.7, 3.1 Hz, 1H), 3.03 (t, J = 12 Hz, |
| | 1H), 2.87 (td, J = 11.7, 3.1 Hz, 1H), 1.96 (dd, J = 10.8, 2.9 Hz, |
| | 1H), 1.13 (s, 3H), 0.53-0.46 (m, 1H), 0.43-0.32 (m, 3H); 1H |
| | not observed (NH or OH). |
| 243 | MS m/z 417.5 [M + H]⁺; ¹H NMR (500 MHz, DMSO-d₆) δ: |
| | 11.09 (s, 1H), 9.22 (dd, J = 7.0, 1.7 Hz, 1H), 9.15 (s, 1H), 8.94- |
| | 8.86 (m, 1H), 8.81 (s, 1H), 8.78 (s, 1H), 8.73-8.72 (m, 1H), |
| | 7.96-7.92 (m, 2H), 7.74 (d, J = 8.2, 1H), 7.17 (dd, J = 7.1, 4.1 |
| | Hz, 1H), 4.85 (d, J = 12.5 Hz, 1H), 4.78 (d, J = 13.9 Hz, 1H), |
| | 3.56-3.26 (m, 2H), 3.22-3.18 (m, 3H), 1.98 (q, J = 6.7 Hz, 1H), |
| | 1.07 (dd, J = 6.9, 3.9 Hz, 6H); 1H from HCl salt. |
| 244 | MS m/z 416.4 [M + H]⁺; ¹H NMR (500 MHz, DMSO-d₆) δ: |
| | 11.21 (s, 1H), 9.06 (s, 1H), 8.77 (d, J = 7.0 Hz, 1H), 8.43 (s, |
| | 1H), 8.01 (d, J = 9.0 Hz, 1H), 7.94 (d, J = 8.5 Hz, 1H), 7.39 |
| | (dd, J = 9.0, 6.8 Hz, 1H), 7.33-7.32 (m, 2H), 7.00 (t, J = 6.9 |
| | Hz, 1H), 4.64 (d, J = 11.4 Hz, 1H), 4.54 (d, J = 12.6 Hz, 1H), |
| | 3.21-2.90 (m, 2H), 2.86-2.61 (m, 2H), 2.37-2.33 (m, 2H), 1.72- |
| | 1.62 (m, 1H), 0.98 (d, J = 6.8 Hz, 5H); 1 H not observed (NH |
| | or OH). |
| 263 | MS m/z 434.4 [M + H]⁺; ¹H NMR (500 MHz, DMSO-d₆) δ: |
| | 11.16 (br s, 1H), 9.16 (s, 1H), 9.04 (br s, 2H), 8.37 (s, 1H), |
| | 8.23 (d, J = 9.1 Hz, 1H), 8.09 (d, J = 9.7 Hz, 1H), 8.03 (d, J = |
| | 8.1 Hz, 1H), 7.51 (d, J = 8.2 Hz, 1H), 7.47 (s, 1H), 4.87 (d, J = |
| | 13.7 Hz, 1H), 4.80 (d, J = 14.1 Hz, 1H), 3.51-3.44 (m, 2H), |
| | 3.31-3.00 (m, 3H), 2.07-1.97 (m, 1H), 1.08 (dd, J = 6.8, 4.6 Hz, |
| | 6H); 1H from HCl salt. |
| 264 | MS m/z 435.4 [M + H]⁺; ¹H NMR (500 MHz, DMSO-d₆) δ: |
| | 11.29 (br s, 1H), 9.52 (d, J = 2.3 Hz, 1H), 9.44 (br s, 1H), 9.25 |
| | (br s, 1H), 9.17 (s, 1H), 8.82 (d, J = 2.3 Hz, 1H), 8.06 (d, J = |
| | 8.1 Hz, 1H), 7.59 (d, J = 8.0 Hz, 1H), 7.56 (s, 1H), 4.87 (d, J = |
| | 14.2 Hz, 1H), 4.79 (d, , J = 14.3 Hz, 1H), 3.61-3.39 (m, 3H), |
| | 3.29 (dd, J = 14.0, 11.1 Hz, 1H), 3.16-3.13 (m, 1H), 2.06 (h, |
| | J = 6.8 Hz, 1H), 1.09 (t, J = 6.0 Hz, 6H); 1H from HCl salt. |

### Example 3

### Preparation of Compound 142

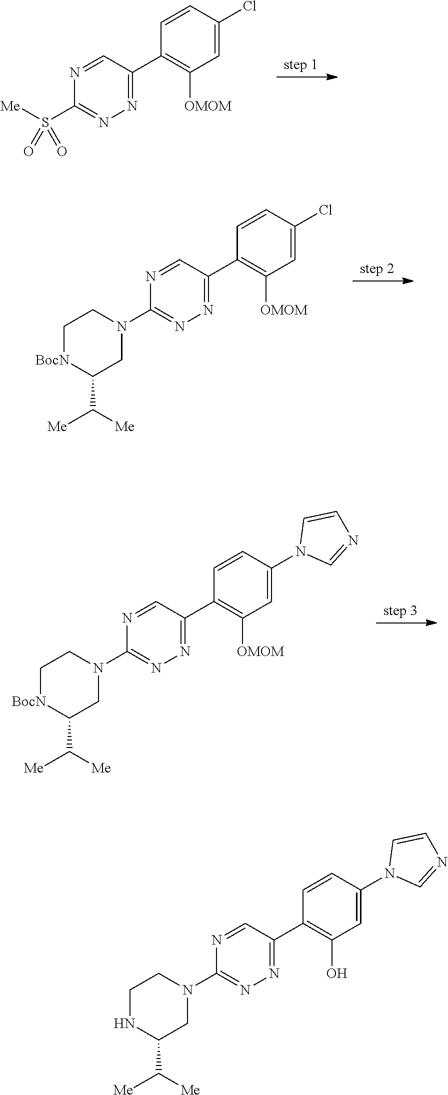

Step 1: To a solution of 6-(4-chloro-2-(methoxymethoxy)phenyl)-3-(methylsulfonyl)-1,2,4-triazine (1.0 g, 3.2 mmol) and (S)-1-Boc-2-Isopropylpiperazine (873 mg, 3.81 mmol) in ACN (6 mL) was added DIPEA (1.1 mL, 6.4 mmol). The mixture was heated at 70 for 3 h until UPLC showed complete consumption of the starting material. The reaction mixture was then cooled to room temperature, concentrated and purified by silica gel column chromatography eluting with a gradient CH₂Cl₂/MeOH (0-15% MeOH) to afford tert-butyl (S)-4-(6-(4-chloro-2-(methoxymethoxy)phenyl)-1,2,4-triazin-3-yl)-2-isopropylpiperazine-1-carboxylate (1.02 g, 67% yield) as an off white foam. MS m/z 478.4 [M+H]⁺.

Step 2: A mixture of Pd₂(dba)₃ (10.0 mg, 0.01 mmol), Me4tButylXphos (10 mg, 0.01 mmol), 1,4-dioxane (0.2 mL) and toluene (0.8 mL) was purged with Ar and then heated at 120° C. for 10 minutes. The reaction was cooled down to rt and then transferred into the vial containing tert-butyl (S)-4-(6-(4-chloro-2-(methoxymethoxy)phenyl)-1,2,4-triazin-3-yl)-2-isopropylpiperazine-1-carboxylate (100 mg, 0.21 mmol), K₃PO₄ (90 mg, 0.42 mmol) and imidazole (28 mg, 0.42 mmol). The combined mixture was then purged with Ar and then heated at 120° C. for 4 h. The reaction mixture was then cooled to room temperature, concentrated and purified by silica gel column chromatography eluting with a gradient CH₂Cl₂/MeOH (0-10% MeOH) to afford tert-butyl (S)-4-(6-(4-(1H-imidazol-1-yl)-2-(methoxymethoxy)phenyl)-1,2,4-triazin-3-yl)-2-isopropylpiperazine-1-carboxylate (76 mg, 71% yield) as a yellow solid. MS m/z 510.4 [M+H]⁺.

Step 3: To a solution of tert-butyl (S)-4-(6-(4-(1H-imidazol-1-yl)-2-(methoxymethoxy)phenyl)-1,2,4-triazin-3-yl)-2-isopropylpiperazine-1-carboxylate (76 mg, 0.15 mmol) in methanol (2 mL) was added HCl (4 mol/L) in 1,4-dioxane (0.2 mL, 0.8 mmol). The reaction was stirred at room temperature for 2 h, concentrated and purified by silica gel column chromatography, eluting with a gradient CH₂Cl₂/MeOH/NH₄OH (10-30% MeOH/NH₄OH) to afford (S)-5-(1H-imidazol-1-yl)-2-(3-(3-isopropylpiperazin-1-yl)-1,2,4-triazin-6-yl)phenol (30 mg, 47% yield).

MS m/z 366.3 [M+H]⁺; ¹H NMR (500 MHz, methanol-d₄) δ: 9.14 (s, 1H), 8.22 (s, 1H), 8.01 (d, J=8.4 Hz, 1H), 7.61 (s, 1H), 7.22-7.17 (m, 3H), 5.08 (br d, J=13.6 Hz, 1H), 5.02-4.93 (m, 1H), 3.59-3.45 (m, 2H), 3.31-3.23 (m, 2H), 3.18-3.11 (m, 1H), 2.11-2.02 (m, 1H), 1.19 (d, J=6.9 Hz, 6H); 2Hs not observed (NH and OH).

Using the procedure described for Example 3, above, additional compounds described herein may be prepared by substituting the appropriate starting material, suitable reagents and reaction conditions, obtaining compounds such as those selected from:

| Cpd | Data |
|---|---|
| 159 | MS m/z 430.5 [M + H]⁺; ¹H NMR (500 MHz, methanol-d₄) δ: |
| | 9.18 (s, 1H), 8.35 (br s, 1H), 8.07 (d, J = 8.5 Hz, 1H), 8.04 (d, |
| | J = 8.5 Hz, 1H), 7.86 (d, J = 3.4 Hz, 1H), 7.19-7.25 (m, 2H), |
| | 6.79 (d, J = 3.4 Hz, 1H), 5.07 (br d, J = 13.7 Hz, 1H), 4.97 (br |
| | d, J = 14.3 Hz, 1H), 3.49-3.55 (m, 1H), 3.40-3.48 (m, 1H), |
| | 3.20-3.30 (m, 2H), 3.11 (ddd, J = 10.6, 7.4, 3.1 Hz, 1H), 2.67 |
| | (s, 3H), 1.96-2.08 (m, 1H), 1.18 (d, J = 7.0 Hz, 6H); 2Hs not |
| | observed (NH and OH). |
| 54 | MS m/z 353.3 [M + H]⁺; ¹H NMR (500 MHz, methanol-d₄) δ: |
| | 9.13 (s, 1 H), 8.01 (s, 1 H), 7.85 (s, 1 H), 7.84 (d, J = 8.09 Hz, |
| | 1 H), 7.20 (d, J = 6.56 Hz, 1 H), 7.16 (d, J = 1.53 Hz, 1H), 5.05 |
| | (dd, J = 14.34, 2.75 Hz, 2H), 3.48 (m, 2H), 3.04 (dd, J = 14.50, |
| | 11.60 Hz, 2H), 1.44 (d, J = 6.56 Hz, 6H); 2Hs not observed |
| | (NH and OH). |
| 52 | MS m/z 367.4 [M + H]⁺; ¹H NMR (500 MHz, methanol-d₄) δ: |
| | 9.08 (s, 1H), 7.97 (d, J = 9.3 Hz, 1H), 7.96 (s, 2H), 7.70-7.73 |
| | (m, 2H), 4.69 (br d, J = 14.6 Hz, 2H), 2.90 (dd, J = 13.4, 11.1 |
| | Hz, 2H), 2.34-2.41 (m, 5H), 1.26 (s, 3H), 1.25 (s, 3H); 1H not |
| | observed (OH). |
| 45 | MS m/z 355.3 [M + H]⁺; ¹H NMR (500 MHz, methanol-d₄) δ: |
| | 9.08 (s, 1H), 7.97 (d, J = 9.0 Hz, 1H), 7.96 (s, 2H), 7.71 (s, |
| | 2H), 4.79 (br d, J = 11.4 Hz, 1H), 4.67 (d, J = 12.8 Hz, 1H), |
| | 3.57-3.69 (m, 2H), 3.14-3.22 (m, 2H), 2.85-2.96 (m, 3H); 3Hs |
| | not observed (NH and 2 OHs). |
| 274 | MS m/z 436.5 [M + H]⁺; ¹H NMR (500 MHz, methanol-d₄) δ: |
| | 9.05 (s, 1H), 8.45-8.60 (s, 1H), 7.73-7.83 (m, 1H), 6.70-6.79 |
| | (m, 1H), 6.59-6.66 (m, 1H), 4.89-4.95 (m, 1H), 4.74-4.82 (m, |
| | 1H), 4.56 (s, 2H), 4.21-4.29 (m, 2H), 3.88-3.97 (m, 2H), 3.34- |
| | 3.38 (m, 1H), 3.21-3.30 (m, 1H), 2.97-3.10 (m, 2H), 2.74-2.86 |
| | (m, 1H), 2.38 (s, 3H), 1.82-1.93 (m, 1H), 1.05-1.18 (m, 6H); |
| | 2Hs not observed (NH and OH). |
| 155 | MS m/z 431.5 [M + H]⁺; ¹H NMR (500 MHz, DMSO-d₆) δ: |
| | 9.59 (br d, J = 9.5 Hz, 1H), 9.34 (br d, J = 8.9 Hz, 1H), 9.08 (s, |
| | 1H), 8.60 (s, 1H), 8.52 (d, J = 8.9 Hz, 1H), 8.04 (d, J = 8.5 Hz, |
| | 1H), 7.59 (d, J = 9.2 Hz, 1H), 7.56 (d, J = 2.1 Hz, 1H), 7.47 |
| | (dd, J = 8.5, 2.1 Hz, 1H), 4.86 (br d, J = 13.4 Hz, 1H), 4.78 (br |
| | d, J = 14.0 Hz, 1H), 3.47-3.56 (m, 1H), 3.39-3.45 (m, 1H), 3.29 |
| | (dd, J = 14.0, 11.3 Hz, 1H), 3.09-3.18 (m, 2H), 2.73 (s, 3H), |
| | 2.01-2.12 (m, 1H), 1.04-1.12 (m,6H). |
| 237 | MS m/z 393.3 [M + H]⁺; ¹H NMR (500 MHz, DMSO-d₆) δ: |
| | 11.30 (s, 1H), 8.98 (s, 1H), 8.14 (s, 1H), 7.99 (d, J = 8.5 Hz, |
| | 1H), 7.68 (s, 1H), 7.63 (d, J = 8.5 Hz, 1H), 4.69-4.51 (m, 2H), |
| | 2.76-2.44 (m, 4H), 2.29-2.21 (m, 1H), 2.04-1.98 (m, 2H), 1.91- |
| | 1.76 (m, 4H), 1.09 (d, J = 5.0 Hz, 3H); 2Hs not observed (NH |
| | and OH). |
| 162 | MS m/z 418.5 [M + H]⁺; ¹H NMR (500 MHz, DMSO-d₆) δ: |
| | 9.76 (br, d, J = 9.46 Hz, 1H), 9.56 (s, 1H), 9.46 (br, d, J = 9.54 |
| | Hz, 1H), 9.25 (s, 1H), 9.08 (s, 1H), 8.76 (s, 1H), 8.03-8.06 (m, |
| | 3H), 7.93 (dd, J = 8.7, 1.98 Hz, 1H), 4.77 (br d, J = 14.04 Hz, |
| | 2H), 4.85 (br, d, J = 13.43 Hz, 1H), 3.52-3.57 (m, 1H), 3.38- |
| | 3.41 (m, 1H), 3.27-3.30 (m, 2H), 2.05-2.12 (m, 1H), 1.07-1.10 |
| | (t, J = 6.82 Hz, 6H); 1H from HCl salt. |
| 183 | MS m/z 379.5 [M + H]⁺; ¹H NMR (500 MHz, DMSO-d₆) δ: |
| | 9.77 (br s, 1H), 9.42-9.43 (m, 1H), 9.05 (s, 1H), 7.98 (d, J = |
| | 8.54 Hz, 1H), 7.92 (s, 1H), 7.68 (d, J = 2.14 Hz, 1H), 7.58 (dd, |
| | J = 8.54, 2.14 Hz, 1H), 4.68-4.77 (m, 2H), 3.41-3.58 (m, 2H), |
| | 3.03-3.10 (m, 1H), 2.65-2.69 (m, 1H), 2.38 (s, 2H), 2.27 (s, |
| | 3H), 1.05-1.14 (m, 1H), 0.60-0.68 (m, 2H), 0.42-0.44 (m, 1H). |
| 184 | MS m/z 381.4 [M + H]⁺; ¹H NMR (500 MHz, DMSO-d₆) δ: |
| | 9.45 (s, 1H), 9.27 (s, 1H), 9.05 (s, 1H), 7.98 (d, J = 8.54 Hz, |
| | 1H), 7.92 (s, 1H), 7.68-7.70 (m, 1H), 7.58 (dd, J = 8.54, 2.14 |
| | Hz, 1H), 4.76-4.86 (m, 2H) 3.44-3.53 (m, 3H), 3.39-3.42 (m, |
| | 1H), 3.24-3.29 (m, 1H), 2.38 (s, 3H), 2.02-2.27 (m, 1H), 1.06- |
| | 1.09 (m, 6H). |
| 11 | MS m/z 365.3 [M + H]⁺; ¹H NMR (500 MHz, methanol-d₄) δ: |
| | 9.08 (s, 1H), 7.99 (d, J = 9.2 Hz, 1H), 7.96 (s, 2H), 7.67-7.74 |
| | (m, 2H), 4.82 (br d, J = 13.5 Hz, 1H), 4.71 (d, J = 13.6 Hz, |
| | 1H), 3.11-3.23 (m, 2H), 3.01 (dd, J = 12.9, 10.2 Hz, 1H), 2.82 |
| | (td, J = 12.9, 3.5 Hz, 1H), 1.96 (td, J = 10.2, 3.5 Hz, 1H), 0.83- |
| | 0.96 (m, 1H), 0.58-0.65 (m, 2H), 0.31-0.42 (m, 2H); 2Hs not |
| | observed (NH and OH). |
| 14 | MS m/z 383.5 [M + H]⁺; ¹H NMR (500 MHz, methanol-d₄) δ: |
| | 9.09 (s, 1H), 8.00 (d, J = 9.0 Hz, 1H), 7.96 (s, 2H), 7.70-7.75 |
| | (m, 2H), 4.95 (d, J = 13.5 Hz, 1H), 4.79 (d, J = 13.5 Hz, 1H), |
| | 3.17-3.27 (m, 1H), 3.11 (td, J = 13.2, 3.7 Hz, 1H), 2.86-2.96 |
| | (m, 2H), 2.64-2.75 (m, 1H), 1.32 (d, J = 13.0 Hz, 6H); 3Hs not |
| | observed (NH and 2OHs). |
| 18 | MS m/z 381.5 [M + H]⁺; ¹H NMR (500 MHz, methanol-d₄) δ: |
| | 9.06 (s, 1H), 7.89-8.03 (m, 3H), 7.66 (s, 2H), 4.91 (d, J = 12.7 |
| | Hz, 1H), 4.74 (br d, J = 12.7 Hz, 1H), 3.57-3.80 (m, 1H), 3.00- |
| | 3.22 (m, 2H), 2.85 (br t, J = 11.1 Hz, 1H), 2.45 (d, J = 11.1 Hz, |
| | 1H), 1.06 (s, 9H); 2Hs not observed (NH and OH). |
| 132 | MS m/z 353.3 [M + H]⁺; ¹H NMR (500 MHz, methanol-d₄) δ: |
| | 9.09 (s, 1H), 7.99 (d, J = 9.0 Hz, 1H), 7.96 (s, 2H), 7.69-7.74 |
| | (m, 2H), 4.81 (br d, J = 12.8 Hz, 1H), 4.74 (d, J = 12.9 Hz, |
| | 1H), 3.16-3.25 (m, 2H), 2.90-2.97 (m, 1H), 2.83-2.89 (m, 1H), |
| | 2.72-2.81 (m, 1H), 1.59 (quin, J = 7.4 Hz, 2H), 1.08 (t, J = 7.4 |
| | Hz, 3H); 2Hs not observed (NH and OH). |
| 153 | MS m/z 365.3 [M + H]⁺; ¹H NMR (500 MHz, methanol-d₄) δ: |
| | 9.09 (s, 1H), 7.98-8.02 (m, 1H), 7.96 (s, 2H), 7.68-7.75 (m, |
| | 2H), 4.93 (d, J = 11.1 Hz, 1H), 4.84 (d, J = 14.3 Hz, 1H), 3.14- |
| | 3.28 (m, 3H), 2.85-2.93 (m, 1H), 2.29 (d, J = 9.0 Hz, 2H), |
| | 2.13-2.23 (m, 1H), 1.83-2.05 (m, 3H), 1.51-1.63 (m, 1H); 1H |
| | not observed (OH). |
| 154 | MS m/z 367.3 [M + H]⁺; ¹H NMR (500 MHz, methanol-d₄) δ: |
| | 9.17 (s, 1H), 7.99-8.05 (m, 1H), 7.97 (s, 2H), 7.73-7.76 (m, |
| | 2H), 5.04 (d, J = 14.6 Hz, 1H), 4.94 (d, J = 11.4 Hz, 1H), 3.50 |
| | (d, J = 12.2 Hz, 1H), 3.46 (br d, J = 9.6 Hz, 1H), 3.21-3.29 (m, |
| | 2H), 3.12-3.20 (m, 1H), 1.96-2.08 (m, 1H), 1.18 (d, J = 6.9 Hz, |
| | 6H); 2Hs not observed (NH and OH). |

### Example 4

### Preparation of Compound 180

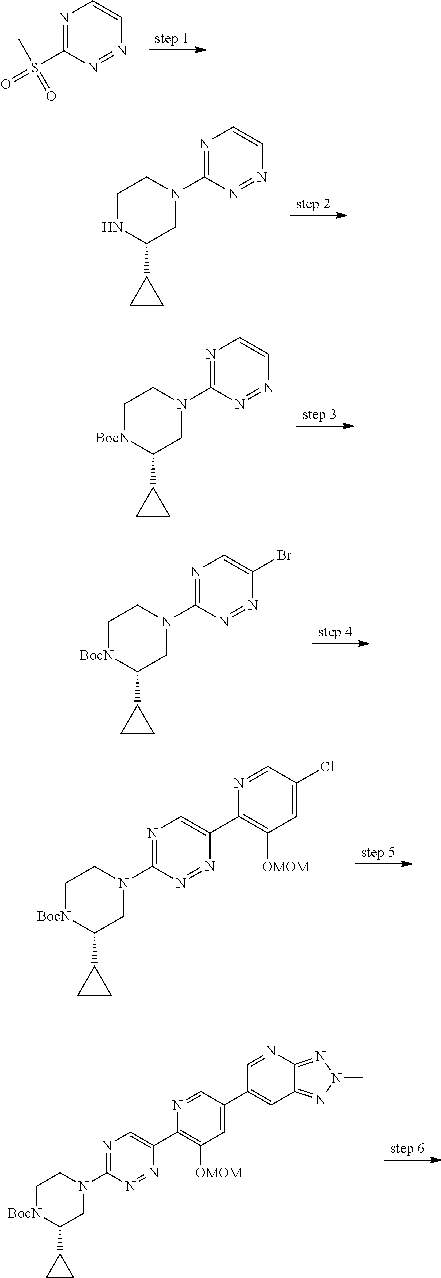

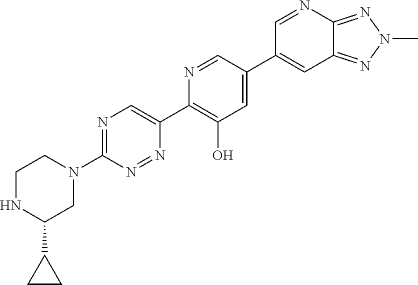 [SMILES: Cn1nc2cc(-c3cnc(-c4cnc(N5CCN[C@@H](C6CC6)C5)nn4)c(O)c3)cnc2n1]

Step 1: To a flask containing a stirbar 3-(methylsulfonyl)-1,2,4-triazine (3.3 g, 21.0 mmol, 1.0 equiv.) and the HCl salt of (S)-2-cyclopropylpiperazine (5.0 g, 25.1 mmol, 1.2 equiv.) was added dmf (100 mL) followed by DIPEA (11 mL, 63.0 mmol, 3.0 equiv.) and was allowed to stir for 12 h at 22° C. After, the solvent was removed and the crude oil was purified by silica gel chromatography using a gradient from 100% CH₂Cl₂ up to 20% MeOH/CH₂Cl₂ to afford (S)-3-(3-cyclopropylpiperazin-1-yl)-1,2,4-triazine (4.1 g, 20.1 mmol, 96% yield) as a brown solid. MS m/z 206.3 [M+H]⁺.

Step 2: To a flask containing (S)-3-(3-cyclopropylpiperazin-1-yl)-1,2,4-triazine (4.1 g, 20.1 mmol, 1.0 equiv.) was added Boc₂O (6.0 g, 27.0 mmol, 1.3 equiv.), NEt₃ (8.6 mL, 68.0 mmol, 3.0 equiv.) and CH₂Cl₂ (100 mL). Next, DMAP (0.5 g, 4.0 mmol, 0.2 equiv.) was added and the mixture was allowed to stir at 22° C. for 12 h. The solvent was removed and the crude oil was purified by silica gel chromatography using a gradient from 100% hexanes up to 50% EtOAc/hexanes to afford tert-butyl (S)-2-cyclopropyl-4-(1,2,4-triazin-3-yl)piperazine-1-carboxylate (3.4 g, 11.3 mmol, 55% yield) as an oil. MS m/z 306.3 [M+H]⁺.

Step 3: To a flask containing tert-butyl (S)-2-cyclopropyl-4-(1,2,4-triazin-3-yl)piperazine-1-carboxylate (3.4 g, 11.3 mmol, 1.0 equiv.) was added acetonitrile (90 mL) and water (30 mL) and was added NBS (2.4 g, 13.0 mmol, 1.2 equiv.) and the mixture was allowed to stir for 12 h at 22° C. Next, the mixture was diluted with EtOAc and water. The organic layer was separated, dried over MgSO₄, filtered and concentrated. Purified by silica gel chromatography using a gradient from 100% hexanes up to 50% EtOAc/hexanes to afford tert-butyl (S)-4-(6-bromo-1,2,4-triazin-3-yl)-2-cyclopropylpiperazine-1-carboxylate (2.9 g, 7.5 mmol, 67% yield) as an orange oil. MS m/z 384.1, 386.1 [M+H]⁺.

Step 4: To a flask containing tert-butyl (S)-4-(6-bromo-1,2,4-triazin-3-yl)-2-cyclopropylpiperazine-1-carboxylate (1.0 g, 2.6 mmol, 1.0 equiv.), tributyl(4-chloro-2-(methoxymethoxy)phenyl)stannane (1.4 g, 3.0 mmol, 1.2 equiv.), PdCl₂(PPh₃)₂ (0.2 g, 0.3 mmol, 0.1 equiv.), CuI (0.1 g, 0.5 mmol, 0.2 equiv.) was added dioxane (20 mL) and purged with Ar. The flask was allowed to stir for 2 h at 100° C. under Ar. Once cooled, the mixture was filtered and the filtrate was concentrated and purified by silica gel chromatography using a gradient from 100% hexanes up to 50% EtOAc/hexanes to afford tert-butyl (S)-4-(6-(5-chloro-3-(methoxymethoxy)pyridin-2-yl)-1,2,4-triazin-3-yl)-2-cyclopropylpiperazine-1-carboxylate (0.4 g, 0.9 mmol, 34% yield) as a yellow oil. MS m/z 477.1, 479.1 [M+H]⁺.

Step 5: To a vial containing tert-butyl (S)-4-(6-(5-chloro-3-(methoxymethoxy)pyridin-2-yl)-1,2,4-triazin-3-yl)-2-cyclopropylpiperazine-1-carboxylate (0.05 g, 0.1 mmol, 1.0 equiv.) was added (2-methyl-2H-[1,2,3]triazolo[4,5-b]pyridin-6-yl)boronic acid (0.04 g, 0.2 mmol, 1.5 equiv.), K₂CO₃ (0.05 g, 0.4 mmol, 3.0 equiv.), XPhos Pd G₃ (0.01 g, 0.01 mmol, 0.1 equiv.) dioxane (1 mL) and water (1 mL). The mixture was allowed to stir at 100° C. for 1 h. Once cooled, the mixture was purified by silica gel chromatography using a gradient from 100% hexanes to 50% EtOAc/hexanes to afford tert-butyl (S)-2-cyclopropyl-4-(6-(3-(methoxymethoxy)-5-(2-methyl-2H-[1,2,3]triazolo[4,5-b]pyridin-6-yl)pyridin-2-yl)-1,2,4-triazin-3-yl)piperazine-1-carboxylate (0.02 g, 0.03 mmol, 28% yield) as a yellow solid. MS m/z 475.4 [M+H]⁺.

Step 6: To a vial containing tert-butyl (S)-2-cyclopropyl-4-(6-(3-(methoxymethoxy)-5-(2-methyl-2H-[1,2,3]triazolo[4,5-b]pyridin-6-yl)pyridin-2-yl)-1,2,4-triazin-3-yl)piperazine-1-carboxylate (0.02 g, 0.03 mmol, 1.0 equiv.) was dissolved in MeOH (1 mL) and was added 4.0 M HCl/dioxane. The mixture was allowed to stir for 1 h at 22° C. Next, the mixture was concentrated and purified by silica gel chromatography using a gradient from 100% CH₂Cl₂ up to 15% MeOH/CH₂Cl₂ to afford the HCl salt of (S)-2-(3-(3-cyclopropylpiperazin-1-yl)-1,2,4-triazin-6-yl)-5-(2-methyl-2H-[1,2,3]triazolo[4,5-b]pyridin-6-yl)pyridin-3-ol (0.01 g, 0.02 mmol, 58% yield) as a white solid upon drying. MS m/z 431.4 [M+H]⁺; ¹H NMR (500 MHz, DMSO-d₆) δ: 12.02 (s, 1H), 9.45 (s, 1H), 9.25 (s, 1H), 9.16 (s, 1H), 8.90 (s, 1H), 8.78 (s, 1H), 7.99 (s, 1H), 4.77 (d, J=14.0 Hz, 1H), 4.72 (d, J=15.0 Hz, 1H), 4.60 (s, 3H), 3.54-3.46 (m, 3H), 3.11 (t, J=11.5 Hz, 1H), 2.70 (t, J=9.0 Hz, 1H), 1.09-1.02 (m, 1H), 0.72-0.64 (m, 2H), 0.57-0.54 (m, 1H), 0.46-0.43 (m, 1H).

Using the procedure described for Example 4, above, additional compounds described herein may be prepared by substituting the appropriate starting material, suitable reagents and reaction conditions, obtaining compounds such as those selected from:

| Cpd | Data |
|---|---|
| 181 | MS m/z 447.4 [M + H]⁺; ¹H NMR (500 MHz, DMSO-d₆) δ: |
| | 12.02 (s, 1H), 11.13 (s, 1H), 9.36 (s, 1H), 8.65 (d, J = 2.0 Hz, |
| | 1H), 8.58 (d, J = 2.0 Hz, 1H), 8.03 (d, J = 1.0 Hz, 1H), 7.75 (d, |
| | J = 2.0 Hz, 1H), 7.55 (d, J = 14.5 Hz, 1H), 4.68 (d, J = 12.0 Hz, |
| | 1H), 4.60 (d, J = 12.5 Hz, 1H), 4.23 (s, 3H), 3.25-3.08 (m, 3H), |
| | 2.79 (t, J = 11.5 Hz, 1H), 2.23-2.12 (m, 1H), 0.93-0.85 (m, |
| | 1H), 0.56-0.48 (m, 2H), 0.39-0.36 (m, 1H), 0.33-0.30 (m, 1H). |
| 157 | MS m/z 449.5 [M + H]⁺; ¹H (500 MHz, DMSO-d₆) δ: 12.07 (s, |
| | 1H), 9.34 (s, 1H), 8.64 (d, J = 2.0 Hz, 1H), 8.58 (d, J = 2.0 Hz, |
| | 1H), 8.03 (s, 1H), 7.77 (d, J = 2.0 Hz, 1H), 7.55 (d, J = 14.0 |
| | Hz, 1H), 4.67-4.55 (m, 2H), 4.23 (s, 3H), 3.05 (t, J = 11.5 Hz, |
| | 2H), 2.81 (t, J = 11.5 Hz, 1H), 2.70 (t, J = 12.0 Hz, 1H), 2.43- |
| | 2.36 (m, 1H), 1.70-1.63 (m, 1H), 0.97 (d, J = 7.0 Hz, 6H); 1H |
| | not observed (NH or OH). |
| 121 | MS m/z 433.4 [M + H]⁺; ¹H NMR (500 MHz, DMSO-d₆) δ: |
| | 12.04 (s, 1H), 9.35 (s, 1H), 8.65 (s, 1H), 8.38 (s, 1H), 7.77 (s, |
| | 1H), 7.69 (s, 1H), 6.98 (s, 1H), 4.17 (s, 3H), 4.02 (s, 3H), 3.90 |
| | (t, J = 5.0 Hz, 4H), 2.45 (t, J = 5.0 Hz, 4H), 2.25 (s, 3H). |
| 122 | MS m/z 421.4 [M + H]⁺; ¹H NMR (500 MHz, DMSO-d₆) δ: |
| | 11.55 (s, 1H), 9.41 (s, 1H), 8.98 (s, 1H), 8.66 (s, 1H), 7.85 (s, |
| | 1H), 7.84 (d, J = 12.0 Hz, 1H), 7.82 (s, 1H), 4.80 (m, 4H), 3.16 |
| | (s, 3H), 2.78 (m, 4H), 2.39 (s, 3H). |
| 124 | MS m/z 339.2 [M + H]⁺; ¹H NMR (500 MHz, DMSO-d₆) δ: |
| | 13.21 (s, 1H), 12.00 (s, 1H), 9.31 (s, 1H), 8.57 (s, 1H), 8.41 (s, |
| | 1H), 8.10 (s, 1H), 7.67 (s, 1H), 3.88 (s, 2H), 3.30 (s, 2H), 2.49 |
| | (s, 3H), 2.45 (s, 2H), 2.24 (s, 2H). |
| 126 | MS m/z 417.4 [M + H]⁺; ¹H NMR (500 MHz, DMSO-d₆) δ: |
| | 12.04 (s, 1H), 11.59 (s, 1H), 9.42 (s, 1H), 9.20 (s, 1H), 8.69 (s, |
| | 1H), 7.99 (s, 1H), 7.87 (s, 1H), 4.87-4.76 (m, 2H), 3.66-3.48 |
| | (m, 4H), 3.22-3.10 (m, 2H), 2.79 (s, 3H), 2.64 (s, 3H), 2.52 (s, |
| | 3H). |

### Example 5

### Preparation of Compound 175

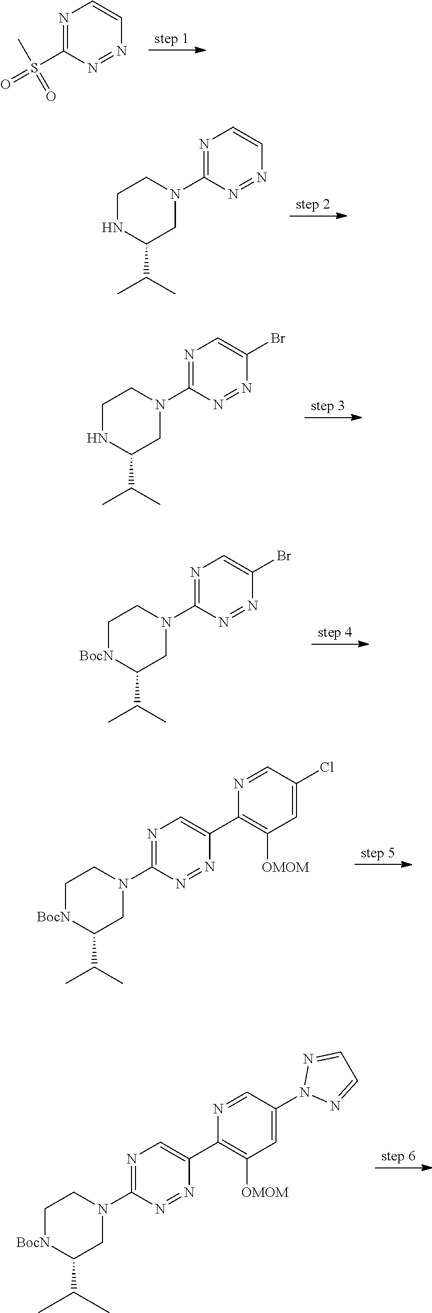

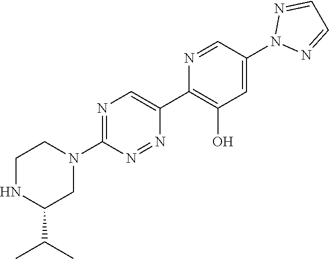 [SMILES: CC(C)[C@H]1CN(c2ncc(-c3ncc(-n4nccn4)cc3O)nn2)CCN1]

Step 1: To a solution of 3-(methylsulfonyl)-1,2,4-triazine (3.0 g, 18.8 mmol) and the HCl salt of (S)-2-isopropylpiperazine (4.5 g, 22.6 mmol) in DMF (100 mL) was added DIPEA (33 mL, 187 mmol). The reaction mixture was stirred at rt for 12 h until UPLC showed complete conversion. The solvent was removed, and the crude oil was purified by silica gel chromatography eluting with a gradient CH₂Cl₂/MeOH (0-20% MeOH) to afford (S)-3-(3-isopropylpiperazin-1-yl)-1,2,4-triazine (1.8 g, 46% yield) as a brown solid. MS m/z 208.3 [M+H]⁺.

Step 2: To a solution of (S)-3-(3-isopropylpiperazin-1-yl)-1,2,4-triazine (1.8 g, 8.7 mmol) in water (100 mL) and MeOH (25 mL) was added bromine (0.7 mL, 13.0 mmol) dropwise and the mixture was allowed to stir for 1 h. Saturated aqueous sodium thiosulfate was added followed by water and EtOAc. The aqueous layer was then extracted with EtOAc 3 times. The organic layer was washed with water, brine and dried over MgSO₄, filtered and concentrated. The crude oil was purified by silica gel chromatography eluting with a gradient CH₂Cl₂/MeOH (0-10% MeOH) to afford (S)-6-bromo-3-(3-isopropylpiperazin-1-yl)-1,2,4-triazine (1.7 g, 69% yield) as a brown solid. MS m/z 286.1, 288.1 [M+H]⁺.

Step 3: To a flask containing (S)-6-bromo-3-(3-isopropylpiperazin-1-yl)-1,2,4-triazine (1.7 g, 6.0 mmol), Et₃N (2.5 mL, 18.0 mmol) and Boc₂O (1.7 g, 7.8 mmol) was added CH₂Cl₂ (100 mL). DMAP (0.2 g, 1.2 mmol) was added and the mixture was allowed to stir at rt for 12 h. The solvent was removed, and the crude oil was purified by silica gel chromatography eluting with a gradient hexanes/EtOAc (0-100% EtOAc) to afford tert-butyl (S)-4-(6-bromo-1,2,4-triazin-3-yl)-2-isopropylpiperazine-1-carboxylate (2.1 g, 90% yield) as a white solid. MS m/z 386.1, 388.1 [M+H]⁺.

Step 4: To a vial containing tert-butyl (S)-4-(6-bromo-1,2,4-triazin-3-yl)-2-isopropylpiperazine-1-carboxylate (1.3 g, 3.4 mmol) were added tributyl(4-chloro-2-(methoxymethoxy)phenyl)stannane (1.9 g, 4.1 mmol), CuI (0.1 g, 0.7 mmol) and PdCl₂(PPh₃)₂ (0.2 g, 0.3 mmol) followed by dioxane (10 mL). The mixture was purged with Ar and stirred at 100° C. for 2 h. The crude mixture was filtered and concentrated. The residual oil was purified by silica gel chromatography eluting with a gradient hexanes/EtOAc (0-100% EtOAc) to afford tert-butyl (S)-4-(6-(5-chloro-3-(methoxymethoxy)pyridin-2-yl)-1,2,4-triazin-3-yl)-2-isopropylpiperazine-1-carboxylate (0.8 g, 51% yield) as a colorless oil. MS m/z 479.2, 481.2 [M+H]⁺.

Step 5: To a vial containing tBuXPhos (0.01 g, 0.01 mmol), Pd₂(dba)₃ (0.01 g, 0.01 mmol) was added toluene (1 mL) and the mixture was purged with Ar. The reaction was heated to 110° C. for 10 min, then cooled to rt. The catalyst solution was added to a vial containing tert-butyl (S)-4-(6-(5-chloro-3-(methoxymethoxy)pyridin-2-yl)-1,2,4-triazin-3-yl)-2-isopropylpiperazine-1-carboxylate (0.06 g, 0.1 mmol), triazole (0.01 g, 0.15 mmol), K₃PO₄ (0.05 g, 0.2 mmol) in toluene (5 mL). The reaction mixture was allowed to stir at 110° C. for 4 h. The crude mixture was filtered, concentrated and purified eluting with a gradient hexanes/EtOAc (0-50% EtOAc) to afford tert-butyl (S)-2-isopropyl-4-(6-(3-(methoxymethoxy)-5-(2H-1,2,3-triazol-2-yl)pyridin-2-yl)-1,2,4-triazin-3-yl)piperazine-1-carboxylate (0.04 g, 72% yield) as a colorless oil. MS m/z 512.5 [M+H]+

Step 6: To a vial containing tert-butyl (S)-2-isopropyl-4-(6-(3-(methoxymethoxy)-5-(2H-1,2,3-triazol-2-yl)pyridin-2-yl)-1,2,4-triazin-3-yl)piperazine-1-carboxylate (0.04 g, 0.08 mmol) and a stirbar was added MeOH (2 mL). 4.0 M HCl/dioxane (2 mL) was added and the mixture was stirred at rt for 1 h. The solvent was removed and the crude solid was purified by silica gel chromatography using a gradient CH₂Cl₂/MeOH (0-10% MeOH) to afford (S)-2-(3-(3-isopropylpiperazin-1-yl)-1,2,4-triazin-6-yl)-5-(2H-1,2,3-triazol-2-yl)pyridin-3-ol (0.02 g, 73% yield) as a white solid upon drying. MS m/z 368.3 [M+H]⁺; ¹H NMR (500 MHz, DMSO-d₆) δ: 9.26 (s, 1H), 8.90 (d, J=2.0 Hz, 1H), 8.24 (s, 2H), 7.93 (d, J=2.0 Hz, 1H), 4.65 (d, J=11.0 Hz, 1H), 4.57 (d, J=11.0 Hz, 1H), 3.05 (t, J=11.0 Hz, 2H), 2.82 (t, J=11.5 Hz, 1H), 2.70 (t, J=10.5 Hz, 1H), 2.39-2.36 (m, 1H), 1.71-1.60 (m, 1H), 0.97 (d, J=6.5 Hz, 6H); 2Hs not observed (NH and OH).

Using the procedure described for Example 5, above, additional compounds described herein may be prepared by substituting the appropriate starting material, suitable reagents and reaction conditions, obtaining compounds such as:

| Cpd | Data |
|---|---|
| 182 | MS m/z 366.3 [M + H]⁺; ¹H NMR (500 MHz, DMSO-d₆) δ: |
| | 12.14 (s, 1H), 9.34 (s, 1H), 9.09 (s, 2H), 8.94 (s, 1H), 8.26 (s, |
| | 2H), 7.97 (s, 1H), 4.77 (d, J = 14.5 Hz, 1H), 4.71 (d, J = 14.0 |
| | Hz, 1H), 3.53-3.44 (m, 3H), 3.17-3.08 (m, 1H), 2.74-2.68 (m, |
| | 1H), 1.06-1.00 (m, 1H), 0.72-0.63 (m, 2H), 0.57-0.53 (m, 1H), |
| | 0.47-0.42 (m, 1H); 1H from HCl salt. |

### Biological Examples

The following in vitro biological examples demonstrate the usefulness of the compounds of the present description for treating Huntington's disease.

To describe in more detail and assist in understanding the present description, the following non-limiting biological examples are offered to more fully illustrate the scope of the description and are not to be construed as specifically limiting the scope thereof. Such variations of the present description that may be now known or later developed, which would be within the purview of one skilled in the art to ascertain, are considered to fall within the scope of the present description and as hereinafter claimed.

Compounds of Formula (I) were tested using the Meso Scale Discovery (MSD) Assay provided in International Application No. PCT/US2016/066042, filed on Dec. 11, 2016 and claiming priority to U.S. Provisional Application 62/265,652 filed on Dec. 10, 2015, the entire contents of which are incorporated herein by reference.

The Endogenous Huntingtin Protein assay used in Biological Example 1 was developed using the ELISA-based MSD electrochemiluminescence assay platform.

### Biological Example 1

### Endogenous Huntingtin Protein Assay

Meso Scale Discovery (MSD) 96-well or 384-well plates were coated overnight at 4° C. with MW1 (expanded polyglutamine) or MAB2166 monoclonal antibody (for capture) at a concentration of 1 μg/mL in PBS (30 μL per well). Plates were then washed three times with 300 μL wash buffer (0.05% Tween-20 in PBS) and blocked (100 μL blocking buffer; 5% BSA in PBS) for 4-5 hours at room temperature with rotational shaking and then washed three times with wash buffer.

Samples (25 μL) were transferred to the antibody-coated MSD plate and incubated overnight at 4° C. After removal of the lysates, the plate was washed three times with wash buffer, and 25 μL of #56565 (Cell signaling; rabbit monoclonal) secondary antibody (diluted to 0.25 μg/mL in 0.05% Tween-20 in blocking buffer) was added to each well and incubated with shaking for 1 Hour at room temperature. Following incubation with the secondary antibody, the wells were rinsed with wash buffer after which 25 μL of goat anti-rabbit SULFO TAG secondary detection antibody (required aspect of the MSD system) (diluted to 0.25 μg/mL in 0.05% Tween-20 in blocking buffer) was added to each well and incubated with shaking for 1 hour at room temperature. After rinsing three times with wash buffer, 150 μL of read buffer T with surfactant (MSD) were added to each empty well, and the plate was imaged on a SI 6000 imager (MSD) according to manufacturers' instructions provided for 96- or 384-well plates. The resulting IC₅₀ values (nM) for compounds tested are provided in Tables 2 and 3.

TABLE 2
IC₅₀ (nM) Values for Compunds 1-284

| Cpd | IC50 (nM) |
|---|---|
| 1 | 2.1 |
| 2 | 3.9 |
| 3 | 4.4 |
| 4 | 4.5 |
| 5 | 6 |
| 6 | 6.7 |
| 7 | 6.9 |
| 8 | 8.7 |
| 9 | 9.5 |
| 10 | 9.5 |
| 11 | 9.6 |
| 12 | 10.9 |
| 13 | 11.4 |
| 14 | 12.2 |
| 15 | 12.8 |
| 16 | 13.2 |
| 17 | 14.1 |
| 18 | 15.1 |
| 19 | 17.1 |
| 20 | 17.1 |
| 21 | 20.8 |
| 22 | 22.3 |
| 23 | 22.7 |
| 24 | 23.4 |
| 25 | 24.6 |
| 26 | 24.9 |
| 27 | 25.1 |
| 28 | 26.2 |
| 29 | 28.5 |
| 30 | 33.3 |
| 31 | 34.3 |
| 32 | 40.2 |
| 33 | 41.4 |
| 34 | 42 |
| 35 | 46.1 |
| 36 | 47.7 |
| 37 | 50.7 |
| 38 | 56.2 |
| 39 | 63.7 |
| 40 | 68.2 |
| 41 | 109.8 |
| 42 | 110.9 |
| 43 | 116.2 |
| 44 | 137.7 |
| 45 | 141.5 |
| 46 | 150.3 |
| 47 | 154.5 |
| 48 | 166.9 |
| 49 | 256.1 |
| 50 | 312.2 |
| 51 | 329.9 |
| 52 | 335.5 |
| 53 | 435.1 |
| 54 | 439.8 |
| 55 | 547.9 |
| 56 | 654.2 |
| 57 | 668.3 |
| 58 | 678.1 |
| 59 | 875.6 |
| 60 | 1007.2 |
| 61 | 1437.5 |
| 62 | 2390.1 |
| 63 | inactive |
| 64 | inactive |
| 65 | 1.6 |
| 66 | 3.7 |
| 67 | 2.1 |
| 68 | 2.2 |
| 69 | 3.6 |
| 70 | 3.8 |
| 71 | 4.0 |
| 72 | 4.1 |
| 73 | 4.1 |
| 74 | 4.3 |
| 75 | 4.3 |
| 76 | 4.3 |
| 77 | 4.9 |
| 78 | 5.1 |
| 79 | 5.2 |
| 80 | 5.3 |
| 81 | 5.4 |
| 82 | 5.5 |
| 83 | 5.6 |
| 83 | 5.6 |
| 84 | 5.9 |
| 85 | 6.0 |
| 86 | 6.0 |
| 87 | 6.4 |
| 88 | 6.5 |
| 89 | 6.7 |
| 90 | 6.8 |
| 91 | 7.3 |
| 92 | 7.9 |
| 94 | 8.3 |
| 95 | 8.3 |
| 96 | 8.6 |
| 97 | 9.8 |
| 98 | 10.2 |
| 99 | 10.5 |
| 100 | 10.9 |
| 101 | 13.0 |
| 102 | 13.4 |
| 103 | 13.4 |
| 104 | 14.6 |
| 105 | 15.3 |
| 106 | 16.5 |
| 107 | 16.8 |
| 108 | 19.8 |
| 109 | 22.1 |
| 110 | 24.9 |
| 111 | 27.5 |
| 112 | 34.0 |
| 113 | 36.0 |
| 114 | 54.4 |
| 115 | 179.5 |
| 116 | 678.5 |
| 117 | inactive |
| 118 | inactive |
| 119 | inactive |
| 120 | inactive |
| 121 | 16.1 |
| 122 | 41.2 |
| 123 | 11.2 |
| 124 | 608.6 |
| 125 | 7.3 |
| 126 | 100.0 |
| 127 | 672.3 |
| 128 | 26.7 |
| 129 | 18.6 |
| 130 | 12.0 |
| 131 | 7.6 |
| 132 | 38.9 |
| 133 | 13.0 |
| 134 | 14.9 |
| 135 | 184.7 |
| 136 | 50.5 |
| 137 | 7.5 |
| 138 | 19.9 |
| 139 | 18.0 |
| 140 | 5.8 |
| 141 | 2.3 |
| 142 | 60.3 |
| 143 | 634.4 |
| 144 | 466.3 |
| 145 | 479.5 |
| 146 | 538.8 |
| 147 | 38.8 |
| 148 | 53.4 |
| 149 | 90.7 |
| 150 | 138.7 |
| 151 | 204.4 |
| 152 | 15.0 |
| 153 | 438.4 |
| 154 | 18.7 |
| 155 | 19.5 |
| 156 | 12.5 |
| 157 | 7.8 |
| 158 | 5.6 |
| 159 | 62.2 |
| 160 | 788.6 |
| 161 | 60.5 |
| 162 | 25.4 |
| 163 | 4.2 |
| 164 | 4.0 |
| 165 | 123.6 |
| 166 | 191.7 |
| 167 | 14.2 |
| 168 | 1.7 |
| 169 | 354.7 |
| 170 | 21.5 |
| 171 | 14.1 |
| 172 | 4.0 |
| 173 | 21.3 |
| 174 | 607.3 |
| 175 | 235.6 |
| 176 | 619.2 |
| 177 | 29.0 |
| 178 | 299.9 |
| 179 | 113.2 |
| 180 | 27.6 |
| 181 | 6.2 |
| 182 | 173.1 |
| 183 | 18.8 |
| 184 | 49.0 |
| 185 | 158.1 |
| 186 | 77.7 |
| 187 | 401.2 |
| 188 | 105.4 |
| 189 | 461.2 |
| 190 | 166.4 |
| 191 | 621.0 |
| 192 | 34.2 |
| 193 | 4.8 |
| 194 | 19.3 |
| 195 | 9.1 |
| 196 | 491.5 |
| 197 | 11.6 |
| 198 | 12.5 |
| 199 | 525.7 |
| 200 | 30.1 |
| 201 | 11.1 |
| 202 | 3.7 |
| 203 | 42.5 |
| 204 | 162.5 |
| 205 | 903.6 |
| 206 | 33.7 |
| 207 | 10.0 |
| 210 | 975.4 |
| 212 | 3.3 |
| 213 | 7.7 |
| 214 | 36.6 |
| 215 | 113.5 |
| 216 | 218.4 |
| 217 | 67.9 |
| 218 | 73.8 |
| 219 | 13.7 |
| 220 | 88.3 |
| 221 | 4.4 |
| 222 | 9.4 |
| 223 | 7.0 |
| 224 | 232.1 |
| 225 | 13.4 |
| 226 | 130.1 |
| 227 | 37.3 |
| 228 | 73.1 |
| 229 | 18.2 |
| 230 | 11.3 |
| 231 | 21.9 |
| 232 | 147.1 |
| 233 | 17.7 |
| 234 | 8.7 |
| 235 | 8.1 |
| 236 | 9.5 |
| 237 | 103.5 |
| 238 | 55.1 |
| 239 | 12.4 |
| 240 | 171.1 |
| 241 | 5.7 |
| 242 | 106.0 |
| 243 | 218.0 |
| 244 | 381.5 |
| 245 | 11.5 |
| 246 | 28.4 |
| 247 | 201.2 |
| 248 | 74.9 |
| 249 | 318.9 |
| 250 | 140.0 |
| 251 | 18.6 |
| 252 | 16.5 |
| 253 | 375.5 |
| 254 | 555.6 |
| 255 | 340.7 |
| 256 | 12.4 |
| 257 | 19.8 |
| 258 | 10.8 |
| 259 | 337.8 |
| 260 | 417.3 |
| 261 | 992.7 |
| 262 | 94.2 |
| 263 | 214.1 |
| 264 | 125.1 |
| 265 | 291.7 |
| 266 | 19.4 |
| 267 | 145.0 |
| 268 | 124.5 |
| 269 | 53.9 |
| 270 | 29.2 |
| 271 | 943.4 |
| 272 | 356.5 |
| 273 | 645.7 |
| 274 | 291.0 |
| 275 | 755.2 |
| 276 | 3.7 |
| 277 | 24.8 |
| 278 | 8.3 |
| 279 | 5.2 |
| 280 | 4.5 |
| 281 | 198.2 |
| 282 | 131.0 |
| 283 | 7.3 |
| 284 | 91.4 |

TABLE 3
IC₅₀ (nM) Values for Selected Compounds from WO/2019/191229 A1

| WO/2019/191229 A1 | | IC₅₀ (nM) |
|---|---|---|
| 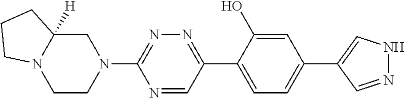 [SMILES: Oc1cc(-c2cn[nH]c2)ccc1-c1cnc(N2CCN3CCC[C@H]3C2)nn1] | Cpd 116 | 91.4 |
| 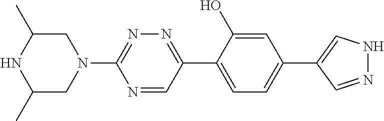 [SMILES: CC1CN(c2ncc(-c3ccc(-c4cn[nH]c4)cc3O)nn2)CC(C)N1] | Cpd 262 | 72.8 |
| 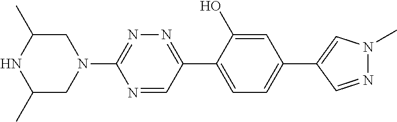 [SMILES: CC1CN(c2ncc(-c3ccc(-c4cnn(C)c4)cc3O)nn2)CC(C)N1] | Cpd 263 | 352.1 |
| 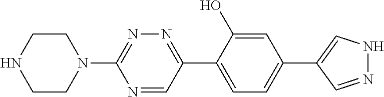 [SMILES: Oc1cc(-c2cn[nH]c2)ccc1-c1cnc(N2CCNCC2)nn1] | Cpd 328 | 106.3 |
| 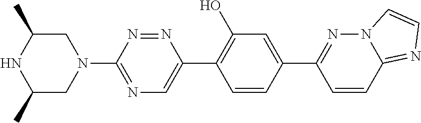 [SMILES: C[C@@H]1CN(c2ncc(-c3ccc(-c4ccc5nccn5n4)cc3O)nn2)C[C@H](C)N1] | Cpd 385 | 33.1 |

In Table 2, where the compounds are reported as “inactive,” the results were above the detection limit of the assay, and the compounds are considered to be inactive.

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or particular aspects described herein. It is intended that the appended claims be interpreted to include all such equivalents.

1. A compound of Formula (I), or a form thereof: 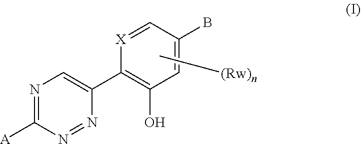
wherein:
A is selected from the group consisting of: 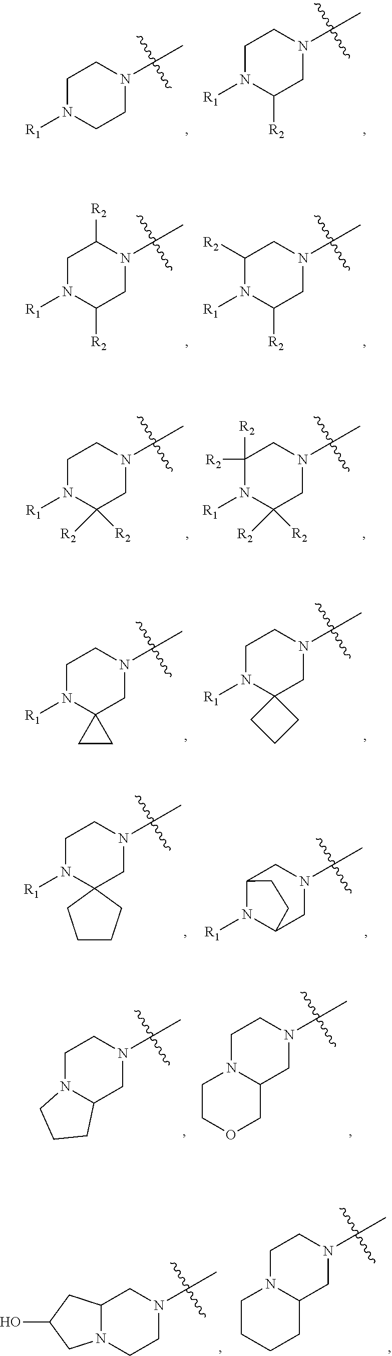
and any stereoisomer thereof;
R₁ is selected from the group consisting of hydrogen, C₁₋₄alkyl, and C₃₋₆cycloalkyl;
R₂ is independently selected from the group consisting of halogen, C₁₋₄alkyl, deutero-C₁₋₄alkyl, halo-C₁₋₄alkyl, hydroxyl-C₁₋₄alkyl, C₁₋₄alkoxy-C₁₋₄alkyl, C₂₋₄alkenyl, C₃₋₆cycloalkyl, phenyl, pyridinyl, and heterocyclyl, wherein heterocyclyl is a 3- to 6-membered carbon atom ring structure radical containing 1 or 2 heteroatom ring members selected from N, O, and S, and
wherein each instance of C₁₋₄alkyl, C₃₋₆cycloalkyl, phenyl, pyridinyl, and heterocyclyl is optionally substituted with one or two R₃ substituents;
R₃ is independently selected from the group consisting of halogen, hydroxyl, C₁₋₄alkyl, C₁₋₄alkoxy, and C₃₋₆cycloalkyl;
B is selected from the group consisting of:
phenyl optionally substituted with one or two independently selected R₄ substituents;
heteroaryl, wherein heteroaryl is a 5- or 6-membered monocyclic aromatic carbon atom ring structure radical containing 1, 2, or 3 heteroatoms selected from N, O, and S, optionally substituted with one R₄ substituent, or wherein heteroaryl is a 9- or 10-membered bicyclic aromatic carbon atom ring structure radical having 1, 2, 3, or 4 heteroatom ring members independently selected from N, O, or S, optionally substituted with one or two independently selected R₄ substituents; and
heterocyclyl, wherein heterocyclyl is a 8- to 10-membered bicyclic aromatic carbon atom ring structure radical containing 1, 2, 3, or 4 heteroatom ring members independently selected from N, O, or S, optionally substituted with one or two independently selected R₄ substituents;
R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, amino, C₁₋₄alkyl-amino, (C₁₋₄alkyl)₂-amino, C₃₋₆cycloalkyl, and heterocyclyl, wherein heterocyclyl is a 3- to 6-membered monocyclic carbon atom ring structure radical containing 1 or 2 heteroatom ring members independently selected from N, O, or S;
X is selected from the group consisting of CH, CF, and N;
R_{w} is selected from the group consisting of halogen, hydroxyl, cyano, C₁₋₄alkyl, deutero-C₁₋₄alkyl, halo-C₁₋₄alkyl, amino, C₁₋₄alkyl-amino, (C₁₋₄alkyl)₂-amino, C₁₋₄alkoxy, and halo-C₁₋₄alkoxy; and
n is selected from the group consisting of 0 or 1;
wherein a form of the compound is selected from the group consisting of a salt, hydrate, solvate, and tautomer form thereof.

2. The compound of claim 1, wherein B is a 5- or 6-membered monocyclic aromatic carbon atom ring structure radical containing 1, 2, or 3 heteroatoms selected from N, O, and S.

3. The compound of claim 1, wherein B is a 9- or 10-membered bicyclic aromatic ring system having 1, 2, 3, or 4 heteroatom ring members independently selected from N, O, or S.

4. The compound of claim 1, wherein B is heterocyclyl, wherein heterocyclyl is a 8- to 10-membered bicyclic aromatic carbon atom ring structure radical containing 1, 2, 3, or 4 heteroatom ring members independently selected from N, O, or S.

5. The compound of claim 1, wherein B is selected from the group consisting of:
phenyl optionally substituted with one or two independently selected R₄ substituents;
heteroaryl,
wherein heteroaryl is a 5- or 6-membered monocyclic aromatic carbon atom ring structure radical containing at least one N atom, or
wherein heteroaryl is a 9-membered bicyclic carbon atom ring structure radical containing at least 2 N atoms; and
heterocyclyl,
wherein heterocyclyl is a 8- or 9-membered bicyclic carbon atom ring structure radical containing at least one N atom.

6. The compound of claim 1, wherein B is selected from the group consisting of:
phenyl optionally substituted with one R₄ substituent;
heteroaryl,
wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or
wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents; and
heterocyclyl,
wherein heterocyclyl is a 8- or 9-membered bicyclic carbon atom ring structure radical containing 2, 3 or 4 N, and optionally, when the ring structure contains 2 or 3 N, containing a second heteroatom ring member selected from O or S, optionally substituted with one or two independently selected R₄ substituents

7. The compound of claim 1, wherein B is heteroaryl or heterocycl,
wherein heteroaryl is a 5- or 6-membered monocyclic aromatic carbon atom ring structure radical containing 1, 2, or 3 N atoms, optionally substituted with one R₄ substituent, or
wherein heteroaryl is a 9-membered bicyclic carbon atom ring structure radical containing 2, 3 or 4 N atoms, and optionally substituted with one or two independently selected R₄ substituents; and
wherein heterocyclyl is a 8- or 9-membered bicyclic carbon atom ring structure radical containing 2, 3 or 4 N, and optionally substituted with one or two independently selected R₄ substituents.

8. The compound of claim 1, wherein B is heteroaryl,
wherein heteroaryl is a 5- or 6-membered monocyclic aromatic carbon atom ring structure radical containing 1, 2, or 3 N atoms, and optionally substituted with one R₄ substituent, or
wherein heteroaryl is a 9-membered bicyclic carbon atom ring structure radical containing 2, 3 or 4 N atoms, and optionally substituted with one or two independently selected R₄ substituents.

9. The compound of claim 1, wherein A may be selected from the group consisting of:

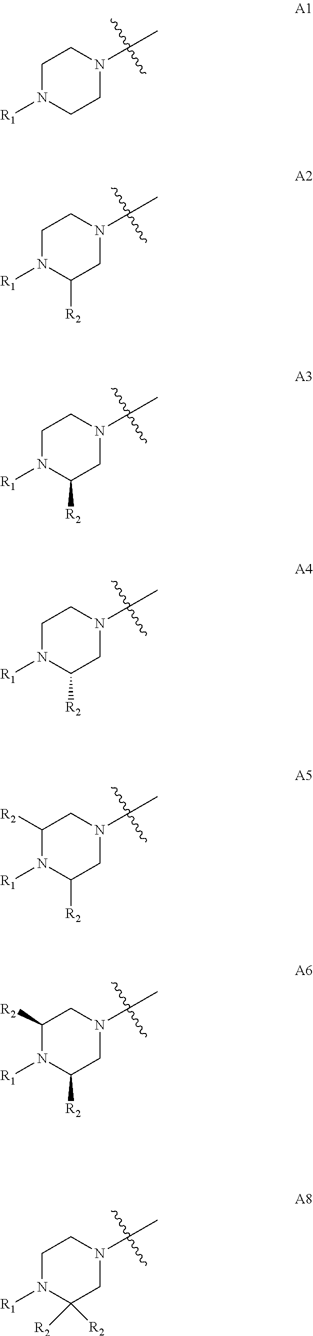

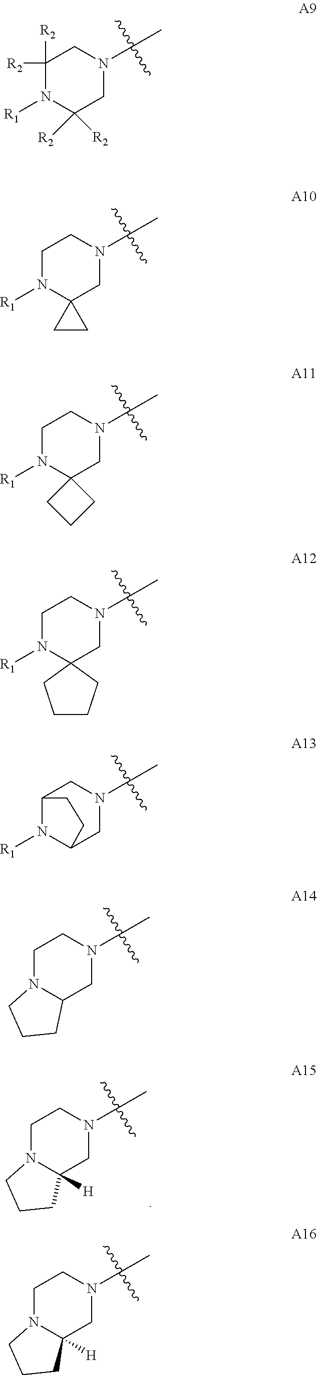

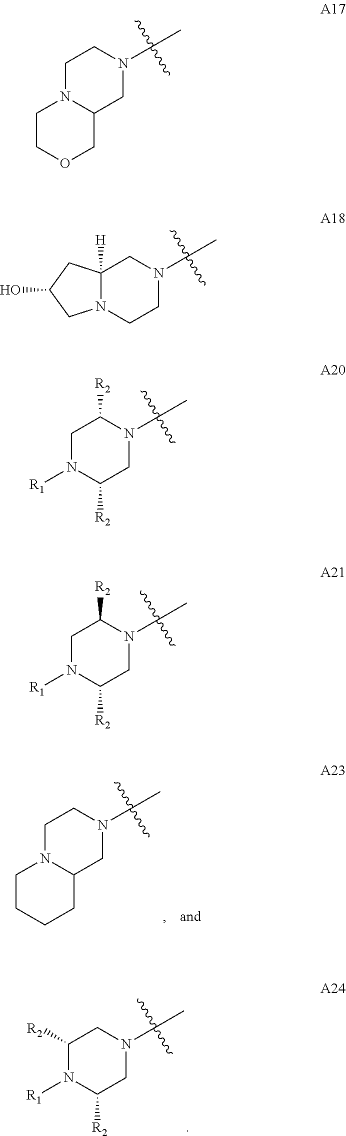

10-14. (canceled)

15. The compound of claim 1, wherein X is CH.

16. The compound of claim 1, wherein X is N.

17-18. (canceled)

19. The compound of claim 1, wherein R₂ is C₁₋₄alkyl, halo-C₁₋₄alkyl, hydroxyl-C₁₋₄alkyl, cyclopropyl, cyclobutyl, phenyl, or oxetanyl, each optionally substituted with one or two R₃ substituents.

20-21. (canceled)

22. The compound of claim 1, wherein R₄ is halogen, cyano, C₁₋₄alkyl, deutero-C₁₋₄alkyl, halo-C₁₋₄alkyl, C₁₋₄alkoxy, deutero-C₁₋₄alkoxy, C₁₋₄alkyl-amino, C₃₋₆cycloalkyl, and heterocyclyl.

23-25. (canceled)

26. The compound of claim 1, wherein:
n is 0;
X is C;
R₂ is C₁₋₄alkyl, halo-C₁₋₄alkyl, hydroxyl-C₁₋₄alkyl, cyclopropyl, cyclobutyl, phenyl, or oxetanyl, each optionally substituted with one or two R₃ substituents;
R₄ is selected from the group consisting of halogen, cyano, C₁₋₄alkyl, deutero-C₁₋₄alkyl, halo-C₁₋₄alkyl, C₁₋₄alkoxy, deutero-C₁₋₄alkoxy, C₁₋₄alkyl-amino, C₃₋₆cycloalkyl, and heterocyclyl;
R₁ is hydrogen or C₁₋₄alkyl;
A is selected from the group consisting of: 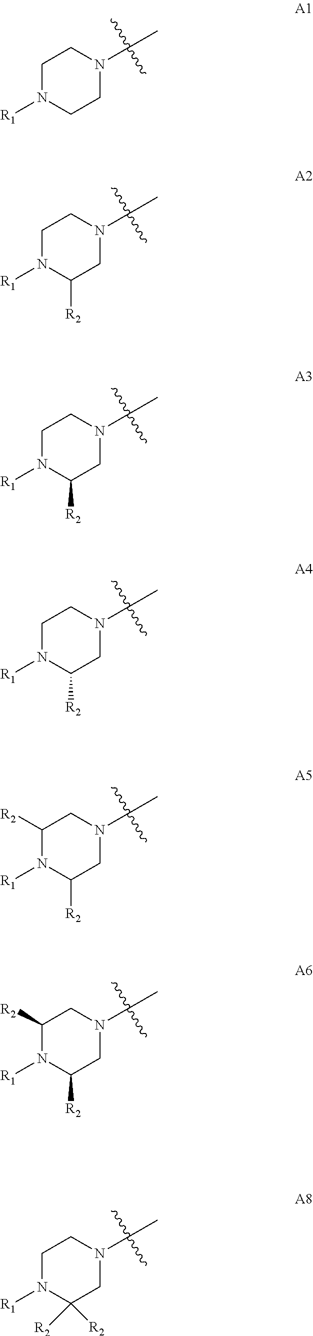 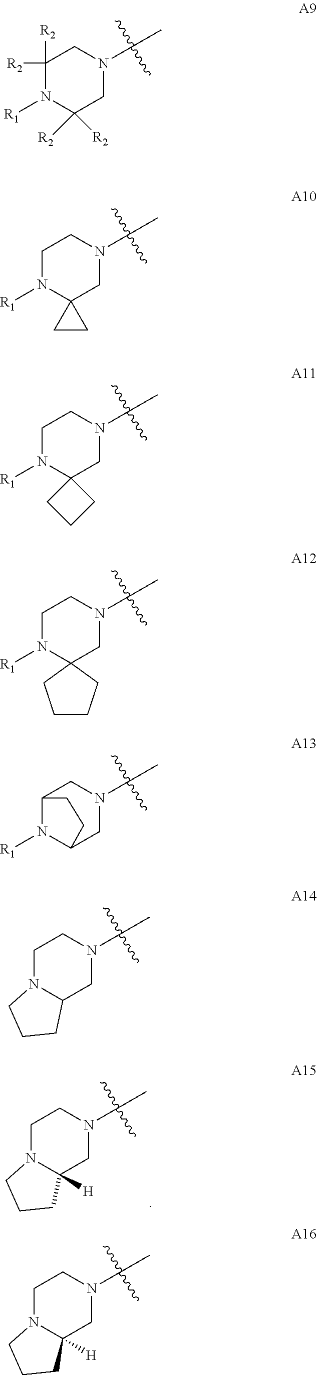 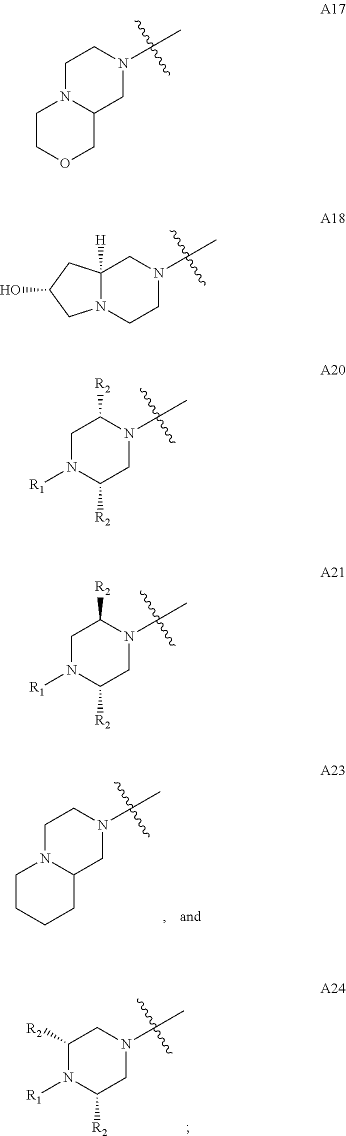
B is selected from the group consisting of:
phenyl unsubstituted or substituted with one R₄ substituent;
heteroaryl,
wherein heteroaryl is a 5- or 6-membered monocyclic aromatic carbon atom ring structure radical containing 1, 2, or 3 N atoms, and optionally, when the ring structure contains 1 or 2 N, a second heteroatom selected from O, and S, optionally substituted with one R₄ substituent, or
wherein heteroaryl is a 9-membered bicyclic carbon atom ring structure radical containing 2, 3 or 4 N atoms, optionally, when the ring structure contains 2 or 3 N, containing a second heteroatom ring member selected from O or S, and optionally substituted with one or two independently selected R₄ substituents; and
heterocyclyl,
wherein heterocyclyl is a 8- or 9-membered bicyclic carbon atom ring structure radical containing 2, 3 or 4 N, and optionally, when the ring structure contains 2 or 3 N, containing a second heteroatom ring member selected from O or S, optionally substituted with one or two independently selected R₄ substituents

27. A compound selected from the group consisting of:
2-{3-[(3S)-3-cyclopropylpiperazin-1-yl]-1,2,4-triazin-6-yl}-5-(3-fluoro-1H-pyrazol-4-yl)phenol;
2-{3-[(3S)-3-cyclopropylpiperazin-1-yl]-1,2,4-triazin-6-yl}-5-{6-[(²H₃)methyloxy]pyrimidin-4-yl}phenol;
5-(3-fluoro-1H-pyrazol-4-yl)-2-{3-[(3S)-3-(propan-2-yl)piperazin-1-yl]-1,2,4-triazin-6-yl}phenol;
5-(3-fluoro-1H-pyrazol-4-yl)-2-{3-[3-(2-hydroxypropan-2-yl)piperazin-1-yl]-1,2,4-triazin-6-yl}phenol;
2-[3-(3-cyclopropylpiperazin-1-yl)-1,2,4-triazin-6-yl]-5-(1H-pyrazol-4-yl)phenol;
2-{3-[3-(1-hydroxycyclopropyl)piperazin-1-yl]-1,2,4-triazin-6-yl}-5-(1H-pyrazol-4-yl)phenol;
2-{3-[(3R)-3-cyclopropylpiperazin-1-yl]-1,2,4-triazin-6-yl}-5-(3-fluoro-1H-pyrazol-4-yl)phenol;
2-{3-[3-(2-hydroxypropan-2-yl)piperazin-1-yl]-1,2,4-triazin-6-yl}-5-{6-[(²H₃)methyloxy]pyrimidin-4-yl}phenol;
2-{3-[(3S)-3-cyclopropylpiperazin-1-yl]-1,2,4-triazin-6-yl}-5-[1-(²H₃)methyl-1H-pyrazol-4-yl]phenol;
5-{6-[(²H₃)methyloxy]pyrimidin-4-yl}-2-{3-[(3S)-3-(propan-2-yl)piperazin-1-yl]-1,2,4-triazin-6-yl}phenol;
2-{3-[(3S)-3-cyclopropylpiperazin-1-yl]-1,2,4-triazin-6-yl}-5-(2H-1,2,3-triazol-2-yl)phenol;
2-{3-[(3S)-3-cyclopropylpiperazin-1-yl]-1,2,4-triazin-6-yl}-5-(1-methyl-1H-pyrazol-4-yl)phenol;
2-{3-[3-(2-hydroxypropan-2-yl)piperazin-1-yl]-1,2,4-triazin-6-yl}-5-(1H-pyrazol-4-yl)phenol;
2-{3-[3-(2-hydroxypropan-2-yl)piperazin-1-yl]-1,2,4-triazin-6-yl}-5-(2H-1,2,3-triazol-2-yl)phenol;
5-(3-fluoro-1H-pyrazol-4-yl)-2-{3-[3-(propan-2-yl)piperazin-1-yl]-1,2,4-triazin-6-yl}phenol;
2-{3-[(3S)-3-tert-butylpiperazin-1-yl]-1,2,4-triazin-6-yl}-5-(1H-pyrazol-4-yl)phenol;
2-{3-[(3S)-3-tert-butylpiperazin-1-yl]-1,2,4-triazin-6-yl}-5-{6-[(²H₃)methyloxy]pyrimidin-4-yl}phenol;
2-{3-[(3S)-3-tert-butylpiperazin-1-yl]-1,2,4-triazin-6-yl}-5-(2H-1,2,3-triazol-2-yl)phenol;
2-{3-[3-(2-hydroxypropan-2-yl)piperazin-1-yl]-1,2,4-triazin-6-yl}-5-[1-(²H₃)methyl-1H-pyrazol-4-yl]phenol;
2-{3-[(3S)-3-(propan-2-yl)piperazin-1-yl]-1,2,4-triazin-6-yl}-5-(1H-pyrazol-4-yl)phenol;
2-{3-[(3S)-3-tert-butylpiperazin-1-yl]-1,2,4-triazin-6-yl}-5-(1-methyl-1H-pyrazol-4-yl)phenol;
2-{3-[3-(propan-2-yl)piperazin-1-yl]-1,2,4-triazin-6-yl}-5-(1H-pyrazol-4-yl)phenol;
2-{3-[(3S)-3-ethylpiperazin-1-yl]-1,2,4-triazin-6-yl}-5-(1H-pyrazol-4-yl)phenol;
2-[3-(3-ethylpiperazin-1-yl)-1,2,4-triazin-6-yl]-5-(1H-pyrazol-4-yl)phenol;
5-(3-fluoro-1H-pyrazol-4-yl)-2-{3-[(3R)-3-(2-hydroxypropan-2-yl)piperazin-1-yl]-1,2,4-triazin-6-yl}phenol;
2-{3-[3-(2-hydroxypropan-2-yl)-4-methylpiperazin-1-yl]-1,2,4-triazin-6-yl}-5-{6-[(²H₃)methyloxy]pyrimidin-4-yl}phenol;
2-[3-(3-cyclopropyl-4-methylpiperazin-1-yl)-1,2,4-triazin-6-yl]-5-(1H-pyrazol-4-yl)phenol;
3-fluoro-5-(6-methoxypyrimidin-4-yl)-2-{3-[(3S)-3-(propan-2-yl)piperazin-1-yl]-1,2,4-triazin-6-yl}phenol;
2-{3-[3-(1-methoxycyclopropyl)piperazin-1-yl]-1,2,4-triazin-6-yl}-5-(1H-pyrazol-4-yl)phenol;
2-[3-(3-cyclobutylpiperazin-1-yl)-1,2,4-triazin-6-yl]-5-(1H-pyrazol-4-yl)phenol;
2-[3-(3-propylpiperazin-1-yl)-1,2,4-triazin-6-yl]-5-(1H-pyrazol-4-yl)phenol;
2-[3-(3-cyclopropylpiperazin-1-yl)-1,2,4-triazin-6-yl]-3-fluoro-5-(5-fluoro-1H-pyrazol-4-yl)phenol;
2-{3-[3-(butan-2-yl)piperazin-1-yl]-1,2,4-triazin-6-yl}-5-(1H-pyrazol-4-yl)phenol;
2-{3-[4-methyl-3-(propan-2-yl)piperazin-1-yl]-1,2,4-triazin-6-yl}-5-(1H-pyrazol-4-yl)phenol;
5-(1-methyl-1H-pyrazol-4-yl)-2-{3-[3-(propan-2-yl)piperazin-1-yl]-1,2,4-triazin-6-yl}phenol;
2-{3-[3-(2,2-difluorocyclopropyl)piperazin-1-yl]-1,2,4-triazin-6-yl}-5-(1H-pyrazol-4-yl)phenol;
2-{3-[(3S)-3-propylpiperazin-1-yl]-1,2,4-triazin-6-yl}-5-(1H-pyrazol-4-yl)phenol;
2-[3-(3-ethenylpiperazin-1-yl)-1,2,4-triazin-6-yl]-5-(1H-pyrazol-4-yl)phenol;
2-[3-(3-ethylpiperazin-1-yl)-1,2,4-triazin-6-yl]-5-[1-(²H₃)methyl-1H-pyrazol-4-yl]phenol;
2-{3-[(3R)-3-(propan-2-yl)piperazin-1-yl]-1,2,4-triazin-6-yl}-5-(1H-pyrazol-4-yl)phenol;
5-[1-(²H₃)methyl-1H-pyrazol-4-yl]-2-{3-[3-(propan-2-yl)piperazin-1-yl]-1,2,4-triazin-6-yl}phenol;
2-[3-(3-methylpiperazin-1-yl)-1,2,4-triazin-6-yl]-5-(1H-pyrazol-4-yl)phenol;
2-[3-(6,9-diazaspiro[4.5]decan-9-yl)-1,2,4-triazin-6-yl]-5-(1H-pyrazol-4-yl)phenol;
5-(2-methylpyridin-4-yl)-2-{3-[3-(propan-2-yl)piperazin-1-yl]-1,2,4-triazin-6-yl}phenol;
2-{3-[(3S)-3-(hydroxymethyl)piperazin-1-yl]-1,2,4-triazin-6-yl}-5-(2H-1,2,3-triazol-2-yl)phenol;
2-{3-[3-(2-methylpropyl)piperazin-1-yl]-1,2,4-triazin-6-yl}-5-(1H-pyrazol-4-yl)phenol;
5-[1-(²H₃)methyl-1H-pyrazol-4-yl]-2-{3-[(3S)-3-(propan-2-yl)piperazin-1-yl]-1,2,4-triazin-6-yl}phenol;
2-{3-[(3R)-3-ethylpiperazin-1-yl]-1,2,4-triazin-6-yl}-5-(1H-pyrazol-4-yl)phenol;
2-{3-[(3R,5S)-3,5-dimethylpiperazin-1-yl]-1,2,4-triazin-6-yl}-5-[1-(²H₃)methyl-1H-pyrazol-4-yl]phenol;
2-[3-(5,8-diazaspiro[3.5]nonan-8-yl)-1,2,4-triazin-6-yl]-5-(1H-pyrazol-4-yl)phenol;
5-[1-(²H₃)methyl-1H-pyrazol-4-yl]-2-{3-[(3R,5S)-3,4,5-trimethylpiperazin-1-yl]-1,2,4-triazin-6-yl}phenol;
5-(2H-1,2,3-triazol-2-yl)-2-{3-[(3R,5S)-3,4,5-trimethylpiperazin-1-yl]-1,2,4-triazin-6-yl}phenol;
2-{3-[(3R)-3-(methoxymethyl)piperazin-1-yl]-1,2,4-triazin-6-yl}-5-(1H-pyrazol-4-yl)phenol;
2-{3-[(3R,5S)-3,5-dimethylpiperazin-1-yl]-1,2,4-triazin-6-yl}-5-(2H-1,2,3-triazol-2-yl)phenol;
2-[3-(4,7-diazaspiro[2.5]octan-7-yl)-1,2,4-triazin-6-yl]-5-(1H-pyrazol-4-yl)phenol;
2-[3-(3,3-dimethylpiperazin-1-yl)-1,2,4-triazin-6-yl]-5-(1H-pyrazol-4-yl)phenol;
2-[3-(4,7-diazaspiro[2.5]octan-7-yl)-1,2,4-triazin-6-yl]-5-(3-fluoro-1H-pyrazol-4-yl)phenol;
2-[3-(8-methyl-3,8-diazabicyclo[3.2.1]octan-3-yl)-1,2,4-triazin-6-yl]-5-[1-(²H₃)methyl-1H-pyrazol-4-yl]phenol;
(7R,8aS)-2-{6-[2-hydroxy-4-(1H-pyrazol-4-yl)phenyl]-1,2,4-triazin-3-yl}octahydropyrrolo[1,2-a]pyrazin-7-ol;
2-{3-[4-(propan-2-yl)piperazin-1-yl]-1,2,4-triazin-6-yl}-5-(1H-pyrazol-4-yl)phenol;
2-[3-(3-phenylpiperazin-1-yl)-1,2,4-triazin-6-yl]-5-(1H-pyrazol-4-yl)phenol;
5-(1H-pyrazol-4-yl)-2-{3-[3-(pyridin-4-yl)piperazin-1-yl]-1,2,4-triazin-6-yl}phenol;
2-[3-(4-cyclopropylpiperazin-1-yl)-1,2,4-triazin-6-yl]-5-(1H-pyrazol-4-yl)phenol;
2-[3-(hexahydropyrazino[2,1-c][1,4]oxazin-8(1H)-yl)-1,2,4-triazin-6-yl]-5-(1H-pyrazol-4-yl)phenol;
5-(7-fluoro-2-methyl-2H-indazol-5-yl)-2-{3-[3-(hydroxymethyl)piperazin-1-yl]-1,2,4-triazin-6-yl}phenol;
2-{3-[(3R,5S)-3,5-dimethylpiperazin-1-yl]-1,2,4-triazin-6-yl}-5-(8-fluoro-2-methylimidazo[1,2-a]pyridin-6-yl)phenol;
5-(7-fluoro-2-methyl-2H-indazol-5-yl)-2-[3-(3-methylpiperazin-1-yl)-1,2,4-triazin-6-yl]phenol;
2-[3-(3-ethylpiperazin-1-yl)-1,2,4-triazin-6-yl]-5-(8-fluoro-2-methylimidazo[1,2-a]pyridin-6-yl)phenol;
5-(2,8-dimethylimidazo[1,2-b]pyridazin-6-yl)-2-{3-[(3R,5S)-3,5-dimethylpiperazin-1-yl]-1,2,4-triazin-6-yl}phenol;
5-(8-methoxy-2-methyl[1,2,4]triazolo[1,5-b]pyridazin-6-yl)-2-{3-[(3S)-3-(propan-2-yl)piperazin-1-yl]-1,2,4-triazin-6-yl}phenol;
5-(7-fluoro-2-methyl-2H-indazol-5-yl)-2-{3-[3-(propan-2-yl)piperazin-1-yl]-1,2,4-triazin-6-yl}phenol;
2-{3-[(3R,5S)-3,5-dimethylpiperazin-1-yl]-1,2,4-triazin-6-yl}-5-(2-methyl-2H-indazol-5-yl)phenol;
5-(8-fluoro-2-methylimidazo[1,2-a]pyridin-6-yl)-2-[3-(4-methylpiperazin-1-yl)-1,2,4-triazin-6-yl]phenol;
2-[3-(3-ethylpiperazin-1-yl)-1,2,4-triazin-6-yl]-5-(7-fluoro-2-methyl-2H-indazol-5-yl)phenol;
2-{3-[(3R,5S)-3,5-dimethylpiperazin-1-yl]-1,2,4-triazin-6-yl}-5-(2-methylimidazo[1,2-b]pyridazin-6-yl)phenol;
2-{3-[(3R,5R)-3,5-dimethylpiperazin-1-yl]-1,2,4-triazin-6-yl}-5-(7-fluoro-2-methyl-2H-indazol-5-yl)phenol;
2-[3-(4-ethylpiperazin-1-yl)-1,2,4-triazin-6-yl]-5-(8-fluoro-2-methylimidazo[1,2-a]pyridin-6-yl)phenol;
5-(2,8-dimethyl[1,2,4]triazolo[1,5-a]pyrazin-6-yl)-2-{3-[(3S)-3-(propan-2-yl)piperazin-1-yl]-1,2,4-triazin-6-yl}phenol;
2-{3-[(3R,5S)-3,5-dimethylpiperazin-1-yl]-1,2,4-triazin-6-yl}-5-(7-fluoro-2-methyl-2H-indazol-5-yl)phenol;
5-(4-{3-[(3R,5S)-3,5-dimethylpiperazin-1-yl]-1,2,4-triazin-6-yl}-3-hydroxyphenyl)-2-methyl-2H-indazole-7-carbonitrile;
5-(7-fluoro-2-methyl-2H-indazol-5-yl)-2-{3-[(3S)-3-(propan-2-yl)piperazin-1-yl]-1,2,4-triazin-6-yl}phenol;
5-(2-methylimidazo[1,2-b]pyridazin-6-yl)-2-[3-(4-methylpiperazin-1-yl)-1,2,4-triazin-6-yl]phenol;
2-{3-[(3R,5S)-3,5-dimethylpiperazin-1-yl]-1,2,4-triazin-6-yl}-5-(2,8-dimethyl[1,2,4]triazolo[1,5-b]pyridazin-6-yl)phenol;
2-{3-[(3S)-3-cyclopropylpiperazin-1-yl]-1,2,4-triazin-6-yl}-5-(6,8-dimethyl-7H-purin-2-yl)phenol;
5-(2-methyl[1,2,4]triazolo[1,5-a]pyridin-6-yl)-2-{3-[(3S)-3-(propan-2-yl)piperazin-1-yl]-1,2,4-triazin-6-yl}phenol;
2-{3-[(3R,5S)-3,5-dimethylpiperazin-1-yl]-1,2,4-triazin-6-yl}-5-(2-methylimidazo[1,2-a]pyrazin-6-yl)phenol;
6-(4-{3-[(3R,5S)-3,5-dimethylpiperazin-1-yl]-1,2,4-triazin-6-yl}-3-hydroxyphenyl)-2-methylimidazo[1,2-a]pyridine-8-carbonitrile;
5-(2-methyl-2H-indazol-5-yl)-2-{3-[(3S)-3-(propan-2-yl)piperazin-1-yl]-1,2,4-triazin-6-yl}phenol;
2-{3-[(3R)-4-ethyl-3-methylpiperazin-1-yl]-1,2,4-triazin-6-yl}-5-(8-fluoro-2-methylimidazo[1,2-a]pyridin-6-yl)phenol;
2-{3-[(3R,5S)-3,5-dimethylpiperazin-1-yl]-1,2,4-triazin-6-yl}-5-(8-methoxy-2-methyl[1,2,4]triazolo[1,5-b]pyridazin-6-yl)phenol;
5-(2-methyl[1,2,4]triazolo[1,5-a]pyrazin-6-yl)-2-{3-[(3S)-3-(propan-2-yl)piperazin-1-yl]-1,2,4-triazin-6-yl}phenol;
5-(2,8-dimethyl[1,2,4]triazolo[1,5-b]pyridazin-6-yl)-2-{3-[(3S)-3-(propan-2-yl)piperazin-1-yl]-1,2,4-triazin-6-yl}phenol;
5-(8-methoxy-2-methyl[1,2,4]triazolo[1,5-a]pyrazin-6-yl)-2-{3-[(3S)-3-(propan-2-yl)piperazin-1-yl]-1,2,4-triazin-6-yl}phenol;
5-(8-fluoro-2-methylimidazo[1,2-a]pyridin-6-yl)-2-{3-[(3R,5S)-3,4,5-trimethylpiperazin-1-yl]-1,2,4-triazin-6-yl}phenol;
5-(7-fluoro-2-methyl-2H-indazol-5-yl)-2-[3-(hexahydropyrrolo[1,2-a]pyrazin-2(1H)-yl)-1,2,4-triazin-6-yl]phenol;
5-(2,8-dimethylimidazo[1,2-a]pyrazin-6-yl)-2-{3-[(3S)-3-(propan-2-yl)piperazin-1-yl]-1,2,4-triazin-6-yl}phenol;
2-{3-[(3R,5S)-3,5-dimethylpiperazin-1-yl]-1,2,4-triazin-6-yl}-5-(2-methyl-2H-pyrazolo[3,4-b]pyridin-5-yl)phenol;
5-(2,8-dimethylimidazo[1,2-b]pyridazin-6-yl)-2-{3-[(3R)-3-methylpiperazin-1-yl]-1,2,4-triazin-6-yl}phenol;
5-(7-fluoro-2-methyl-2H-indazol-5-yl)-2-{3-[(8aS)-hexahydropyrrolo[1,2-a]pyrazin-2(1H)-yl]-1,2,4-triazin-6-yl}phenol;
2-{3-[(3R,5S)-3,5-dimethylpiperazin-1-yl]-1,2,4-triazin-6-yl}-5-(2-methyl[1,2,4]triazolo[1,5-b]pyridazin-6-yl)phenol;
2-{3-[(3R,5S)-3,5-dimethylpiperazin-1-yl]-1,2,4-triazin-6-yl}-5-(2,8-dimethyl[1,2,4]triazolo[1,5-a]pyrazin-6-yl)phenol;
5-(imidazo[1,2-b]pyridazin-6-yl)-2-{3-[(3S)-3-(propan-2-yl)piperazin-1-yl]-1,2,4-triazin-6-yl}phenol;
5-(2,8-dimethylimidazo[1,2-a]pyrazin-6-yl)-2-{3-[(3R,5S)-3,5-dimethylpiperazin-1-yl]-1,2,4-triazin-6-yl}phenol;
5-(2,8-dimethylimidazo[1,2-b]pyridazin-6-yl)-2-[3-(4-methylpiperazin-1-yl)-1,2,4-triazin-6-yl]phenol;
5-(7-fluoro-2-methyl-2H-indazol-5-yl)-2-{3-[(8aR)-hexahydropyrrolo[1,2-a]pyrazin-2(1H)-yl]-1,2,4-triazin-6-yl}phenol;
2-{3-[(3R,5S)-3,5-dimethylpiperazin-1-yl]-1,2,4-triazin-6-yl}-5-(2-methyl[1,2,4]triazolo[1,5-a]pyrazin-6-yl)phenol;
2-{3-[(3R)-3,4-dimethylpiperazin-1-yl]-1,2,4-triazin-6-yl}-5-(8-fluoro-2-methylimidazo[1,2-a]pyridin-6-yl)phenol;
6-(3-hydroxy-4-{3-[(3R,5S)-3,4,5-trimethylpiperazin-1-yl]-1,2,4-triazin-6-yl}phenyl)-2-methylimidazo[1,2-b]pyridazine-8-carbonitrile;
5-(8-cyclopropyl-2-methyl[1,2,4]triazolo[1,5-a]pyrazin-6-yl)-2-{3-[(3R,5S)-3,5-dimethylpiperazin-1-yl]-1,2,4-triazin-6-yl}phenol;
2-[3-(4-methylpiperazin-1-yl)-1,2,4-triazin-6-yl]-5-(2-methyl-2H-pyrazolo[3,4-b]pyridin-5-yl)phenol;
5-(2,8-dimethylimidazo[1,2-b]pyridazin-6-yl)-2-{3-[(3R,5S)-3,4,5-trimethylpiperazin-1-yl]-1,2,4-triazin-6-yl}phenol;
2-{3-[(3R,5S)-3,5-dimethylpiperazin-1-yl]-1,2,4-triazin-6-yl}-5-(2-methyl[1,2,4]triazolo[1,5-a]pyrimidin-6-yl)phenol;
5-(imidazo[1,2-a]pyrazin-6-yl)-2-{3-[(3S)-3-(propan-2-yl)piperazin-1-yl]-1,2,4-triazin-6-yl}phenol;
5-(imidazo[1,2-a]pyridin-6-yl)-2-{3-[(3S)-3-(propan-2-yl)piperazin-1-yl]-1,2,4-triazin-6-yl}phenol;
2-{3-[(3S)-3-(propan-2-yl)piperazin-1-yl]-1,2,4-triazin-6-yl}-5-([1,2,4]triazolo[4,3-a]pyridin-6-yl)phenol;
2-{3-[(3R,5S)-3,5-dimethylpiperazin-1-yl]-1,2,4-triazin-6-yl}-5-(4,6-dimethyl[1,3]thiazolo[5,4-c]pyridin-2-yl)phenol;
2-{3-[(3R,5S)-3,5-dimethylpiperazin-1-yl]-1,2,4-triazin-6-yl}-5-(5,7-dimethyl[1,2,4]triazolo[1,5-a]pyrimidin-2-yl)phenol;
2-{3-[(3R,5S)-3,5-dimethylpiperazin-1-yl]-1,2,4-triazin-6-yl}-5-[2-(trifluoromethyl)imidazo[1,2-b]pyridazin-6-yl]phenol;
2-{3-[(3R,5S)-3,5-dimethylpiperazin-1-yl]-1,2,4-triazin-6-yl}-5-(6-methyl[1,3]thiazolo[4,5-b]pyrazin-2-yl)phenol;
2-{3-[(3R,5S)-3,5-dimethylpiperazin-1-yl]-1,2,4-triazin-6-yl}-5-(5-methylfuro[3,2-b]pyridin-2-yl)phenol;
5-(7-methoxy-2-methyl-2H-indazol-5-yl)-2-[3-(4-methylpiperazin-1-yl)-1,2,4-triazin-6-yl]pyridin-3-ol;
5-(8-fluoro-2-methylimidazo[1,2-a]pyridin-6-yl)-2-[3-(4-methylpiperazin-1-yl)-1,2,4-triazin-6-yl]pyridin-3-ol;
2-{3-[(3S)-3-ethylpiperazin-1-yl]-1,2,4-triazin-6-yl}-5-(3-fluoro-1H-pyrazol-4-yl)phenol;
2-[3-(4-methylpiperazin-1-yl)-1,2,4-triazin-6-yl]-5-(1H-pyrazol-4-yl)pyridin-3-ol;
2-{3-[(3S)-3-tert-butylpiperazin-1-yl]-1,2,4-triazin-6-yl}-5-(3-fluoro-1H-pyrazol-4-yl)phenol;
5-(2,8-dimethylimidazo[1,2-a]pyridin-6-yl)-2-[3-(4-methylpiperazin-1-yl)-1,2,4-triazin-6-yl]pyridin-3-ol;
3-methyl-2-{3-[(3S)-3-(propan-2-yl)piperazin-1-yl]-1,2,4-triazin-6-yl}-5-(1H-pyrazol-4-yl)phenol;
2-{3-[(3S)-3-(propan-2-yl)piperazin-1-yl]-1,2,4-triazin-6-yl}-5-([1,2,4]triazolo[1,5-a]pyrazin-6-yl)phenol;
2-{3-[(3S)-3-tert-butylpiperazin-1-yl]-1,2,4-triazin-6-yl}-5-([1,2,4]triazolo[1,5-a]pyrazin-6-yl)phenol;
2-{3-[(3S)-3-cyclopropylpiperazin-1-yl]-1,2,4-triazin-6-yl}-5-([1,2,4]triazolo[1,5-a]pyrazin-6-yl)phenol;
5-(2,8-dimethylimidazo[1,2-a]pyridin-6-yl)-2-{3-[(3S)-3-(propan-2-yl)piperazin-1-yl]-1,2,4-triazin-6-yl}phenol;
2-{3-[(3S)-3-ethylpiperazin-1-yl]-1,2,4-triazin-6-yl}-5-(2H-1,2,3-triazol-2-yl)phenol;
2-{3-[(3S)-3-ethylpiperazin-1-yl]-1,2,4-triazin-6-yl}-5-{6-[(²H₃)methyloxy]pyrimidin-4-yl}phenol;
2-{3-[3-(1-methylcyclopropyl)piperazin-1-yl]-1,2,4-triazin-6-yl}-5-(1H-pyrazol-4-yl)phenol;
2-[3-(3,8-diazabicyclo[3.2.1]octan-3-yl)-1,2,4-triazin-6-yl]-5-(1H-pyrazol-4-yl)phenol;
2-{3-[(3R)-3-cyclopropylpiperazin-1-yl]-1,2,4-triazin-6-yl}-5-(1H-pyrazol-4-yl)phenol;
2-{3-[(3S)-3-cyclopropylpiperazin-1-yl]-1,2,4-triazin-6-yl}-5-(1H-pyrazol-4-yl)phenol;
5-(8-ethyl-2-methylimidazo[1,2-a]pyridin-6-yl)-2-{3-[(3S)-3-(propan-2-yl)piperazin-1-yl]-1,2,4-triazin-6-yl}phenol;
5-[2-methyl-8-(trifluoromethyl)imidazo[1,2-a]pyridin-6-yl]-2-{3-[(3S)-3-(propan-2-yl)piperazin-1-yl]-1,2,4-triazin-6-yl}phenol;
5-(2,7-dimethyl-2H-indazol-5-yl)-2-{3-[(3S)-3-(propan-2-yl)piperazin-1-yl]-1,2,4-triazin-6-yl}phenol;
5-(2-methylimidazo[1,2-a]pyrazin-6-yl)-2-{3-[(3S)-3-(propan-2-yl)piperazin-1-yl]-1,2,4-triazin-6-yl}phenol;
5-(1H-imidazol-1-yl)-2-{3-[(3S)-3-(propan-2-yl)piperazin-1-yl]-1,2,4-triazin-6-yl}phenol;
5-(6-methylpyrazin-2-yl)-2-{3-[(3S)-3-(propan-2-yl)piperazin-1-yl]-1,2,4-triazin-6-yl}phenol;
2-{3-[(3S)-3-(propan-2-yl)piperazin-1-yl]-1,2,4-triazin-6-yl}-5-(pyrazin-2-yl)phenol;
5-(5-methylpyrazin-2-yl)-2-{3-[(3S)-3-(propan-2-yl)piperazin-1-yl]-1,2,4-triazin-6-yl}phenol;
5-(1H-pyrazol-4-yl)-2-{3-[3-(2,2,2-trifluoroethyl)piperazin-1-yl]-1,2,4-triazin-6-yl}phenol;
5-(2-methyl[1,2,4]triazolo[1,5-a]pyridin-7-yl)-2-{3-[(3S)-3-(propan-2-yl)piperazin-1-yl]-1,2,4-triazin-6-yl}phenol;
2-{3-[rac-(3S,5R)-3-ethyl-5-methylpiperazin-1-yl]-1,2,4-triazin-6-yl}-5-(1H-pyrazol-4-yl)phenol;
5-(6-methylpyrimidin-4-yl)-2-{3-[(3RS)-3-(propan-2-yl)piperazin-1-yl]-1,2,4-triazin-6-yl}phenol;
5-(6-ethylpyrimidin-4-yl)-2-{3-[(3S)-3-(propan-2-yl)piperazin-1-yl]-1,2,4-triazin-6-yl}phenol;
2-{3-[(3S)-3-(propan-2-yl)piperazin-1-yl]-1,2,4-triazin-6-yl}-5-(pyrimidin-2-yl)phenol;
4-fluoro-5-[1-(²H₃)methyl-1H-pyrazol-4-yl]-2-{3-[(3S)-3-(propan-2-yl)piperazin-1-yl]-1,2,4-triazin-6-yl}phenol;
2-{3-[(8aS)-hexahydropyrrolo[1,2-a]pyrazin-2(1H)-yl]-1,2,4-triazin-6-yl}-5-(2H-1,2,3-triazol-2-yl)phenol;
2-{3-[(3S)-3-(propan-2-yl)piperazin-1-yl]-1,2,4-triazin-6-yl}-5-(2H-1,2,3-triazol-2-yl)phenol;
5-(5-methyl-1H-pyrazolo[4,3-b]pyridin-1-yl)-2-{3-[(3S)-3-(propan-2-yl)piperazin-1-yl]-1,2,4-triazin-6-yl}phenol;
2-{3-[(3S)-3-tert-butylpiperazin-1-yl]-1,2,4-triazin-6-yl}-4-fluoro-5-[1-(²H₃)methyl-1H-pyrazol-4-yl]phenol;
5-(7-fluoro-2-methyl-2H-indazol-5-yl)-2-{3-[(3S)-3-(propan-2-yl)piperazin-1-yl]-1,2,4-triazin-6-yl}pyridin-3-ol;
4-fluoro-2-{3-[(3S)-3-(propan-2-yl)piperazin-1-yl]-1,2,4-triazin-6-yl}-5-(1H-pyrazol-4-yl)phenol;
5-(5-methyl-1H-pyrrolo[3,2-b]pyridin-1-yl)-2-{3-[(3S)-3-(propan-2-yl)piperazin-1-yl]-1,2,4-triazin-6-yl}phenol;
5-(6-methylpyridin-3-yl)-2-{3-[(3S)-3-(propan-2-yl)piperazin-1-yl]-1,2,4-triazin-6-yl}phenol;
2-{3-[(3S)-3-(propan-2-yl)piperazin-1-yl]-1,2,4-triazin-6-yl}-5-(pyridin-4-yl)phenol;
2-{3-[(3S)-3-(propan-2-yl)piperazin-1-yl]-1,2,4-triazin-6-yl}-5-(1H-pyrazolo[3,4-d]pyrimidin-1-yl)phenol;
5-(3-chloro-1H-pyrazol-4-yl)-2-{3-[(3S)-3-cyclopropylpiperazin-1-yl]-1,2,4-triazin-6-yl}phenol;
2-{3-[(3S)-3-tert-butylpiperazin-1-yl]-1,2,4-triazin-6-yl}-4-fluoro-5-(1H-pyrazol-4-yl)phenol;
2-{3-[(3S)-3-methylpiperazin-1-yl]-1,2,4-triazin-6-yl}-5-(1H-pyrazol-4-yl)phenol;
2-{3-[(3R)-3-methylpiperazin-1-yl]-1,2,4-triazin-6-yl}-5-(1H-pyrazol-4-yl)phenol;
2-{3-[(3R,5S)-3,5-dimethylpiperazin-1-yl]-1,2,4-triazin-6-yl}-5-(2-methyl[1,2,4]triazolo[1,5-a]pyrazin-6-yl)phenol;
2-{3-[(3S)-3-cyclopropylpiperazin-1-yl]-1,2,4-triazin-6-yl}-5-(2-methyl-2H-[1,2,3]triazolo[4,5-b]pyridin-6-yl)phenol;
2-{3-[(3S)-3-cyclopropylpiperazin-1-yl]-1,2,4-triazin-6-yl}-5-(1-methyl-1H-[1,2,3]triazolo[4,5-b]pyridin-6-yl)phenol;
2-{3-[(3S)-3-cyclopropylpiperazin-1-yl]-1,2,4-triazin-6-yl}-5-(1,2,4-thiadiazol-5-yl)phenol;
2-{3-[(3S)-3-cyclopropylpiperazin-1-yl]-1,2,4-triazin-6-yl}-5-(1-methyl-1H-[1,2,3]triazolo[4,5-b]pyridin-5-yl)phenol;
2-{3-[(3S)-3-cyclopropylpiperazin-1-yl]-1,2,4-triazin-6-yl}-5-(2-methyl-2H-[1,2,3]triazolo[4,5-c]pyridin-6-yl)phenol;
2-{3-[(3S)-3-cyclopropylpiperazin-1-yl]-1,2,4-triazin-6-yl}-5-(3-methyl-3H-[1,2,3]triazolo[4,5-b]pyridin-5-yl)phenol;
2-{3-[(3S)-3-cyclopropylpiperazin-1-yl]-1,2,4-triazin-6-yl}-5-(1-methyl-1H-[1,2,3]triazolo[4,5-c]pyridin-6-yl)phenol;
2-{3-[(3S)-3-(propan-2-yl)piperazin-1-yl]-1,2,4-triazin-6-yl}-5-(2H-1,2,3-triazol-2-yl)pyridin-3-ol;
2-{3-[(2S,5S)-2,5-dimethylpiperazin-1-yl]-1,2,4-triazin-6-yl}-5-(1H-pyrazol-4-yl)phenol;
2-{3-[(3S)-3-cyclopropylpiperazin-1-yl]-1,2,4-triazin-6-yl}-5-(pyridin-4-yl)phenol;
2-{3-[(3S)-3-cyclopropylpiperazin-1-yl]-1,2,4-triazin-6-yl}-5-(3-fluoropyridin-4-yl)phenol;
4-{3-[(3S)-3-cyclopropylpiperazin-1-yl]-1,2,4-triazin-6-yl}-4′-(methylamino)[1,1′-biphenyl]-3-ol;
2-{3-[(3S)-3-cyclopropylpiperazin-1-yl]-1,2,4-triazin-6-yl}-5-(2-methyl-2H-[1,2,3]triazolo[4,5-b]pyridin-6-yl)pyridin-3-ol;
2-{3-[(3S)-3-cyclopropylpiperazin-1-yl]-1,2,4-triazin-6-yl}-5-(7-fluoro-2-methyl-2H-indazol-5-yl)pyridin-3-ol;
2-{3-[(3S)-3-cyclopropylpiperazin-1-yl]-1,2,4-triazin-6-yl}-5-(2H-1,2,3-triazol-2-yl)pyridin-3-ol;
2-{3-[(3R)-3-cyclopropylpiperazin-1-yl]-1,2,4-triazin-6-yl}-5-(4-methyl-2H-1,2,3-triazol-2-yl)phenol;
5-(4-methyl-2H-1,2,3-triazol-2-yl)-2-{3-[(3S)-3-(propan-2-yl)piperazin-1-yl]-1,2,4-triazin-6-yl}phenol;
2-{3-[(3S)-3-(propan-2-yl)piperazin-1-yl]-1,2,4-triazin-6-yl}-5-(1,3-thiazol-2-yl)phenol;
5-[4-(difluoromethyl)-1,3-thiazol-2-yl]-2-{3-[(3S)-3-(propan-2-yl)piperazin-1-yl]-1,2,4-triazin-6-yl}phenol;
2-{3-[4-methyl-3-(oxetan-3-yl)piperazin-1-yl]-1,2,4-triazin-6-yl}-5-(1H-pyrazol-4-yl)phenol;
5-(4-chloro-1,3-thiazol-2-yl)-2-{3-[(3S)-3-(propan-2-yl)piperazin-1-yl]-1,2,4-triazin-6-yl}phenol;
5-(5-chloro-1,3-thiazol-2-yl)-2-{3-[(3S)-3-(propan-2-yl)piperazin-1-yl]-1,2,4-triazin-6-yl}phenol;
2-{3-[(2R,5S)-2,5-dimethylpiperazin-1-yl]-1,2,4-triazin-6-yl}-5-(1H-pyrazol-4-yl)phenol;
2-{3-[(2S,5R)-2,5-dimethylpiperazin-1-yl]-1,2,4-triazin-6-yl}-5-(1H-pyrazol-4-yl)phenol;
2-{3-[(3S)-3-(propan-2-yl)piperazin-1-yl]-1,2,4-triazin-6-yl}-5-([1,3]thiazolo[5,4-b]pyridin-2-yl)phenol;
2-{3-[(3S)-3-cyclopropylpiperazin-1-yl]-1,2,4-triazin-6-yl}-5-(pyrimidin-4-yl)phenol;
2-{3-[(3S)-3-cyclopropylpiperazin-1-yl]-1,2,4-triazin-6-yl}-5-(3-methyl[1,2,3]triazolo[1,5-a]pyridin-6-yl)phenol;
2-{3-[(3S)-3-cyclopropylpiperazin-1-yl]-1,2,4-triazin-6-yl}-5-(1-methylimidazo[1,5-a]pyridin-6-yl)phenol;
2-{3-[(3S)-3-cyclopropylpiperazin-1-yl]-1,2,4-triazin-6-yl}-5-(3-methylimidazo[1,5-a]pyridin-7-yl)phenol;
5-(5-fluoro-1,3-thiazol-2-yl)-2-{3-[(3S)-3-(propan-2-yl)piperazin-1-yl]-1,2,4-triazin-6-yl}phenol;
2-{3-[(3R,5S)-3,5-dimethylpiperazin-1-yl]-1,2,4-triazin-6-yl}-5-(2-methyl-2H-[1,2,3]triazolo[4,5-b]pyridin-6-yl)phenol;
2-{3-[(3R,5S)-3,5-dimethylpiperazin-1-yl]-1,2,4-triazin-6-yl}-5-(2-methyl-2H-[1,2,3]triazolo[4,5-c]pyridin-6-yl)phenol;
5-(5-cyclopropyl-1,3,4-oxadiazol-2-yl)-2-{3-[(3S)-3-(propan-2-yl)piperazin-1-yl]-1,2,4-triazin-6-yl}phenol;
5-(4-methoxy-1,3-thiazol-2-yl)-2-{3-[(3S)-3-(propan-2-yl)piperazin-1-yl]-1,2,4-triazin-6-yl}phenol;
2-{3-[rac-(3R,5S)-3,5-dimethylpiperazin-1-yl]-1,2,4-triazin-6-yl}-5-(2-methyl-2H-[1,2,3]triazolo[4,5-b]pyridin-5-yl)phenol;
2-{3-[(3S)-3-cyclopropylpiperazin-1-yl]-1,2,4-triazin-6-yl}-5-(2-methyl-2H-[1,2,3]triazolo[4,5-b]pyridin-5-yl)phenol;
2-[3-(octahydro-2H-pyrido[1,2-a]pyrazin-2-yl)-1,2,4-triazin-6-yl]-5-(1H-pyrazol-4-yl)phenol;
5-(5-methoxy-1,3,4-thiadiazol-2-yl)-2-{3-[(3S)-3-(propan-2-yl)piperazin-1-yl]-1,2,4-triazin-6-yl}phenol;
5-[5-(difluoromethyl)-1,3,4-thiadiazol-2-yl]-2-{3-[(3S)-3-(propan-2-yl)piperazin-1-yl]-1,2,4-triazin-6-yl}phenol;
2-{3-[rac-(3R,5S)-3,5-dimethylpiperazin-1-yl]-1,2,4-triazin-6-yl}-5-(3-methyl[1,2,3]triazolo[1,5-a]pyridin-6-yl)phenol;
5-(2,6-dimethoxypyrimidin-4-yl)-2-{3-[(3S)-3-(propan-2-yl)piperazin-1-yl]-1,2,4-triazin-6-yl}phenol;
2-{3-[(3S)-3-cyclopropylpiperazin-1-yl]-1,2,4-triazin-6-yl}-5-(1-ethyl-1H-pyrazol-4-yl)phenol;
2-{3-[(3RS)-3-cyclopropylpiperazin-1-yl]-1,2,4-triazin-6-yl}-5-(2-fluoropyridin-4-yl)phenol;
5-[6-(azetidin-1-yl)pyrimidin-4-yl]-2-{3-[(3RS)-3-cyclopropylpiperazin-1-yl]-1,2,4-triazin-6-yl}phenol;
2-{3-[(3S)-3-tert-butylpiperazin-1-yl]-1,2,4-triazin-6-yl}-5-(2-methyl-2H-[1,2,3]triazolo[4,5-b]pyridin-6-yl)phenol;
5-(2-methyl-2H-[1,2,3]triazolo[4,5-b]pyridin-6-yl)-2-{3-[(3S)-3-(propan-2-yl)piperazin-1-yl]-1,2,4-triazin-6-yl}phenol;
2-{3-[(3S)-3-cyclopropylpiperazin-1-yl]-1,2,4-triazin-6-yl}-5-(1,2,4-thiadiazol-3-yl)phenol;
2-{3-[(3S,5R)-3-ethyl-5-methylpiperazin-1-yl]-1,2,4-triazin-6-yl}-5-(2-methyl-2H-[1,2,3]triazolo[4,5-b]pyridin-6-yl)phenol;
2-{3-[(3R,5S)-3-cyclopropyl-5-methylpiperazin-1-yl]-1,2,4-triazin-6-yl}-5-(5-fluoro-1H-pyrazol-4-yl)phenol;
2-{3-[(3S,5R)-3-methyl-5-(propan-2-yl)piperazin-1-yl]-1,2,4-triazin-6-yl}-5-(1H-pyrazol-4-yl)phenol;
5-(5-fluoro-1H-pyrazol-4-yl)-2-{3-[(3S,5R)-3-methyl-5-(propan-2-yl)piperazin-1-yl]-1,2,4-triazin-6-yl}phenol;
2-{3-[(3R,5S)-3-ethyl-5-methylpiperazin-1-yl]-1,2,4-triazin-6-yl}-5-(5-fluoro-1H-pyrazol-4-yl)phenol;
2-{3-[(3S,5R)-3-cyclopropyl-5-methylpiperazin-1-yl]-1,2,4-triazin-6-yl}-5-(2,6-dimethoxypyrimidin-4-yl)phenol;
2-{3-[(3R,5S)-3-cyclopropyl-5-methylpiperazin-1-yl]-1,2,4-triazin-6-yl}-5-(2,6-dimethoxypyrimidin-4-yl)phenol;
2-{3-[(3S,5R)-3-cyclopropyl-5-methylpiperazin-1-yl]-1,2,4-triazin-6-yl}-5-(5-fluoro-1H-pyrazol-4-yl)phenol;
5-(5-fluoro-1H-pyrazol-4-yl)-2-{3-[(3R,5S)-3-methyl-5-(propan-2-yl)piperazin-1-yl]-1,2,4-triazin-6-yl}phenol;
2-{3-[(3S,5R)-3-ethyl-5-methylpiperazin-1-yl]-1,2,4-triazin-6-yl}-5-(5-fluoro-1H-pyrazol-4-yl)phenol;
2-{3-[(3R,5S)-3-cyclopropyl-5-methylpiperazin-1-yl]-1,2,4-triazin-6-yl}-5-(1H-pyrazol-4-yl)phenol;
2-{3-[(3S,5R)-3-cyclopropyl-5-methylpiperazin-1-yl]-1,2,4-triazin-6-yl}-5-(1H-pyrazol-4-yl)phenol;
2-{3-[(3R,5S)-3-cyclopropyl-5-methylpiperazin-1-yl]-1,2,4-triazin-6-yl}-5-(6-methoxypyrimidin-4-yl)phenol;
5-(6-methoxypyrimidin-4-yl)-2-{3-[(3S,5R)-3-methyl-5-(propan-2-yl)piperazin-1-yl]-1,2,4-triazin-6-yl}phenol;
2-{3-[(3R,5S)-3-ethyl-5-methylpiperazin-1-yl]-1,2,4-triazin-6-yl}-5-(6-methoxypyrimidin-4-yl)phenol;
2-{3-[(3R,5S)-3-methyl-5-(propan-2-yl)piperazin-1-yl]-1,2,4-triazin-6-yl}-5-(1H-pyrazol-4-yl)phenol;
5-(6-methoxypyrimidin-4-yl)-2-{3-[(3R,5S)-3-methyl-5-(propan-2-yl)piperazin-1-yl]-1,2,4-triazin-6-yl}phenol;
2-{3-[(3S,5R)-3-cyclobutyl-5-methylpiperazin-1-yl]-1,2,4-triazin-6-yl}-5-(1H-pyrazol-4-yl)phenol;
2-{3-[(3S)-3-cyclopropylpiperazin-1-yl]-1,2,4-triazin-6-yl}-5-(5-methyl-1,3-oxazol-2-yl)phenol;
2-{3-[(3S)-3-cyclopropylpiperazin-1-yl]-1,2,4-triazin-6-yl}-5-(1,3-oxazol-2-yl)phenol;
2-{3-[(3S,5R)-3-ethyl-5-methylpiperazin-1-yl]-1,2,4-triazin-6-yl}-5-(6-methoxypyrimidin-4-yl)phenol;
2-{3-[(3S,5R)-3-cyclopropyl-5-methylpiperazin-1-yl]-1,2,4-triazin-6-yl}-5-(6-methoxypyrimidin-4-yl)phenol;
2-{3-[(3S,5R)-3-cyclobutyl-5-methylpiperazin-1-yl]-1,2,4-triazin-6-yl}-5-(3-fluoro-1H-pyrazol-4-yl)phenol;
2-{3-[(3S,5R)-3-cyclobutyl-5-methylpiperazin-1-yl]-1,2,4-triazin-6-yl}-5-(2H-1,2,3-triazol-2-yl)phenol;
2-{3-[(3S,5R)-3-cyclobutyl-5-methylpiperazin-1-yl]-1,2,4-triazin-6-yl}-5-[1-(²H₃)methyl-1H-pyrazol-4-yl]phenol;
2-{3-[(3S)-3-(propan-2-yl)piperazin-1-yl]-1,2,4-triazin-6-yl}-5-(1,2,4-thiadiazol-3-yl)phenol;
2-{3-[(3S,5R)-3-cyclobutyl-5-methylpiperazin-1-yl]-1,2,4-triazin-6-yl}-5-(1-methyl-1H-pyrazol-3-yl)phenol;
2-{3-[(3S)-3-(1-methylcyclopropyl)piperazin-1-yl]-1,2,4-triazin-6-yl}-5-(2-methyl-2H-[1,2,3]triazolo[4,5-b]pyridin-6-yl)phenol;
2-{3-[(3R)-3-(1-methylcyclopropyl)piperazin-1-yl]-1,2,4-triazin-6-yl}-5-(2-methyl-2H-[1,2,3]triazolo[4,5-b]pyridin-6-yl)phenol;
2-{3-[(3S)-3-(propan-2-yl)piperazin-1-yl]-1,2,4-triazin-6-yl}-5-(pyrazolo[1,5-a]pyrimidin-3-yl)phenol;
2-{3-[(3S)-3-(propan-2-yl)piperazin-1-yl]-1,2,4-triazin-6-yl}-5-(pyrazolo[1,5-a]pyridin-3-yl)phenol;
2-{3-[(3S)-3-cyclopropylpiperazin-1-yl]-1,2,4-triazin-6-yl}-5-(5-methyl-1,2,4-thiadiazol-3-yl)phenol;
2-{3-[(3S)-3-cyclopropylpiperazin-1-yl]-1,2,4-triazin-6-yl}-5-(2-methyl-1,3-thiazol-4-yl)phenol;
2-{3-[(3S,5R)-3-tert-butyl-5-methylpiperazin-1-yl]-1,2,4-triazin-6-yl}-5-(1H-pyrazol-4-yl)phenol;
2-{3-[(3S,5R)-3-tert-butyl-5-methylpiperazin-1-yl]-1,2,4-triazin-6-yl}-5-(3-fluoro-1H-pyrazol-4-yl)phenol;
2-{3-[(3S,5R)-3-ethenyl-5-methylpiperazin-1-yl]-1,2,4-triazin-6-yl}-5-(1H-pyrazol-4-yl)phenol;
2-{3-[(3S)-3-(propan-2-yl)piperazin-1-yl]-1,2,4-triazin-6-yl}-5-(1,2-thiazol-4-yl)phenol;
2-{3-[(3S)-3-cyclopropylpiperazin-1-yl]-1,2,4-triazin-6-yl}-5-(2-methoxypyridin-4-yl)phenol;
2-{3-[(3S)-3-cyclopropylpiperazin-1-yl]-1,2,4-triazin-6-yl}-5-(1,2-thiazol-3-yl)phenol;
5-(4-methyl-1,2-thiazol-5-yl)-2-{3-[(3S)-3-(propan-2-yl)piperazin-1-yl]-1,2,4-triazin-6-yl}phenol;
2-{3-[(3S,5R)-3-tert-butyl-5-methylpiperazin-1-yl]-1,2,4-triazin-6-yl}-5-(1,2,4-thiadiazol-3-yl)phenol;
2-{3-[(3S,5R)-3-tert-butyl-5-methylpiperazin-1-yl]-1,2,4-triazin-6-yl}-5-(6-methoxypyrimidin-4-yl)phenol;
2-{3-[(3S,5R)-3-cyclopropyl-5-methylpiperazin-1-yl]-1,2,4-triazin-6-yl}-5-(2-methoxypyridin-4-yl)phenol;
5-(2-methoxypyridin-4-yl)-2-{3-[(3R,5S)-3-methyl-5-(propan-2-yl)piperazin-1-yl]-1,2,4-triazin-6-yl}phenol;
2-{3-[(3S)-3-tert-butylpiperazin-1-yl]-1,2,4-triazin-6-yl}-5-(1,2,4-thiadiazol-3-yl)phenol;
5-(1-methyl-1H-1,2,4-triazol-3-yl)-2-{3-[(3S)-3-(propan-2-yl)piperazin-1-yl]-1,2,4-triazin-6-yl}phenol;
5-(1-methyl-1H-1,2,3-triazol-4-yl)-2-{3-[(3S)-3-(propan-2-yl)piperazin-1-yl]-1,2,4-triazin-6-yl}phenol;
5-(1-methyl-1H-1,2,3-triazol-5-yl)-2-{3-[(3S)-3-(propan-2-yl)piperazin-1-yl]-1,2,4-triazin-6-yl}phenol;
5-(2-methyl-2H-1,2,3-triazol-4-yl)-2-{3-[(3S)-3-(propan-2-yl)piperazin-1-yl]-1,2,4-triazin-6-yl}phenol;
5-(2,1,3-benzothiadiazol-5-yl)-2-{3-[(3S)-3-(propan-2-yl)piperazin-1-yl]-1,2,4-triazin-6-yl}phenol;
2-{3-[(3S)-3-(propan-2-yl)piperazin-1-yl]-1,2,4-triazin-6-yl}-5-([1,2,5]thiadiazolo[3,4-b]pyridin-6-yl)phenol;
2-{3-[(3S)-3-(propan-2-yl)piperazin-1-yl]-1,2,4-triazin-6-yl}-5-(1,2,5-thiadiazol-3-yl)phenol;
2-{3-[(3S,5R)-3-ethyl-5-methylpiperazin-1-yl]-1,2,4-triazin-6-yl}-5-(2-methoxypyridin-4-yl)phenol;
2-{3-[(3R,5S)-3-ethyl-5-methylpiperazin-1-yl]-1,2,4-triazin-6-yl}-5-(2-methoxypyridin-4-yl)phenol;
2-{3-[(3S,5R)-3-ethyl-5-methylpiperazin-1-yl]-1,2,4-triazin-6-yl}-5-(1,2,4-thiadiazol-5-yl)phenol;
2-{3-[(3R,5S)-3-ethyl-5-methylpiperazin-1-yl]-1,2,4-triazin-6-yl}-5-(1,2,4-thiadiazol-5-yl)phenol;
2-{3-[(3R,5S)-3-methyl-5-(propan-2-yl)piperazin-1-yl]-1,2,4-triazin-6-yl}-5-(1,2,4-thiadiazol-5-yl)phenol;
2-{3-[(3S,5R)-3-methyl-5-(propan-2-yl)piperazin-1-yl]-1,2,4-triazin-6-yl}-5-(1,2,4-thiadiazol-5-yl)phenol;
2-{3-[(3S,5R)-3-cyclopropyl-5-methylpiperazin-1-yl]-1,2,4-triazin-6-yl}-5-(1,2,4-thiadiazol-5-yl)phenol;
2-{3-[(3R,5S)-3-cyclopropyl-5-methylpiperazin-1-yl]-1,2,4-triazin-6-yl}-5-(1,2,4-thiadiazol-5-yl)phenol;
2-{3-[(3S)-3-(propan-2-yl)piperazin-1-yl]-1,2,4-triazin-6-yl}-5-(1,2-thiazol-5-yl)phenol;
5-(2-methoxy-6-methylpyridin-4-yl)-2-{3-[(3S)-3-(propan-2-yl)piperazin-1-yl]-1,2,4-triazin-6-yl}phenol;
2-(3-hydroxy-4-{3-[(3S)-3-(propan-2-yl)piperazin-1-yl]-1,2,4-triazin-6-yl}phenyl)-1,3-thiazole-5-carbonitrile;
2-(3-hydroxy-4-{3-[(3S)-3-(propan-2-yl)piperazin-1-yl]-1,2,4-triazin-6-yl}phenyl)-1,3-thiazole-4-carbonitrile;
5-(2-methyl-5,6-dihydro[1,2,4]triazolo[1,5-a]pyrazin-7(8H)-yl)-2-{3-[(3S)-3-(propan-2-yl)piperazin-1-yl]-1,2,4-triazin-6-yl}phenol;
2-{3-[(3S)-3-cyclopropylpiperazin-1-yl]-1,2,4-triazin-6-yl}-5-(1-methyl-2,3-dihydro-1H-imidazo[1,2-b]pyrazol-7-yl)phenol;
2-{3-[(3S)-3-cyclopropylpiperazin-1-yl]-1,2,4-triazin-6-yl}-5-(5,6-dihydro-4H-pyrrolo[1,2-b]pyrazol-3-yl)phenol;
2-{3-[(3S)-3-cyclopropylpiperazin-1-yl]-1,2,4-triazin-6-yl}-5-(4,5,6,7-tetrahydropyrazolo[1,5-a]pyridin-3-yl)phenol;
2-{3-[(3R)-3-cyclopropylpiperazin-1-yl]-1,2,4-triazin-6-yl}-5-(4,5,6,7-tetrahydropyrazolo[1,5-a]pyridin-3-yl)phenol;
5-(5,6-dihydro-4H-pyrrolo[1,2-b]pyrazol-3-yl)-2-{3-[(3S)-3-(propan-2-yl)piperazin-1-yl]-1,2,4-triazin-6-yl}phenol;
5-(6,7-dihydro-5H-pyrrolo[1,2-a]imidazol-3-yl)-2-{3-[(3S)-3-(propan-2-yl)piperazin-1-yl]-1,2,4-triazin-6-yl}phenol;
5-(5,6-dihydro-4H-pyrrolo[1,2-b]pyrazol-3-yl)-2-{3-[(3S)-3-(1-methylcyclopropyl)piperazin-1-yl]-1,2,4-triazin-6-yl}phenol;
5-(5,6-dihydro-4H-pyrrolo[1,2-b]pyrazol-3-yl)-2-{3-[(3R)-3-(1-methylcyclopropyl)piperazin-1-yl]-1,2,4-triazin-6-yl}phenol;
5-(6,7-dihydro-5H-pyrazolo[5,1-b][1,3]oxazin-3-yl)-2-{3-[(3S)-3-(propan-2-yl)piperazin-1-yl]-1,2,4-triazin-6-yl}phenol;
3-fluoro-5-(5-fluoro-1H-pyrazol-4-yl)-2-{3-[(3S)-3-(propan-2-yl)piperazin-1-yl]-1,2,4-triazin-6-yl}phenol; and
2-[3-(hexahydropyrrolo[1,2-a]pyrazin-2(1H)-yl)-1,2,4-triazin-6-yl]-5-(1H-pyrazol-4-yl)phenol;
wherein the form of the compound is selected from the group consisting of a salt, hydrate, solvate, and tautomer form thereof.

28. The compound of claim 27, wherein the form of the compound is a salt form or a hydrate, solvate, and tautomer thereof selected from the group consisting of:
2-{3-[(3S)-3-cyclopropylpiperazin-1-yl]-1,2,4-triazin-6-yl}-5-(3-fluoro-1H-pyrazol-4-yl)phenol dihydrochloride;
2-{3-[(3S)-3-cyclopropylpiperazin-1-yl]-1,2,4-triazin-6-yl}-5-{6-[(²H₃)methyloxy]pyrimidin-4-yl}phenol dihydrochloride;
5-(3-fluoro-1H-pyrazol-4-yl)-2-{3-[(3S)-3-(propan-2-yl)piperazin-1-yl]-1,2,4-triazin-6-yl}phenol dihydrochloride;
5-(3-fluoro-1H-pyrazol-4-yl)-2-{3-[3-(2-hydroxypropan-2-yl)piperazin-1-yl]-1,2,4-triazin-6-yl}phenol dihydrochloride;
2-[3-(3-cyclopropylpiperazin-1-yl)-1,2,4-triazin-6-yl]-5-(1H-pyrazol-4-yl)phenol dihydrochloride;
2-{3-[3-(1-hydroxycyclopropyl)piperazin-1-yl]-1,2,4-triazin-6-yl}-5-(1H-pyrazol-4-yl)phenol diformate;
2-{3-[(3R)-3-cyclopropylpiperazin-1-yl]-1,2,4-triazin-6-yl}-5-(3-fluoro-1H-pyrazol-4-yl)phenol dihydrochloride;
2-{3-[3-(2-hydroxypropan-2-yl)piperazin-1-yl]-1,2,4-triazin-6-yl}-5-{6-[(²H₃)methyloxy]pyrimidin-4-yl}phenol dihydrochloride;
2-{3-[(3S)-3-cyclopropylpiperazin-1-yl]-1,2,4-triazin-6-yl}-5-[1-(²H₃)methyl-1H-pyrazol-4-yl]phenol dihydrochloride;
5-{6-[(²H₃)methyloxy]pyrimidin-4-yl}-2-{3-[(3S)-3-(propan-2-yl)piperazin-1-yl]-1,2,4-triazin-6-yl}phenol dihydrochloride;
2-{3-[(3S)-3-cyclopropylpiperazin-1-yl]-1,2,4-triazin-6-yl}-5-(2H-1,2,3-triazol-2-yl)phenol dihydrochloride;
2-{3-[(3S)-3-cyclopropylpiperazin-1-yl]-1,2,4-triazin-6-yl}-5-(1-methyl-1H-pyrazol-4-yl)phenol dihydrochloride;
2-{3-[3-(2-hydroxypropan-2-yl)piperazin-1-yl]-1,2,4-triazin-6-yl}-5-(1H-pyrazol-4-yl)phenol dihydrochloride;
2-{3-[3-(2-hydroxypropan-2-yl)piperazin-1-yl]-1,2,4-triazin-6-yl}-5-(2H-1,2,3-triazol-2-yl)phenol dihydrochloride;
5-(3-fluoro-1H-pyrazol-4-yl)-2-{3-[3-(propan-2-yl)piperazin-1-yl]-1,2,4-triazin-6-yl}phenol dihydrochloride;
2-{3-[(3S)-3-tert-butylpiperazin-1-yl]-1,2,4-triazin-6-yl}-5-(1H-pyrazol-4-yl)phenol dihydrochloride;
2-{3-[(3S)-3-tert-butylpiperazin-1-yl]-1,2,4-triazin-6-yl}-5-{6-[(²H₃)methyloxy]pyrimidin-4-yl}phenol dihydrochloride;
2-{3-[(3S)-3-tert-butylpiperazin-1-yl]-1,2,4-triazin-6-yl}-5-(2H-1,2,3-triazol-2-yl)phenol dihydrochloride;
2-{3-[3-(2-hydroxypropan-2-yl)piperazin-1-yl]-1,2,4-triazin-6-yl}-5-[1-(²H₃)methyl-1H-pyrazol-4-yl]phenol dihydrochloride;
2-{3-[(3S)-3-tert-butylpiperazin-1-yl]-1,2,4-triazin-6-yl}-5-(1-methyl-1H-pyrazol-4-yl)phenol dihydrochloride;
2-[3-(3-ethylpiperazin-1-yl)-1,2,4-triazin-6-yl]-5-(1H-pyrazol-4-yl)phenol dihydrochloride;
5-(3-fluoro-1H-pyrazol-4-yl)-2-{3-[(3R)-3-(2-hydroxypropan-2-yl)piperazin-1-yl]-1,2,4-triazin-6-yl}phenol dihydrochloride;
2-{3-[3-(2-hydroxypropan-2-yl)-4-methylpiperazin-1-yl]-1,2,4-triazin-6-yl}-5-{6-[(²H₃)methyloxy]pyrimidin-4-yl}phenol dihydrochloride;
2-[3-(3-cyclopropyl-4-methylpiperazin-1-yl)-1,2,4-triazin-6-yl]-5-(1H-pyrazol-4-yl)phenol dihydrochloride;
3-fluoro-5-(6-methoxypyrimidin-4-yl)-2-{3-[(3S)-3-(propan-2-yl)piperazin-1-yl]-1,2,4-triazin-6-yl}phenol formate;
2-{3-[3-(1-methoxycyclopropyl)piperazin-1-yl]-1,2,4-triazin-6-yl}-5-(1H-pyrazol-4-yl)phenol diformate;
2-[3-(3-propylpiperazin-1-yl)-1,2,4-triazin-6-yl]-5-(1H-pyrazol-4-yl)phenol dihydrochloride;
2-[3-(3-cyclopropylpiperazin-1-yl)-1,2,4-triazin-6-yl]-3-fluoro-5-(5-fluoro-1H-pyrazol-4-yl)phenol formate;
2-{3-[3-(butan-2-yl)piperazin-1-yl]-1,2,4-triazin-6-yl}-5-(1H-pyrazol-4-yl)phenol dihydrochloride;
5-(1-methyl-1H-pyrazol-4-yl)-2-{3-[3-(propan-2-yl)piperazin-1-yl]-1,2,4-triazin-6-yl}phenol dihydrochloride;
2-{3-[3-(2,2-difluorocyclopropyl)piperazin-1-yl]-1,2,4-triazin-6-yl}-5-(1H-pyrazol-4-yl)phenol dihydrochloride;
2-[3-(3-ethenylpiperazin-1-yl)-1,2,4-triazin-6-yl]-5-(1H-pyrazol-4-yl)phenol dihydrochloride;
2-[3-(3-ethylpiperazin-1-yl)-1,2,4-triazin-6-yl]-5-[1-(²H₃)methyl-1H-pyrazol-4-yl]phenol dihydrochloride;
5-[1-(²H₃)methyl-1H-pyrazol-4-yl]-2-{3-[3-(propan-2-yl)piperazin-1-yl]-1,2,4-triazin-6-yl}phenol dihydrochloride;
2-[3-(6,9-diazaspiro[4.5]decan-9-yl)-1,2,4-triazin-6-yl]-5-(1H-pyrazol-4-yl)phenol dihydrochloride;
5-(2-methylpyridin-4-yl)-2-{3-[3-(propan-2-yl)piperazin-1-yl]-1,2,4-triazin-6-yl}phenol dihydrochloride;
2-{3-[(3S)-3-(hydroxymethyl)piperazin-1-yl]-1,2,4-triazin-6-yl}-5-(2H-1,2,3-triazol-2-yl)phenol dihydrochloride;
5-[1-(²H₃)methyl-1H-pyrazol-4-yl]-2-{3-[(3S)-3-(propan-2-yl)piperazin-1-yl]-1,2,4-triazin-6-yl}phenol dihydrochloride;
2-{3-[(3R,5S)-3,5-dimethylpiperazin-1-yl]-1,2,4-triazin-6-yl}-5-[1-(²H₃)methyl-1H-pyrazol-4-yl]phenol dihydrochloride;
2-[3-(5,8-diazaspiro[3.5]nonan-8-yl)-1,2,4-triazin-6-yl]-5-(1H-pyrazol-4-yl)phenol dihydrochloride;
5-[1-(²H₃)methyl-1H-pyrazol-4-yl]-2-{3-[(3R,5S)-3,4,5-trimethylpiperazin-1-yl]-1,2,4-triazin-6-yl}phenol dihydrochloride;
5-(2H-1,2,3-triazol-2-yl)-2-{3-[(3R,5S)-3,4,5-trimethylpiperazin-1-yl]-1,2,4-triazin-6-yl}phenol dihydrochloride;
2-{3-[(3R,5S)-3,5-dimethylpiperazin-1-yl]-1,2,4-triazin-6-yl}-5-(2H-1,2,3-triazol-2-yl)phenol dihydrochloride;
2-[3-(4,7-diazaspiro[2.5]octan-7-yl)-1,2,4-triazin-6-yl]-5-(1H-pyrazol-4-yl)phenol dihydrochloride;
2-[3-(3,3-dimethylpiperazin-1-yl)-1,2,4-triazin-6-yl]-5-(1H-pyrazol-4-yl)phenol dihydrochloride;
2-[3-(4,7-diazaspiro[2.5]octan-7-yl)-1,2,4-triazin-6-yl]-5-(3-fluoro-1H-pyrazol-4-yl)phenol dihydrochloride;
2-[3-(8-methyl-3,8-diazabicyclo[3.2.1]octan-3-yl)-1,2,4-triazin-6-yl]-5-[1-(²H₃)methyl-1H-pyrazol-4-yl]phenol dihydrochloride;
(7R,8aS)-2-{6-[2-hydroxy-4-(1H-pyrazol-4-yl)phenyl]-1,2,4-triazin-3-yl}octahydropyrrolo[1,2-a]pyrazin-7-ol dihydrochloride;
2-[3-(3-phenylpiperazin-1-yl)-1,2,4-triazin-6-yl]-5-(1H-pyrazol-4-yl)phenol dihydrochloride;
5-(1H-pyrazol-4-yl)-2-{3-[3-(pyridin-4-yl)piperazin-1-yl]-1,2,4-triazin-6-yl}phenol dihydrochloride;
5-(7-fluoro-2-methyl-2H-indazol-5-yl)-2-{3-[3-(hydroxymethyl)piperazin-1-yl]-1,2,4-triazin-6-yl}phenol dihydrochloride;
2-{3-[(3R,5S)-3,5-dimethylpiperazin-1-yl]-1,2,4-triazin-6-yl}-5-(8-fluoro-2-methylimidazo[1,2-a]pyridin-6-yl)phenol dihydrochloride;
5-(7-fluoro-2-methyl-2H-indazol-5-yl)-2-[3-(3-methylpiperazin-1-yl)-1,2,4-triazin-6-yl]phenol dihydrochloride;
2-[3-(3-ethylpiperazin-1-yl)-1,2,4-triazin-6-yl]-5-(8-fluoro-2-methylimidazo[1,2-a]pyridin-6-yl)phenol dihydrochloride;
5-(2,8-dimethylimidazo[1,2-b]pyridazin-6-yl)-2-{3-[(3R,5S)-3,5-dimethylpiperazin-1-yl]-1,2,4-triazin-6-yl}phenol dihydrochloride;
5-(8-methoxy-2-methyl[1,2,4]triazolo[1,5-b]pyridazin-6-yl)-2-{3-[(3S)-3-(propan-2-yl)piperazin-1-yl]-1,2,4-triazin-6-yl}phenol dihydrochloride;
5-(7-fluoro-2-methyl-2H-indazol-5-yl)-2-{3-[3-(propan-2-yl)piperazin-1-yl]-1,2,4-triazin-6-yl}phenol dihydrochloride;
5-(8-fluoro-2-methylimidazo[1,2-a]pyridin-6-yl)-2-[3-(4-methylpiperazin-1-yl)-1,2,4-triazin-6-yl]phenol dihydrochloride;
2-[3-(3-ethylpiperazin-1-yl)-1,2,4-triazin-6-yl]-5-(7-fluoro-2-methyl-2H-indazol-5-yl)phenol dihydrochloride;
2-[3-(4-ethylpiperazin-1-yl)-1,2,4-triazin-6-yl]-5-(8-fluoro-2-methylimidazo[1,2-a]pyridin-6-yl)phenol dihydrochloride;
5-(2,8-dimethyl[1,2,4]triazolo[1,5-a]pyrazin-6-yl)-2-{3-[(3S)-3-(propan-2-yl)piperazin-1-yl]-1,2,4-triazin-6-yl}phenol dihydrochloride;
5-(4-{3-[(3R,5S)-3,5-dimethylpiperazin-1-yl]-1,2,4-triazin-6-yl}-3-hydroxyphenyl)-2-methyl-2H-indazole-7-carbonitrile dihydrochloride;
5-(7-fluoro-2-methyl-2H-indazol-5-yl)-2-{3-[(3S)-3-(propan-2-yl)piperazin-1-yl]-1,2,4-triazin-6-yl}phenol dihydrochloride;
2-{3-[(3R,5S)-3,5-dimethylpiperazin-1-yl]-1,2,4-triazin-6-yl}-5-(2,8-dimethyl[1,2,4]triazolo[1,5-b]pyridazin-6-yl)phenol dihydrochloride;
2-{3-[(3S)-3-cyclopropylpiperazin-1-yl]-1,2,4-triazin-6-yl}-5-(6,8-dimethyl-7H-purin-2-yl)phenol diformate;
5-(2-methyl[1,2,4]triazolo[1,5-a]pyridin-6-yl)-2-{3-[(3S)-3-(propan-2-yl)piperazin-1-yl]-1,2,4-triazin-6-yl}phenol dihydrochloride;
2-{3-[(3R,5S)-3,5-dimethylpiperazin-1-yl]-1,2,4-triazin-6-yl}-5-(2-methylimidazo[1,2-a]pyrazin-6-yl)phenol dihydrochloride;
5-(2-methyl-2H-indazol-5-yl)-2-{3-[(3S)-3-(propan-2-yl)piperazin-1-yl]-1,2,4-triazin-6-yl}phenol dihydrochloride;
2-{3-[(3R)-4-ethyl-3-methylpiperazin-1-yl]-1,2,4-triazin-6-yl}-5-(8-fluoro-2-methylimidazo[1,2-a]pyridin-6-yl)phenol dihydrochloride;
2-{3-[(3R,5S)-3,5-dimethylpiperazin-1-yl]-1,2,4-triazin-6-yl}-5-(8-methoxy-2-methyl[1,2,4]triazolo[1,5-b]pyridazin-6-yl)phenol dihydrochloride;
5-(2-methyl[1,2,4]triazolo[1,5-a]pyrazin-6-yl)-2-{3-[(3S)-3-(propan-2-yl)piperazin-1-yl]-1,2,4-triazin-6-yl}phenol dihydrochloride;
5-(2,8-dimethyl[1,2,4]triazolo[1,5-b]pyridazin-6-yl)-2-{3-[(3S)-3-(propan-2-yl)piperazin-1-yl]-1,2,4-triazin-6-yl}phenol dihydrochloride;
5-(8-methoxy-2-methyl[1,2,4]triazolo[1,5-a]pyrazin-6-yl)-2-{3-[(3S)-3-(propan-2-yl)piperazin-1-yl]-1,2,4-triazin-6-yl}phenol dihydrochloride;
5-(8-fluoro-2-methylimidazo[1,2-a]pyridin-6-yl)-2-{3-[(3R,5S)-3,4,5-trimethylpiperazin-1-yl]-1,2,4-triazin-6-yl}phenol dihydrochloride;
5-(7-fluoro-2-methyl-2H-indazol-5-yl)-2-[3-(hexahydropyrrolo[1,2-a]pyrazin-2(1H)-yl)-1,2,4-triazin-6-yl]phenol dihydrochloride;
5-(2,8-dimethylimidazo[1,2-a]pyrazin-6-yl)-2-{3-[(3S)-3-(propan-2-yl)piperazin-1-yl]-1,2,4-triazin-6-yl}phenol dihydrochloride;
5-(2,8-dimethylimidazo[1,2-b]pyridazin-6-yl)-2-{3-[(3R)-3-methylpiperazin-1-yl]-1,2,4-triazin-6-yl}phenol dihydrochloride;
2-{3-[(3R,5S)-3,5-dimethylpiperazin-1-yl]-1,2,4-triazin-6-yl}-5-(2-methyl[1,2,4]triazolo[1,5-b]pyridazin-6-yl)phenol dihydrochloride;
2-{3-[(3R,5S)-3,5-dimethylpiperazin-1-yl]-1,2,4-triazin-6-yl}-5-(2,8-dimethyl[1,2,4]triazolo[1,5-a]pyrazin-6-yl)phenol dihydrochloride;
5-(imidazo[1,2-b]pyridazin-6-yl)-2-{3-[(3S)-3-(propan-2-yl)piperazin-1-yl]-1,2,4-triazin-6-yl}phenol dihydrochloride;
5-(2,8-dimethylimidazo[1,2-a]pyrazin-6-yl)-2-{3-[(3R,5S)-3,5-dimethylpiperazin-1-yl]-1,2,4-triazin-6-yl}phenol dihydrochloride;
5-(2,8-dimethylimidazo[1,2-b]pyridazin-6-yl)-2-[3-(4-methylpiperazin-1-yl)-1,2,4-triazin-6-yl]phenol dihydrochloride;
5-(7-fluoro-2-methyl-2H-indazol-5-yl)-2-{3-[(8aR)-hexahydropyrrolo[1,2-a]pyrazin-2(1H)-yl]-1,2,4-triazin-6-yl}phenol dihydrochloride;
2-{3-[(3R,5S)-3,5-dimethylpiperazin-1-yl]-1,2,4-triazin-6-yl}-5-(2-methyl[1,2,4]triazolo[1,5-a]pyrazin-6-yl)phenol dihydrochloride;
2-{3-[(3R)-3,4-dimethylpiperazin-1-yl]-1,2,4-triazin-6-yl}-5-(8-fluoro-2-methylimidazo[1,2-a]pyridin-6-yl)phenol dihydrochloride;
6-(3-hydroxy-4-{3-[(3R,5S)-3,4,5-trimethylpiperazin-1-yl]-1,2,4-triazin-6-yl}phenyl)-2-methylimidazo[1,2-b]pyridazine-8-carbonitrile dihydrochloride;
5-(8-cyclopropyl-2-methyl[1,2,4]triazolo[1,5-a]pyrazin-6-yl)-2-{3-[(3R,5S)-3,5-dimethylpiperazin-1-yl]-1,2,4-triazin-6-yl}phenol dihydrochloride;
5-(2,8-dimethylimidazo[1,2-b]pyridazin-6-yl)-2-{3-[(3R,5S)-3,4,5-trimethylpiperazin-1-yl]-1,2,4-triazin-6-yl}phenol dihydrochloride;
2-{3-[(3R,5S)-3,5-dimethylpiperazin-1-yl]-1,2,4-triazin-6-yl}-5-(2-methyl[1,2,4]triazolo[1,5-a]pyrimidin-6-yl)phenol dihydrochloride;
5-(imidazo[1,2-a]pyrazin-6-yl)-2-{3-[(3S)-3-(propan-2-yl)piperazin-1-yl]-1,2,4-triazin-6-yl}phenol dihydrochloride;
5-(imidazo[1,2-a]pyridin-6-yl)-2-{3-[(3S)-3-(propan-2-yl)piperazin-1-yl]-1,2,4-triazin-6-yl}phenol dihydrochloride;
2-{3-[(3S)-3-(propan-2-yl)piperazin-1-yl]-1,2,4-triazin-6-yl}-5-([1,2,4]triazolo[4,3-a]pyridin-6-yl)phenol dihydrochloride;
2-{3-[(3R,5S)-3,5-dimethylpiperazin-1-yl]-1,2,4-triazin-6-yl}-5-(4,6-dimethyl[1,3]thiazolo[5,4-c]pyridin-2-yl)phenol dihydrochloride;
2-{3-[(3R,5S)-3,5-dimethylpiperazin-1-yl]-1,2,4-triazin-6-yl}-5-(5,7-dimethyl[1,2,4]triazolo[1,5-a]pyrimidin-2-yl)phenol dihydrochloride;
2-{3-[(3R,5S)-3,5-dimethylpiperazin-1-yl]-1,2,4-triazin-6-yl}-5-[2-(trifluoromethyl)imidazo[1,2-b]pyridazin-6-yl]phenol dihydrochloride;
2-{3-[(3R,5S)-3,5-dimethylpiperazin-1-yl]-1,2,4-triazin-6-yl}-5-(6-methyl[1,3]thiazolo[4,5-b]pyrazin-2-yl)phenol dihydrochloride;
2-{3-[(3R,5S)-3,5-dimethylpiperazin-1-yl]-1,2,4-triazin-6-yl}-5-(5-methylfuro[3,2-b]pyridin-2-yl)phenol dihydrochloride;
5-(7-methoxy-2-methyl-2H-indazol-5-yl)-2-[3-(4-methylpiperazin-1-yl)-1,2,4-triazin-6-yl]pyridin-3-ol hydrochloride;
5-(8-fluoro-2-methylimidazo[1,2-a]pyridin-6-yl)-2-[3-(4-methylpiperazin-1-yl)-1,2,4-triazin-6-yl]pyridin-3-ol hydrochloride;
2-{3-[(3S)-3-ethylpiperazin-1-yl]-1,2,4-triazin-6-yl}-5-(3-fluoro-1H-pyrazol-4-yl)phenol dihydrochloride;
2-[3-(4-methylpiperazin-1-yl)-1,2,4-triazin-6-yl]-5-(1H-pyrazol-4-yl)pyridin-3-ol hydrochloride;
2-{3-[(3S)-3-tert-butylpiperazin-1-yl]-1,2,4-triazin-6-yl}-5-(3-fluoro-1H-pyrazol-4-yl)phenol dihydrochloride;
5-(2,8-dimethylimidazo[1,2-a]pyridin-6-yl)-2-[3-(4-methylpiperazin-1-yl)-1,2,4-triazin-6-yl]pyridin-3-ol hydrochloride;
3-methyl-2-{3-[(3S)-3-(propan-2-yl)piperazin-1-yl]-1,2,4-triazin-6-yl}-5-(1H-pyrazol-4-yl)phenol formate;
2-{3-[(3S)-3-(propan-2-yl)piperazin-1-yl]-1,2,4-triazin-6-yl}-5-([1,2,4]triazolo[1,5-a]pyrazin-6-yl)phenol dihydrochloride;
2-{3-[(3S)-3-tert-butylpiperazin-1-yl]-1,2,4-triazin-6-yl}-5-([1,2,4]triazolo[1,5-a]pyrazin-6-yl)phenol dihydrochloride;
2-{3-[(3S)-3-cyclopropylpiperazin-1-yl]-1,2,4-triazin-6-yl}-5-([1,2,4]triazolo[1,5-a]pyrazin-6-yl)phenol dihydrochloride;
5-(2,8-dimethylimidazo[1,2-a]pyridin-6-yl)-2-{3-[(3S)-3-(propan-2-yl)piperazin-1-yl]-1,2,4-triazin-6-yl}phenol dihydrochloride;
2-{3-[(3S)-3-ethylpiperazin-1-yl]-1,2,4-triazin-6-yl}-5-(2H-1,2,3-triazol-2-yl)phenol dihydrochloride;
2-{3-[(3S)-3-ethylpiperazin-1-yl]-1,2,4-triazin-6-yl}-5-{6-[(²H₃)methyloxy]pyrimidin-4-yl}phenol dihydrochloride;
2-{3-[3-(1-methylcyclopropyl)piperazin-1-yl]-1,2,4-triazin-6-yl}-5-(1H-pyrazol-4-yl)phenol formate;
2-[3-(3,8-diazabicyclo[3.2.1]octan-3-yl)-1,2,4-triazin-6-yl]-5-(1H-pyrazol-4-yl)phenol diformate;
2-{3-[(3R)-3-cyclopropylpiperazin-1-yl]-1,2,4-triazin-6-yl}-5-(1H-pyrazol-4-yl)phenol dihydrochloride;
2-{3-[(3S)-3-cyclopropylpiperazin-1-yl]-1,2,4-triazin-6-yl}-5-(1H-pyrazol-4-yl)phenol dihydrochloride;
5-(8-ethyl-2-methylimidazo[1,2-a]pyridin-6-yl)-2-{3-[(3S)-3-(propan-2-yl)piperazin-1-yl]-1,2,4-triazin-6-yl}phenol dihydrochloride;
5-[2-methyl-8-(trifluoromethyl)imidazo[1,2-a]pyridin-6-yl]-2-{3-[(3S)-3-(propan-2-yl)piperazin-1-yl]-1,2,4-triazin-6-yl}phenol dihydrochloride;
5-(2,7-dimethyl-2H-indazol-5-yl)-2-{3-[(3S)-3-(propan-2-yl)piperazin-1-yl]-1,2,4-triazin-6-yl}phenol dihydrochloride;
5-(2-methylimidazo[1,2-a]pyrazin-6-yl)-2-{3-[(3S)-3-(propan-2-yl)piperazin-1-yl]-1,2,4-triazin-6-yl}phenol dihydrochloride;
5-(1H-pyrazol-4-yl)-2-{3-[3-(2,2,2-trifluoroethyl)piperazin-1-yl]-1,2,4-triazin-6-yl}phenol diformate;
5-(2-methyl[1,2,4]triazolo[1,5-a]pyridin-7-yl)-2-{3-[(3S)-3-(propan-2-yl)piperazin-1-yl]-1,2,4-triazin-6-yl}phenol dihydrochloride;
2-{3-[rac-(3S,5R)-3-ethyl-5-methylpiperazin-1-yl]-1,2,4-triazin-6-yl}-5-(1H-pyrazol-4-yl)phenol dihydrochloride;
5-(6-methylpyrimidin-4-yl)-2-{3-[(3RS)-3-(propan-2-yl)piperazin-1-yl]-1,2,4-triazin-6-yl}phenol dihydrochloride;
4-fluoro-5-[1-(²H₃)methyl-1H-pyrazol-4-yl]-2-{3-[(3S)-3-(propan-2-yl)piperazin-1-yl]-1,2,4-triazin-6-yl}phenol formate;
2-{3-[(8aS)-hexahydropyrrolo[1,2-a]pyrazin-2(1H)-yl]-1,2,4-triazin-6-yl}-5-(2H-1,2,3-triazol-2-yl)phenol dihydrochloride;
2-{3-[(3S)-3-(propan-2-yl)piperazin-1-yl]-1,2,4-triazin-6-yl}-5-(2H-1,2,3-triazol-2-yl)phenol dihydrochloride;
5-(5-methyl-1H-pyrazolo[4,3-b]pyridin-1-yl)-2-{3-[(3S)-3-(propan-2-yl)piperazin-1-yl]-1,2,4-triazin-6-yl}phenol dihydrochloride;
2-{3-[(3S)-3-tert-butylpiperazin-1-yl]-1,2,4-triazin-6-yl}-4-fluoro-5-[1-(²H₃)methyl-1H-pyrazol-4-yl]phenol formate;
5-(7-fluoro-2-methyl-2H-indazol-5-yl)-2-{3-[(3S)-3-(propan-2-yl)piperazin-1-yl]-1,2,4-triazin-6-yl}pyridin-3-ol hydrochloride;
4-fluoro-2-{3-[(3S)-3-(propan-2-yl)piperazin-1-yl]-1,2,4-triazin-6-yl}-5-(1H-pyrazol-4-yl)phenol formate;
5-(5-methyl-1H-pyrrolo[3,2-b]pyridin-1-yl)-2-{3-[(3S)-3-(propan-2-yl)piperazin-1-yl]-1,2,4-triazin-6-yl}phenol formate;
2-{3-[(3S)-3-(propan-2-yl)piperazin-1-yl]-1,2,4-triazin-6-yl}-5-(1H-pyrazolo[3,4-d]pyrimidin-1-yl)phenol dihydrochloride;
5-(3-chloro-1H-pyrazol-4-yl)-2-{3-[(3S)-3-cyclopropylpiperazin-1-yl]-1,2,4-triazin-6-yl}phenol dihydrochloride;
2-{3-[(3S)-3-tert-butylpiperazin-1-yl]-1,2,4-triazin-6-yl}-4-fluoro-5-(1H-pyrazol-4-yl)phenol formate;
2-{3-[(3S)-3-methylpiperazin-1-yl]-1,2,4-triazin-6-yl}-5-(1H-pyrazol-4-yl)phenol dihydrochloride;
2-{3-[(3R)-3-methylpiperazin-1-yl]-1,2,4-triazin-6-yl}-5-(1H-pyrazol-4-yl)phenol dihydrochloride;
2-{3-[(3S)-3-cyclopropylpiperazin-1-yl]-1,2,4-triazin-6-yl}-5-(2-methyl-2H-[1,2,3]triazolo[4,5-b]pyridin-6-yl)phenol dihydrochloride;
2-{3-[(3S)-3-cyclopropylpiperazin-1-yl]-1,2,4-triazin-6-yl}-5-(1-methyl-1H-[1,2,3]triazolo[4,5-b]pyridin-6-yl)phenol dihydrochloride;
2-{3-[(3S)-3-cyclopropylpiperazin-1-yl]-1,2,4-triazin-6-yl}-5-(1-methyl-1H-[1,2,3]triazolo[4,5-b]pyridin-5-yl)phenol dihydrochloride;
2-{3-[(3S)-3-cyclopropylpiperazin-1-yl]-1,2,4-triazin-6-yl}-5-(2-methyl-2H-[1,2,3]triazolo[4,5-c]pyridin-6-yl)phenol dihydrochloride;
2-{3-[(3S)-3-cyclopropylpiperazin-1-yl]-1,2,4-triazin-6-yl}-5-(3-methyl-3H-[1,2,3]triazolo[4,5-b]pyridin-5-yl)phenol dihydrochloride;
2-{3-[(3S)-3-cyclopropylpiperazin-1-yl]-1,2,4-triazin-6-yl}-5-(1-methyl-1H-[1,2,3]triazolo[4,5-c]pyridin-6-yl)phenol dihydrochloride;
2-{3-[(3S)-3-(propan-2-yl)piperazin-1-yl]-1,2,4-triazin-6-yl}-5-(2H-1,2,3-triazol-2-yl)pyridin-3-ol dihydrochloride;
2-{3-[(2S,5S)-2,5-dimethylpiperazin-1-yl]-1,2,4-triazin-6-yl}-5-(1H-pyrazol-4-yl)phenol dihydrochloride;
2-{3-[(3S)-3-cyclopropylpiperazin-1-yl]-1,2,4-triazin-6-yl}-5-(pyridin-4-yl)phenol dihydrochloride;
2-{3-[(3S)-3-cyclopropylpiperazin-1-yl]-1,2,4-triazin-6-yl}-5-(3-fluoropyridin-4-yl)phenol dihydrochloride;
4-{3-[(3S)-3-cyclopropylpiperazin-1-yl]-1,2,4-triazin-6-yl}-4′-(methylamino)[1,1′-biphenyl]-3-ol dihydrochloride;
2-{3-[(3S)-3-cyclopropylpiperazin-1-yl]-1,2,4-triazin-6-yl}-5-(2-methyl-2H-[1,2,3]triazolo[4,5-b]pyridin-6-yl)pyridin-3-ol trifluoroacetate;
2-{3-[(3S)-3-cyclopropylpiperazin-1-yl]-1,2,4-triazin-6-yl}-5-(7-fluoro-2-methyl-2H-indazol-5-yl)pyridin-3-ol hydrochloride;
2-{3-[(3S)-3-cyclopropylpiperazin-1-yl]-1,2,4-triazin-6-yl}-5-(2H-1,2,3-triazol-2-yl)pyridin-3-ol trifluoroacetate;
2-{3-[(3R)-3-cyclopropylpiperazin-1-yl]-1,2,4-triazin-6-yl}-5-(4-methyl-2H-1,2,3-triazol-2-yl)phenol dihydrochloride;
5-(4-methyl-2H-1,2,3-triazol-2-yl)-2-{3-[(3S)-3-(propan-2-yl)piperazin-1-yl]-1,2,4-triazin-6-yl}phenol dihydrochloride;
2-{3-[4-methyl-3-(oxetan-3-yl)piperazin-1-yl]-1,2,4-triazin-6-yl}-5-(1H-pyrazol-4-yl)phenol formate;
2-{3-[(2R,5S)-2,5-dimethylpiperazin-1-yl]-1,2,4-triazin-6-yl}-5-(1H-pyrazol-4-yl)phenol or enantiomer trifluoroacetate;
2-{3-[(3S)-3-(propan-2-yl)piperazin-1-yl]-1,2,4-triazin-6-yl}-5-([1,3]thiazolo[5,4-b]pyridin-2-yl)phenol trifluoroacetate;
2-{3-[(3S)-3-cyclopropylpiperazin-1-yl]-1,2,4-triazin-6-yl}-5-(3-methyl[1,2,3]triazolo[1,5-a]pyridin-6-yl)phenol formate;
2-{3-[(3S)-3-cyclopropylpiperazin-1-yl]-1,2,4-triazin-6-yl}-5-(1-methylimidazo[1,5-a]pyridin-6-yl)phenol formate;
2-{3-[(3S)-3-cyclopropylpiperazin-1-yl]-1,2,4-triazin-6-yl}-5-(3-methylimidazo[1,5-a]pyridin-7-yl)phenol formate;
2-{3-[(3S)-3-cyclopropylpiperazin-1-yl]-1,2,4-triazin-6-yl}-5-(2-methyl-2H-[1,2,3]triazolo[4,5-b]pyridin-5-yl)phenol dihydrochloride;
2-{3-[(3S)-3-cyclopropylpiperazin-1-yl]-1,2,4-triazin-6-yl}-5-(1-ethyl-1H-pyrazol-4-yl)phenol dihydrochloride;
2-{3-[(3S)-3-tert-butylpiperazin-1-yl]-1,2,4-triazin-6-yl}-5-(2-methyl-2H-[1,2,3]triazolo[4,5-b]pyridin-6-yl)phenol dihydrochloride;
5-(2-methyl-2H-[1,2,3]triazolo[4,5-b]pyridin-6-yl)-2-{3-[(3S)-3-(propan-2-yl)piperazin-1-yl]-1,2,4-triazin-6-yl}phenol dihydrochloride;
2-{3-[(3S)-3-cyclopropylpiperazin-1-yl]-1,2,4-triazin-6-yl}-5-(1,2,4-thiadiazol-3-yl)phenol trifluoroacetate;
2-{3-[(3S,5R)-3-cyclobutyl-5-methylpiperazin-1-yl]-1,2,4-triazin-6-yl}-5-(1H-pyrazol-4-yl)phenol dihydrochloride;
2-{3-[(3S)-3-cyclopropylpiperazin-1-yl]-1,2,4-triazin-6-yl}-5-(5-methyl-1,3-oxazol-2-yl)phenol trifluoroacetate;
2-{3-[(3S)-3-cyclopropylpiperazin-1-yl]-1,2,4-triazin-6-yl}-5-(1,3-oxazol-2-yl)phenol trifluoroacetate;
2-{3-[(3S,5R)-3-cyclobutyl-5-methylpiperazin-1-yl]-1,2,4-triazin-6-yl}-5-(3-fluoro-1H-pyrazol-4-yl)phenol dihydrochloride;
2-{3-[(3S,5R)-3-cyclobutyl-5-methylpiperazin-1-yl]-1,2,4-triazin-6-yl}-5-(2H-1,2,3-triazol-2-yl)phenol dihydrochloride;
2-{3-[(3S,5R)-3-cyclobutyl-5-methylpiperazin-1-yl]-1,2,4-triazin-6-yl}-5-[1-(²H₃)methyl-1H-pyrazol-4-yl]phenol hydrochloride;
2-{3-[(3S)-3-(propan-2-yl)piperazin-1-yl]-1,2,4-triazin-6-yl}-5-(1,2,4-thiadiazol-3-yl)phenol trifluoroacetate;
2-{3-[(3S,5R)-3-cyclobutyl-5-methylpiperazin-1-yl]-1,2,4-triazin-6-yl}-5-(1-methyl-1H-pyrazol-3-yl)phenol hydrochloride;
2-{3-[(3S)-3-(1-methylcyclopropyl)piperazin-1-yl]-1,2,4-triazin-6-yl}-5-(2-methyl-2H-[1,2,3]triazolo[4,5-b]pyridin-6-yl)phenol hydrochloride;
2-{3-[(3R)-3-(1-methylcyclopropyl)piperazin-1-yl]-1,2,4-triazin-6-yl}-5-(2-methyl-2H-[1,2,3]triazolo[4,5-b]pyridin-6-yl)phenol hydrochloride;
2-{3-[(3S)-3-(propan-2-yl)piperazin-1-yl]-1,2,4-triazin-6-yl}-5-(pyrazolo[1,5-a]pyrimidin-3-yl)phenol trifluoroacetate;
2-{3-[(3S)-3-cyclopropylpiperazin-1-yl]-1,2,4-triazin-6-yl}-5-(5-methyl-1,2,4-thiadiazol-3-yl)phenol trifluoroacetate;
2-{3-[(3S)-3-cyclopropylpiperazin-1-yl]-1,2,4-triazin-6-yl}-5-(2-methyl-1,3-thiazol-4-yl)phenol trifluoroacetate;
2-{3-[(3S,5R)-3-tert-butyl-5-methylpiperazin-1-yl]-1,2,4-triazin-6-yl}-5-(1H-pyrazol-4-yl)phenol dihydrochloride;
2-{3-[(3S,5R)-3-tert-butyl-5-methylpiperazin-1-yl]-1,2,4-triazin-6-yl}-5-(3-fluoro-1H-pyrazol-4-yl)phenol dihydrochloride;
2-{3-[(3S,5R)-3-ethenyl-5-methylpiperazin-1-yl]-1,2,4-triazin-6-yl}-5-(1H-pyrazol-4-yl)phenol dihydrochloride;
2-{3-[(3S)-3-(propan-2-yl)piperazin-1-yl]-1,2,4-triazin-6-yl}-5-(1,2-thiazol-4-yl)phenol hydrochloride;
2-{3-[(3S)-3-cyclopropylpiperazin-1-yl]-1,2,4-triazin-6-yl}-5-(2-methoxypyridin-4-yl)phenol trifluoroacetate;
2-{3-[(3S)-3-cyclopropylpiperazin-1-yl]-1,2,4-triazin-6-yl}-5-(1,2-thiazol-3-yl)phenol trifluoroacetate;
2-{3-[(3S,5R)-3-tert-butyl-5-methylpiperazin-1-yl]-1,2,4-triazin-6-yl}-5-(1,2,4-thiadiazol-3-yl)phenol hydrochloride;
2-{3-[(3S,5R)-3-tert-butyl-5-methylpiperazin-1-yl]-1,2,4-triazin-6-yl}-5-(6-methoxypyrimidin-4-yl)phenol hydrochloride;
5-(1-methyl-1H-1,2,3-triazol-4-yl)-2-{3-[(3S)-3-(propan-2-yl)piperazin-1-yl]-1,2,4-triazin-6-yl}phenol dihydrochloride;
5-(1-methyl-1H-1,2,3-triazol-5-yl)-2-{3-[(3S)-3-(propan-2-yl)piperazin-1-yl]-1,2,4-triazin-6-yl}phenol dihydrochloride;
5-(2-methyl-2H-1,2,3-triazol-4-yl)-2-{3-[(3S)-3-(propan-2-yl)piperazin-1-yl]-1,2,4-triazin-6-yl}phenol dihydrochloride;
5-(2,1,3-benzothiadiazol-5-yl)-2-{3-[(3S)-3-(propan-2-yl)piperazin-1-yl]-1,2,4-triazin-6-yl}phenol hydrochloride;
2-{3-[(3S)-3-(propan-2-yl)piperazin-1-yl]-1,2,4-triazin-6-yl}-5-([1,2,5]thiadiazolo[3,4-b]pyridin-6-yl)phenol dihydrochloride;
2-{3-[(3S)-3-(propan-2-yl)piperazin-1-yl]-1,2,4-triazin-6-yl}-5-(1,2,5-thiadiazol-3-yl)phenol trifluoroacetate;
2-{3-[(3S)-3-(propan-2-yl)piperazin-1-yl]-1,2,4-triazin-6-yl}-5-(1,2-thiazol-5-yl)phenol dihydrochloride;
5-(2-methoxy-6-methylpyridin-4-yl)-2-{3-[(3S)-3-(propan-2-yl)piperazin-1-yl]-1,2,4-triazin-6-yl}phenol dihydrochloride;
2-(3-hydroxy-4-{3-[(3S)-3-(propan-2-yl)piperazin-1-yl]-1,2,4-triazin-6-yl}phenyl)-1,3-thiazole-5-carbonitrile hydrochloride;
2-(3-hydroxy-4-{3-[(3S)-3-(propan-2-yl)piperazin-1-yl]-1,2,4-triazin-6-yl}phenyl)-1,3-thiazole-4-carbonitrile hydrochloride;
5-(2-methyl-5,6-dihydro[1,2,4]triazolo[1,5-a]pyrazin-7(8H)-yl)-2-{3-[(3S)-3-(propan-2-yl)piperazin-1-yl]-1,2,4-triazin-6-yl}phenol formate;
2-{3-[(3S)-3-cyclopropylpiperazin-1-yl]-1,2,4-triazin-6-yl}-5-(1-methyl-2,3-dihydro-1H-imidazo[1,2-b]pyrazol-7-yl)phenol dihydrochloride;
2-{3-[(3S)-3-cyclopropylpiperazin-1-yl]-1,2,4-triazin-6-yl}-5-(5,6-dihydro-4H-pyrrolo[1,2-b]pyrazol-3-yl)phenol dihydrochloride;
2-{3-[(3S)-3-cyclopropylpiperazin-1-yl]-1,2,4-triazin-6-yl}-5-(4,5,6,7-tetrahydropyrazolo[1,5-a]pyridin-3-yl)phenol dihydrochloride; or
2-{3-[(3R)-3-cyclopropylpiperazin-1-yl]-1,2,4-triazin-6-yl}-5-(4,5,6,7-tetrahydropyrazolo[1,5-a]pyridin-3-yl)phenol or enantiomer dihydrochloride; and
3-fluoro-5-(5-fluoro-1H-pyrazol-4-yl)-2-{3-[(3S)-3-(propan-2-yl)piperazin-1-yl]-1,2,4-triazin-6-yl}phenol formate.

29-75. (canceled)

76. A method for treating or ameliorating HD in a subject in need thereof comprising, administering to the subject an effective amount of the compound of claim 1.

77-79. (canceled)
